(19) United States
(12) Patent Application Publication
(10) Pub. No.: US 2024/0148794 A1
(43) Pub. Date: May 9, 2024

(54) LNP COMPOSITIONS COMPRISING PAYLOADS FOR IN VIVO THERAPY

(71) Applicant: ModernaTX, Inc., Cambridge, MA (US)

(72) Inventors: David Alvarez, Brookline, MA (US); Jorge Rodrigo Mora, Newton, MA (US)

(21) Appl. No.: 18/546,145

(22) PCT Filed: Feb. 11, 2022
(86) PCT No.: PCT/US2022/016182

§ 371 Date: Aug. 11, 2023

Related U.S. Application Data
(60) Provisional application No. 63/193,565, filed on May 26, 2021.
(60) Provisional application No. 63/149,006, filed on Feb. 12, 2021.

(51) Int. Cl. A61K 35/28; A61K 9/127; A61K 9/51; A61K 48/00; A61P 7/00; C12N 5/0789

(52) U.S. Cl. CPC A61K 35/28; A61K 9/1271; A61K 9/5146; A61K 48/0041; A61P 7/00; C12N 5/0647

(57) ABSTRACT

The disclosure features methods of modifying a cell or tissue in vivo with lipid nanoparticle (LNP) compositions comprising a payload. The LNP compositions of the present disclosure can modify a parameter associated with the cell or tissue; or modify a component associated with the cell or tissue. Further disclosed are methods of treating a subject having a disease, a disorder, a mutation, or a single nucleotide polymorphism (SNP), comprising administering to the subject an effective amount of an LNP composition comprising a payload. Also disclosed herein are LNP compositions comprising a payload and methods of making the same.

### CROSS-REFERENCE TO RELATED APPLICATIONS

This application claims priority to U.S. Provisional Applications 63/149,006, filed on Feb. 12, 2021, and 63/193,565, filed on May 26, 2021. The contents of the aforementioned applications are hereby incorporated by reference in their entirety.

### SEQUENCE LISTING

The instant application contains a Sequence Listing which has been submitted electronically in ASCII format and is hereby incorporated by reference in its entirety. Said ASCII copy, created on Feb. 8, 2022, is named M2180-7010WO_SL.txt and is 55,705 bytes in size.

### BACKGROUND

In vivo manipulation, e.g., modification, of cells represents a continuing medical challenge. Modifying a cell in vivo, e.g., for delivery of nucleic acid molecules such as mRNA, or delivery of other payloads, is challenging for many reasons, including the difficulty in specifically targeting cells of interest in vivo to modify them. Thus, there exists a need to develop methods and compositions that can target desired cells and deliver a payload for modifying the cells in vivo. In particular, modification of stem or progenitor cells to thereby modify many cell types in a particular lineage in vivo would be of tremendous benefit.

### SUMMARY

The present disclosure provides, inter alia, methods of modifying a cell, e.g., a stem cell or a progenitor cell, in vivo with lipid nanoparticle (LNP) compositions comprising a payload. Also disclosed herein are methods of modifying a tissue in vivo with lipid nanoparticle (LNP) compositions comprising a payload. In an embodiment, the LNP composition modifies a parameter associated with the cell or tissue and/or or modifies a component associated with the cell or tissue. Further disclosed herein are methods of treating a subject having a disease, a disorder, a mutation, or a single nucleotide polymorphism (SNP), comprising administering to the subject an effective amount of an LNP composition comprising a payload. In an embodiment, the LNP composition results in a modification of a cell (e.g., stem cell or progenitor cell) in the subject, e.g., modification of a component associated with the cell and/or a parameter associated with the cell. In an embodiment, the delivery of the payload to the cell results in a change to a genotype, a phenotype, and/or a function of the cell. Also disclosed herein are LNP compositions comprising a payload for use, e.g., in the in vivo modification of a cell (e.g., stem cell or progenitor cell) or tissue, and methods of making the same. In one embodiment, an LNP of the disclosure does not include an additional targeting moiety, e.g., it transfects (e.g., at least 10%, 20%, 30%, 40%, 50%, 60%, 70%, 80%, 90%, or 95%) of stem or progenitor cells (e.g., HSPCs) without an additional targeting moiety. In one embodiment, the cell is a common myeloid progenitor cell. In another embodiment, the cell is a common lymphoid progenitor cell. In yet another embodiment, the cell is a multipotent stem cell. In yet another embodiment, the cell is a multipotent progenitor cell. In an embodiment, the cell is a hematopoietic stem and progenitor cell (HSPC). Additional aspects and embodiments of the disclosure are described in further detail below.

### In Vivo Methods of Modifying a Cell or Tissue and Related Methods

In an aspect, disclosed herein is a method of modifying a cell (e.g., stem or progenitor cell), e.g., modifying a parameter associated with the cell or a component associated with the cell, comprising contacting the cell with a lipid nanoparticle (LNP) composition comprising a payload, thereby modifying the cell. In an embodiment, contacting the cell with the LNP (e.g., administration of the LNP composition) modifies a parameter associated with the cell, e.g., as described herein. In an embodiment, contacting the cell with the LNP (e.g., administration of the LNP composition) modifies a component associated with the cell, e.g., as described herein.

In another aspect, disclosed herein is a method of modifying a tissue, e.g., modifying a parameter associated with the tissue or a component associated with the tissue, comprising contacting the cell with a lipid nanoparticle (LNP) composition comprising a payload. In an embodiment, contacting the cell with the LNP (e.g., administration of the LNP composition) modifies a parameter associated with the tissue, e.g., as described herein. In an embodiment, contacting the cell with the LNP (e.g., administration of the LNP composition) modifies a component associated with the tissue, e.g., as described herein.

In yet another aspect, provided herein is a method of treating a subject having a disease, a disorder, a mutation, or a single nucleotide polymorphism (SNP), comprising administering to the subject an effective amount of an LNP composition comprising a payload, wherein said LNP composition results in a modification of a cell (e.g., stem or progenitor cell) in the subject, e.g., modification of a component associated with the cell or a parameter associated with the cell, thereby treating the subject. In an embodiment, administration of the LNP composition modifies a parameter associated with the cell, e.g., as described herein. In an embodiment, administration of the LNP composition modifies a component associated with the cell, e.g., as described herein.

In an aspect, the disclosure provides a method of ameliorating a symptom of a subject having a disease, a disorder, a mutation, or a single nucleotide polymorphism (SNP), comprising administering to the subject an effective amount of an LNP composition comprising a payload, wherein said LNP composition results in a modification of a cell (e.g., stem or progenitor cell) in the subject, e.g., modification of a component associated with the cell or a parameter associated with the cell, thereby ameliorating the symptom of the subject. In an embodiment, administration of the LNP composition modifies a parameter associated with the cell, e.g., as described herein. In an embodiment, administration of the LNP composition modifies a component associated with the cell, e.g., as described herein.

In yet another aspect, provided herein is a method of delivering an LNP composition comprising a payload to a cell (e.g., stem cell or progenitor cell), or tissue, e.g., in a subject, comprising contacting the cell, or tissue with the LNP composition.

In an aspect, the disclosure provides a method of contacting a cell (e.g., stem cell or progenitor cell) or tissue, e.g., in a subject, comprising contacting the cell or tissue with an LNP composition comprising a payload. In an embodiment, the LNP does not comprise an additional targeting moiety.

In another aspect, provided herein is an LNP composition comprising a payload which affects a parameter or component of a stem or progenitor cell, e.g., a common myeloid progenitor cell, a common lymphoid progenitor cell, a multipotent progenitor cell, or a multipotent stem cell. In an embodiment, the LNP does not include an additional targeting moiety, e.g., it transfects (e.g., at least 10%, 20%, 30%, 40%, 50%, 60%, 70%, 80%, 90%, or 95%) of stem or progenitor cells (e.g., HSPCs) without an additional targeting moiety. In an embodiment, the progenitor cell is an HSPC, e.g., an HSC or HPC. In a preferred embodiment, embodiment, the payload produces a change in a hemoglobinopathy, a clotting factor disorder, a blood cell disorder, or an immune cell disorder in a subject.

In an embodiment, the payload affects (e.g., modifies) a genotypic parameter, a phenotypic parameter, and/or a functional parameter of an HSPC, e.g., a common myeloid progenitor cell, a common lymphoid progenitor cell, or a multipotent hematopoietic stem or progenitor cell. In an embodiment, the payload modifies the production, structure, and/or activity of a hemoglobin molecule, thereby producing a change in a hemoglobinopathy. In an embodiment, the payload modifies the production, structure, and/or activity of a clotting factor, thereby producing a change in a clotting factor disorder. In an embodiment, the payload modifies the production, structure, and/or activity of a molecule associated with a blood cell disorder, thereby producing a change in the blood cell disorder. In an embodiment, the payload modifies the production, structure, and/or activity of a molecule associated with an immune cell disorder, thereby producing a change in the immune cell disorder.

In one embodiment, the payload comprises a nucleic acid molecule (e.g., an mRNA). In an embodiment, the payload comprises a genetic modulator, an epigenetic modulator, or an RNA modulator. In an embodiment, the genetic modulator comprises a system which modifies a nucleic acid sequence in a DNA molecule, e.g., introducing an insertion, a deletion, a mutation (e.g., a missense mutation, a silent mutation or a nonsense mutation), a duplication, or an inversion, or any combination thereof. In an embodiment, the genetic modulator comprises a DNA base editor, CRISPR/Cas gene editing system, a zinc finger nuclease (ZFN) system, a Transcription activator-like effector nuclease (TALEN) system, a meganuclease system, or a transposase system, or any combination thereof. In an embodiment, the epigenetic modulator comprises a molecule that modifies chromatin architecture, methylates DNA, and/or modifies a histone. In an embodiment, the RNA modulator comprises a molecule that alters the expression and/or activity; stability or compartmentalization of an RNA molecule.

In a preferred embodiment, the LNP composition comprises an amino lipid comprising a compound of Formula (I-I), a phospholipid comprising DSPC, a structural lipid comprising cholesterol, and a PEG lipid comprising a compound of Formula (VI-D).

In another aspect, provided herein is a modified cell, e.g., a modified stem cell or progenitor cell, e.g., a modified HSPC (e.g., a modified HSC or a modified HPC), made according to a method described herein.

In yet another aspect, the disclosure provides a frozen preparation of a modified cell, e.g., a modified stem or progenitor cell, e.g., a modified HSPC (e.g., a modified HSC or a modified HPC), made according to a method described herein.

In an aspect provided herein is a composition comprising the modified cell described herein, or a frozen preparation of a modified cell described herein, for use in treating a subject having a disease or disorder, e.g., a disease or disorder described herein.

In an aspect provided herein is a composition comprising a modified cell described herein, or a frozen preparation of a modified cell described herein, for use in ameliorating a symptom of a subject having a disease or disorder, e.g., a disease or disorder described herein.

In an embodiment, the modified cell is autologous to the subject. In an embodiment, the modified cell is allogeneic to the subject.

In yet another aspect, the disclosure provides a composition or reaction mixture comprising: (a) a population of stem or progenitor cells, e.g., HSPCs (e.g., HSCs, HPCs, or a combination thereof); and (b) an LNP composition comprising a payload which can modify the stem or progenitor cell, e.g., a component associated with the stem or progenitor cell or a parameter associated with the stem or progenitor cell, e.g., as described herein. In an embodiment, the LNP does not include an additional targeting moiety.

In an aspect, provided herein is a pharmaceutical composition comprising a modified cell, e.g., modified HSPC (e.g., modified HSC or modified HPC), and an LNP comprising a payload which can modify the cell, e.g., a component associated with the cell or a parameter associated with the cell, e.g., as described herein. In an embodiment, the LNP does not include an additional targeting moiety.

In another aspect, provided herein is a kit comprising a modified cell, e.g., modified HSPC (e.g., modified HSC or modified HPC), and an LNP comprising a payload which can modify the cell, e.g., a component associated with the cell or a parameter associated with the cell, e.g., as described herein.

In an embodiment of any of the methods, compositions, or cells disclosed herein, administration or delivery of the LNP composition results in a modification of the cell, or tissue, e.g., a component associated with the cell or tissue, or a parameter associated with the cell or tissue. In an embodiment, administration or delivery of the LNP composition modifies a parameter associated with the cell, e.g., as described herein. In an embodiment, administration or delivery of the LNP composition modifies a component associated with the cell, e.g., as described herein. In an embodiment, the LNP composition comprises a payload that modifies a genotype, a phenotype, and/or a function of the cell, e.g., by modifying a parameter or component associated with the cell, e.g., as described herein. In an embodiment, the LNP composition does not include an additional targeting moiety, e.g., it transfects (e.g., at least 10%, 20%, 30%, 40%, 50%, 60%, 70%, 80%, 90%, or 95%) of stem or progenitor cells (e.g., HSPCs) without an additional targeting moiety.

In an embodiment, the component associated with the cell or tissue comprises: (1) a nucleic acid associated with the cell or fragment thereof, e.g., DNA (e.g., exonic, intronic, intergenic, telomeric, promoter, enhancer, insulator, repressor, coding, or non-coding) or RNA (e.g., mRNA, rRNA, tRNA, regulatory RNA, non-coding RNA, long non-coding RNA (lncRNA), guide RNA (gRNA), small interfering RNA (siRNA), short hairpin RNA (shRNA), piwi-interacting RNA (piRNA), small nucleolar RNA (snoRNA), small nuclear RNA (snRNA), extracellular RNA (exRNA), small Cajal body-specific RNA (scaRNA), or microRNA (miRNA)); (2) a peptide or protein associated with the cell or fragment thereof; (3) a lipid component associated with the cell or fragment thereof, or a combination thereof. In an embodiment, the component comprises: (1) a nucleic acid associated with the cell or fragment thereof, e.g., DNA (e.g., exonic, intronic, intergenic, telomeric, promoter, enhancer, insulator, repressor, coding, or non-coding) or RNA (e.g., mRNA, rRNA, tRNA, regulatory RNA, non-coding RNA, long non-coding RNA (lncRNA), guide RNA (gRNA), small interfering RNA (siRNA), short hairpin RNA (shRNA), piwi-interacting RNA (piRNA), small nucleolar RNA (snoRNA), small nuclear RNA (snRNA), extracellular RNA (exRNA), small Cajal body-specific RNA (scaRNA), or microRNA (miRNA)). In an embodiment, the component comprises DNA. In an embodiment, the component comprises RNA. In an embodiment, the component comprises (2) a peptide or protein associated with the cell or fragment thereof. In an embodiment, the component comprises (3) a lipid component associated with the cell or fragment thereof.

In an embodiment of any of the methods, compositions, or cells disclosed herein, the component is endogenous to the cell.

In an embodiment of any of the methods, compositions, or cells disclosed herein, the component is exogenous to the cell, e.g., has been introduced into the cell by a method known in the art, e.g., electroporation, transformation, vector-based delivery, viral delivery or lipid-based delivery.

In an embodiment of any of the methods, compositions, or cells disclosed herein, the parameter associated with the cell or tissue comprises a genotypic parameter, a phenotypic parameter, a functional parameter, an expression parameter, a signaling parameter, or a combination thereof. In an embodiment, the parameter associated with the cell or tissue comprises a genotypic parameter, e.g., a genotype of the cell. In an embodiment, the parameter associated with the cell or tissue comprises a phenotypic parameter, e.g., a phenotype of the cell. In another embodiment, the parameter associated with the cell or tissue comprises a functional parameter, e.g., a function of the cell (e.g., the ability to produce a protein or to divide). In an embodiment, the parameter associated with the cell or tissue comprises an expression parameter. In an embodiment, the parameter associated with the cell or tissue comprises a signaling parameter.

In an embodiment of any of the methods, compositions, or cells disclosed herein, the genotypic parameter comprises a genotype of the cell. In an embodiment, the genotype comprises the presence or absence a gene or allele, or a modification of a gene or allele, e.g., a germline or somatic mutation, or a polymorphism, in the gene or allele. In an embodiment, the genotype is associated with a phenotype of the cell, e.g., a phenotype descried herein. In an embodiment, the genotype is associated with a function of the cell, e.g., a function descried herein.

In an embodiment of any of the methods, compositions, or cells disclosed herein, the phenotypic parameter comprises a phenotype of the cell. In an embodiment, the phenotype comprises expression and/or activity of a molecule, e.g., cell surface protein, lipid or adhesion molecule, on the surface of the cell. In an embodiment, the phenotype is associated with a genotype of the cell, e.g., a genotype descried herein. In an embodiment, the phenotype is associated with a function of the cell, e.g., a function descried herein.

In an embodiment of any of the methods, compositions, or cells disclosed herein, the functional parameter comprises a function of the cell. In an embodiment, the function comprises the ability of the cell to produce a protein or an RNA. In an embodiment, the function comprises the ability of the cell to proliferate, divide, and/or renew. In an embodiment, the function comprises the ability of the cell to differentiate, e.g., into one or more cell types in a lineage. In an embodiment, the function is associated with a genotype of the cell, e.g., a genotype descried herein. In an embodiment, the function is associated with a phenotype of the cell, e.g., a phenotype descried herein.

In an embodiment of any of the methods, compositions, or cells disclosed herein, the expression parameter comprises one, two, three, four or all of the following: (a) expression level (e.g., of polypeptide or protein, or polynucleotide or nucleic acid, e.g., mRNA); (b) activity (e.g., of polypeptide or protein, or polynucleotide or nucleic acid, e.g., mRNA), (c) post-translational modification of polypeptide or protein; (d) folding (e.g., of polypeptide or protein, or polynucleotide or nucleic acid, e.g., mRNA), and/or (e) stability (e.g., of polypeptide or protein, or polynucleotide or nucleic acid, e.g., mRNA). In an embodiment, the expression parameter comprises(a) expression level (e.g., of polypeptide or protein, or polynucleotide or nucleic acid, e.g., mRNA). In an embodiment, the expression parameter comprises, (b) activity (e.g., of polypeptide or protein, or polynucleotide or nucleic acid, e.g., mRNA). In an embodiment, the expression parameter comprises, (c) post-translational modification of polypeptide or protein. In an embodiment, the expression parameter comprises, (d) folding (e.g., of polypeptide or protein, or polynucleotide or nucleic acid, e.g., mRNA). In an embodiment, the expression parameter comprises, (e) stability (e.g., of polypeptide or protein, or polynucleotide or nucleic acid, e.g., mRNA).

In an embodiment of any of the methods, compositions, or cells disclosed herein, the signaling parameter comprises one, two, three, four or all of the following: (1) modulation of a signaling pathway, e.g., a cellular signaling pathway; (2) cell fate modulation; (3) modulation of expression level (e.g., of polypeptide or protein, or polynucleotide or nucleic acid, e.g., mRNA); (4) modulation of activity (e.g., of polypeptide or protein, or polynucleotide or nucleic acid, e.g., mRNA), and/or (5) modulation of stability e.g., of polypeptide or protein, or polynucleotide or nucleic acid, e.g., mRNA). In an embodiment, the signaling parameter comprises (1) modulation of a signaling pathway, e.g., a cellular signaling pathway. In an embodiment, the signaling parameter comprises (2) cell fate modulation. In an embodiment, the signaling parameter comprises (3) modulation of expression level (e.g., of polypeptide or protein, or polynucleotide or nucleic acid, e.g., mRNA). In an embodiment, the signaling parameter comprises (4) modulation of activity (e.g., of polypeptide or protein, or polynucleotide or nucleic acid, e.g., mRNA). In an embodiment, the signaling parameter comprises (5) modulation of stability e.g., of polypeptide or protein, or polynucleotide or nucleic acid, e.g., mRNA).

In an embodiment of any of the methods, compositions, or cells disclosed herein, the cell is contacted in vitro, in vivo or ex vivo with the LNP composition. In an embodiment, the cell is contacted in vitro with the LNP formulation. In an embodiment, the cell is contacted ex vivo with the LNP formulation. In an embodiment, the cell is contacted in vivo with the LNP formulation.

In an embodiment of any of the methods, compositions, or cells disclosed herein, the cell or tissue modified with an LNP composition disclosed herein, e.g., modified cell, e.g., modified stem or progenitor cell, e.g., modified HSPC (e.g., modified HSC or modified HPC), has a characteristic disclosed herein. In an embodiment, the LNP does not include an additional targeting moiety, e.g., it transfects (e.g., at least 10%, 20%, 30%, 40%, 50%, 60%, 70%, 80%, 90%, or 95%) of stem or progenitor cells (e.g., HSPCs) without an additional targeting moiety.

In an embodiment of any of the methods, compositions, or cells disclosed herein, the modified cell, e.g., modified stem or progenitor cell, e.g., modified HSPC, has one, two, three, four, five or all of the following functional characteristics: (i) ability to self-renew; (ii) unlimited proliferative potential; (iii) ability to enter and/or exit a quiescent state, e.g., a cell state where no proliferation occurs, e.g., GO phase of the cell cycle; (iv) ability to differentiate into any hematopoietic lineage, e.g., myeloid and/or lymphoid lineages, e.g., common lymphoid progenitor (CLP) or a differentiated cell thereof, and/or common myeloid progenitor (CMP) or a differentiated cell thereof; (v) ability to repopulate any hematopoietic lineage, e.g., myeloid and/or lymphoid lineages, e.g., common lymphoid progenitor (CLP) or a differentiated cell thereof, and/or common myeloid progenitor (CMP) or a differentiated cell thereof, e.g., in an organism; and/or (vi) ability to form colony forming units (CFU). In an embodiment, the modified cell, e.g., modified stem or progenitor cell, e.g., modified HSPC, has (i) the ability to self-renew. In an embodiment, the modified cell, e.g., modified stem or progenitor cell, e.g., modified HSPC, has (ii) unlimited proliferative potential. In an embodiment, the modified cell, e.g., modified stem or progenitor cell, e.g., modified HSPC, has (iii) the ability to enter and/or exit a quiescent state, e.g., a cell state where no proliferation occurs, e.g., GO phase of the cell cycle. In an embodiment, the modified cell, e.g., modified stem or progenitor cell, e.g., modified HSPC, has (iv) the ability to differentiate into any hematopoietic lineage, e.g., myeloid and/or lymphoid lineages, e.g., common lymphoid progenitor (CLP) or a differentiated cell thereof, and/or common myeloid progenitor (CMP) or a differentiated cell thereof. In an embodiment, the modified cell, e.g., modified stem or progenitor cell, e.g., modified HSPC, has (v) ability to repopulate any hematopoietic lineage, e.g., myeloid and/or lymphoid lineages, e.g., common lymphoid progenitor (CLP) or a differentiated cell thereof, and/or common myeloid progenitor (CMP) or a differentiated cell thereof; e.g., in an organism. In an embodiment, the modified cell, e.g., modified stem or progenitor cell, e.g., modified HSPC, has (vi) the ability to form colony forming units (CFU).

In an embodiment, the modified HSPC has the ability to form CFU, e.g., as measured in an ex-vivo colony-forming unit (CFU) assay, e.g., as described in Example 2. In an embodiment, the CFU ability is compared to an otherwise similar HSC which has not been contacted with an LNP, or has been contacted with a different LNP.

In an embodiment, the modified HSPC has the ability to differentiate into myeloid cells, e.g., as measured in an ex-vivo colony-forming unit (CFU) assay, e.g., as described in Example 2, or as measured in a lineage tracing experiment, e.g., as described in Example 3 (e.g., FIG. 3D). In an embodiment the ability of the modified HSPC to differentiate into myeloid cells is compared to an otherwise similar HSC which has not been contacted with an LNP, or has been contacted with a different LNP.

In an embodiment, the modified HSPC has the ability to differentiate into lymphoid cells, e.g., as measured in a lineage tracing experiment, e.g., as described in Example 3 (e.g., FIG. 3C). In an embodiment, the ability of the modified HSPC to differentiate into lymphoid cells is compared to an otherwise similar HSPC which has not been contacted with an LNP, or has been contacted with a different LNP.

In an embodiment, the modified HSPC has the ability to differentiate into an erythrocyte cell or a platelet, e.g., as described in Example 3 (e.g., FIGS. 3A-3B). In an embodiment, the ability of the modified HSPC to differentiate into an erythrocyte cell or a platelet is compared to an otherwise similar HSC which has not been contacted with an LNP, or has been contacted with a different LNP. In an embodiment, the modified HSPC differentiates into an erythrocyte cell or a platelet in vivo. In an embodiment, the modified HSPC differentiates into an erythrocyte cell or a platelet in vitro.

In an embodiment, the modified HSPC persists, e.g., in vivo, for at least 1, 2, 3, 4, 5, 6, 7, 10, 15, 20, 25, 30, 45, 60, 90, 120, 180, 240, 300, or 365 days or more. In an embodiment, the in vivo persistence of the modified HSPC results in differentiation into one or more cells, e.g., cells in the myeloid and/or cells in the lymphoid lineage, e.g., as shown in Example 3.

In an embodiment of any of the methods, compositions, or cells disclosed herein, the modified cell (e.g., modified stem or progenitor cell, e.g., modified HSPC) is a human cell, and has one, two, three, four, five, six, seven, eight, or all of the following expression characteristics: (i) expression of CD45, e.g., detectable expression of CD45, e.g., cell surface expression of CD45; (ii) expression of CD34, e.g., detectable expression of CD34, e.g., cell surface expression of CD34; (iii) expression of CD38, e.g., detectable expression of CD38, e.g., cell surface expression of CD38; (iv) expression of CD90 e.g., detectable expression of CD90, e.g., cell surface expression of CD90; (v) expression of CD133 e.g., detectable expression of CD133, e.g., cell surface expression of CD133; (vi) expression of CD45RA, e.g., detectable expression of CD45RA, e.g., cell surface expression of CD45RA; (vii) no detectable or low expression of markers associated with primitive progenitor cells, e.g., common myeloid progenitor (CMP), megakaryocyte erythroid progenitor (MEP), granulocyte-macrophage progenitor (GMP) and/or common lymphoid progenitor (CLP); (viii) no detectable or low expression of markers associated with lineage committed cells, e.g., TCP, NKP, GP, MP, EP and/or MkP; or (ix) no detectable or low expression of markers associated with one, two or all cell lineage markers of (vii)-(viii), e.g., lineage negative (Lin-). In an embodiment, the modified cell is a modified human HSPC and has (i) expression of CD45, e.g., detectable expression of CD45, e.g., cell surface expression of CD45. In an embodiment, the modified cell is a modified human HSPC and has (ii) expression of CD34, e.g., detectable expression of CD34, e.g., cell surface expression of CD34. In an embodiment, the modified cell is a modified human HSPC and has (iii) expression of CD38, e.g., detectable expression of CD38, e.g., cell surface expression of CD38. In an embodiment, the modified cell is a modified human HSPC and has (iv) expression of CD90 e.g., detectable expression of CD90, e.g., cell surface expression of CD90. In an embodiment, the modified cell is a modified human HSPC and has (v) expression of CD133 e.g., detectable expression of CD133, e.g., cell surface expression of CD133. In an embodiment, the modified cell is a modified human HSPC and has (vi) expression of CD45RA, e.g., detectable expression of CD45RA, e.g., cell surface expression of CD45RA. In an embodiment, the modified cell is a modified human HSPC and has (vii) no detectable or low expression of markers associated with primitive progenitor cells, e.g., CMP, MEP, GMP and/or CLP. In an embodiment, the modified cell is a modified human HSPC and has (viii) no detectable or low expression of markers associated with lineage committed cells, e.g., TCP, NKP, GP, MP, EP and/or MkP. In an embodiment, the modified cell is a modified human HSPC and has (ix) no detectable or low expression of markers associated with one, two or all cell lineage markers of (vii)-(viii), e.g., lineage negative (Lin-).

In an embodiment, the modified human HSPC expresses any one of (i)-(vi). In an embodiment, the modified human HSPC expresses any two of (i)-(vi). In an embodiment, the modified human HSPC expresses any three of (i)-(vi). In an embodiment, the modified human HSPC expresses all of (i)-(vi).

In an embodiment, the modified human HSPC has no detectable or low expression of (vii) or (viii). In an embodiment, the modified human HSPC has no detectable or low expression of both (vii) and (viii), e.g., wherein the human HSPC is a lineage negative HSPC.

In an embodiment of any of the methods, compositions, or cells disclosed herein, the modified cell (e.g., modified stem or progenitor cell, e.g., modified HSPC) is a non-human primate (NHP) cell and has one, two, three, four, five, six, seven, eight, or all of the following expression characteristics: (i) expression of CD45, e.g., detectable expression of CD45, e.g., cell surface expression of CD45; (ii) expression of CD34, e.g., detectable expression of CD34, e.g., cell surface expression of CD34; (iii) expression of c-Kit (CD117), e.g., detectable expression of c-Kit (CD117), e.g., cell surface expression of c-Kit (CD117); (iv) expression of CD90 e.g., detectable expression of CD90, e.g., cell surface expression of CD90; (v) expression of CD123 e.g., detectable expression of CD123, e.g., cell surface expression of CD123; (vi) expression of CD45RA, e.g., detectable expression of CD45RA, e.g., cell surface expression of CD45RA; (vii) no detectable or low expression of markers associated with primitive progenitor cells, e.g., CMP, MEP, GMP and/or CLP; (viii) no detectable or low expression of markers associated with lineage committed cells, e.g., TCP, NKP, GP, MP, EP and/or MkP; or (ix) no detectable or low expression of markers associated with one, two or all cell lineage markers of (vii)-(viii), e.g., lineage negative (Lin-).

In an embodiment, the modified cell is a modified NHP HSPC and has (i) expression of CD45, e.g., detectable expression of CD45, e.g., cell surface expression of CD45. In an embodiment, the modified cell is a modified NHP HSPC and has (ii) expression of CD34, e.g., detectable expression of CD34, e.g., cell surface expression of CD34. In an embodiment, the modified cell is a modified NHP HSPC and has (iii) expression of c-Kit (CD117), e.g., detectable expression of c-Kit (CD117), e.g., cell surface expression of c-Kit (CD117). In an embodiment, the modified cell is a modified NHP HSPC and has (iv) expression of CD90 e.g., detectable expression of CD90, e.g., cell surface expression of CD90. In an embodiment, the modified cell is a modified NHP HSPC and has (v) expression of CD123 e.g., detectable expression of CD123, e.g., cell surface expression of CD123. In an embodiment, the modified cell is a modified NHP HSPC and has (vi) expression of CD45RA, e.g., detectable expression of CD45RA, e.g., cell surface expression of CD45RA. In an embodiment, the modified cell is a modified NHP HSPC and has (vii) no detectable or low expression of markers associated with primitive progenitor cells, e.g., CMP, MEP, GMP and/or CLP. In an embodiment, the modified cell is a modified NHP HSPC and has (viii) no detectable or low expression of markers associated with lineage committed cells, e.g., TCP, NKP, GP, MP, EP and/or MkP. In an embodiment, the modified cell is a modified NHP HSPC and has (ix) no detectable or low expression of markers associated with one, two or all cell lineage markers of (vii)-(viii), e.g., lineage negative (Lin-).

In an embodiment, the modified NHP HSPC expresses any one of (i)-(vi). In an embodiment, the modified NHP HSPC expresses any two of (i)-(vi). In an embodiment, the modified NHP HSPC expresses any three of (i)-(vi). In an embodiment, the modified NHP HSPC expresses all of (i)-(vi).

In an embodiment, the modified NHP HSPC has no detectable or low expression of (vii) or (viii). In an embodiment, the modified NHP HSPC has no detectable or low expression of both (vii) and (viii), e.g., wherein the NHP HSPC is a lineage negative HSPC.

In an embodiment of any of the methods, compositions, or cells disclosed herein, the modified cell, (e.g., modified stem or progenitor cell, e.g., modified HSPC) is a modified mouse cell and has one, two, three, four, five, six, seven or all of the following expression characteristics: (i) expression of CD34, e.g., detectable expression of CD34, e.g., cell surface expression of CD34; (ii) expression of CD150, e.g., detectable expression of CD150, e.g., cell surface expression of CD150; (iii) expression of Sca-1 e.g., detectable expression of Sca-1, e.g., cell surface expression of Sca-1; (iv) expression of c-kit e.g., detectable expression of c-KIT, e.g., cell surface expression of c-kit; (v) no detectable or low expression of markers associated with primitive progenitor cells, e.g., CMP and/or CLP; (vi) no detectable or low expression of markers associated with committed precursor cells, e.g., MEP, GM, TNK and/or BCP; (vii) no detectable or low expression of markers associated with lineage committed cells, e.g., TCP, NKP, GP, MP, EP and/or MkP; or (viii) no detectable or low expression of markers associated with one, two or all cell lineage markers of (v)-(vii), e.g., lineage negative (Lin-).

In an embodiment, the modified cell is a modified mouse HSPC and has (i) expression of CD34, e.g., detectable expression of CD34, e.g., cell surface expression of CD34. In an embodiment, the modified cell is a modified mouse HSPC and has (ii) expression of CD150 e.g., detectable expression of CD150, e.g., cell surface expression of CD150. In an embodiment, the modified cell is a modified mouse HSPC and has (iii) expression of Sca-1 e.g., detectable expression of Sca-1, e.g., cell surface expression of Sca-1. In an embodiment, the modified cell is a modified mouse HSPC and has (iv) expression of c-kit e.g., detectable expression of c-KIT, e.g., cell surface expression of c-kit. In an embodiment, the modified cell is a modified mouse HSPC and has (v) no detectable or low expression of markers associated with primitive progenitor cells, e.g., CMP and/or CLP. In an embodiment, the modified cell is a modified mouse HSPC and has (vi) no detectable or low expression of markers associated with committed precursor cells, e.g., MEP, GM, TNK and/or BCP. In an embodiment, the modified cell is a modified mouse HSPC and has (vii) no detectable or low expression of markers associated with lineage committed cells, e.g., TCP, NKP, GP, MP, EP and/or MkP. In an embodiment, the modified cell is a modified mouse HSPC and has (viii) no detectable or low expression of markers associated with one, two or all cell lineage markers of (v)-(vii), e.g., lineage negative (Lin-).

In an embodiment, the modified mouse HSPC expresses any one of (i)-(iv). In an embodiment, the modified mouse HSPC expresses any two of (i)-(iv). In an embodiment, the modified mouse HSPC expresses any three of (i)-(iv). In an embodiment, the modified mouse HSPC expresses all of (i)-(iv).

In an embodiment, the modified mouse HSPC has no detectable or low expression of any one of (v)-(vii). In an embodiment, the modified mouse HSPC has no detectable or low expression of any two of (v)-(vii). In an embodiment, the modified mouse HSPC has no detectable or low expression of all of (v)-(vii), e.g., wherein the mouse HSPC is a lineage negative HSPC.

In an embodiment, the modified mouse HSPC expresses c-Kit and Scal, e.g., a c-KIT+ and Sca-1+HSC. In an embodiment, the modified mouse HSPC expresses c-Kit and Scal, e.g., a c-KIT+ and Sca-1+HSC, and has no detectable expression or low expression of any one, two or all of (v)-(vii).

In an embodiment of any of the methods, compositions, or cells disclosed herein, the modified cell (e.g., modified stem or progenitor cell, e.g., modified HSPC) is a modified human cell and has one, two, three, four, five, six, seven, eight, or all of the following expression characteristics: (i) expression (e.g., detectable expression, e.g., cell surface expression) of a human ortholog or equivalent of NHP CD45; (ii) expression (e.g., detectable expression, e.g., cell surface expression) of a human ortholog or equivalent of NHP CD34; (iii) expression (e.g., detectable expression, e.g., cell surface expression) of a human ortholog or equivalent of NHP c-Kit (CD117); (iv) expression (e.g., detectable expression, e.g., cell surface expression) of a human ortholog or equivalent of NHP CD90; (v) expression (e.g., detectable expression, e.g., cell surface expression) of a human ortholog or equivalent of NHP CD123; (vi) expression (e.g., detectable expression, e.g., cell surface expression) of a human ortholog or equivalent of NHP CD45RA; (vii) no detectable or low expression of markers associated with primitive progenitor cells, e.g., a human ortholog or equivalent of NHP CMP, MEP, GMP and/or CLP; (viii) no detectable or low expression of markers associated with lineage committed cells, e.g., a human ortholog or equivalent of NHP TCP, NKP, GP, MP, EP and/or MkP; or (ix) no detectable or low expression of markers associated with one, two or all cell lineage markers of (vii)-(viii), e.g., lineage negative (Lin-), or a human ortholog or equivalent thereof.

In an embodiment of any of the methods, compositions, or cells disclosed herein, the modified cell, (e.g., modified stem or progenitor cell, e.g., modified HSPC) is a modified human cell and has one, two, three, four, five, six, seven or all of the following expression characteristics: (i) expression (e.g., detectable expression, e.g., cell surface expression) of a human ortholog or equivalent of mouse CD34; (ii) expression (e.g., detectable expression, e.g., cell surface expression) of a human ortholog or equivalent of mouse CD150; (iii) expression (e.g., detectable expression, e.g., cell surface expression) of a human ortholog or equivalent of mouse Sca-1; (iv) expression (e.g., detectable expression, e.g., cell surface expression) of a human ortholog or equivalent of mouse c-kit; (v) no detectable or low expression of markers associated with primitive progenitor cells, e.g., a human ortholog or equivalent of mouse CMP and/or CLP; (vi) no detectable or low expression of markers associated with committed precursor cells, e.g., a human ortholog or equivalent of mouse MEP, GM, TNK and/or BCP; (vii) no detectable or low expression of markers associated with lineage committed cells, e.g., a human ortholog or equivalent of mouse TCP, NKP, GP, MP, EP and/or MkP; or (viii) no detectable or low expression of markers associated with one, two or all cell lineage markers of (v)-(vii), e.g., lineage negative (Lin-), or a human ortholog or equivalent thereof. In an embodiment, the modified human HSPC expresses human orthologs or equivalents of mouse c-Kit and Scal. In an embodiment, the modified human HSPC expresses human orthologs or equivalents of mouse c-Kit and Scal, and has no detectable expression or low expression of any one, two or all of (v)-(vii).

### Hematopoietic Stem and Progenitor Cells for In Vivo Modification

In an embodiment, any of the methods disclosed herein comprise in vivo modification of a stem or progenitor cell, e.g., a hematopoietic stem and progenitor cell (HSPC). In an embodiment, any of the methods disclosed herein comprise in vivo gene editing of a stem and progenitor cell (HSPC), e.g., a hematopoietic stem or progenitor cell (HSPC). In an embodiment, the stem or progenitor cell comprises an HSPC or a population of HSPCs (e.g., a population of HSCs, HPCs, or a combination thereof). In an embodiment, the HSPC comprises an HSPC derived from an embryonic stem or progenitor cell or an HSPC derived from an induced pluripotent stem or progenitor cell.

In an embodiment of any of the methods, compositions, or cells disclosed herein, the cell is an HSPC, e.g., a multipotent HSC or a multipotent HPC. In an embodiment of any of the methods, compositions, or cells disclosed herein, the cell is a common myeloid progenitor cell, a common lymphoid progenitor cell, a multipotent progenitor cell, or a multipotent stem cell.

In an embodiment of any of the methods, compositions, or cells disclosed herein, the HSPC has one, two, three, four, five or all of the following functional characteristics: (i) ability to self-renew; (ii) unlimited proliferative potential; (iii) ability to enter and/or exit a quiescent state, e.g., a cell state where no proliferation occurs, e.g., GO phase of the cell cycle; (iv) ability to differentiate into any hematopoietic lineage, e.g., myeloid and/or lymphoid lineages, e.g., common lymphoid progenitor (CLP) or a differentiated cell thereof, and/or common myeloid progenitor (CMP) or a differentiated cell thereof, (v) ability to repopulate any hematopoietic lineage, e.g., myeloid and/or lymphoid lineages, e.g., common lymphoid progenitor (CLP) or a differentiated cell thereof; and/or common myeloid progenitor (CMP) or a differentiated cell thereof, e.g., in an organism; and/or (vi) ability to form colony forming units (CFU). In an embodiment, the HSPC has (i) the ability to self-renew. In an embodiment, the HSPC has (ii) unlimited proliferative potential. In an embodiment, the HSPC has (iii) the ability to enter and/or exit a quiescent state, e.g., a cell state where no proliferation occurs, e.g., GO phase of the cell cycle. In an embodiment, the HSPC has (iv) the ability to differentiate into any hematopoietic lineage, e.g., myeloid and/or lymphoid lineages, e.g., common lymphoid progenitor (CLP) or a differentiated cell thereof; and/or common myeloid progenitor (CMP) or a differentiated cell thereof. In an embodiment, the HSPC has (v) ability to repopulate any hematopoietic lineage, e.g., myeloid and/or lymphoid lineages, e.g., common lymphoid progenitor (CLP) or a differentiated cell thereof; and/or common myeloid progenitor (CMP) or a differentiated cell thereof, e.g., in an organism. In an embodiment, the HSPC has (vi) the ability to form colony forming units (CFU).

In an embodiment of any of the methods or compositions disclosed herein, the HSPC is a human HSPC, and has one, two, three, four, five, six, seven, eight, or all of the following expression characteristics: (i) expression of CD45, e.g., detectable expression of CD45, e.g., cell surface expression of CD45; (ii) expression of CD34, e.g., detectable expression of CD34, e.g., cell surface expression of CD34; (iii) expression of CD38, e.g., detectable expression of CD38, e.g., cell surface expression of CD38; (iv) expression of CD90 e.g., detectable expression of CD90, e.g., cell surface expression of CD90; (v) expression of CD133 e.g., detectable expression of CD133, e.g., cell surface expression of CD133; (vi) expression of CD45RA, e.g., detectable expression of CD45RA, e.g., cell surface expression of CD45RA; (vii) no detectable or low expression of markers associated with primitive progenitor cells, e.g., CMP, MEP, GMP and/or CLP; (viii) no detectable or low expression of markers associated with lineage committed cells, e.g., TCP, NKP, GP, MP, EP and/or MkP; or (ix) no detectable or low expression of markers associated with one, two or all cell lineage markers of (vii)-(viii), e.g., lineage negative (Lin-).

In an embodiment, the HSPC is a human HSPC and has (i) expression of CD45, e.g., detectable expression of CD45, e.g., cell surface expression of CD45. In an embodiment, the HSPC is a human HSPC and has (ii) expression of CD34, e.g., detectable expression of CD34, e.g., cell surface expression of CD34. In an embodiment, the HSPC is a human HSPC and has (iii) expression of CD38, e.g., detectable expression of CD38, e.g., cell surface expression of CD38. In an embodiment, the HSPC is a human HSPC and has (iv) expression of CD90 e.g., detectable expression of CD90, e.g., cell surface expression of CD90. In an embodiment, the HSPC is a human HSPC and has (v) expression of CD133 e.g., detectable expression of CD133, e.g., cell surface expression of CD133. In an embodiment, the HSPC is a human HSPC and has (vi) expression of CD45RA, e.g., detectable expression of CD45RA, e.g., cell surface expression of CD45RA. In an embodiment, the HSPC is a human HSPC and has (vii) no detectable or low expression of markers associated with primitive progenitor cells, e.g., CMP, MEP, GMP and/or CLP. In an embodiment, the HSPC is a human HSPC and has (viii) no detectable or low expression of markers associated with lineage committed cells, e.g., TCP, NKP, GP, MP, EP and/or MkP. In an embodiment, the HSPC is a human HSPC and has (ix) no detectable or low expression of markers associated with one, two or all cell lineage markers of (vii)-(viii), e.g., lineage negative (Lin-).

In an embodiment, the human HSPC expresses any one of (i)-(vi). In an embodiment, the human HSPC expresses any two of (i)-(vi). In an embodiment, the human HSPC expresses any three of (i)-(vi). In an embodiment, the human HSPC expresses all of (i)-(vi).

In an embodiment, the human HSPC has no detectable or low expression of (vii) or (viii). In an embodiment, the human HSPC has no detectable or low expression of both (vii) and (viii), e.g., wherein the human HSPC is a lineage negative HSPC.

In an embodiment of any of the methods and compositions disclosed herein, the HSPC is an NHP HSPC and has one, two, three, four, five, six, seven, eight, or all of the following expression characteristics: (i) expression of CD45, e.g., detectable expression of CD45, e.g., cell surface expression of CD45; (ii) expression of CD34, e.g., detectable expression of CD34, e.g., cell surface expression of CD34; (iii) expression of c-Kit (CD117), e.g., detectable expression of c-Kit (CD117), e.g., cell surface expression of c-Kit (CD117); (iv) expression of CD90 e.g., detectable expression of CD90, e.g., cell surface expression of CD90; (v) expression of CD123 e.g., detectable expression of CD123, e.g., cell surface expression of CD123; (vi) expression of CD45RA, e.g., detectable expression of CD45RA, e.g., cell surface expression of CD45RA; (vii) no detectable or low expression of markers associated with primitive progenitor cells, e.g., CMP, MEP, GMP and/or CLP; (viii) no detectable or low expression of markers associated with lineage committed cells, e.g., TCP, NKP, GP, MP, EP and/or MkP; or (ix) no detectable or low expression of markers associated with one, two or all cell lineage markers of (vii)-(viii), e.g., lineage negative (Lin-).

In an embodiment, the HSPC is an NHP HSPC and has (i) expression of CD45, e.g., detectable expression of CD45, e.g., cell surface expression of CD45. In an embodiment, the HSPC is an NHP HSPC and has (ii) expression of CD34, e.g., detectable expression of CD34, e.g., cell surface expression of CD34. In an embodiment, the HSPC is an NHP HSPC and has (iii) expression of c-Kit (CD117), e.g., detectable expression of c-Kit (CD117), e.g., cell surface expression of c-Kit (CD117). In an embodiment, the HSPC is an NHP HSPC and has (iv) expression of CD90 e.g., detectable expression of CD90, e.g., cell surface expression of CD90. In an embodiment, the HSPC is an NHP HSPC and has (v) expression of CD123 e.g., detectable expression of CD123, e.g., cell surface expression of CD123. In an embodiment, the HSPC is an NHP HSPC and has (vi) expression of CD45RA, e.g., detectable expression of CD45RA, e.g., cell surface expression of CD45RA. In an embodiment, the HSPC is an NHP HSPC and has (vii) no detectable or low expression of markers associated with primitive progenitor cells, e.g., CMP, MEP, GMP and/or CLP. In an embodiment, the HSPC is an NHP HSPC and has (viii) no detectable or low expression of markers associated with lineage committed cells, e.g., TCP, NKP, GP, MP, EP and/or MkP. In an embodiment, the HSPC is an NHP HSPC and has (ix) no detectable or low expression of markers associated with one, two or all cell lineage markers of (vii)-(viii), e.g., lineage negative (Lin-).

In an embodiment, the NHP HSPC expresses any one of (i)-(vi). In an embodiment, the NHP HSPC expresses any two of (i)-(vi). In an embodiment, the NHP HSPC expresses any three of (i)-(vi). In an embodiment, the NHP HSPC expresses all of (i)-(vi).

In an embodiment, the NHP HSPC has no detectable or low expression of (vii) or (viii). In an embodiment, the NHP HSPC has no detectable or low expression of both (vii) and (viii), e.g., wherein the NHP HSPC is a lineage negative HSPC.

In an embodiment of any of the methods and compositions disclosed herein, the HSPC is a mouse HSPC and has one, two, three, four, five, six, seven or all of the following expression characteristics: (i) expression of CD34, e.g., detectable expression of CD34, e.g., cell surface expression of CD34; (ii) expression of CD150 e.g., detectable expression of CD150, e.g., cell surface expression of CD150; (iii) expression of Sca-1 e.g., detectable expression of Sca-1, e.g., cell surface expression of Sca-1; (iv) expression of c-kit e.g., detectable expression of c-KIT, e.g., cell surface expression of c-kit; (v) no detectable or low expression of markers associated with primitive progenitor cells, e.g., CMP and/or CLP; (vi) no detectable or low expression of markers associated with committed precursor cells, e.g., MEP, GM, TNK and/or BCP; (vii) no detectable or low expression of markers associated with lineage committed cells, e.g., TCP, NKP, GP, MP, EP and/or MkP; or (viii) no detectable or low expression of markers associated with one, two or all cell lineage markers of (viii)-(x), e.g., lineage negative (Lin-).

In an embodiment, the HSPC is a mouse HSPC and has (i) expression of CD34, e.g., detectable expression of CD34, e.g., cell surface expression of CD34. In an embodiment, the HSPC is a mouse HSPC and has (ii) expression of CD150 e.g., detectable expression of CD150, e.g., cell surface expression of CD150. In an embodiment, the HSPC is a mouse HSPC and has (iii) expression of Sca-1 e.g., detectable expression of Sca-1, e.g., cell surface expression of Sca-1. In an embodiment, the HSPC is a mouse HSPC and has (iv) expression of c-kit e.g., detectable expression of c-KIT, e.g., cell surface expression of c-kit. In an embodiment, the HSPC is a mouse HSPC and has (v) no detectable or low expression of markers associated with primitive progenitor cells, e.g., CMP and/or CLP. In an embodiment, the HSPC is a mouse HSPC and has (vi) no detectable or low expression of markers associated with committed precursor cells, e.g., MEP, GM, TNK and/or BCP. In an embodiment, the HSPC is a mouse HSPC and has (vii) no detectable or low expression of markers associated with lineage committed cells, e.g., TCP, NKP, GP, MP, EP and/or MkP. In an embodiment, the HSPC is a mouse HSPC and has (viii) no detectable or low expression of markers associated with one, two or all cell lineage markers of (v)-(vii), e.g., lineage negative (Lin-).

In an embodiment of any of the methods, compositions, or cells disclosed herein, the mouse HSPC has no detectable expression or low expression of any one of (v)-(vii). In an embodiment of any of the methods, compositions, or cells disclosed herein, the mouse HSPC has no detectable expression or low expression of any two of (v)-(vii). In an embodiment of any of the methods, compositions, or cells disclosed herein, the mouse HSPC has no detectable expression or low expression of all of (v)-(vii), e.g., wherein the HSPC is a lineage negative HSPC.

In an embodiment of any of the methods, compositions, or cells disclosed herein, the mouse HSPC expresses c-Kit and Scal, e.g., a c-KIT+ and Sca-1+HSC. In an embodiment of any of the methods, compositions, or cells disclosed herein, the mouse HSPC expresses c-Kit and Scal, e.g., a c-KIT+ and Sca-1+HSC, and the mouse HSPC has no detectable expression or low expression of any one, any two or all of (v)-(vii).

In an embodiment of any of the methods, compositions, or cells disclosed herein, the modified cell (e.g., modified stem or progenitor cell, e.g., modified HSPC is a modified human cell and has one, two, three, four, five, six, seven, eight, or all of the following expression characteristics: (i) expression (e.g., detectable expression, e.g., cell surface expression) of a human ortholog or equivalent of NHP CD45; (ii) expression (e.g., detectable expression, e.g., cell surface expression) of a human ortholog or equivalent of NHP CD34; (iii) expression (e.g., detectable expression, e.g., cell surface expression) of a human ortholog or equivalent of NHP c-Kit (CD117); (iv) expression (e.g., detectable expression, e.g., cell surface expression) of a human ortholog or equivalent of NHP CD90; (v) expression (e.g., detectable expression, e.g., cell surface expression) of a human ortholog or equivalent of NHP CD123; (vi) expression (e.g., detectable expression, e.g., cell surface expression) of a human ortholog or equivalent of NHP CD45RA; (vii) no detectable or low expression of markers associated with primitive progenitor cells, e.g., a human ortholog or equivalent of NHP CMP, MEP, GMP and/or CLP; (viii) no detectable or low expression of markers associated with lineage committed cells, e.g., a human ortholog or equivalent of NHP TCP, NKP, GP, MP, EP and/or MkP; or (ix) no detectable or low expression of markers associated with one, two or all cell lineage markers of (vii)-(viii), e.g., lineage negative (Lin-), or a human ortholog or equivalent thereof.

In an embodiment of any of the methods, compositions, or cells disclosed herein, the modified cell, (e.g., modified stem or progenitor cell, e.g., modified HSPC) is a modified human cell and has one, two, three, four, five, six, seven or all of the following expression characteristics: (i) expression (e.g., detectable expression, e.g., cell surface expression) of a human ortholog or equivalent of mouse CD34; (ii) expression (e.g., detectable expression, e.g., cell surface expression) of a human ortholog or equivalent of mouse CD150; (iii) expression (e.g., detectable expression, e.g., cell surface expression) of a human ortholog or equivalent of mouse Sca-1; (iv) expression (e.g., detectable expression, e.g., cell surface expression) of a human ortholog or equivalent of mouse c-kit; (v) no detectable or low expression of markers associated with primitive progenitor cells, e.g., a human ortholog or equivalent of mouse CMP and/or CLP; (vi) no detectable or low expression of markers associated with committed precursor cells, e.g., a human ortholog or equivalent of mouse MEP, GM, TNK and/or BCP; (vii) no detectable or low expression of markers associated with lineage committed cells, e.g., a human ortholog or equivalent of mouse TCP, NKP, GP, MP, EP and/or MkP; or (viii) no detectable or low expression of markers associated with one, two or all cell lineage markers of (v)-(vii), e.g., lineage negative (Lin-), or a human ortholog or equivalent thereof. In an embodiment, the modified human HSPC expresses human orthologs or equivalents of mouse c-Kit and Scal. In an embodiment, the modified human HSPC expresses human orthologs or equivalents of mouse c-Kit and Scal, and has no detectable expression or low expression of any one, two or all of (v)-(vii).

In an embodiment of any of the methods, compositions, or cells disclosed herein, prior to contacting the cell with the LNP composition, the cell (e.g., population of cells) is isolated from a subject and expanded, enriched and/or cultured in vitro. In an embodiment of any of the methods, compositions, or cells disclosed herein, the expanded, enriched and/or cultured cell, e.g., population of cells, is administered into a host, e.g., an autologous or allogeneic host.

### Payload for In Vivo Modification of Cell or Tissue

In an embodiment of any of the methods, compositions, or cells disclosed herein, the LNP composition comprises a payload, e.g., as described herein. In an embodiment, the payload modifies, e.g., increases or decreases, the component or parameter associated with the cell or tissue, resulting in a modified cell, e.g., modified HSPC, or tissue. In an embodiment, the payload comprises a nucleic acid molecule, a peptide molecule, a lipid molecule, a low molecular weight molecule, or a combination thereof. In an embodiment, the payload affects a parameter or component of a stem or progenitor cell, e.g., a common myeloid progenitor cell, a common lymphoid progenitor cell, a multipotent progenitor cell, or a multipotent stem cell. In an embodiment, the progenitor cell is an HSPC, e.g., an HSC or HPC. In a preferred embodiment, the payload produces an alteration in a hemoglobinopathy, a clotting factor disorder, a blood cell disorder, or an immune cell disorder in a subject.

In an embodiment, the payload comprises a nucleic acid molecule comprising a DNA molecule, e.g., double stranded DNA; single stranded DNA; or plasmid DNA. In an embodiment, the payload comprises a nucleic acid molecule comprising an RNA molecule, e.g., mRNA, rRNA, tRNA, regulatory RNA, non-coding RNA, long non-coding RNA (lncRNA), guide RNA (gRNA), piwi-interacting RNA (piRNA), small nucleolar RNA (snoRNA), small nuclear RNA (snRNA), extracellular RNA (exRNA), small Cajal body-specific RNA (scaRNA), microRNA (miRNA), circular RNA, or an RNAi molecule, e.g., small interfering RNA (siRNA) or short hairpin (shRNA). In an embodiment, the payload comprises the payload comprises mRNA. In an embodiment, the mRNA comprises at least one chemical modification. In an embodiment, the chemical modification is selected from the group consisting of pseudouridine, N1-methylpseudouridine, 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methyluridine, 5-methoxyuridine, and 2′-O-methyl uridine. In an embodiment, the chemical modification is selected from the group consisting of pseudouridine, N1-methylpseudouridine, 5-methylcytosine, 5-methoxyuridine, and a combination thereof. In an embodiment, the chemical modification is N1-methylpseudouridine. In an embodiment, the mRNA comprises fully modified N1-methylpseudouridine.

In an embodiment, the payload comprises a peptide molecule, e.g., as described herein.

In an embodiment, the payload comprises a lipid molecule, e.g., as described herein. In an embodiment, the payload comprises a low molecular weight molecule, e.g., as described herein.

In an embodiment of any of the methods, compositions, or cells disclosed herein, the payload comprises a genetic modulator (e.g., a modulator that genetically alters the cell or tissue); an epigenetic modulator (e.g., a modulator that epigenetically alters the cell or tissue); an RNA modulator (e.g., a modulator that alters an RNA molecule in the cell or tissue); a peptide modulator (e.g., a modulator that alters a peptide molecule in the cell or tissue); a lipid modulator (e.g., a modulator that alters a lipid molecule in the cell or tissue); or a combination thereof.

In an embodiment, the payload comprises a genetic modulator (e.g., a modulator that genetically alters the cell or tissue). In an embodiment the genetic modulator comprises a system which modifies a nucleic acid sequence in a DNA molecule, e.g., by altering a nucleobase, e.g., introducing an insertion, a deletion, a mutation (e.g., a missense mutation, a silent mutation or a nonsense mutation), a duplication, or an inversion, or any combination thereof. In an embodiment, the genetic modulator comprises a DNA base editor, CRISPR/Cas gene editing system, a zinc finger nuclease (ZFN) system, a Transcription activator-like effector nuclease (TALEN) system, a meganuclease system, or a transposase system, or any combination thereof.

In an embodiment, the genetic modulator comprises a template DNA. In an embodiment, the genetic modulator does not comprise a template DNA. In an embodiment, the genetic modulator comprises a template RNA. In an embodiment, the genetic modulator does not comprise a template RNA.

In an embodiment, the genetic modulator is a CRISPR/Cas gene editing system. In an embodiment, the CRISPR/Cas gene editing system comprises a guide RNA (gRNA) molecule comprising a targeting sequence specific to a sequence of a target gene and a peptide having nuclease activity, e.g., endonuclease activity, e.g., a Cas protein or a fragment or a variant thereof, e.g., a Cas9 protein, a fragment or a variant thereof; a Cas3 protein, a fragment or a variant thereof; a Cas12a protein, a fragment or a variant thereof; a Cas12e protein, a fragment or a variant thereof; a Cas 13 protein, a fragment or a variant thereof, or a Cas14 protein, a fragment or a variant thereof.

In an embodiment, the CRISPR/Cas gene editing system comprises a gRNA molecule comprising a targeting sequence specific to a sequence of a target gene, and a nucleic acid encoding a peptide having nuclease activity, e.g., endonuclease activity, e.g., a Cas protein or a fragment or variant thereof, e.g., a Cas9 protein, a fragment or a variant thereof; a Cas3 protein, a fragment or a variant thereof; a Cas12a protein, a fragment or a variant thereof; a Cas12e protein, a fragment or a variant thereof; a Cas13 protein, a fragment or a variant thereof; or a Cas14 protein, a fragment or a variant thereof.

In an embodiment, the CRISPR/Cas gene editing system comprises a nucleic acid encoding a gRNA molecule comprising a targeting sequence specific to a sequence of a target gene, and a Cas9 protein, a fragment or a variant thereof.

In an embodiment, the CRISPR/Cas gene editing system comprises a nucleic acid encoding a gRNA molecule comprising a targeting sequence specific to a sequence of a target gene, and a nucleic acid encoding a Cas9 protein, a fragment or a variant thereof.

In an embodiment, the CRISPR/Cas gene editing system further comprises a template DNA. In an embodiment, the CRISPR/Cas gene editing system further comprises a template RNA. In an embodiment, the CRISPR/Cas gene editing system further comprises a reverse transcriptase.

In an embodiment of any of the methods, compositions, or cells disclosed herein, the genetic modulator is a zinc finger nuclease (ZFN) system. In an embodiment, the ZFN system comprises a peptide having: a Zinc finger DNA binding domain, a fragment or a variant thereof, and/or nuclease activity, e.g., endonuclease activity. In an embodiment, the ZFN system comprises a peptide having a Zn finger DNA binding domain. In an embodiment, the Zn finger binding domain comprises 1, 2, 3, 4, 5, 6, 7, 8 or more Zinc fingers. In an embodiment, the ZFN system comprises a peptide having nuclease activity e.g., endonuclease activity. In an embodiment, the peptide having nuclease activity is a type-II restriction 1-like endonuclease, e.g., a FokI endonuclease. In an embodiment, the ZFN system comprises a nucleic acid encoding a peptide having: a zinc finger DNA binding domain, a fragment or a variant thereof, and/or nuclease activity, e.g., endonuclease activity.

In an embodiment, the ZFN system comprises a nucleic acid encoding a peptide having a Zn finger DNA binding domain. In an embodiment, the Zn finger binding domain comprises 1, 2, 3, 4, 5, 6, 7, 8 or more Zinc fingers. In an embodiment, the ZFN system comprises a nucleic acid encoding a peptide having nuclease activity e.g., endonuclease activity. In an embodiment, the peptide having nuclease activity is a type-II restriction 1-like endonuclease, e.g., a FokI endonuclease.

In an embodiment, the system further comprises a template, e.g., template DNA.

In an embodiment of any of the methods, compositions, or cells disclosed herein, the genetic modulator is a Transcription activator-like effector nuclease (TALEN) system. In an embodiment, the system comprises a peptide having: a Transcription activator-like (TAL) effector DNA binding domain, a fragment or a variant thereof; and/or nuclease activity, e.g., endonuclease activity. In an embodiment, the system comprises a peptide having a TAL effector DNA binding domain, a fragment or a variant thereof. In an embodiment, the system comprises a peptide having nuclease activity, e.g., endonuclease activity. In an embodiment, the peptide having nuclease activity is a type-II restriction 1-like endonuclease, e.g., a FokI endonuclease.

In an embodiment, the system comprises a nucleic acid encoding a peptide having: a Transcription activator-like (TAL) effector DNA binding domain, a fragment or a variant thereof, and/or nuclease activity, e.g., endonuclease activity. In an embodiment, the system comprises a nucleic acid encoding a peptide having a Transcription activator-like (TAL) effector DNA binding domain, a fragment or a variant thereof. In an embodiment, the system comprises a nucleic acid encoding a peptide having nuclease activity, e.g., endonuclease activity. In an embodiment, the peptide having nuclease activity is a type-II restriction 1-like endonuclease, e.g., a FokI endonuclease.

In an embodiment, the system further comprises a template, e.g., a template DNA.

In an embodiment of any of the methods, compositions, or cells disclosed herein, the genetic modulator is a meganuclease system. In an embodiment, the meganuclease system comprises a peptide having a DNA binding domain and nuclease activity, e.g., a homing endonuclease. In an embodiment, the homing endonuclease comprises a LAGLIDADG endonuclease (SEQ ID NO: 270), GIY-YIG endonuclease, HNH endonuclease, His-Cys box endonuclease or a PD-(D/E)XK endonuclease, or a fragment or variant thereof, e.g., as described in Silva G. et al, (2011) Curr Gene Therapy 11(1): 11-27.

In an embodiment, the meganuclease system comprises a nucleic acid encoding a peptide having a DNA binding domain and nuclease activity, e.g., a homing endonuclease. In an embodiment, the homing endonuclease comprises a LAGLIDADG endonuclease (SEQ ID NO: 270), GIY-YIG endonuclease, HNH endonuclease, His-Cys box endonuclease or a PD-(D/E)XK endonuclease, or a fragment or variant thereof, e.g., as described in Silva G. et al, (2011) Curr Gene Therapy 11(1): 11-27.

In an embodiment, the system further comprises a template, e.g., a template DNA.

In an embodiment of any of the methods, compositions, or cells disclosed herein, the genetic modulator is a transposase system. In an embodiment, the transposase system comprises a nucleic acid sequence encoding a peptide having reverse transcriptase and/or nuclease activity, e.g., a retrotransposon, e.g., an LTR retrotransposon or a non-LTR retrotransposon. In an embodiment, the transposase system comprises a template, e.g., an RNA template.

In an embodiment of any of the methods, compositions, or cells disclosed herein, the payload comprises an epigenetic modulator (e.g., a modulator that epigenetically alters the cell or tissue). In an embodiment, the epigenetic modulator comprises a molecule that modifies chromatin architecture, methylates DNA, and/or modifies a histone. In an embodiment, the epigenetic modulator is a molecule that modifies chromatin architecture, e.g., a SWI/SNF remodeling complex or a component thereof. In an embodiment, the epigenetic modulator is a molecule that methylates DNA, e.g., a DNA methyltransferase, a fragment or variant thereof (e.g., DNMT1, DNMT2 DNMT3A, DNMT3B, DNMT3L, or M. SssI); a polycomb repressive complex or a component thereof, e.g., PRC1 or PRC2, or PR-DUB, or a fragment or a variant thereof, a demethylase, or a fragment or a variant thereof (e.g., Tet1, Tet2 or Tet3). In an embodiment, the epigenetic modulator is a molecule that modifies a histone, e.g., methylates and/or acetylates a histone, e.g., a histone modifying enzyme or a fragment or a variant thereof, e.g., HMT, HDM, HAT, or HDAC.

In an embodiment of any of the methods, compositions, or cells disclosed herein, the payload comprises an RNA modulator (e.g., a modulator that alters an RNA molecule in the cell or tissue). In an embodiment, the RNA modulator comprises a molecule that alters the expression and/or activity; stability or compartmentalization of an RNA molecule. In an embodiment, the RNA modulator comprises an RNA molecule, e.g., mRNA, rRNA, tRNA, regulatory RNA, non-coding RNA, long non-coding RNA (lncRNA), guide RNA (gRNA), piwi-interacting RNA (piRNA), small nucleolar RNA (snoRNA), small nuclear RNA (snRNA), extracellular RNA (exRNA), small Cajal body-specific RNA (scaRNA), microRNA (miRNA), circular RNA, or an RNAi molecule, e.g., small interfering RNA (siRNA) or small hairpin RNA (shRNA). In an embodiment, the RNA modulator comprises a DNA molecule. In an embodiment, the RNA modulator comprises a low molecular weight molecule. In an embodiment, the RNA modulator comprises a peptide, e.g., an RNA binding protein, a fragment, or a variant thereof; or an enzyme, or a fragment or variant thereof. In an embodiment, the RNA modulator comprises an RNA base editor system. In an embodiment, the RNA base editor system comprises: a deaminase, e.g., an RNA-specific adenosine deaminase (ADAR); a Cas protein, a fragment or a variant thereof, and/or a guide RNA. In an embodiment, the RNA base editor system further comprises a template, e.g., a DNA or RNA template.

In an embodiment of any of the methods disclosed herein the payload comprises a peptide modulator (e.g., a modulator that alters a peptide molecule in the cell or tissue). In an embodiment, the payload comprises a lipid modulator (e.g., a modulator that alters a lipid molecule in the cell or tissue); or a combination thereof.

In an embodiment, the payload comprises a therapeutic payload or a prophylactic payload. In an embodiment, the therapeutic payload or prophylactic payload comprises a secreted protein, a membrane-bound protein, or an intercellular protein; or an mRNA encoding a secreted protein, a membrane-bound protein; or an intercellular protein. In an embodiment, the therapeutic payload or prophylactic payload comprises a protein, polypeptide, or peptide.

### Additional Features of Compositions and Methods Disclosed Herein

Additional features of any of the LNP compositions, pharmaceutical composition comprising said LNPs, methods or compositions for use disclosed herein include the following embodiments.

In an embodiment of any of the methods, compositions, or cells disclosed herein, the disease or disorder is selected from the group consisting of a hemoglobinopathy, a clotting factor disorder, a blood cell disorder, and an immune cell disorder.

In an embodiment of any of the methods disclosed herein the subject is a mammal, e.g., human.

In some embodiments of any of the methods or compositions disclosed herein, the LNP composition comprises: (i) an ionizable lipid, e.g., an amino lipid; (ii) a sterol or other structural lipid; (iii) a non-cationic helper lipid or phospholipid; and (iv) a PEG-lipid. In some embodiments, the ionizable lipid comprises a compound of Formula (I). In some embodiments, the ionizable lipid comprises a compound of Formula (I-I). In some embodiments, the ionizable lipid comprises a compound of Formula (I-II). In some embodiments, the ionizable lipid comprises a compound of Formula (I-III). In some embodiments, the ionizable lipid comprises a compound of Formula (I-IV). In some embodiments, the ionizable lipid comprises a compound of Formula (Ia). In some embodiments, the ionizable lipid comprises a compound of Formula (Ib). In some embodiments, the ionizable lipid comprises a compound of Formula (Ic). In some embodiments, the ionizable lipid comprises a compound of Formula (II). In some embodiments, the ionizable lipid comprises a compound of Formula (II-I).

In some embodiments of any of the LNP compositions, methods or uses disclosed herein, the polynucleotide comprises an mRNA. In some embodiments, the mRNA comprises at least one chemical modification, e.g., as described herein. In an embodiment, the chemical modification is selected from the group consisting of pseudouridine, N1-methylpseudouridine, 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methyluridine, 5-methoxyuridine, and 2′-O-methyl uridine. In an embodiment, the chemical modification is selected from the group consisting of pseudouridine, N1-methylpseudouridine, 5-methylcytosine, 5-methoxyuridine, and a combination thereof. In an embodiment, the chemical modification is N1-methylpseudouridine. In an embodiment, each mRNA in the lipid nanoparticle comprises fully modified N1-methylpseudouridine.

In some embodiments of any of the LNP compositions, methods or uses disclosed herein, the LNP is formulated for intravenous, subcutaneous, intramuscular, intranasal, intraocular, or pulmonary delivery. In some embodiments, the LNP is formulated for intravenous delivery. In some embodiments, the LNP is formulated for subcutaneous delivery. In some embodiments, the LNP is formulated for intramuscular delivery. In some embodiments, the LNP is formulated for intranasal delivery. In some embodiments, the LNP is formulated for intraocular delivery. In some embodiments, the LNP is formulated for pulmonary delivery. In an embodiment, the delivery is a single delivery. In an embodiment, the delivery is a repeat delivery.

In some embodiments of any of the LNP compositions, methods or uses disclosed herein, the LNP further comprising a pharmaceutically acceptable carrier or excipient.

In an embodiment of any of the LNP compositions, methods or compositions for use disclosed herein, the LNP composition comprises: (i) an ionizable lipid, e.g., an amino lipid; (ii) a sterol or other structural lipid; (iii) a non-cationic helper lipid or phospholipid; and, optionally, (iv) a PEG-lipid.

In an embodiment of any of the LNP compositions, methods or compositions for use disclosed herein, the LNP composition comprises an ionizable lipid comprising an amino lipid. In an embodiment, the ionizable lipid comprises a compound of any one of Formulae (I), (I-I), (I-II), (I-III), (I-IV), (Ia), (Ib), (Ic), (II), or (II-I). In an embodiment, the ionizable lipid comprises a compound of Formula (I). In an embodiment, the ionizable lipid comprises a compound of Formula (I-I). In an embodiment, the ionizable lipid comprises a compound of Formula (I-II). In an embodiment, the ionizable lipid comprises a compound of Formula (I-III). In an embodiment, the ionizable lipid comprises a compound of Formula (I-IV). In an embodiment, the ionizable lipid comprises a compound of Formula (Ia). In an embodiment, the ionizable lipid comprises a compound of Formula (Ib). In an embodiment, the ionizable lipid comprises a compound of Formula (Ic). In an embodiment, the ionizable lipid comprises a compound of Formula (II). In an embodiment, the ionizable lipid comprises a compound of Formula (II-I).

In an embodiment of any of the LNP compositions, methods or compositions for use disclosed herein, the LNP composition comprises a non-cationic helper lipid or phospholipid comprising a compound selected from the group consisting of 1,2-distearoyl-sn-glycero-3-phosphocholine (DSPC), 1,2-Distearoyl-sn-glycero-3-phosphoethanolamine (DSPE), 1,2-dioleoyl-sn-glycero-3-phosphoethanolamine (DOPE), 1,2-dilinoleoyl-sn-glycero-3-phosphocholine (DLPC), 1,2-dimyristoyl-sn-gly cero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether P C), 1-oleoyl-2 cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine,1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin, and mixtures thereof. In an embodiment, the phospholipid is DSPC, e.g., a variant of DSPC, e.g., a compound of Formula (IV).

In an embodiment of any of the LNP compositions, methods or compositions for use disclosed herein, the LNP composition comprises a structural lipid. In one embodiment, the structural lipid is a phytosterol or a combination of a phytosterol and cholesterol. In one embodiment, the phytosterol is selected from the group consisting of 0-sitosterol, stigmasterol, 0-sitostanol, campesterol, brassicasterol, and combinations thereof.

In one embodiment, the structural lipid can be selected from the group including but not limited to, cholesterol, fecosterol, sitosterol, ergosterol, campesterol, stigmasterol, brassicasterol, tomatidine, tomatine, ursolic acid, alpha-tocopherol, hopanoids, phytosterols, steroids, and mixtures thereof. In some embodiments, the structural lipid is a sterol. As defined herein, “sterols” are a subgroup of steroids consisting of steroid alcohols. In certain embodiments, the structural lipid is a steroid. In certain embodiments, the structural lipid is cholesterol. In certain embodiments, the structural lipid is an analog of cholesterol. In certain embodiments, the structural lipid is alpha-tocopherol.

In one embodiment, the structural lipid is selected from selected from β-sitosterol and cholesterol. In an embodiment, the structural lipid is β-sitosterol. In an embodiment, the structural lipid is cholesterol.

In an embodiment of any of the LNP compositions, methods or compositions for use disclosed herein, the LNP composition comprises a PEG lipid. In one embodiment, the PEG-lipid is selected from the group consisting of a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof.

In an embodiment, the PEG lipid is selected from the group consisting of a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof. In an embodiment, the PEG lipid is selected from the group consisting of PEG-c-DOMG, PEG-DMG, PEG-DLPE, PEG-DMPE, PEG-DPPC and PEG-DSPE lipid. In an embodiment, the PEG-lipid is PEG-DMG.

In an embodiment, the PEG lipid is chosen from a compound of. Formula (V), Formula (VI-A), Formula (VI-B), Formula (VI-C) or Formula (VI-D). In an embodiment, the PEG-lipid is a compound of Formula (VI-A). In an embodiment, the PEG-lipid is a compound of Formula (VI-B). In an embodiment, the PEG-lipid is a compound of Formula (VI-C). In an embodiment, the PEG-lipid is a compound of Formula (VI-D).

In an embodiment, the LNP composition comprises an amino lipid comprising a compound of Formula (I-I) and a PEG lipid comprising a compound of Formula (VI-D). In a preferred embodiment, the LNP composition comprises an amino lipid comprising a compound of Formula (I-I), a phospholipid comprising DSPC, a structural lipid comprising cholesterol, and a PEG lipid comprising a compound of Formula (VI-D).

In an embodiment of any of the LNP compositions, methods or cells disclosed herein, the LNP comprises about 20 mol % to about 60 mol % ionizable lipid, about 5 mol % to about 25 mol % non-cationic helper lipid or phospholipid, about 25 mol % to about 55 mol % sterol or other structural lipid, and about 0.5 mol % to about 15 mol % PEG lipid. In one embodiment of the LNPs or methods of the disclosure, the LNP comprises about 35 mol % to about 55 mol % ionizable lipid, about 5 mol % to about 25 mol % non-cationic helper lipid or phospholipid, about 30 mol % to about 40 mol % sterol or other structural lipid, and about 0 mol % to about 10 mol % PEG lipid. In one embodiment of the LNPs or methods of the disclosure, the LNP comprises about 50 mol % ionizable lipid, about 10 mol % non-cationic helper lipid or phospholipid, about 38.5 mol % sterol or other structural lipid, and about 1.5 mol % PEG lipid. In one embodiment of the LNPs or methods of the disclosure, the LNP comprises about 49.83 mol % ionizable lipid, about 9.83 mol % non-cationic helper lipid or phospholipid, about 30.33 mol % sterol or other structural lipid, and about 2.0 mol % PEG lipid.

In an embodiment of any of the LNP compositions, methods or cells disclosed herein, the LNP comprises about 45 mol % to about 50 mol % ionizable lipid. In one embodiment of the LNPs or methods of the disclosure, the LNP comprises about 45.5 mol % to about 49.5 mol % ionizable lipid. In one embodiment of the LNPs or methods of the disclosure, the LNP comprises about 46 mol % to about 49 mol % ionizable lipid. In one embodiment of the LNPs or methods of the disclosure, the LNP comprises about 46.5 mol % to about 48.5 mol % ionizable lipid. In one embodiment of the LNPs or methods of the disclosure, the LNP comprises about 47 mol % to about 48 mol % ionizable lipid.

In an embodiment of any of the LNP compositions, methods or cells disclosed herein, the LNP comprises about 45 mol % to about 49.5 mol % ionizable lipid. In one embodiment of the LNPs or methods of the disclosure, the LNP comprises about 45 mol % to about 49 mol % ionizable lipid. In one embodiment of the LNPs or methods of the disclosure, the LNP comprises about 45 mol % to about 48.5 mol % ionizable lipid. In one embodiment of the LNPs or methods of the disclosure, the LNP comprises about 45 mol % to about 48 mol % ionizable lipid. In one embodiment of the LNPs or methods of the disclosure, the LNP comprises about 45 mol % to about 47.5 mol % ionizable lipid. In one embodiment of the LNPs or methods of the disclosure, the LNP comprises about 45 mol % to about 47 mol % ionizable lipid. In one embodiment of the LNPs or methods of the disclosure, the LNP comprises about 45 mol % to about 46.5 mol % ionizable lipid. In one embodiment of the LNPs or methods of the disclosure, the LNP comprises about 45 mol % to about 46 mol % ionizable lipid. In one embodiment of the LNPs or methods of the disclosure, the LNP comprises about 45 mol % to about 45.5 mol % ionizable lipid.

In an embodiment of any of the LNP compositions, methods or cells disclosed herein, the LNP comprises about 45.5 mol % to about 50 mol % ionizable lipid. In one embodiment of the LNPs or methods of the disclosure, the LNP comprises about 46 mol % to about 50 mol % ionizable lipid. In one embodiment of the LNPs or methods of the disclosure, the LNP comprises about 46.5 mol % to about 50 mol % ionizable lipid. In one embodiment of the LNPs or methods of the disclosure, the LNP comprises about 47 mol % to about 50 mol % ionizable lipid. In one embodiment of the LNPs or methods of the disclosure, the LNP comprises about 47.5 mol % to about 50 mol % ionizable lipid. In one embodiment of the LNPs or methods of the disclosure, the LNP comprises about 48 mol % to about 50 mol % ionizable lipid. In one embodiment of the LNPs or methods of the disclosure, the LNP comprises about 48.5 mol % to about 50 mol % ionizable lipid. In one embodiment of the LNPs or methods of the disclosure, the LNP comprises about 49 mol % to about 50 mol % ionizable lipid. In one embodiment of the LNPs or methods of the disclosure, the LNP comprises about 49.5 mol % to about 50 mol % ionizable lipid.

In an embodiment of any of the LNP compositions, methods or cells disclosed herein, the LNP comprises about 45 mol % to about 46 mol % ionizable lipid. In one embodiment of the LNPs or methods of the disclosure, the LNP comprises about 45.5 mol % to about 46.5 mol % ionizable lipid. In one embodiment of the LNPs or methods of the disclosure, the LNP comprises about 46 mol % to about 47 mol % ionizable lipid. In one embodiment of the LNPs or methods of the disclosure, the LNP comprises about 46.5 mol % to about 47.5 mol % ionizable lipid. In one embodiment of the LNPs or methods of the disclosure, the LNP comprises about 47 mol % to about 48 mol % ionizable lipid. In one embodiment of the LNPs or methods of the disclosure, the LNP comprises about 47.5 mol % to about 48.5 mol % ionizable lipid. In one embodiment of the LNPs or methods of the disclosure, the LNP comprises about 48 mol % to about 49 mol % ionizable lipid. In one embodiment of the LNPs or methods of the disclosure, the LNP comprises about 48.5 mol % to about 49.5 mol % ionizable lipid. In one embodiment of the LNPs or methods of the disclosure, the LNP comprises about 49 mol % to about 50 mol % ionizable lipid.

In an embodiment of any of the LNP compositions, methods or cells disclosed herein, the LNP comprises about 45 mol % ionizable lipid. In one embodiment of the LNPs or methods of the disclosure, the LNP comprises about 45.5 mol % ionizable lipid. In one embodiment of the LNPs or methods of the disclosure, the LNP comprises about 46 mol % ionizable lipid. In one embodiment of the LNPs or methods of the disclosure, the LNP comprises about 46.5 mol % ionizable lipid. In one embodiment of the LNPs or methods of the disclosure, the LNP comprises about 47 mol % ionizable lipid. In one embodiment of the LNPs or methods of the disclosure, the LNP comprises about 47.5 mol % ionizable lipid. In one embodiment of the LNPs or methods of the disclosure, the LNP comprises about 48 mol % ionizable lipid. In one embodiment of the LNPs or methods of the disclosure, the LNP comprises about 48.5 mol % ionizable lipid. In one embodiment of the LNPs or methods of the disclosure, the LNP comprises about 49 mol % ionizable lipid. In one embodiment of the LNPs or methods of the disclosure, the LNP comprises about 49.5 mol % ionizable lipid. In one embodiment of the LNPs or methods of the disclosure, the LNP comprises about 50 mol % ionizable lipid.

In an embodiment of any of the LNP compositions, methods or cells disclosed herein, the LNP comprises about 1 mol % to about 5 mol % PEG lipid. In one embodiment of the LNPs or methods of the disclosure, the LNP comprises about 1.5 mol % to about 4.5 mol % PEG lipid. In one embodiment of the LNPs or methods of the disclosure, the LNP comprises about 2 mol % to about 4 mol % PEG lipid. In one embodiment of the LNPs or methods of the disclosure, the LNP comprises about 2.5 mol % to about 3.5 mol % PEG lipid.

In an embodiment of any of the LNP compositions, methods or cells disclosed herein, the LNP comprises about 1 mol % to about 4.5 mol % PEG lipid. In one embodiment of the LNPs or methods of the disclosure, the LNP comprises about 1 mol % to about 4 mol % PEG lipid. In one embodiment of the LNPs or methods of the disclosure, the LNP comprises about 1 mol % to about 3.5 mol % PEG lipid. In one embodiment of the LNPs or methods of the disclosure, the LNP comprises about 1 mol % to about 3 mol % PEG lipid. In one embodiment of the LNPs or methods of the disclosure, the LNP comprises about 1 mol % to about 2.5 mol % PEG lipid. In one embodiment of the LNPs or methods of the disclosure, the LNP comprises about 1 mol % to about 2 mol % PEG lipid. In one embodiment of the LNPs or methods of the disclosure, the LNP comprises about 1 mol % to about 1.5 mol % PEG lipid.

In an embodiment of any of the LNP compositions, methods or cells disclosed herein, the LNP comprises about 1.5 mol % to about 5 mol % PEG lipid. In one embodiment of the LNPs or methods of the disclosure, the LNP comprises about 2 mol % to about 5 mol % PEG lipid. In one embodiment of the LNPs or methods of the disclosure, the LNP comprises about 2.5 mol % to about 5 mol % PEG lipid. In one embodiment of the LNPs or methods of the disclosure, the LNP comprises about 3 mol % to about 5 mol % PEG lipid. In one embodiment of the LNPs or methods of the disclosure, the LNP comprises about 3.5 mol % to about 5 mol % PEG lipid. In one embodiment of the LNPs or methods of the disclosure, the LNP comprises about 4 mol % to about 5 mol % PEG lipid. In one embodiment of the LNPs or methods of the disclosure, the LNP comprises about 4.5 mol % to about 5 mol % PEG lipid.

In one embodiment of the LNPs or methods of the disclosure, the LNP comprises about 1 mol % to about 2 mol % PEG lipid. In one embodiment of the LNPs or methods of the disclosure, the LNP comprises about 1.5 mol % to about 2.5 mol % PEG lipid. In one embodiment of the LNPs or methods of the disclosure, the LNP comprises about 2 mol % to about 3 mol % PEG lipid. In one embodiment of the LNPs or methods of the disclosure, the LNP comprises about 3.5 mol % to about 4.5 mol % PEG lipid. In one embodiment of the LNPs or methods of the disclosure, the LNP comprises about 4 mol % to about 5 mol % PEG lipid.

In an embodiment of any of the LNP compositions, methods or cells disclosed herein, the LNP comprises about 1 mol % PEG lipid. In one embodiment of the LNPs or methods of the disclosure, the LNP comprises about 1.5 mol % PEG lipid. In one embodiment of the LNPs or methods of the disclosure, the LNP comprises about 2 mol % PEG lipid. In one embodiment of the LNPs or methods of the disclosure, the LNP comprises about 2.5 mol % PEG lipid. In one embodiment of the LNPs or methods of the disclosure, the LNP comprises about 3 mol % PEG lipid. In one embodiment of the LNPs or methods of the disclosure, the LNP comprises about 3.5 mol % PEG lipid. In one embodiment of the LNPs or methods of the disclosure, the LNP comprises about 4 mol % PEG lipid. In one embodiment of the LNPs or methods of the disclosure, the LNP comprises about 4.5 mol % PEG lipid. In one embodiment of the LNPs or methods of the disclosure, the LNP comprises about 5 mol % PEG lipid.

In one embodiment, the mol % sterol or other structural lipid is 18.5% phytosterol and the total mol % structural lipid is 38.5%. In one embodiment, the mol % sterol or other structural lipid is 28.5% phytosterol and the total mol % structural lipid is 38.5%.

In one embodiment of the LNPs, or methods of the disclosure, the LNP comprises about 50 mol % of a compound of Formula (I) and about 10 mol % non-cationic helper lipid or phospholipid. In one embodiment of the LNPs, or methods of the disclosure, the LNP comprises about 50 mol % of a compound of Formula (I-I) and about 10 mol % non-cationic helper lipid or phospholipid. In one embodiment of the LNPs, or methods of the disclosure, the LNP comprises about 50 mol % of a compound of Formula (I-II) and about 10 mol % non-cationic helper lipid or phospholipid. In one embodiment of the LNPs, or methods of the disclosure, the LNP comprises about 50 mol % of a compound of Formula (I-III) and about 10 mol % non-cationic helper lipid or phospholipid. In one embodiment of the LNPs, or methods of the disclosure, the LNP comprises about 50 mol % of a compound of Formula (I-IV) and about 10 mol % non-cationic helper lipid or phospholipid. In one embodiment of the LNPs or methods of the disclosure, the LNP comprises 50 mol % of a compound of Formula (Ia) and about 10 mol % non-cationic helper lipid or phospholipid. In one embodiment of the LNPs, or methods of the disclosure, the LNP comprises about 50 mol % of a compound of Formula (Ib) and 10 mol % non-cationic helper lipid or phospholipid. In one embodiment of the LNPs or methods of the disclosure, the LNP comprises 50 mol % of a compound of Formula (Ic) and 10 mol % non-cationic helper lipid or phospholipid.

In one embodiment of the LNPs or methods of the disclosure, the LNP comprises 50 mol % of a compound of Formula (II) and 10 mol % non-cationic helper lipid or phospholipid. In one embodiment of the LNPs or methods of the disclosure, the LNP comprises 50 mol % of a compound of Formula (II-I) and 10 mol % non-cationic helper lipid or phospholipid.

In one embodiment of the LNPs or methods of the disclosure, the LNP comprises about 49.83 mol % of a compound of Formula (I), about 9.83 mol % non-cationic helper lipid or phospholipid, about 30.33 mol % sterol or other structural lipid, and about 2.0 mol % PEG lipid. In one embodiment of the LNPs or methods of the disclosure, the LNP comprises about 49.83 mol % of a compound of Formula (I-I), about 9.83 mol % non-cationic helper lipid or phospholipid, about 30.33 mol % sterol or other structural lipid, and about 2.0 mol % PEG lipid. In one embodiment of the LNPs or methods of the disclosure, the LNP comprises about 49.83 mol % of a compound of Formula (I-II), about 9.83 mol % non-cationic helper lipid or phospholipid, about 30.33 mol % sterol or other structural lipid, and about 2.0 mol % PEG lipid. In one embodiment of the LNPs or methods of the disclosure, the LNP comprises about 49.83 mol % of a compound of Formula (I-III), about 9.83 mol % non-cationic helper lipid or phospholipid, about 30.33 mol % sterol or other structural lipid, and about 2.0 mol % PEG lipid. In one embodiment of the LNPs or methods of the disclosure, the LNP comprises about 49.83 mol % of a compound of Formula (I-IV), about 9.83 mol % non-cationic helper lipid or phospholipid, about 30.33 mol % sterol or other structural lipid, and about 2.0 mol % PEG lipid. In one embodiment of the LNPs or methods of the disclosure, the LNP comprises about 49.83 mol % of a compound of Formula (Ia), about 9.83 mol % non-cationic helper lipid or phospholipid, about 30.33 mol % sterol or other structural lipid, and about 2.0 mol % PEG lipid. In one embodiment of the LNPs or methods of the disclosure, the LNP comprises about 49.83 mol % of a compound of Formula (Ib), about 9.83 mol % non-cationic helper lipid or phospholipid, about 30.33 mol % sterol or other structural lipid, and about 2.0 mol % PEG lipid. In one embodiment of the LNPs or methods of the disclosure, the LNP comprises about 49.83 mol % of a compound of Formula (Ic), about 9.83 mol % non-cationic helper lipid or phospholipid, about 30.33 mol % sterol or other structural lipid, and about 2.0 mol % PEG lipid.

In one embodiment of the LNPs or methods of the disclosure, the LNP comprises about 49.83 mol % of a compound of Formula (II), about 9.83 mol % non-cationic helper lipid or phospholipid, about 30.33 mol % sterol or other structural lipid, and about 2.0 mol % PEG lipid. In one embodiment of the LNPs or methods of the disclosure, the LNP comprises about 49.83 mol % of a compound of Formula (II-I), about 9.83 mol % non-cationic helper lipid or phospholipid, about 30.33 mol % sterol or other structural lipid, and about 2.0 mol % PEG lipid.

In an embodiment, an LNP of the disclosure does not include an additional targeting moiety, e.g., it transfects (e.g., at least 10%, 20%, 30%, 40%, 50%, 60%, 70%, 80%, 90%, or 95%) of stem or progenitor cells (e.g., HSPCs) without an additional targeting moiety.

In an embodiment of any of the LNP compositions, methods or uses disclosed herein, the LNP is formulated for intravenous, subcutaneous, intramuscular, intraocular, intranasal, or pulmonary delivery. In an embodiment, the LNP is formulated for intravenous delivery. In an embodiment, the LNP is formulated for subcutaneous delivery. In an embodiment, the LNP is formulated for intramuscular delivery. In an embodiment, the LNP is formulated for intraocular delivery. In an embodiment, the LNP is formulated for intranasal delivery. In an embodiment, the LNP is formulated for pulmonary delivery. In an embodiment, the delivery is a single delivery. In an embodiment, the delivery is a repeat delivery.

Additional features of any of the aforesaid LNP compositions or methods of using said LNP compositions, include one or more of the following enumerated embodiments. Those skilled in the art will recognize or be able to ascertain using no more than routine experimentation, many equivalents to the specific embodiments of the invention described herein. Such equivalents are intended to be encompassed by the following enumerated embodiments.

### OTHER EMBODIMENTS OF THE DISCLOSURE

1. A method of modifying a cell (e.g., stem or progenitor cell), e.g., modifying a parameter associated with the cell or a component associated with the cell, e.g., in a subject, comprising contacting the cell with a lipid nanoparticle (LNP) composition comprising a payload, thereby modifying the cell.
2. A method of modifying a tissue, e.g., modifying a parameter associated with the tissue or a component associated with the tissue, e.g., in a subject, comprising contacting the cell with a lipid nanoparticle (LNP) composition comprising a payload.
3. A method of treating a subject having a disease, a disorder, a mutation, or a single nucleotide polymorphism (SNP), comprising administering to the subject an effective amount of an LNP composition comprising a payload, wherein said LNP composition results in a modification of a cell (e.g., stem or progenitor cell) in the subject, e.g., modification of a component associated with the cell or a parameter associated with the cell, thereby treating the subject.
4. A method of ameliorating a symptom of a subject having a disease, a disorder, a mutation, or a single nucleotide polymorphism (SNP), comprising administering to the subject an effective amount of an LNP composition comprising a payload, wherein said LNP composition results in a modification of a cell (e.g., stem or progenitor cell) in the subject, e.g., modification of a component associated with the cell or a parameter associated with the cell, thereby ameliorating the symptom of the subject.
5. A method of delivering an LNP composition comprising a payload to a cell (e.g., stem or progenitor cell), or tissue, e.g., in a subject, comprising contacting the cell, or tissue with the LNP composition.
6. A method of contacting a cell (e.g., stem or progenitor cell) or tissue, e.g., in a subject, comprising contacting the cell or tissue with an LNP composition comprising a payload.
7. The method of any one of the preceding embodiments, wherein the LNP composition results in a modification of a genotype, a phenotype, and/or a function of the cell or tissue.
8. The method of any one of the preceding embodiments, wherein the LNP composition results in a modification of the cell, or tissue, e.g., a component associated with the cell or tissue, or a parameter associated with the cell or tissue.
9. The method of any one of the preceding embodiments, wherein the component comprises: (1) a nucleic acid associated with the cell or a fragment thereof, e.g., a DNA (e.g., exonic, intronic, intergenic, telomeric, promoter, enhancer, insulator, repressor, coding, or non-coding) or an RNA (e.g., mRNA, rRNA, tRNA, regulatory RNA, non-coding RNA, long non-coding RNA (lncRNA), guide RNA (gRNA), piwi-interacting RNA (piRNA), small nucleolar RNA (snoRNA), small nuclear RNA (snRNA), extracellular RNA (exRNA), small Cajal body-specific RNA (scaRNA), microRNA (miRNA), circular RNA, or an RNAi molecule, e.g., small interfering RNA (siRNA) or small hairpin RNA (shRNA)); (2) a peptide or protein associated with the cell or a fragment thereof; (3) a lipid component associated with the cell or a fragment thereof, or a combination thereof.
10. The method of any one of the preceding embodiments, wherein the component comprises a DNA.
11. The method of any one of the preceding embodiments, wherein the component comprises an RNA.
12. The method of any one of the preceding embodiments, wherein the component comprises a peptide or protein associated with the cell or fragment thereof.
13. The method of any one of the preceding embodiments, wherein the component comprises a lipid component associated with the cell or fragment thereof.
14. The method of any one of the preceding embodiments, wherein the component is endogenous to the cell.
15. The method of any one of the preceding embodiments, wherein the component is exogenous to the cell, e.g., has been introduced into the cell by a method known in the field, e.g., transformation, electroporation, viral based delivery or lipid-based delivery.
16. The method of any one of the preceding embodiments, wherein the parameter comprises a genotypic parameter, a phenotypic parameter, a functional parameter, an expression parameter, a signaling parameter, or any combination thereof.
17. The method of any one of the preceding embodiments, wherein the genotypic parameter comprises a genotype of the cell, e.g., the presence or absence a gene or allele, or a modification of a gene or allele, e.g., a germline or somatic mutation, or a polymorphism, in the gene or allele.
18. The method of any one of the preceding embodiments, wherein the phenotypic parameter comprises a phenotype of the cell, e.g., expression and/or activity of a molecule, e.g., cell surface protein, lipid or adhesion molecule, on the surface of the cell.
19. The method of any one of the preceding embodiments, wherein the functional parameter comprises a function of the cell, e.g., the ability of the cell to produce a gene product (e.g., a protein), the ability of the cell to proliferate, divide, and/or renew, and/or the ability of the cell to differentiate, e.g., into one or more cell types in a lineage.
20. The method of any one of the preceding embodiments, wherein the expression parameter comprises one, two, three, four or all of the following:
(a) expression level (e.g., of polypeptide or protein, or nucleic acid (e.g., mRNA));
(b) activity (e.g., of polypeptide or protein, or nucleic acid (e.g., mRNA)),
(c) post-translational modification of polypeptide or protein;
(d) folding (e.g., of polypeptide or protein, or nucleic acid (e.g., mRNA)), and/or
(e) stability (e.g., of polypeptide or protein, or nucleic acid (e.g., mRNA)), 21. The method of any one of the preceding embodiments, wherein the signaling parameter comprises one, two, three, four or all of the following:
(1) modulation of a signaling pathway, e.g., a cellular signaling pathway;
(2) cell fate modulation;
(3) modulation of expression level (e.g., of polypeptide or protein, or nucleic acid (e.g., mRNA));
(4) modulation of activity (e.g., of polypeptide or protein, or nucleic acid (e.g., mRNA)), and/or
(5) modulation of stability e.g., of polypeptide or protein, or nucleic acid (e.g., mRNA)).
   22. The method of any one of the preceding embodiments, wherein the cell is contacted in vitro, in vivo, or ex vivo with the LNP composition.
   23. The method of any one of the preceding embodiments, wherein the cell is contacted in vivo with the LNP formulation.
   24. The method of any one of the preceding embodiments, wherein the cell is contacted ex vivo with the LNP formulation.
   25. The method of any one of the preceding embodiments, wherein the cell is a stem or progenitor cell, e.g., a hematopoietic stem and progenitor cell (HSPC), e.g., an HSPC derived from an embryonic stem or progenitor cell or an HSPC derived from an induced pluripotent stem or progenitor cell.
   26. The method of any one of the preceding embodiments, wherein the cell is a common myeloid progenitor cell, a common lymphoid progenitor cell, a multipotent progenitor cell, or a multipotent stem cell.
   27. The method of any one of the preceding embodiments, wherein the cell is an HSPC, e.g., a multipotent HSC or multipotent HPC.
   28. The method of embodiment 27, wherein the HSPC has one, two, three, four, five or all of the following functional characteristics:
i. ability to self-renew;
ii. unlimited proliferative potential;
iii. ability to enter and/or exit a quiescent state, e.g., a cell state where no proliferation occurs, e.g., GO phase of the cell cycle;
iv. ability to differentiate into any hematopoietic lineage, e.g., myeloid and/or lymphoid lineages, e.g., common lymphoid progenitor (CLP) or a differentiated cell thereof, and/or common myeloid progenitor (CMP) or a differentiated cell thereof;
v. ability to repopulate any hematopoietic lineage, e.g., myeloid and/or lymphoid lineages, e.g., common lymphoid progenitor (CLP) or a differentiated cell thereof, and/or common myeloid progenitor (CMP) or a differentiated cell thereof; e.g., in an organism;
vi. ability to form colony forming units (CFU).
   29. The method of embodiment 27 or 28, wherein the HSPC that has one, two, three, four, five, six, seven, eight or all of the following expression characteristics:
i. expression of CD45, e.g., detectable expression of CD45, e.g., cell surface expression of CD45;
ii. expression of CD34, e.g., detectable expression of CD34, e.g., cell surface expression of CD34;
iii. expression of CD38, e.g., detectable expression of CD38, e.g., cell surface expression of CD38;
iv. expression of CD90 e.g., detectable expression of CD90, e.g., cell surface expression of CD90;
v. expression of CD133 e.g., detectable expression of CD133, e.g., cell surface expression of CD133;
vi. expression of CD45RA, e.g., detectable expression of CD45RA, e.g., cell surface expression of CD45RA;
vii. no detectable or low expression of markers associated with primitive progenitor cells, e.g., CMP, MEP, GMP and/or CLP;
viii. no detectable or low expression of markers associated with lineage committed cells, e.g., TCP, NKP, GP, MP, EP and/or MkP; or
ix. no detectable or low expression of markers associated with one, two or all cell lineage markers of (vii)-(viii), e.g., lineage negative (Lin-).
   30. The method of embodiment 29, wherein the human HSPC has any one of (i)-(vi).
   31. The method of embodiment 29, wherein the human HSPC has any two of (i)-(vi).
   32. The method of embodiment 29, wherein the human HSPC has any three of (i)-(vi).
   33. The method of embodiment 29, wherein the human HSPC has all of (i)-(vi).
   34. The method of any one of the embodiments 29-33, wherein the human HSPC has no detectable or low expression of (vii) or (viii).
   35. The method of any one of the embodiments 29-34, wherein the human HSPC has no detectable or low expression of both (vii) and (viii), e.g., wherein the human HSPC is a lineage negative HSPC.
   36. The method of embodiment 28 or 29, wherein the HSPC has any one, or all, or a combination of the functional characteristics of embodiment 28 and the HSPC has any one, or all, or a combination of the expression characteristics of embodiment 29.
   37. The method of any one of the preceding embodiments, wherein prior to contacting the cell with the LNP composition, the cell (e.g., population of cells) is isolated from a subject and expanded, enriched and/or cultured in vitro.
   38. The method of any one of the preceding embodiments, wherein the expanded, enriched and/or cultured cell, e.g., population of cells, is administered into a host, e.g., an autologous or allogeneic host.
   39. The method of any one of the preceding embodiments, wherein the modified cell (e.g., population of modified cells) is a modified HSPC (e.g., a population of modified HSPCs).
   40. The method of embodiment 39, wherein the modified HSPC has one, two, three, four, five or all of the following functional characteristics:
i. ability to self-renew;
ii. unlimited proliferative potential;
iii. ability to enter and/or exit a quiescent state, e.g., a cell state where no proliferation occurs, e.g., GO phase of the cell cycle;
iv. ability to differentiate into any hematopoietic lineage, e.g., myeloid and/or lymphoid lineages, e.g., common lymphoid progenitor (CLP) or a differentiated cell thereof, and/or common myeloid progenitor (CMP) or a differentiated cell thereof;
v. ability to repopulate any hematopoietic lineage, e.g., myeloid and/or lymphoid lineages, e.g., common lymphoid progenitor (CLP) or a differentiated cell thereof, and/or common myeloid progenitor (CMP) or a differentiated cell thereof; e.g., in an organism; or
vi. ability to form colony forming units (CFU).
   41. The method of embodiment 39 or 40, wherein the modified HSPC has the ability to form CFU, e.g., as measured in an ex-vivo colony-forming unit (CFU) assay, e.g., as described in Example 2, or as measured in a lineage tracing experiment, e.g., as described in Example 3, e.g., as compared to an otherwise similar HSPC which has not been contacted with an LNP, or has been contacted with a different LNP.
   42. The method of any one of embodiments 39-41, wherein the modified HSPC has the ability to differentiate into myeloid cells, e.g., as measured in an ex-vivo colony-forming unit (CFU) assay, e.g., as described in Example 2, or as measured in a lineage tracing experiment, e.g., as described in Example 3, e.g., as compared to an otherwise similar HSC which has not been contacted with an LNP, or has been contacted with a different LNP.
   43. The method of any one of embodiments 39-42, wherein the modified HSPC has the ability to differentiate into lymphoid cells, e.g., as measured in lineage tracing experiments, e.g., as described in Example 3, e.g., as compared to an otherwise similar HSC which has not been contacted with an LNP, or has been contacted with a different LNP.
   44. The method of any one of embodiments 39-43, wherein the modified HSPC has the ability to differentiate into an erythrocyte cell or a platelet, e.g., as shown in Example 3, e.g., as compared to an otherwise similar HSPC which has not been contacted with an LNP, or has been contacted with a different LNP.
   45. The method of embodiment 44, wherein the modified HSPC differentiates into an erythrocyte cell or a platelet in vivo.
   46. The method of embodiment 44, wherein the modified HSPC differentiates into an erythrocyte cell or a platelet in vitro.
   47. The method of any one of embodiments 39-46, wherein the modified HSPC has the ability to differentiate into a neutrophil, a monocyte, a B cell, or a T cell (e.g., a CD4+ T cell or a CD8+ T cell), e.g., as shown in Example 3, e.g., as compared to an otherwise similar HSPC which has not been contacted with an LNP, or has been contacted with a different LNP.
   48. The method of embodiment 47, wherein the modified HSPC differentiates into a neutrophil, a monocyte, a B cell, or a T cell (e.g., a CD4+ T cell or a CD8+ T cell) in vivo.
   49. The method of embodiment 47, wherein the modified HSPC differentiates into a neutrophil, a monocyte, a B cell, or a T cell (e.g., a CD4+ T cell or a CD8+ T cell) in vitro.
   50. The method of any of embodiments 39-49, wherein the modified HSPC persists, e.g., in vivo, for at least 1, 2, 3, 4, 5, 6, 7, 10, 15, 20, 25, 30, 45, 60, 90, 120, 180, 240, 300, or 365 days or more.
   51. The method of embodiment 50, wherein the in vivo persistence of the modified HSPC results in differentiation into one or more cells, e.g., cells in the myeloid and/or cells in the lymphoid lineage, e.g., as shown in Example 3.
   52. The method of any one of embodiments 39-51, wherein the modified HSPC that has one, two, three, four, five, six, seven or all of the following expression characteristics:
i. expression of CD45, e.g., detectable expression of CD45, e.g., cell surface expression of CD45;
ii. expression of CD34, e.g., detectable expression of CD34, e.g., cell surface expression of CD34;
iii. expression of CD38, e.g., detectable expression of CD38, e.g., cell surface expression of CD38;
iv. expression of CD90 e.g., detectable expression of CD90, e.g., cell surface expression of CD90;
v. expression of CD133 e.g., detectable expression of CD133, e.g., cell surface expression of CD133;
vi. expression of CD45RA, e.g., detectable expression of CD45RA, e.g., cell surface expression of CD45RA;
vii. no detectable or low expression of markers associated with primitive progenitor cells, e.g., CMP, MEP, GMP and/or CLP;
viii. no detectable or low expression of markers associated with lineage committed cells, e.g., TCP, NKP, GP, MP, EP and/or MkP; or
ix. no detectable or low expression of markers associated with one, two or all cell lineage markers of (vii)-(viii), e.g., lineage negative (Lin-).
   53. The method of embodiment 52, wherein the human HSPC has any one of (i)-(vi).
   54. The method of embodiment 52, wherein the human HSPC has any two of (i)-(vi).
   55. The method of embodiment 52, wherein the human HSPC has any three of (i)-(vi).
   56. The method of embodiment 52, wherein the human HSPC has all of (i)-(vi).
   57. The method of any one of the embodiments 52-56, wherein the human HSPC has no detectable or low expression of (vii) or (viii).
   58. The method of any one of the embodiments 52-57, wherein the human HSPC has no detectable or low expression of both (vii) and (viii), e.g., wherein the human HSPC is a lineage negative HSPC.
   59. The method of any one of embodiments 39-58, wherein the modified HSPC has any one, or all, or a combination of the functional characteristics of embodiment 40 and the modified HSPC has any one, or all, or a combination of the expression characteristics of embodiment 52.
   60. The method of any one of the preceding embodiments, wherein the LNP composition comprising the payload modifies, e.g., increases or decreases, a genotype, a phenotype, and/or a function of the cell or tissue, resulting in a modified cell, e.g., modified HSPC, or tissue.
   61. The method of any one of the preceding embodiments, wherein the LNP composition comprising the payload modifies, e.g., increases or decreases, the component or parameter of the cell or tissue, resulting in a modified cell, e.g., modified HSPC, or tissue.
   62. The method of any one of the preceding embodiments, wherein the payload comprises a nucleic acid molecule, a protein, polypeptide or peptide molecule, a lipid molecule, a low molecular weight molecule, or a combination thereof.
   63. The method of embodiment 62, wherein the payload comprises a nucleic acid molecule comprising a DNA molecule, e.g., double stranded DNA; single stranded DNA; plasmid DNA.
   64. The method of embodiment 62 or 63, wherein the payload comprises a nucleic acid molecule comprising an RNA molecule, e.g., mRNA, rRNA, tRNA, regulatory RNA, non-coding RNA, long non-coding RNA (lncRNA), guide RNA (gRNA), piwi-interacting RNA (piRNA), small nucleolar RNA (snoRNA), small nuclear RNA (snRNA), extracellular RNA (exRNA), small Cajal body-specific RNA (scaRNA), microRNA (miRNA), circular RNA, or an RNAi molecule, e.g., small interfering (siRNA) or small hairpin RNA (shRNA).
   65. The method of embodiment 62 or 63, wherein the payload comprises an mRNA.
   66. The method of embodiment 65, wherein the mRNA comprises at least one chemical modification.
   67. The method of embodiment 66, wherein the chemical modification is selected from the group consisting of pseudouridine, N1-methylpseudouridine, 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methyluridine, 5-methoxyuridine, and 2′-O-methyl uridine.
   68. The method of embodiment 66, wherein the chemical modification is selected from the group consisting of pseudouridine, N1-methylpseudouridine, 5-methylcytosine, 5-methoxyuridine, and a combination thereof.
   69. The method of embodiment 66, wherein the chemical modification is N1-methylpseudouridine.
   70. The method of any of embodiments 65-69, wherein the mRNA comprises fully modified N1-methylpseudouridine.
   71. The method of any of embodiments 62-70, wherein the payload comprises a protein, polypeptide, or peptide molecule.
   72. The method of any of embodiments 62-71, wherein the payload comprises a lipid molecule, e.g., as described herein.
   73. The method of any of embodiments 62-72, wherein the payload comprises a low molecular weight molecule, e.g., as described herein.
   74. The method of any one of the preceding embodiments, wherein the payload comprises a genetic modulator (e.g., a modulator that genetically alters the cell or tissue); an epigenetic modulator (e.g., a modulator that epigenetically alters the cell or tissue); an RNA modulator (e.g., a modulator that alters an RNA molecule in the cell or tissue); a peptide modulator (e.g., a modulator that alters a peptide molecule in the cell or tissue); a lipid modulator (e.g., a modulator that alters a lipid molecule in the cell or tissue); or a combination thereof.
   75. The method of any one of the preceding embodiments, wherein the payload comprises a genetic modulator (e.g., a modulator that genetically alters the cell or tissue).
   76. The method of embodiment 74 or 75, wherein the genetic modulator comprises a system which modifies a nucleic acid sequence in a DNA molecule, e.g., by altering a nucleobase, e.g., introducing an insertion, a deletion, a mutation (e.g., a missense mutation, a silent mutation or a nonsense mutation), a duplication, or an inversion, or any combination thereof.
   77. The method of any one of embodiments 74-76, wherein the genetic modulator comprises a DNA base editor, a CRISPR/Cas gene editing system, a zinc finger nuclease (ZFN) system, a transcription activator-like effector nuclease (TALEN) system, a meganuclease system, or a transposase system, or any combination thereof, e.g., a combination of a CRISPR/Cas gene editing system and a transposase system.
   78. The method of any one of embodiments 74-77, wherein the genetic modulator comprises a template DNA.
   79. The method of any one of embodiments 74-77, wherein the genetic modulator does not comprise a template DNA.
   80. The method of any one of embodiments 74-79, wherein the genetic modulator comprises a template RNA.
   81. The method of any one of embodiments 74-79, wherein the genetic modulator does not comprise a template RNA.
   82. The method of any one of embodiments 74-81, wherein the genetic modulator comprises a CRISPR/Cas gene editing system.
   83. The method of embodiment 82, wherein the CRISPR/Cas gene editing system comprises a guide RNA (gRNA) molecule comprising a targeting sequence specific to a sequence of a target gene and a peptide having nuclease activity, e.g., endonuclease activity, e.g., a Cas protein or a fragment (e.g., biologically active fragment) or a variant thereof, e.g., a Cas9 protein, a fragment (e.g., biologically active fragment) or a variant thereof, a Cas3 protein, a fragment (e.g., biologically active fragment) or a variant thereof, a Cas12a protein, a fragment (e.g., biologically active fragment) (e.g., biologically active fragment) or a variant thereof, a Cas 12e protein, a fragment (e.g., biologically active fragment) or a variant thereof; a Cas 13 protein, a fragment (e.g., biologically active fragment) or a variant thereof, or a Cas14 protein, a fragment (e.g., biologically active fragment) or a variant thereof.
   84. The method of embodiment 82 or 83, wherein the CRISPR/Cas gene editing system comprises a gRNA molecule comprising a targeting sequence specific to a sequence of a target gene, and a nucleic acid encoding a peptide having nuclease activity, e.g., endonuclease activity, e.g., a Cas protein or a fragment (e.g., biologically active fragment) or variant thereof, e.g., a Cas9 protein, a fragment (e.g., biologically active fragment) or a variant thereof, a Cas3 protein, a fragment (e.g., biologically active fragment) or a variant thereof, a Cas12a protein, a fragment (e.g., biologically active fragment) or a variant thereof, a Cas 12e protein, a fragment (e.g., biologically active fragment) or a variant thereof, a Cas 13 protein, a fragment (e.g., biologically active fragment) or a variant thereof; or a Cas14 protein, a fragment (e.g., biologically active fragment) or a variant thereof.
   85. The method of embodiment 82 or 83, wherein the CRISPR/Cas gene editing system comprises a nucleic acid encoding a gRNA molecule comprising a targeting sequence specific to a sequence of a target gene, and a Cas9 protein, a fragment (e.g., biologically active fragment) or a variant thereof.
   86. The method of embodiment 82 or 83, wherein the CRISPR/Cas gene editing system comprises a nucleic acid encoding a gRNA molecule comprising a targeting sequence specific to a sequence of a target gene, and a nucleic acid encoding a Cas9 protein, a fragment (e.g., biologically active fragment) or a variant thereof.
   87. The method of any one of embodiments 82-86, wherein the CRISPR/Cas gene editing system further comprises a template DNA.
   88. The method of any one of embodiments 82-87, wherein the CRISPR/Cas gene editing system further comprises a template RNA.
   89. The method of any one of embodiments 82-88, wherein the CRISPR/Cas gene editing system further comprises a reverse transcriptase.
   90. The method of any one of embodiment 74-81, wherein the genetic modulator comprises a zinc finger nuclease (ZFN) system.
   91. The method of embodiment 90, wherein the ZFN system comprises a peptide having: a zinc finger DNA binding domain, a fragment (e.g., biologically active fragment) or a variant thereof, and/or nuclease activity, e.g., endonuclease activity.
   92. The method of embodiment 90 or 91, wherein the ZFN system comprises a peptide having a zinc finger DNA binding domain.
   93. The method of embodiment 92, wherein the zinc finger binding domain comprises 1, 2, 3, 4, 5, 6, 7, 8 or more zinc fingers, e.g., 3 or 6 zinc fingers.
   94 The method of any one of embodiments 90-93, wherein the ZFN system comprises a peptide having nuclease activity, e.g., endonuclease activity.
   95. The method of embodiment 94, wherein the peptide having nuclease activity is a type-II restriction 1-like endonuclease, e.g., a FokI endonuclease.
   96. The method of embodiment 90, wherein the ZFN system comprises a nucleic acid encoding a peptide having: a zinc finger DNA binding domain, a fragment (e.g., biologically active fragment) or a variant thereof; and/or nuclease activity, e.g., endonuclease activity.
   97. The method of embodiment 96, wherein the ZFN system comprises a nucleic acid encoding a peptide having a zinc finger DNA binding domain.
   98. The method of embodiment 97, wherein the zinc finger binding domain comprises 1, 2, 3, 4, 5, 6, 7, 8 or more zinc fingers, e.g., 3 or 6 zinc fingers.
   99. The method of embodiment 96 or 97, wherein the ZFN system comprises a nucleic acid encoding a peptide having nuclease activity, e.g., endonuclease activity.
   100. The method of embodiment 99, wherein the peptide having nuclease activity is a type-II restriction 1-like endonuclease, e.g., a FokI endonuclease.
   101. The method of any one of embodiments 90-100, wherein the ZFN system further comprises a template, e.g., template DNA.
   102. The method of embodiment 74-81, wherein the genetic modulator is a transcription activator-like effector nuclease (TALEN) system.
   103. The method of embodiment 102, wherein the TALEN system comprises a peptide having: a transcription activator-like (TAL) effector DNA binding domain, a fragment (e.g., biologically active fragment) or a variant thereof; and/or nuclease activity, e.g., endonuclease activity.
   104. The method of embodiment 102 or 103, wherein the TALEN system comprises a peptide having a TAL effector DNA binding domain, a fragment (e.g., biologically active fragment) or a variant thereof.
   105. The method of embodiment 102 or 103, wherein the TALEN system comprises a peptide having nuclease activity, e.g., endonuclease activity.
   106. The method of embodiment 105, wherein the peptide having nuclease activity is a type-II restriction 1-like endonuclease, e.g., a FokI endonuclease.
   107. The method of embodiment 102, wherein the TALEN system comprises a nucleic acid encoding a peptide having: a transcription activator-like (TAL) effector DNA binding domain, a fragment (e.g., biologically active fragment) or a variant thereof; and/or nuclease activity, e.g., endonuclease activity.
   108. The method of embodiment 107, wherein the TALEN system comprises a nucleic acid encoding a peptide having a transcription activator-like (TAL) effector DNA binding domain, a fragment (e.g., biologically active fragment) or a variant thereof.
   109. The method of embodiment 107, wherein the TALEN system comprises a nucleic acid encoding a peptide having nuclease activity, e.g., endonuclease activity.
   110. The method of embodiment 109, wherein the peptide having nuclease activity is a type-II restriction 1-like endonuclease, e.g., a FokI endonuclease.
   111. The method of any one of embodiments 102-110, wherein the TALEN system further comprises a template, e.g., a template DNA.
   112. The method of any one of embodiments 74-81, wherein the genetic modulator comprises a meganuclease system.
   113. The method of embodiment 112, wherein the meganuclease system comprises a peptide having a DNA binding domain and nuclease activity, e.g., a homing endonuclease.
   114. The method of embodiment 113, wherein the homing endonuclease comprises a LAGLIDADG endonuclease (SEQ ID NO: 270), GIY-YIG endonuclease, HNH endonuclease, His-Cys box endonuclease or a PD-(D/E)XK endonuclease, or a fragment (e.g., biologically active fragment) or variant thereof, e.g., as described in Silva G. et al, (2011) Curr Gene Therapy 11(1): 11-27.
   115. The method of embodiment 112, wherein the meganuclease system comprises a nucleic acid encoding a peptide having a DNA binding domain and nuclease activity, e.g., a homing endonuclease.
   116. The method of embodiment 115, wherein the homing endonuclease comprises a LAGLIDADG endonuclease (SEQ ID NO: 270), GIY-YIG endonuclease, HNH endonuclease, His-Cys box endonuclease or a PD-(D/E)XK endonuclease, or a fragment (e.g., biologically active fragment) or variant thereof, e.g., as described in Silva G. et al, (2011) Curr Gene Therapy 11(1): 11-27.
   117. The method of any one of embodiments 112-116, wherein the system further comprises a template, e.g., a template DNA.
   118. The method of any one of embodiments 74-117, wherein the genetic modulator comprises a transposase system.
   119. The method of embodiment 118, wherein the transposase system comprises a nucleic acid sequence encoding a peptide having reverse transcriptase and/or nuclease activity, e.g., a retrotransposon, e.g., an LTR retrotransposon or a non-LTR retrotransposon.
   120. The method of embodiment 118 or 119, wherein the transposase system comprises a template, e.g., an RNA template.
   121. The method of any one of the preceding embodiments, wherein the payload comprises an epigenetic modulator (e.g., a modulator that epigenetically alters the cell or tissue).
   122. The method of embodiment 121, wherein the epigenetic modulator comprises a molecule that modifies chromatin architecture, methylates DNA, and/or modifies a histone.
   123. The method of embodiment 121 or 122, wherein the epigenetic modulator comprises a molecule that modifies chromatin architecture, e.g., a SWI/SNF remodeling complex or a component thereof.
   124. The method of any one of embodiments 121-123, wherein the epigenetic modulator comprises a molecule that methylates DNA, e.g., a DNA methyltransferase, a fragment (e.g., biologically active fragment) or variant thereof (e.g., DNMT1, DNMT2 DNMT3A, DNMT3B, DNMT3L, or M. SssI); a polycomb repressive complex or a component thereof, e.g., PRC1 or PRC2, or PR-DUB, or a fragment (e.g., biologically active fragment) or a variant thereof; a demethylase, or a fragment (e.g., biologically active fragment) or a variant thereof (e.g., Tet1, Tet2 or Tet3).
   125. The method of any one of embodiments 121-124, wherein the epigenetic modulator comprises a molecule that modifies a histone, e.g., methylates and/or acetylates a histone, e.g., a histone modifying enzyme or a fragment (e.g., biologically active fragment) or a variant thereof, e.g., HMT, HDM, HAT, or HDAC.
   126. The method of any one of the preceding embodiments, wherein the payload comprises an RNA modulator (e.g., a modulator that alters an RNA molecule in the cell or tissue).
   127. The method of embodiment 126, wherein the RNA modulator comprises a molecule that alters the expression and/or activity; stability or compartmentalization of an RNA molecule.
   128. The method of embodiment 126 or 127, wherein the RNA modulator comprises an RNA molecule, e.g., mRNA, rRNA, tRNA, regulatory RNA, non-coding RNA, long non-coding RNA (lncRNA), guide RNA (gRNA), piwi-interacting RNA (piRNA), small nucleolar RNA (snoRNA), small nuclear RNA (snRNA), extracellular RNA (exRNA), small Cajal body-specific RNA (scaRNA), microRNA (miRNA), circular RNA, or an RNAi molecule, e.g., small interfering RNA (siRNA) or small hairpin RNA (shRNA).
   129. The method of any of embodiments 126-128, wherein the RNA modulator comprises a DNA molecule.
   130. The method of any of embodiments 126-129, wherein the RNA modulator comprises a low molecular weight molecule.
   131. The method of any of embodiments 126-130, wherein the RNA modulator comprises a peptide, e.g., an RNA binding protein, a fragment (e.g., biologically active fragment), or a variant thereof; or an enzyme, or a fragment (e.g., biologically active fragment) or variant thereof.
   132. The method of any of embodiments 126-131, wherein the RNA modulator comprises an RNA base editor system.
   133. The method of embodiment 132, wherein the RNA base editor system comprises: a deaminase, e.g., an RNA-specific adenosine deaminase (ADAR); a Cas protein, a fragment (e.g., biologically active fragment) or a variant thereof, and/or a guide RNA.
   134. The method of embodiment 132 or 133, wherein the RNA base editor system further comprises a template, e.g., a DNA or RNA template.
   135. The method of any one of the preceding embodiments, wherein payload comprises a peptide modulator (e.g., a modulator that alters a peptide molecule in the cell or tissue).
   136. The method of any one of the preceding embodiments, wherein the payload comprises a lipid modulator (e.g., a modulator that alters a lipid molecule in the cell or tissue); or a combination thereof.
   137. The method of any one of the preceding embodiments, wherein the payload comprises a therapeutic payload or a prophylactic payload.
   138. The method of embodiment 137, wherein the therapeutic payload or prophylactic payload comprises a secreted protein, a membrane-bound protein, or an intercellular protein; or an mRNA encoding a secreted protein, a membrane-bound protein; or an intercellular protein.
   139. The method of embodiment 137 or 138, wherein the therapeutic payload or prophylactic payload comprises a protein, polypeptide, or peptide.
   140. The method of any one of the preceding embodiments, wherein the LNP does not include an additional targeting moiety, e.g., it transfects (e.g., at least 10%, 20%, 30%, 40%, 50%, 60%, 70%, 80%, 90%, or 95%) of stem or progenitor cells (e.g., HSPCs) without an additional targeting moiety.
   141. The method of any one of the preceding embodiments, wherein the subject has a disease or disorder selected from the group consisting of a hemoglobinopathy, a clotting factor disorder, a blood cell disorder, and an immune cell disorder.
   142. The method of embodiment of 141, wherein the subject has a mutation in a hemoglobulin gene and/or has an aberrant expression of a hemoglobulin gene.
   143. The method of embodiment of 141, wherein the subject has a mutation in a gene encoding a clotting factor, and/or has an aberrant expression of a gene encoding a clotting factor.
   144. The method of any one of the preceding embodiments, wherein the subject is a mammal, e.g., human.
   145. An LNP composition for use in the method of any one of the preceding embodiments.
   146. A pharmaceutical composition comprising the LNP composition of embodiment 145.
   147. The LNP composition of embodiment 145, or pharmaceutical composition of embodiment 146, wherein the LNP composition comprises: (i) an ionizable lipid, e.g., an amino lipid; (ii) a sterol or other structural lipid; (iii) a non-cationic helper lipid or phospholipid; and (iv) a PEG-lipid.
   148. The LNP composition or pharmaceutical composition of embodiment 147, wherein the ionizable lipid comprises an amino lipid.
   149. The LNP composition or pharmaceutical composition of embodiment 148, wherein the ionizable lipid comprises a compound of any of Formulae (I), (I-I), (I-II), (I-III), (I-IV), (Ia), (Ib), (Ic), (II), or (II-I).
   150. The LNP composition or pharmaceutical composition of embodiment 147, wherein the ionizable lipid comprises a compound of Formula (I).
   151. The LNP composition or pharmaceutical composition of embodiment 147, wherein the ionizable lipid comprises a compound of Formula (Ia).
   152. The LNP composition or pharmaceutical composition of embodiment 147, wherein the ionizable lipid comprises a compound of Formula (Ib).
   153. The LNP composition or pharmaceutical composition of embodiment 147, wherein the ionizable lipid comprises a compound of Formula (Ic).
   154. The LNP composition or pharmaceutical composition of embodiment 147, wherein the ionizable lipid comprises a compound of Formula (I-I).
   155. The LNP composition or pharmaceutical composition of embodiment 147, wherein the ionizable lipid comprises a compound of Formula (I-II).
   156. The LNP composition or pharmaceutical composition of embodiment 147, wherein the ionizable lipid comprises a compound of Formula (I-III).
   157. The LNP composition or pharmaceutical composition of embodiment 147, wherein the ionizable lipid comprises a compound of Formula (I-IV).
   158. The LNP composition or pharmaceutical composition of embodiment 147, wherein the ionizable lipid comprises a compound of Formula (II).
   159. The LNP composition or pharmaceutical composition of embodiment 147, wherein the ionizable lipid comprises a compound of Formula (II-I).
   160. The LNP composition or pharmaceutical composition of embodiment 147, wherein the non-cationic helper lipid or phospholipid comprises a compound selected from the group consisting of DSPC, DPPC, or DOPC.
   161. The LNP composition or pharmaceutical composition of embodiment 147, wherein the phospholipid is DSPC, e.g., a variant of DSPC, e.g., a compound of Formula (IV).
   162. The LNP composition or pharmaceutical composition of embodiment 147, wherein the structural lipid is chosen from alpha-tocopherol, β-sitosterol or cholesterol.
   163. The LNP composition or pharmaceutical composition of embodiment 147, wherein the structural lipid is alpha-tocopherol.
   164. The LNP composition or pharmaceutical composition of embodiment 147, wherein the structural lipid is β-sitosterol.
   165. The LNP composition or pharmaceutical composition of embodiment 147, wherein the structural lipid is cholesterol.
   166. The LNP composition or pharmaceutical composition of embodiment 147, wherein the PEG lipid is selected from the group consisting of a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof.
   167. The LNP composition or pharmaceutical composition of embodiment 147, wherein the PEG lipid is selected from the group consisting of PEG-c-DOMG, PEG-DMG, PEG-DLPE, PEG-DMPE, PEG-DPPC and PEG-DSPE lipid.
   168. The LNP composition or pharmaceutical composition of embodiment 147, wherein the PEG-lipid is PEG-DMG.
   169. The LNP composition or pharmaceutical composition of embodiment 147, wherein the PEG lipid is chosen from a compound of: Formula (V), Formula (VI-A), Formula (VI-B), Formula (VI-C) or Formula (VI-D).
   170. The LNP composition or pharmaceutical composition of embodiment 147, wherein the PEG lipid is a compound of Formula (VI-A).
   171. The LNP composition or pharmaceutical composition of embodiment 147, wherein the PEG lipid is a compound of Formula (VI-B).
   172. The LNP composition or pharmaceutical composition of embodiment 147, wherein the PEG lipid is a compound of Formula (VI-C).
   173. The LNP composition or pharmaceutical composition of embodiment 147, wherein the PEG lipid is a compound of Formula (VI-D).
   174. The LNP composition or pharmaceutical composition of embodiment 147, wherein the ionizable lipid comprises a compound of Formula (I-I), the phospholipid comprises DSPC, the structural lipid comprises cholesterol. and the PEG lipid comprises a compound of Formula (VI-D).
   175. The LNP composition or pharmaceutical composition of any one of embodiments 145-174, wherein the LNP comprises a molar ratio of about 20-60% ionizable lipid: 5-25% phospholipid: 25-55% cholesterol; and 0.5-15% PEG lipid.
   176. The LNP composition or pharmaceutical composition of embodiment 175, wherein the LNP comprises a molar ratio of about 50% ionizable lipid: about 10% phospholipid: about 38.5% cholesterol; and about 1.5% PEG lipid.
   177. The LNP composition or pharmaceutical composition of embodiment 175, wherein the LNP comprises a molar ratio of about 49.83% ionizable lipid: about 9.83% phospholipid: about 30.33% cholesterol; and about 2.0% PEG lipid.
   178. The LNP composition or pharmaceutical composition of any one of embodiments 145-177, wherein the LNP composition is formulated for intravenous, subcutaneous, intramuscular, intranasal, intraocular, or pulmonary delivery (e.g., single or repeat delivery).
   179. The LNP composition or pharmaceutical composition of any one of embodiments 144-178, wherein the LNP composition is formulated for intravenous delivery (e.g., single or repeat delivery).
   180. The LNP composition or pharmaceutical composition of any one of embodiments 144-179, wherein the LNP composition does not comprise an additional targeting moiety, e.g., it transfects (e.g., at least 10%, 20%, 30%, 40%, 50%, 60%, 70%, 80%, 90%, or 95%) of stem or progenitor cells (e.g., HSPCs) without an additional targeting moiety.
   181. A modified cell, e.g., a modified stem or progenitor cell, e.g., a modified HSPC (e.g., modified HSC or HPC), made according to a method of any one of embodiments 1-144, or by an LNP composition or pharmaceutical composition of any one of embodiments 145-180.
   182. A frozen preparation of a modified cell, e.g., a modified stem or progenitor cell, e.g., a modified HSPC (e.g., modified HSC or HPC), made according to a method of any one of embodiments 1-144, or by an LNP composition or pharmaceutical composition of any one of embodiments 145-180.
   183. The modified cell of 181, or frozen preparation of a modified cell of 182, for use in treating a subject having a disease or disorder.
   184. The modified cell of 181, or frozen preparation of a modified cell of 182, for use in ameliorating a symptom of a subject having a disease or disorder.
   185. The modified cell, or frozen preparation of a modified cell, for use of claim 183 or 184, wherein the disease or disorder is selected from the group consisting of a hemoglobinopathy, a clotting factor disorder, a blood cell disorder, or an immune cell disorder.
   186. The modified cell, or frozen preparation of a modified cell, for use of any one of claims 183-185, wherein the modified cell is autologous to the subject.
   187. The modified cell, or frozen preparation of a modified cell, for use of any one of claims 183-186, wherein the modified cell is allogeneic to the subject.
   188. A composition or reaction mixture comprising:
(a) a population of stem or progenitor cells, e.g., HSPCs (e.g., a population of HSCs, HPCs, or a combination thereof); and
(b) an LNP composition comprising a payload which can modify the stem or progenitor cell, e.g., a component associated with the stem cell or a parameter associated with the stem or progenitor cell, e.g., as described herein.
   189. A pharmaceutical composition comprising a modified cell, e.g., modified HSPC (e.g., modified HSC or HPC), and an LNP comprising a payload which can modify the cell, e.g., as described herein.
   190. The composition or reaction mixture of claim 188 or the pharmaceutical composition of claim 189, wherein the LNP composition does not comprise an additional targeting moiety, e.g., it transfects (e.g., at least 10%, 20%, 30%, 40%, 50%, 60%, 70%, 80%, 90%, or 95%) of stem or progenitor cells (e.g., HSPCs) without an additional targeting moiety.
   191. A kit comprising a modified cell, e.g., modified HSPC (e.g., modified HSC or HPC), and an LNP comprising a payload which can modify the cell, e.g., as described herein.
   192. An LNP composition comprising a payload, wherein, when contacted with a cell (e.g., a stem or progenitor cell), e.g., in a subject (e.g., a subject having a disease, a disorder, a mutation, or a single nucleotide polymorphism (SNP)), the LNP composition results in a modification of the cell, e.g., modification of a parameter associated with the cell or a component associated with the cell, optionally, wherein the LNP composition does not comprise an additional targeting moiety.
   193. An LNP composition comprising a payload, wherein, when administered to a subject (e.g., a subject having a disease, a disorder, a mutation, or a single nucleotide polymorphism (SNP)), the LNP composition results in a modification of a cell (e.g., a stem or progenitor cell), e.g., modification of a parameter associated with the cell or a component associated with the cell, optionally, wherein the LNP composition does not comprise an additional targeting moiety, e.g., it transfects (e.g., at least 10%, 20%, 30%, 40%, 50%, 60%, 70%, 80%, 90%, or 95%) of stem or progenitor cells (e.g., HSPCs) without an additional targeting moiety.
   194. The LNP composition of embodiment 192 or 193, wherein the disease or disorder is selected from the group consisting of a hemoglobinopathy, a clotting factor disorder, a blood cell disorder, and an immune cell disorder.
   195. The LNP composition of any one of embodiments 192-194, wherein the mutation or SNP is associated with, or causes, a disease or disorder selected from the group consisting of a hemoglobinopathy, a clotting factor disorder, a blood cell disorder, and an immune cell disorder.
   196. The LNP composition of any one of embodiments 192-195, wherein the payload alters (e.g., ameliorates) the disease or disorder, e.g., a disease or disorder selected from the group consisting of a hemoglobinopathy, a clotting factor disorder, a blood cell disorder, and an immune cell disorder.
   197. The LNP composition of any one of embodiments 192-196, comprising an amino lipid comprising a compound of Formula (I-I), a phospholipid comprising DSPC, a structural lipid comprising cholesterol, and a PEG lipid comprising a compound of Formula (VI-D).
   198. The LNP composition of any one of embodiments 192-197, wherein the LNP composition results in a modification of a genotype, a phenotype, and/or a function of the cell or tissue.
   199. The LNP composition of any one of embodiments 192-198, wherein the component comprises: (1) a nucleic acid associated with the cell or a fragment thereof, e.g., a DNA (e.g., exonic, intronic, intergenic, telomeric, promoter, enhancer, insulator, repressor, coding, or non-coding) or an RNA (e.g., mRNA, rRNA, tRNA, regulatory RNA, non-coding RNA, long non-coding RNA (lncRNA), guide RNA (gRNA), piwi-interacting RNA (piRNA), small nucleolar RNA (snoRNA), small nuclear RNA (snRNA), extracellular RNA (exRNA), small Cajal body-specific RNA (scaRNA), microRNA (miRNA), circular RNA, or an RNAi molecule, e.g., small interfering RNA (siRNA) or small hairpin RNA (shRNA)); (2) a peptide or protein associated with the cell or a fragment thereof; (3) a lipid component associated with the cell or a fragment thereof, or a combination thereof.
   200. The LNP composition of any one of embodiments 192-199, wherein the component comprises a DNA.
   201. The LNP composition of any one of embodiments 192-200, wherein the component comprises an RNA.
   202. The LNP composition of any one of embodiments 192-201, wherein the component comprises a peptide or protein associated with the cell or fragment thereof.
   203. The LNP composition of any one of embodiments 192-202, wherein the component comprises a lipid component associated with the cell or fragment thereof.
   204. The LNP composition of any one of embodiments 192-203, wherein the component is endogenous to the cell.
   205. The LNP composition of any one of embodiments 192-204, wherein the component is exogenous to the cell, e.g., has been introduced into the cell by a method known in the field, e.g., transformation, electroporation, viral based delivery or lipid-based delivery.
   206. The LNP composition of any one of embodiments 192-205, wherein the parameter comprises a genotypic parameter, a phenotypic parameter, a functional parameter, an expression parameter, a signaling parameter, or any combination thereof.
   207. The LNP composition of embodiment 206, wherein the genotypic parameter comprises a genotype of the cell, e.g., the presence or absence a gene or allele, or a modification of a gene or allele, e.g., a germline or somatic mutation, or a polymorphism, in the gene or allele.
   208. The LNP composition of embodiment 206 or 207, wherein the phenotypic parameter comprises a phenotype of the cell, e.g., expression and/or activity of a molecule, e.g., cell surface protein, lipid or adhesion molecule, on the surface of the cell.
   209. The LNP composition of any one of embodiments 206-208, wherein the functional parameter comprises a function of the cell, e.g., the ability of the cell to produce a gene product (e.g., a protein), the ability of the cell to proliferate, divide, and/or renew, and/or the ability of the cell to differentiate, e.g., into one or more cell types in a lineage.
   210. The LNP composition of any one of embodiments 206-209, wherein the expression parameter comprises one, two, three, four or all of the following:
(a) expression level (e.g., of polypeptide or protein, or nucleic acid (e.g., mRNA));
(b) activity (e.g., of polypeptide or protein, or nucleic acid (e.g., mRNA)),
(c) post-translational modification of polypeptide or protein;
(d) folding (e.g., of polypeptide or protein, or nucleic acid (e.g., mRNA)), and/or
(e) stability (e.g., of polypeptide or protein, or nucleic acid (e.g., mRNA)).
   211. The LNP composition of any one of embodiments 206-210, wherein the signaling parameter comprises one, two, three, four or all of the following:
(1) modulation of a signaling pathway, e.g., a cellular signaling pathway;
(2) cell fate modulation;
(3) modulation of expression level (e.g., of polypeptide or protein, or nucleic acid (e.g., mRNA));
(4) modulation of activity (e.g., of polypeptide or protein, or nucleic acid (e.g., mRNA)), and/or
(5) modulation of stability e.g., of polypeptide or protein, or nucleic acid (e.g., mRNA)).
   212. The LNP composition of any one of embodiments 192-211, wherein the cell is contacted in vitro, in vivo, or ex vivo with the LNP composition.
   213. The LNP composition of any one of embodiments 192-212, wherein the cell is contacted in vivo with the LNP formulation.
   214. The LNP composition of any one of embodiments 192-213, wherein the cell is contacted ex vivo with the LNP formulation.
   215. The LNP composition of any one of embodiments 192-214, wherein the cell is a stem or progenitor cell, e.g., a hematopoietic stem and progenitor cell (HSPC), e.g., an HSPC derived from an embryonic stem or progenitor cell or an HSPC derived from an induced pluripotent stem or progenitor cell.
   216. The LNP composition of any one of embodiments 192-215, wherein the cell is a common myeloid progenitor cell, a common lymphoid progenitor cell, a multipotent progenitor cell, or a multipotent stem cell.
   217. The LNP composition of any one of embodiments 192-216, wherein the cell is an HSPC, e.g., a multipotent HSC or multipotent HPC.
   218. The LNP composition of embodiment 217, wherein the HSPC has one, two, three, four, five or all of the following functional characteristics:
i. ability to self-renew;
ii. unlimited proliferative potential;
iii. ability to enter and/or exit a quiescent state, e.g., a cell state where no proliferation occurs, e.g., GO phase of the cell cycle;
iv. ability to differentiate into any hematopoietic lineage, e.g., myeloid and/or lymphoid lineages, e.g., common lymphoid progenitor (CLP) or a differentiated cell thereof, and/or common myeloid progenitor (CMP) or a differentiated cell thereof;
v. ability to repopulate any hematopoietic lineage, e.g., myeloid and/or lymphoid lineages, e.g., common lymphoid progenitor (CLP) or a differentiated cell thereof, and/or common myeloid progenitor (CMP) or a differentiated cell thereof; e.g., in an organism; or
vi. ability to form colony forming units (CFU).
   219. The LNP composition of embodiment 217 or 218, wherein the HSPC that has one, two, three, four, five, six, seven, eight or all of the following expression characteristics:
i. expression of CD45, e.g., detectable expression of CD45, e.g., cell surface expression of CD45;
ii. expression of CD34, e.g., detectable expression of CD34, e.g., cell surface expression of CD34;
iii. expression of CD38, e.g., detectable expression of CD38, e.g., cell surface expression of CD38;
iv. expression of CD90 e.g., detectable expression of CD90, e.g., cell surface expression of CD90;
v. expression of CD133 e.g., detectable expression of CD133, e.g., cell surface expression of CD133;
vi. expression of CD45RA, e.g., detectable expression of CD45RA, e.g., cell surface expression of CD45RA;
vii. no detectable or low expression of markers associated with primitive progenitor cells, e.g., CMP, MEP, GMP and/or CLP;
viii. no detectable or low expression of markers associated with lineage committed cells, e.g., TCP, NKP, GP, MP, EP and/or MkP; or
ix. no detectable or low expression of markers associated with one, two or all cell lineage markers of (vii)-(viii), e.g., lineage negative (Lin-).
   220. The LNP composition of embodiment 219, wherein the human HSPC has any one of (i)-(vi).
   221. The LNP composition of embodiment 219, wherein the human HSPC has any two of (i)-(vi).
   222. The LNP composition of embodiment 219, wherein the human HSPC has any three of (i)-(vi).
   223. The LNP composition of embodiment 219, wherein the human HSPC has all of (i)-(vi).
   224. The LNP composition of any one of the embodiments 219-223, wherein the human HSPC has no detectable or low expression of (vii) or (viii).
   225. The LNP composition of any one of the embodiments 219-224, wherein the human HSPC has no detectable or low expression of both (vii) and (viii), e.g., wherein the human HSPC is a lineage negative HSPC.
   226. The LNP composition of embodiment 218 or 219, wherein the HSPC has any one, or all, or a combination of the functional characteristics of embodiment 28 and the HSPC has any one, or all, or a combination of the expression characteristics of embodiment 29.
   227. The LNP composition of any one of embodiments 192-226, wherein prior to contacting the cell with the LNP composition, the cell (e.g., population of cells) is isolated from a subject and expanded, enriched and/or cultured in vitro.
   228. The LNP composition of any one of embodiments 192-227, wherein the expanded, enriched and/or cultured cell, e.g., population of cells, is administered into a host, e.g., an autologous or allogeneic host.
   229. The LNP composition of any one of embodiments 192-228, wherein the modified cell (e.g., population of modified cells) is a modified HSPC (e.g., a population of modified HSPCs).
   230. The LNP composition of embodiment 229, wherein the modified HSPC has one, two, three, four, five or all of the following functional characteristics:
i. ability to self-renew;
ii. unlimited proliferative potential;
iii. ability to enter and/or exit a quiescent state, e.g., a cell state where no proliferation occurs, e.g., GO phase of the cell cycle;
iv. ability to differentiate into any hematopoietic lineage, e.g., myeloid and/or lymphoid lineages, e.g., common lymphoid progenitor (CLP) or a differentiated cell thereof; and/or common myeloid progenitor (CMP) or a differentiated cell thereof;
v. ability to repopulate any hematopoietic lineage, e.g., myeloid and/or lymphoid lineages, e.g., common lymphoid progenitor (CLP) or a differentiated cell thereof, and/or common myeloid progenitor (CMP) or a differentiated cell thereof; e.g., in an organism; or
vi. ability to form colony forming units (CFU).
   231. The LNP composition of embodiment 229 or 230, wherein the modified HSPC has the ability to form CFU, e.g., as measured in an ex-vivo colony-forming unit (CFU) assay, e.g., as described in Example 2, or as measured in a lineage tracing experiment, e.g., as described in Example 3, e.g., as compared to an otherwise similar HSPC which has not been contacted with an LNP, or has been contacted with a different LNP.
   232. The LNP composition of any one of embodiments 229-231, wherein the modified HSPC has the ability to differentiate into myeloid cells, e.g., as measured in an ex-vivo colony-forming unit (CFU) assay, e.g., as described in Example 2, or as measured in a lineage tracing experiment, e.g., as described in Example 3, e.g., as compared to an otherwise similar HSC which has not been contacted with an LNP, or has been contacted with a different LNP.
   233. The LNP composition of any one of embodiments 229-232, wherein the modified HSPC has the ability to differentiate into lymphoid cells, e.g., as measured in lineage tracing experiments, e.g., as described in Example 3, e.g., as compared to an otherwise similar HSC which has not been contacted with an LNP, or has been contacted with a different LNP.
   234. The LNP composition of any one of embodiments 229-233, wherein the modified HSPC has the ability to differentiate into an erythrocyte cell or a platelet, e.g., as shown in Example 3, e.g., as compared to an otherwise similar HSPC which has not been contacted with an LNP, or has been contacted with a different LNP.
   235. The LNP composition of embodiment 234, wherein the modified HSPC differentiates into an erythrocyte cell or a platelet in vivo.
   236. The LNP composition of embodiment 234, wherein the modified HSPC differentiates into an erythrocyte cell or a platelet in vitro.
   237. The LNP composition of any one of embodiments 229-236, wherein the modified HSPC has the ability to differentiate into a neutrophil, a monocyte, a B cell, or a T cell (e.g., a CD4+ T cell or a CD8+ T cell), e.g., as shown in Example 3, e.g., as compared to an otherwise similar HSPC which has not been contacted with an LNP, or has been contacted with a different LNP.
   238. The LNP composition of embodiment 237, wherein the modified HSPC differentiates into a neutrophil, a monocyte, a B cell, or a T cell (e.g., a CD4+ T cell or a CD8+ T cell) in vivo.
   239. The LNP composition of embodiment 238, wherein the modified HSPC differentiates into a neutrophil, a monocyte, a B cell, or a T cell (e.g., a CD4+ T cell or a CD8+ T cell) in vitro.
   240. The LNP composition of any of embodiments 229-239, wherein the modified HSPC persists, e.g., in vivo, for at least 1, 2, 3, 4, 5, 6, 7, 10, 15, 20, 25, 30, 45, 60, 90, 120, 180, 240, 300, or 365 days or more.
   241. The LNP composition of embodiment 240, wherein the in vivo persistence of the modified HSPC results in differentiation into one or more cells, e.g., cells in the myeloid and/or cells in the lymphoid lineage, e.g., as shown in Example 3.
   242. The LNP composition of any one of embodiments 229-241, wherein the modified HSPC that has one, two, three, four, five, six, seven or all of the following expression characteristics:
i. expression of CD45, e.g., detectable expression of CD45, e.g., cell surface expression of CD45;
ii. expression of CD34, e.g., detectable expression of CD34, e.g., cell surface expression of CD34;
iii. expression of CD38, e.g., detectable expression of CD38, e.g., cell surface expression of CD38;
iv. expression of CD90 e.g., detectable expression of CD90, e.g., cell surface expression of CD90;
v. expression of CD133 e.g., detectable expression of CD133, e.g., cell surface expression of CD133;
vi. expression of CD45RA, e.g., detectable expression of CD45RA, e.g., cell surface expression of CD45RA;
vii. no detectable or low expression of markers associated with primitive progenitor cells, e.g., CMP, MEP, GMP and/or CLP;
viii. no detectable or low expression of markers associated with lineage committed cells, e.g., TCP, NKP, GP, MP, EP and/or MkP; or
ix. no detectable or low expression of markers associated with one, two or all cell lineage markers of (vii)-(viii), e.g., lineage negative (Lin-).
   243. The LNP composition of embodiment 242, wherein the human HSPC has any one of (i)-(vi).
   244. The LNP composition of embodiment 242, wherein the human HSPC has any two of (i)-(vi).
   245. The LNP composition of embodiment 242, wherein the human HSPC has any three of (i)-(vi).
   246. The LNP composition of embodiment 242, wherein the human HSPC has all of (i)-(vi).
   247. The LNP composition of any one of the embodiments 242-246, wherein the human HSPC has no detectable or low expression of (vii) or (viii).
   248. The LNP composition of any one of the embodiments 242-247, wherein the human HSPC has no detectable or low expression of both (vii) and (viii), e.g., wherein the human HSPC is a lineage negative HSPC.
   249. The LNP composition of any one of embodiments 229-248, wherein the modified HSPC has any one, or all, or a combination of the functional characteristics of embodiment 230 and the modified HSPC has any one, or all, or a combination of the expression characteristics of embodiment 242.
   250. The LNP composition of any one of embodiments 192-249, wherein the payload comprises a nucleic acid molecule, a protein, polypeptide or peptide molecule, a lipid molecule, a low molecular weight molecule, or a combination thereof.
   251. The LNP composition of embodiment 250, wherein the payload comprises a nucleic acid molecule comprising a DNA molecule, e.g., double stranded DNA; single stranded DNA; plasmid DNA.
   252. The LNP composition of embodiment 250 or 251, wherein the payload comprises a nucleic acid molecule comprising an RNA molecule, e.g., mRNA, rRNA, tRNA, regulatory RNA, non-coding RNA, long non-coding RNA (lncRNA), guide RNA (gRNA), piwi-interacting RNA (piRNA), small nucleolar RNA (snoRNA), small nuclear RNA (snRNA), extracellular RNA (exRNA), small Cajal body-specific RNA (scaRNA), microRNA (miRNA), circular RNA, or an RNAi molecule, e.g., small interfering (siRNA) or small hairpin RNA (shRNA).
   253. The LNP composition of embodiment 250 or 251, wherein the payload comprises an mRNA.
   254. The LNP composition of embodiment 253, wherein the mRNA comprises at least one chemical modification.
   255. The LNP composition of embodiment 254, wherein the chemical modification is selected from the group consisting of pseudouridine, N1-methylpseudouridine, 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methyluridine, 5-methoxyuridine, and 2′-O-methyl uridine.
   256. The LNP composition of embodiment 254, wherein the chemical modification is selected from the group consisting of pseudouridine, N1-methylpseudouridine, 5-methylcytosine, 5-methoxyuridine, and a combination thereof.
   257. The LNP composition of embodiment 254, wherein the chemical modification is N1-methylpseudouridine.
   258. The LNP composition of any of embodiments 253-257, wherein the mRNA comprises fully modified N1-methylpseudouridine.
   259. The LNP composition of any of embodiments 253-258, wherein the payload comprises a protein, polypeptide, or peptide molecule.
   260. The LNP composition of any of embodiments 253-259, wherein the payload comprises a lipid molecule, e.g., as described herein.
   261. The LNP composition of any of embodiments 253-260, wherein the payload comprises a low molecular weight molecule, e.g., as described herein.
   262. The LNP composition of any one of embodiments 192-261, wherein the payload comprises a genetic modulator (e.g., a modulator that genetically alters the cell or tissue); an epigenetic modulator (e.g., a modulator that epigenetically alters the cell or tissue); an RNA modulator (e.g., a modulator that alters an RNA molecule in the cell or tissue); a peptide modulator (e.g., a modulator that alters a peptide molecule in the cell or tissue); a lipid modulator (e.g., a modulator that alters a lipid molecule in the cell or tissue); or a combination thereof.
   263. The LNP composition of any one of embodiments 192-262, wherein the payload comprises a genetic modulator (e.g., a modulator that genetically alters the cell or tissue).
   264. The LNP composition of embodiment 262 or 263, wherein the genetic modulator comprises a system which modifies a nucleic acid sequence in a DNA molecule, e.g., by altering a nucleobase, e.g., introducing an insertion, a deletion, a mutation (e.g., a missense mutation, a silent mutation or a nonsense mutation), a duplication, or an inversion, or any combination thereof.
   265. The LNP composition of any one of embodiments 262-264, wherein the genetic modulator comprises a DNA base editor, a CRISPR/Cas gene editing system, a zinc finger nuclease (ZFN) system, a transcription activator-like effector nuclease (TALEN) system, a meganuclease system, or a transposase system, or any combination thereof, e.g., a combination of a CRISPR/Cas gene editing system and a transposase system.
   266. The LNP composition of any one of embodiments 262-265, wherein the genetic modulator comprises a template DNA.
   267. The LNP composition of any one of embodiments 262-265, wherein the genetic modulator does not comprise a template DNA.
   268. The LNP composition of any one of embodiments 262-267, wherein the genetic modulator comprises a template RNA.
   269. The LNP composition of any one of embodiments 262-267, wherein the genetic modulator does not comprise a template RNA.
   270. The LNP composition of any one of embodiments 262-269, wherein the genetic modulator comprises a CRISPR/Cas gene editing system.
   271. The LNP composition of embodiment 270, wherein the CRISPR/Cas gene editing system comprises a guide RNA (gRNA) molecule comprising a targeting sequence specific to a sequence of a target gene and a peptide having nuclease activity, e.g., endonuclease activity, e.g., a Cas protein or a fragment (e.g., biologically active fragment) or a variant thereof, e.g., a Cas9 protein, a fragment (e.g., biologically active fragment) or a variant thereof, a Cas3 protein, a fragment (e.g., biologically active fragment) or a variant thereof, a Cas12a protein, a fragment (e.g., biologically active fragment) (e.g., biologically active fragment) or a variant thereof; a Cas 12e protein, a fragment (e.g., biologically active fragment) or a variant thereof; a Cas 13 protein, a fragment (e.g., biologically active fragment) or a variant thereof; or a Cas14 protein, a fragment (e.g., biologically active fragment) or a variant thereof.
   272. The LNP composition of embodiment 270 or 271, wherein the CRISPR/Cas gene editing system comprises a gRNA molecule comprising a targeting sequence specific to a sequence of a target gene, and a nucleic acid encoding a peptide having nuclease activity, e.g., endonuclease activity, e.g., a Cas protein or a fragment (e.g., biologically active fragment) or variant thereof, e.g., a Cas9 protein, a fragment (e.g., biologically active fragment) or a variant thereof, a Cas3 protein, a fragment (e.g., biologically active fragment) or a variant thereof, a Cas12a protein, a fragment (e.g., biologically active fragment) or a variant thereof, a Cas 12e protein, a fragment (e.g., biologically active fragment) or a variant thereof; a Cas 13 protein, a fragment (e.g., biologically active fragment) or a variant thereof; or a Cas14 protein, a fragment (e.g., biologically active fragment) or a variant thereof.
   273. The LNP composition of embodiment 270 or 271, wherein the CRISPR/Cas gene editing system comprises a nucleic acid encoding a gRNA molecule comprising a targeting sequence specific to a sequence of a target gene, and a Cas9 protein, a fragment (e.g., biologically active fragment) or a variant thereof.
   274. The LNP composition of embodiment 270 or 271, wherein the CRISPR/Cas gene editing system comprises a nucleic acid encoding a gRNA molecule comprising a targeting sequence specific to a sequence of a target gene, and a nucleic acid encoding a Cas9 protein, a fragment (e.g., biologically active fragment) or a variant thereof.
   275. The LNP composition of any one of embodiments 270 or 274, wherein the CRISPR/Cas gene editing system further comprises a template DNA.
   276. The LNP composition of any one of embodiments 270 or 275, wherein the CRISPR/Cas gene editing system further comprises a template RNA.
   277. The LNP composition of any one of embodiments 270 or 276, wherein the CRISPR/Cas gene editing system further comprises a reverse transcriptase.
   278. The LNP composition of any one of embodiment 262-269, wherein the genetic modulator comprises a zinc finger nuclease (ZFN) system.
   279. The LNP composition of embodiment 278, wherein the ZFN system comprises a peptide having: a zinc finger DNA binding domain, a fragment (e.g., biologically active fragment) or a variant thereof; and/or nuclease activity, e.g., endonuclease activity.
   280. The LNP composition of embodiment 278 or 279, wherein the ZFN system comprises a peptide having a zinc finger DNA binding domain.
   281. The LNP composition of embodiment 280, wherein the zinc finger binding domain comprises 1, 2, 3, 4, 5, 6, 7, 8 or more zinc fingers, e.g., 3 or 6 zinc fingers.
   282. The LNP composition of any one of embodiments 278-281, wherein the ZFN system comprises a peptide having nuclease activity, e.g., endonuclease activity.
   283. The LNP composition of embodiment 282, wherein the peptide having nuclease activity is a type-II restriction 1-like endonuclease, e.g., a FokI endonuclease.
   284. The LNP composition of embodiment 278, wherein the ZFN system comprises a nucleic acid encoding a peptide having: a zinc finger DNA binding domain, a fragment (e.g., biologically active fragment) or a variant thereof; and/or nuclease activity, e.g., endonuclease activity.
   285. The LNP composition of embodiment 284, wherein the ZFN system comprises a nucleic acid encoding a peptide having a zinc finger DNA binding domain.
   286. The LNP composition of embodiment 285, wherein the zinc finger binding domain comprises 1, 2, 3, 4, 5, 6, 7, 8 or more zinc fingers, e.g., 3 or 6 zinc fingers.
   287. The LNP composition of embodiment 284 or 285, wherein the ZFN system comprises a nucleic acid encoding a peptide having nuclease activity, e.g., endonuclease activity.
   288. The LNP composition of embodiment 287, wherein the peptide having nuclease activity is a type-II restriction 1-like endonuclease, e.g., a FokI endonuclease.
   289. The LNP composition of any one of embodiments 278-288, wherein the ZFN system further comprises a template, e.g., template DNA.
   290. The LNP composition of embodiment 262-289, wherein the genetic modulator is a transcription activator-like effector nuclease (TALEN) system.
   291. The LNP composition of embodiment 290, wherein the TALEN system comprises a peptide having: a transcription activator-like (TAL) effector DNA binding domain, a fragment (e.g., biologically active fragment) or a variant thereof; and/or nuclease activity, e.g., endonuclease activity.
   292. The LNP composition of embodiment 290 or 291, wherein the TALEN system comprises a peptide having a TAL effector DNA binding domain, a fragment (e.g., biologically active fragment) or a variant thereof.
   293. The LNP composition of embodiment 290 or 291, wherein the TALEN system comprises a peptide having nuclease activity, e.g., endonuclease activity.
   294. The LNP composition of embodiment 293, wherein the peptide having nuclease activity is a type-II restriction 1-like endonuclease, e.g., a FokI endonuclease.
   295. The LNP composition of embodiment 290, wherein the TALEN system comprises a nucleic acid encoding a peptide having: a transcription activator-like (TAL) effector DNA binding domain, a fragment (e.g., biologically active fragment) or a variant thereof; and/or nuclease activity, e.g., endonuclease activity.
   296. The LNP composition of embodiment 295, wherein the TALEN system comprises a nucleic acid encoding a peptide having a transcription activator-like (TAL) effector DNA binding domain, a fragment (e.g., biologically active fragment) or a variant thereof.
   297. The LNP composition of embodiment 295, wherein the TALEN system comprises a nucleic acid encoding a peptide having nuclease activity, e.g., endonuclease activity.
   298. The LNP composition of embodiment 297, wherein the peptide having nuclease activity is a type-II restriction 1-like endonuclease, e.g., a FokI endonuclease.
   299. The LNP composition of any one of embodiments 290-298, wherein the TALEN system further comprises a template, e.g., a template DNA.
   300. The LNP composition of any one of embodiments 262-269, wherein the genetic modulator comprises a meganuclease system.
   301. The LNP composition of embodiment 300, wherein the meganuclease system comprises a peptide having a DNA binding domain and nuclease activity, e.g., a homing endonuclease.
   302. The LNP composition of embodiment 301, wherein the homing endonuclease comprises a LAGLIDADG endonuclease (SEQ ID NO: 270), GIY-YIG endonuclease, HNH endonuclease, His-Cys box endonuclease or a PD-(D/E)XK endonuclease, or a fragment (e.g., biologically active fragment) or variant thereof, e.g., as described in Silva G. et al, (2011) Curr Gene Therapy 11(1): 11-27.
   303. The LNP composition of embodiment 300, wherein the meganuclease system comprises a nucleic acid encoding a peptide having a DNA binding domain and nuclease activity, e.g., a homing endonuclease.
   304. The LNP composition of embodiment 303, wherein the homing endonuclease comprises a LAGLIDADG endonuclease (SEQ ID NO: 270), GIY-YIG endonuclease, HNH endonuclease, His-Cys box endonuclease or a PD-(D/E)XK endonuclease, or a fragment (e.g., biologically active fragment) or variant thereof, e.g., as described in Silva G. et al, (2011) Curr Gene Therapy 11(1): 11-27.
   305. The LNP composition of any one of embodiments 300-304, wherein the system further comprises a template, e.g., a template DNA.
   306. The LNP composition of any one of embodiments 262-305, wherein the genetic modulator comprises a transposase system.
   307. The LNP composition of embodiment 306, wherein the transposase system comprises a nucleic acid sequence encoding a peptide having reverse transcriptase and/or nuclease activity, e.g., a retrotransposon, e.g., an LTR retrotransposon or a non-LTR retrotransposon.
   308. The LNP composition of embodiment 306 or 307, wherein the transposase system comprises a template, e.g., an RNA template.
   309. The LNP composition of any one of embodiments 192-308, wherein the payload comprises an epigenetic modulator (e.g., a modulator that epigenetically alters the cell or tissue).
   310. The LNP composition of embodiment 309, wherein the epigenetic modulator comprises a molecule that modifies chromatin architecture, methylates DNA, and/or modifies a histone.
   311. The LNP composition of embodiment 309 or 310, wherein the epigenetic modulator comprises a molecule that modifies chromatin architecture, e.g., a SWI/SNF remodeling complex or a component thereof.
   312. The LNP composition of any one of embodiments 309-311, wherein the epigenetic modulator comprises a molecule that methylates DNA, e.g., a DNA methyltransferase, a fragment (e.g., biologically active fragment) or variant thereof (e.g., DNMT1, DNMT2 DNMT3A, DNMT3B, DNMT3L, or M. SssI); a polycomb repressive complex or a component thereof, e.g., PRC1 or PRC2, or PR-DUB, or a fragment (e.g., biologically active fragment) or a variant thereof; a demethylase, or a fragment (e.g., biologically active fragment) or a variant thereof (e.g., Tet1, Tet2 or Tet3).
   313. The LNP composition of any one of embodiments 309-312, wherein the epigenetic modulator comprises a molecule that modifies a histone, e.g., methylates and/or acetylates a histone, e.g., a histone modifying enzyme or a fragment (e.g., biologically active fragment) or a variant thereof, e.g., HMT, HDM, HAT, or HDAC.
   314. The LNP composition of any one of embodiments 192-313, wherein the payload comprises an RNA modulator (e.g., a modulator that alters an RNA molecule in the cell or tissue).
   315. The LNP composition of embodiment 314, wherein the RNA modulator comprises a molecule that alters the expression and/or activity; stability or compartmentalization of an RNA molecule.
   316. The LNP composition of embodiment 314 or 315, wherein the RNA modulator comprises an RNA molecule, e.g., mRNA, rRNA, tRNA, regulatory RNA, non-coding RNA, long non-coding RNA (lncRNA), guide RNA (gRNA), piwi-interacting RNA (piRNA), small nucleolar RNA (snoRNA), small nuclear RNA (snRNA), extracellular RNA (exRNA), small Cajal body-specific RNA (scaRNA), microRNA (miRNA), circular RNA, or an RNAi molecule, e.g., small interfering RNA (siRNA) or small hairpin RNA (shRNA).
   317. The LNP composition of any of embodiments 314-316, wherein the RNA modulator comprises a DNA molecule.
   318. The LNP composition of any of embodiments 314-317, wherein the RNA modulator comprises a low molecular weight molecule.
   319. The LNP composition of any of embodiments 314-318, wherein the RNA modulator comprises a peptide, e.g., an RNA binding protein, a fragment (e.g., biologically active fragment), or a variant thereof, or an enzyme, or a fragment (e.g., biologically active fragment) or variant thereof.
   320. The LNP composition of any of embodiments 314-319, wherein the RNA modulator comprises an RNA base editor system.
   321. The LNP composition of embodiment 320, wherein the RNA base editor system comprises: a deaminase, e.g., an RNA-specific adenosine deaminase (ADAR); a Cas protein, a fragment (e.g., biologically active fragment) or a variant thereof; and/or a guide RNA.
   322. The LNP composition of embodiment 320 or 321, wherein the RNA base editor system further comprises a template, e.g., a DNA or RNA template.
   323. The LNP composition of any one of embodiments 192-322, wherein payload comprises a peptide modulator (e.g., a modulator that alters a peptide molecule in the cell or tissue).
   324. The LNP composition of any one of embodiments 192-323, wherein the payload comprises a lipid modulator (e.g., a modulator that alters a lipid molecule in the cell or tissue); or a combination thereof.
   325. The LNP composition of any one of embodiments 192-324, wherein the payload comprises a therapeutic payload or a prophylactic payload.
   326. The LNP composition of embodiment 325, wherein the therapeutic payload or prophylactic payload comprises a secreted protein, a membrane-bound protein, or an intercellular protein; or an mRNA encoding a secreted protein, a membrane-bound protein; or an intercellular protein.
   327. The LNP composition of embodiment 325 or 326, wherein the therapeutic payload or prophylactic payload comprises a protein, polypeptide, or peptide.
   328. The LNP composition of any one of embodiments 192-327, wherein the subject has a mutation in a hemoglobulin gene and/or has an aberrant expression of a hemoglobulin gene.
   329. The LNP composition of any one of embodiments 192-328, wherein the subject has a mutation in a gene encoding a clotting factor, and/or has an aberrant expression of a gene encoding a clotting factor.
   330. The LNP composition of any one of embodiments 192-329, wherein the subject is a mammal, e.g., human.
   331. The LNP composition of any one of embodiments 192-196 or 198-320, wherein the LNP composition comprises: (i) an ionizable lipid, e.g., an amino lipid; (ii) a sterol or other structural lipid; (iii) a non-cationic helper lipid or phospholipid; and (iv) a PEG-lipid.
   332. The LNP composition of embodiment 331, wherein the ionizable lipid comprises an amino lipid.
   333. The LNP composition of embodiment 332, wherein the ionizable lipid comprises a compound of any of Formulae (I), (I-I), (I-II), (I-III), (I-IV), (Ia), (Ib), (Ic), (II), or (II-I).
   334. The LNP composition of embodiment 331, wherein the ionizable lipid comprises a compound of Formula (I).
   335. The LNP composition of embodiment 331, wherein the ionizable lipid comprises a compound of Formula (Ia).
   336. The LNP composition of embodiment 331, wherein the ionizable lipid comprises a compound of Formula (Ib).
   337. The LNP composition of embodiment 331, wherein the ionizable lipid comprises a compound of Formula (Ic).
   338. The LNP composition of embodiment 331, wherein the ionizable lipid comprises a compound of Formula (I-I).
   339. The LNP composition of embodiment 331, wherein the ionizable lipid comprises a compound of Formula (I-II).
   340. The LNP composition of embodiment 331, wherein the ionizable lipid comprises a compound of Formula (I-III).
   341. The LNP composition of embodiment 331, wherein the ionizable lipid comprises a compound of Formula (I-IV).
   342. The LNP composition of embodiment 331, wherein the ionizable lipid comprises a compound of Formula (II).
   343. The LNP composition of embodiment 331, wherein the ionizable lipid comprises a compound of Formula (II-I).
   344. The LNP composition of embodiment 331, wherein the non-cationic helper lipid or phospholipid comprises a compound selected from the group consisting of DSPC, DPPC, or DOPC.
   345. The LNP composition of embodiment 331, wherein the phospholipid is DSPC, e.g., a variant of DSPC, e.g., a compound of Formula (IV).
   346. The LNP composition of embodiment 331, wherein the structural lipid is chosen from alpha-tocopherol, β-sitosterol or cholesterol.
   347. The LNP composition of embodiment 331, wherein the structural lipid is alpha-tocopherol.
   348. The LNP composition of embodiment 331, wherein the structural lipid is β-sitosterol.
   349. The LNP composition of embodiment 331, wherein the structural lipid is cholesterol.
   350. The LNP composition of embodiment 331, wherein the PEG lipid is selected from the group consisting of a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof.
   351. The LNP composition of embodiment 331, wherein the PEG lipid is selected from the group consisting of PEG-c-DOMG, PEG-DMG, PEG-DLPE, PEG-DMPE, PEG-DPPC and PEG-DSPE lipid.
   352. The LNP composition of embodiment 331, wherein the PEG-lipid is PEG-DMG.
   353. The LNP composition of embodiment 331, wherein the PEG lipid is chosen from a compound of: Formula (V), Formula (VI-A), Formula (VI-B), Formula (VI-C) or Formula (VI-D).
   354. The LNP composition of embodiment 331, wherein the PEG lipid is a compound of Formula (VI-A).
   355. The LNP composition of embodiment 331, wherein the PEG lipid is a compound of Formula (VI-B).
   356. The LNP composition of embodiment 331, wherein the PEG lipid is a compound of Formula (VI-C).
   357. The LNP composition of embodiment 331, wherein the PEG lipid is a compound of Formula (VI-D).
   358. The LNP composition of embodiment 331, wherein the ionizable lipid comprises a compound of Formula (I-I), the phospholipid comprises DSPC, the structural lipid comprises cholesterol. and the PEG lipid comprises a compound of Formula (VI-D).
   359. The LNP composition of any one of embodiments 331-358, wherein the LNP comprises a molar ratio of about 20-60% ionizable lipid: 5-25% phospholipid: 25-55% cholesterol; and 0.5-15% PEG lipid.
   360. The LNP composition of embodiment 359, wherein the LNP comprises a molar ratio of about 50% ionizable lipid: about 10% phospholipid: about 38.5% cholesterol; and about 1.5% PEG lipid.
   361. The LNP composition of embodiment 359, wherein the LNP comprises a molar ratio of about 49.83% ionizable lipid: about 9.83% phospholipid: about 30.33% cholesterol; and about 2.0% PEG lipid.
   362. The LNP composition of any one of embodiments 331-361, wherein the LNP composition is formulated for intravenous, subcutaneous, intramuscular, intranasal, intraocular, or pulmonary delivery (e.g., single or repeat delivery).
   363. The LNP composition of any one of embodiments 331-362, wherein the LNP composition is formulated for intravenous delivery (e.g., single or repeat delivery).

### BRIEF DESCRIPTIONS OF THE DRAWINGS

FIGS. 1A-1C show in vivo transfection and Cre-mediated gene editing of HSPC upon injection of Cre-mRNA LNP (LNPcre). FIG. 1A shows TdTomato fluorescence in HSPC (Lineage negative, LSK gate). FIG. 1B shows LSK sub-gates enriched in multi-potent progenitors (MPP), hematopoietic progenitor cells (HPC), or HSC. White: Mice treated with LNPcre; Grey: Mice Tris/sucrose control. FIG. 1C depicts a graph showing % of TdTomato+cells in corresponding HSPC gates. n=3, and data is presented as mean±SEM.

FIG. 2A shows generation of HSPC-derived colony forming units (CFU) upon ex vivo plating of bone marrow cells harvested from Ai14 mice injected intravenously with Cre-mRNA LNP or vehicle (tris/sucrose). Bone marrow cells were harvested from Ai14 mice 48 hours post injection of Cre-mRNA LNP and plated for up to 14 days in methylcellulose based medium enriched with cytokines/growth factors. Confocal microscopy images were taken of the colonies. Images were acquired on the opera Phenix (5× Air objective) at the indicated time points, and show TdTomato fluorescent images (bottom panels), brightfield images (middle panels), and merged (TdTomato+brightfield) images (top panel). FIG. 2B displays graphs showing CFU counts at different time points (left) and % of TdTomato+cells (right) from BM cells harvested from vehicle-treated Ai14 mice or LNPcre-treated Ai14 mice. n=3 wells per condition, and data is presented as mean±SEM.

FIGS. 3A-3C show a progressive increase in TdTomato fluorescent platelets and red blood cells in the peripheral blood circulation of Ai14 mice after intravenous injection of Cre-mRNA LNP. FIG. 3A displays representative flow cytometry plots (top panel) and summary scatter-line graph (bottom panel) that show an increase in the percent (%) of TdTomato fluorescent platelets within the total circulating pool of CD41+platelets post LNPcre delivery over time (n=4 mice up to Day 91, then n=3 for up to ˜231 days or 8 months). FIG. 3B displays representative flow cytometry plots (top panel) and summary scatter-line graph (bottom panel) that show an increase in the percent (%) of TdTomato fluorescent RBC within the total circulating pool of Ter 19+RBC post LNPcre delivery over time (n=4 mice up to Day 91, then n=3 for up to ˜231 days or 8 months). FIG. 3C displays a combined summary plot that shows an increase in the percent (%) of TdTomato fluorescent platelets within the total circulating pool of CD41+platelets and in the percent (%) of TdTomato fluorescent RBC within the total circulating pool of Ter 19+RBC up to 8 months post LNPcre delivery (n=3 mice, mean±SEM).

FIGS. 3D-3G shows a progressive increase in TdTomato fluorescent neutrophils, monocytes, B cells, CD4+ T cells, and CD8+ T cells in the peripheral blood circulation of Ai14 mice after intravenous injection of Cre-mRNA LNP. FIG. 3D displays summary scatter-line graphs that show an increase in the percent (%) of TdTomato fluorescent neutrophils (left panel) and monocytes (right panel) within the total circulating leukocytes post LNPcre delivery over time (n=4 mice up to Day 91, then n=3 for up to ˜231 days or 8 months). FIG. 3E displays a combined summary plot that shows an increase in the percent (%) of TdTomato fluorescent monocytes and in the percent (%) of TdTomato fluorescent neutrophils up to 8 months post LNPcre delivery (n=3 mice, mean±SEM). FIG. 3F displays summary scatter-line graphs that show an increase in the percent (%) of TdTomato fluorescent B cells (left panel), CD4+ T cells (middle panel), and CD8+ T cells (right panel) within the total circulating leukocytes post LNPcre delivery over time (n=4 mice up to Day 91, then n=3 for up to ˜231 days or 8 months). FIG. 3G displays a combined summary plot that shows an increase in the percent (%) of TdTomato fluorescent B cells, the percent (%) of TdTomato fluorescent CD4 T cells, and in the percent (%) of TdTomato fluorescent CD8 T cells, up to 8 months post LNPcre delivery (n=3 mice, mean±SEM).

FIGS. 4A-4C show full hematopoietic reconstitution upon serial bone marrow transplant in irradiated mice. FIG. 4A displays a frequency graph that shows the percent (%) of TdTomato fluorescent cells circulating among platelets and red blood cells in donors, primary transplant recipients, and secondary transplant recipients. FIG. 4B displays a frequency graph that shows the percent (%) of TdTomato fluorescent cells circulating among myeloid cells (monocytes, neutrophils, and eosinophils) in donors, primary transplant recipients, and secondary transplant recipients. FIG. 4C displays a frequency graph that shows the percent (%) of TdTomato fluorescent cells circulating among lymphocytes (B cells, CD4 T cells, CD8 T cells) in donors, primary transplant recipients, and secondary transplant recipients. Boxed area and closed data points refer to blood profile of individual donor mice. Open data points refer to blood profile in the respective recipient mice (CD45.1+). Immune cell subsets were gated on cells of CD45.2+Ai14 donor origin. n=3 for donor mice, and n=13-15 mice for recipients.

FIGS. 5A-5C show the additive cumulative effect of multiple dosing with LNPcre on HSPC delivery and labeling of hematopoietic cells to Ai14 mice. FIG. 5A displays a summary line graph that shows the percent (%) of TdTomato fluorescent cells circulating among platelets (first panel) and red blood cells (second panel) up to ˜195d or ˜6 months post-LNPcre administration. FIG. 5B displays a summary line graph that shows the percent (%) of TdTomato fluorescent cells circulating among monocytes (first panel), neutrophils (second panel), and eosinophils (third panel) up ˜195d or ˜6 months post-LNPcre administration. FIG. 5C displays a summary line graph that shows the percent (%) of TdTomato fluorescent cells circulating among B cells (first panel), CD4 T cells (second panel), CD8 T cells (third panel) up to ˜195d or ˜6 months post-LNPcre administration. In each of the plots, the shaded area represents the injection interval for the administration of LNPcre (starting at day −16). The dotted line at Day 0 indicates the last injection performed for each of the three dosing groups (five injections, three injections, and one injection).

FIG. 6 illustrate the delivery of LNPcre to bone marrow HSPC in non-human primates. shows a plot of the percentage (%) of cells expressing mOX40L reporter among all CD34⁺ bone marrow cells and in HSC-enriched CD34⁺CD90+c-Kit⁺CD45RA⁻CD123⁻HSPC. n=2 for vehicle treated NHP, N=15 for LNPcre treated NHP, and data is presented as mean±SEM.

FIGS. 7A-7C illustrate the delivery of LNP to human HSPC in humanized mice. FIG. 7A shows plots of the increase in percentage (%) mOX40L expression in human HSPC subsets with LNP administration. Representative data of 3 independent experiments. Each dot represents one humanized mouse. n=7 vehicle-treated, 15 LNP-treated mice, and data is presented as mean±SEM. FIG. 7B-7C depict photographic images (FIG. 7B) and graphs of colony count (FIG. 7C) from CFU assays plated with FACS-sorted mOX40L⁺ and mOX40L⁻ human CD34⁺ progenitors from bone marrow of LNP-injected humanized mice. Representative of 2 independent experiments; cells were sorted from n=5-6 humanized mice and plated in duplicate.

### DETAILED DESCRIPTION

In vivo modification of a cell, e.g., stem or progenitor cell, or a tissue, can be a valuable tool for treating a disease or disorder. Gene editing of hematopoietic stem and progenitor cells (HSPC) is a promising approach to treat a large number of serious life-threatening conditions.

While there are currently over 150 ongoing clinical trials testing HSPC gene editing for different conditions, all of them employ ex vivo gene editing. Such ex vivo procedures are costly, technically complex, and associated to substantial morbidity, requiring patient pre-treatment with cytokines to mobilize HSPC and then myeloablation to allow gene-edited HSPC engraftment once the edited HSPC are transplanted back into patients. A major advance would be to enable in vivo gene editing of HSPC. LNPs carrying cargos, e.g., nucleic acid cargos, used to transfect HSPC in vivo, would obviate the need for HSPC isolation, ex vivo gene editing, and conventional bone marrow (BM) transplants.

Disclosed herein, inter alia, is the discovery that LNP composition comprising a payload can result in in vivo modification of a cell, e.g., in vivo gene editing in cells, e.g., stem or progenitor cells, e.g., hematopoietic stem and progenitor cells. In some embodiments, the disclosure provides LNP compositions comprising a payload that can modify a cell, e.g., a stem or progenitor cell, or a tissue, in vivo. In some embodiments, the LNP composition does not include an additional targeting moiety, e.g., it transfects (e.g., at least 10%, 20%, 30%, 40%, 50%, 60%, 70%, 80%, 90%, or 95%) of cells described herein, e.g., stem or progenitor cells (e.g., HSPCs), without an additional targeting moiety. Without wishing to be bound by theory, it is believed that in some embodiments, in vivo methods of modifying a cell or tissue disclosed herein obviate the need for isolation of cells (e.g., HSPCs), ex vivo gene editing and/or bone marrow transplants. The discoveries disclosed herein provide an advance in in vivo modification of a cell, e.g., in vivo gene editing, and in an embodiment, make it possible to treat a vast number of devastating diseases.

Exemplary in vivo gene editing effects of LNP compositions comprising a payload are provided in Examples 1-3. Example 1 demonstrates that hematopoietic stem cells or progenitors thereof can be gene edited in vivo with an LNP composition comprising a payload. Examples 2-3 show the effects of in vivo gene edited hematopoietic stem and progenitor cells with an LNP composition comprising a payload. Example 2 shows the generation of HSPC-derived colony forming units (CFU) from in vivo gene edited hematopoietic stem and progenitor cells, and Example 3 shows that in vivo gene edited hematopoietic stem and progenitor cells can give rise to platelets, erythrocytes, neutrophils, monocytes, B cells, CD4+ T cells, and CD8+ T cells in vivo. Example 4 describes evaluation of stemness potential of in vivo gene edited HSPCs.

Accordingly, disclosed herein are methods of modifying a cell, e.g., a stem or progenitor cell, in vivo with lipid nanoparticle (LNP) compositions comprising a payload. Also disclosed herein are methods of modifying a tissue in vivo with lipid nanoparticle (LNP) compositions comprising a payload. In an embodiment, the LNP compositions modify a parameter associated with the cell or tissue or modify a component associated with the cell or tissue. Further disclosed herein are methods of treating a subject having a disease, a disorder, a mutation, or a single nucleotide polymorphism (SNP), comprising administering to the subject an effective amount of an LNP composition comprising a payload. In an embodiment, the LNP composition results in a modification of a cell (e.g., stem or progenitor cell) in the subject, e.g., modification of a component associated with the cell or a parameter associated with the cell. Also disclosed herein are LNP compositions comprising a payload for use, e.g., in the in vivo modification of a cell or tissue, and methods of making the same. Additional aspects of the disclosure are described in further detail below.

### Definitions

Parameter associated with a cell: The phrase “parameter associated with a cell or tissue” as used herein refers to a genotypic parameter, a phenotypic parameter, a functional parameter, an expression parameter, or a signaling parameter associated with a cell or tissue. In an embodiment, the expression parameter comprises one, two, three, four or all of the following: (a) expression level (e.g., of polypeptide or protein, or polynucleotide or nucleic acid, e.g., mRNA); (b) activity (e.g., of polypeptide or protein, or polynucleotide or nucleic acid, e.g., mRNA), (c) post-translational modification of polypeptide or protein; (d) folding (e.g., of polypeptide or protein, or polynucleotide or nucleic acid, e.g., mRNA), and/or (e) stability (e.g., of polypeptide or protein, or polynucleotide or nucleic acid, e.g., mRNA). In an embodiment, the signaling parameter comprises one, two, three, four or all of the following: (1) modulation of a signaling pathway, e.g., a cellular signaling pathway; (2) cell fate modulation; (3) modulation of expression level (e.g., of polypeptide or protein, or polynucleotide or nucleic acid, e.g., mRNA); (4) modulation of activity (e.g., of polypeptide or protein, or polynucleotide or nucleic acid, e.g., mRNA), and/or (5) modulation of stability e.g., of polypeptide or protein, or polynucleotide or nucleic acid, e.g., mRNA). In an embodiment, the phenotypic parameter comprises expression and/or activity of a molecule, e.g., cell surface protein, lipid or adhesion molecule, on the surface of the cell.

Component associated with a cell: The phrase “component associated with a cell” as used herein refers to a component which is endogenous to (e.g., naturally occurring) a cell or which is exogenous to (e.g., introduced into) a cell. In an embodiment, a component associated with a cell comprises: (1) a nucleic acid associated with the cell or fragment thereof, e.g., DNA (e.g., exonic, intronic, intergenic, telomeric, promoter, enhancer, insulator, repressor, coding, non-coding) or RNA (e.g., mRNA, rRNA, tRNA, regulatory RNA, non-coding RNA, long non-coding RNA (lncRNA), guide RNA (gRNA), piwi-interacting RNA (piRNA), small nucleolar RNA (snoRNA), small nuclear RNA (snRNA), extracellular RNA (exRNA), small Cajal body-specific RNA (scaRNA), micro RNA (miRNA), circular RNA, or an RNAi molecule, e.g., small interfering RNA (siRNA) or small hairpin RNA (shRNA)); (2) a peptide or protein associated with the cell or fragment thereof; (3) a lipid component associated with the cell or fragment thereof, or a combination thereof.

Uridine Content: The terms “uridine content” or “uracil content” are interchangeable and refer to the amount of uracil or uridine present in a certain nucleic acid sequence. Uridine content or uracil content can be expressed as an absolute value (total number of uridine or uracil in the sequence) or relative (uridine or uracil percentage respect to the total number of nucleobases in the nucleic acid sequence).

Alternative nucleoside. An “alternative nucleoside” as that term is used herein, in reference to a nucleotide, nucleoside, or polynucleotide (such as the polynucleotides of the invention, e.g., mRNA molecule), refers to alteration with respect to A, G, U or C ribonucleotides. Generally, herein, these terms are not intended to refer to the ribonucleotide alterations in naturally occurring 5′-terminal mRNA cap moieties. The alterations may be various distinct alterations. In some embodiments, where the polynucleotide is an mRNA, the coding region, the flanking regions and/or the terminal regions (e.g., a 3′-stabilizing region) may contain one, two, or more (optionally different) nucleoside or nucleotide alterations. In some embodiments, an alternative polynucleotide introduced to a cell may exhibit reduced degradation in the cell, as compared to an unaltered polynucleotide.

Administering: As used herein, “administering” refers to a method of delivering a composition to a subject or patient. A method of administration may be selected to target delivery (e.g., to specifically deliver) to a specific region or system of a body. For example, an administration may be parenteral (e.g., subcutaneous, intracutaneous, intravenous, intraperitoneal, intramuscular, intraarticular, intraarterial, intrasynovial, intrasternal, intrathecal, intralesional, or intracranial injection, as well as any suitable infusion technique), oral, trans- or intra-dermal, interdermal, rectal, intravaginal, topical (e.g., by powders, ointments, creams, gels, lotions, and/or drops), mucosal, nasal, buccal, enteral, vitreal, intratumoral, sublingual, intranasal; by intratracheal instillation, bronchial instillation, and/or inhalation; as an oral spray and/or powder, nasal spray, and/or aerosol, and/or through a portal vein catheter. Preferred means of administration are intravenous or subcutaneous.

Approximately, about: As used herein, the terms “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). For example, when used in the context of an amount of a given compound in a lipid component of an LNP, “about” may mean +/−10% of the recited value. For instance, an LNP including a lipid component having about 50% of a given compound may include 45-55% of the compound.

Contacting: As used herein, the term “contacting” means establishing a physical connection between two or more entities. For example, contacting a cell with an mRNA or a lipid nanoparticle composition means that the cell and mRNA or lipid nanoparticle are made to share a physical connection. Methods of contacting cells with external entities both in vivo, in vitro, and ex vivo are well known in the biological arts. In exemplary embodiments of the disclosure, the step of contacting a mammalian cell with a composition (e.g., a nanoparticle, or pharmaceutical composition of the disclosure) is performed in vivo. For example, contacting a lipid nanoparticle composition and a cell (for example, a mammalian cell) which may be disposed within an organism (e.g., a mammal) may be performed by any suitable administration route (e.g., parenteral administration to the organism, including intravenous, intramuscular, intradermal, and subcutaneous administration). For a cell present in vitro, a composition (e.g., a lipid nanoparticle) and a cell may be contacted, for example, by adding the composition to the culture medium of the cell and may involve or result in transfection. Moreover, more than one cell may be contacted by a nanoparticle composition.

Delivering: As used herein, the term “delivering” means providing an entity to a destination. For example, delivering a therapeutic and/or prophylactic to a subject may involve administering a LNP including the therapeutic and/or prophylactic to the subject (e.g., by an intravenous, intramuscular, intradermal, pulmonary or subcutaneous route). Administration of a LNP to a mammal or mammalian cell may involve contacting one or more cells with the lipid nanoparticle.

Encapsulate: As used herein, the term “encapsulate” means to enclose, surround, or encase. In some embodiments, a compound, polynucleotide (e.g., an mRNA), or other composition may be fully encapsulated, partially encapsulated, or substantially encapsulated. For example, in some embodiments, an mRNA of the disclosure may be encapsulated in a lipid nanoparticle, e.g., a liposome.

Encapsulation efficiency: As used herein, “encapsulation efficiency” refers to the amount of a therapeutic and/or prophylactic that becomes part of a LNP, relative to the initial total amount of therapeutic and/or prophylactic used in the preparation of a LNP. For example, if 97 mg of therapeutic and/or prophylactic are encapsulated in a LNP out of a total 100 mg of therapeutic and/or prophylactic initially provided to the composition, the encapsulation efficiency may be given as 97%. As used herein, “encapsulation” may refer to complete, substantial, or partial enclosure, confinement, surrounding, or encasement.

Effective amount: As used herein, the term “effective amount” of an agent is that amount sufficient to effect beneficial or desired results, for example, clinical results, and, as such, an “effective amount” depends upon the context in which it is being applied. For example, in the context of the amount of a target cell delivery potentiating lipid in a lipid composition (e.g., LNP) of the disclosure, an effective amount of a target cell delivery potentiating lipid is an amount sufficient to effect a beneficial or desired result as compared to a lipid composition (e.g., LNP) lacking the target cell delivery potentiating lipid. Non-limiting examples of beneficial or desired results effected by the lipid composition (e.g., LNP) include increasing the percentage of cells transfected and/or increasing the level of expression of a protein encoded by a nucleic acid associated with/encapsulated by the lipid composition (e.g., LNP). In the context of administering a target cell delivery potentiating lipid-containing lipid nanoparticle such that an effective amount of lipid nanoparticles are taken up by target cells in a subject, an effective amount of target cell delivery potentiating lipid-containing LNP is an amount sufficient to effect a beneficial or desired result as compared to an LNP lacking the target cell delivery potentiating lipid. Non-limiting examples of beneficial or desired results in the subject include increasing the percentage of cells transfected, increasing the level of expression of a protein encoded by a nucleic acid associated with/encapsulated by the target cell delivery potentiating lipid-containing LNP and/or increasing a prophylactic or therapeutic effect in vivo of a nucleic acid, or its encoded protein, associated with/encapsulated by the target cell delivery potentiating lipid-containing LNP, as compared to an LNP lacking the target cell delivery potentiating lipid. In some embodiments, a therapeutically effective amount of target cell delivery potentiating lipid-containing LNP is sufficient, when administered to a subject suffering from or susceptible to an infection, disease, disorder, and/or condition, to treat, improve symptoms of, diagnose, prevent, and/or delay the onset of the infection, disease, disorder, and/or condition. In another embodiment, an effective amount of a lipid nanoparticle is sufficient to result in expression of a desired protein in at least about 5%, 10%, 15%, 20%, 25% or more of target cells. For example, an effective amount of target cell delivery potentiating lipid-containing LNP can be an amount that results in transfection of at least 5%, 10%, 15%, 20%, 25%, 30%, or 35% of target cells after a single intravenous injection.

Expression: As used herein, “expression” of a nucleic acid sequence refers to one or more of the following events: (1) production of an RNA template from a DNA sequence (e.g., by transcription); (2) processing of an RNA transcript (e.g., by splicing, editing, 5′ cap formation, and/or 3′ end processing); (3) translation of an RNA into a polypeptide or protein; and (4) post-translational modification of a polypeptide or protein.

Ex vivo: As used herein, the term “ex vivo” refers to events that occur outside of an organism (e.g., animal, plant, or microbe or cell or tissue thereof). Ex vivo events may take place in an environment minimally altered from a natural (e.g., in vivo) environment.

Fragment: A “fragment,” as used herein, refers to a portion. For example, fragments of proteins may include polypeptides obtained by digesting full-length protein isolated from cultured cells or obtained through recombinant DNA techniques. A fragment of a protein can be, for example, a portion of a protein that includes one or more functional domains such that the fragment of the protein retains the functional activity of the protein.

Heterologous: As used herein, “heterologous” indicates that a sequence (e.g., an amino acid sequence or the polynucleotide that encodes an amino acid sequence) is not normally present in a given polypeptide or polynucleotide. For example, an amino acid sequence that corresponds to a domain or motif of one protein may be heterologous to a second protein.

Isolated: As used herein, the term “isolated” refers to a substance or entity that has been separated from at least some of the components with which it was associated (whether in nature or in an experimental setting). Isolated substances may have varying levels of purity in reference to the substances from which they have been associated. Isolated substances and/or entities may be separated from at least about 10%, about 20%, about 30%, about 40%, about 50%, about 60%, about 70%, about 80%, about 90%, or more of the other components with which they were initially associated. In some embodiments, isolated agents are more than about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components.

Liposome: As used herein, by “liposome” is meant a structure including a lipid-containing membrane enclosing an aqueous interior. Liposomes may have one or more lipid membranes. Liposomes include single-layered liposomes (also known in the art as unilamellar liposomes) and multi-layered liposomes (also known in the art as multilamellar liposomes).

Modified: As used herein “modified” refers to a changed state or structure of a molecule of the disclosure, e.g., a change in a composition or structure of a polynucleotide (e.g., mRNA). Molecules, e.g., polynucleotides, may be modified in various ways including chemically, structurally, and/or functionally. For example, molecules, e.g., polynucleotides, may be structurally modified by the incorporation of one or more RNA elements, wherein the RNA element comprises a sequence and/or an RNA secondary structure(s) that provides one or more functions (e.g., translational regulatory activity). Accordingly, molecules, e.g., polynucleotides, of the disclosure may be comprised of one or more modifications (e.g., may include one or more chemical, structural, or functional modifications, including any combination thereof). In one embodiment, polynucleotides, e.g., mRNA molecules, of the present disclosure are modified by the introduction of non-natural nucleosides and/or nucleotides, e.g., as it relates to the natural ribonucleotides A, U, G, and C. Noncanonical nucleotides such as the cap structures are not considered “modified” although they differ from the chemical structure of the A, C, G, U ribonucleotides.

mRNA: As used herein, an “mRNA” refers to a messenger ribonucleic acid. An mRNA may be naturally or non-naturally occurring. For example, an mRNA may include modified and/or non-naturally occurring components such as one or more nucleobases, nucleosides, nucleotides, or linkers. An mRNA may include a cap structure, a chain terminating nucleoside, a stem loop, a polyA sequence, and/or a polyadenylation signal. An mRNA may have a nucleotide sequence encoding a polypeptide. Translation of an mRNA, for example, in vivo translation of an mRNA inside a mammalian cell, may produce a polypeptide. Traditionally, the basic components of an mRNA molecule include at least a coding region, a 5′-untranslated region (5′-UTR), a 3′UTR, a 5′ cap and a polyA sequence. In an embodiment, the mRNA is a circular mRNA.

Nanoparticle: As used herein, “nanoparticle” refers to a particle having any one structural feature on a scale of less than about 1000 nm that exhibits novel properties as compared to a bulk sample of the same material. Routinely, nanoparticles have any one structural feature on a scale of less than about 500 nm, less than about 200 nm, or about 100 nm. Also routinely, nanoparticles have any one structural feature on a scale of from about 50 nm to about 500 nm, from about 50 nm to about 200 nm or from about 70 to about 120 nm. In exemplary embodiments, a nanoparticle is a particle having one or more dimensions of the order of about 1-1000 nm. In other exemplary embodiments, a nanoparticle is a particle having one or more dimensions of the order of about 10-500 nm. In other exemplary embodiments, a nanoparticle is a particle having one or more dimensions of the order of about 50-200 nm. A spherical nanoparticle would have a diameter, for example, of between about 50-100 or 70-120 nanometers. A nanoparticle most often behaves as a unit in terms of its transport and properties. It is noted that novel properties that differentiate nanoparticles from the corresponding bulk material typically develop at a size scale of under 1000 nm, or at a size of about 100 nm, but nanoparticles can be of a larger size, for example, for particles that are oblong, tubular, and the like. Although the size of most molecules would fit into the above outline, individual molecules are usually not referred to as nanoparticles.

Nucleic acid: As used herein, the term “nucleic acid” is used in its broadest sense and encompasses any compound and/or substance that includes a polymer of nucleotides. These polymers are often referred to as polynucleotides. Exemplary nucleic acids or polynucleotides of the disclosure include, but are not limited to, ribonucleic acids (RNAs), deoxyribonucleic acids (DNAs), DNA-RNA hybrids, RNAi-inducing agents, RNAi agents, siRNAs, shRNAs, miRNAs, antisense RNAs, ribozymes, catalytic DNA, RNAs that induce triple helix formation, threose nucleic acids (TNAs), glycol nucleic acids (GNAs), peptide nucleic acids (PNAs), locked nucleic acids (LNAs, including LNA having a β-D-ribo configuration, α-LNA having an α-L-ribo configuration (a diastereomer of LNA), 2′-amino-LNA having a 2′-amino functionalization, and 2′-amino-α-LNA having a 2′-amino functionalization) or hybrids thereof.

Nucleobase: As used herein, the term “nucleobase” (alternatively “nucleotide base” or “nitrogenous base”) refers to a purine or pyrimidine heterocyclic compound found in nucleic acids, including any derivatives or analogs of the naturally occurring purines and pyrimidines that confer improved properties (e.g., binding affinity, nuclease resistance, chemical stability) to a nucleic acid or a portion or segment thereof. Adenine, cytosine, guanine, thymine, and uracil are the nucleobases predominately found in natural nucleic acids. Other natural, non-natural, and/or synthetic nucleobases, as known in the art and/or described herein, can be incorporated into nucleic acids.

Nucleoside Nucleotide: As used herein, the term “nucleoside” refers to a compound containing a sugar molecule (e.g., a ribose in RNA or a deoxyribose in DNA), or derivative or analog thereof, covalently linked to a nucleobase (e.g., a purine or pyrimidine), or a derivative or analog thereof (also referred to herein as “nucleobase”), but lacking an internucleoside linking group (e.g., a phosphate group). As used herein, the term “nucleotide” refers to a nucleoside covalently bonded to an internucleoside linking group (e.g., a phosphate group), or any derivative, analog, or modification thereof that confers improved chemical and/or functional properties (e.g., binding affinity, nuclease resistance, chemical stability) to a nucleic acid or a portion or segment thereof.

Open Reading Frame: As used herein, the term “open reading frame”, abbreviated as “ORF”, refers to a segment or region of an mRNA molecule that encodes a polypeptide. The ORF comprises a continuous stretch of non-overlapping, in-frame codons, beginning with the initiation codon and ending with a stop codon, and is translated by the ribosome.

Patient: As used herein, “patient” refers to a subject who may seek or be in need of treatment, requires treatment, is receiving treatment, will receive treatment, or a subject who is under care by a trained professional for a particular disease or condition. In particular embodiments, a patient is a human patient.

Pharmaceutically acceptable: The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Pharmaceutically acceptable excipient: The phrase “pharmaceutically acceptable excipient,” as used herein, refers any ingredient other than the compounds described herein (for example, a vehicle capable of suspending or dissolving the active compound) and having the properties of being substantially nontoxic and non-inflammatory in a patient. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, and waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E, vitamin C, and xylitol.

Pharmaceutically acceptable salts: As used herein, “pharmaceutically acceptable salts” refers to derivatives of the disclosed compounds wherein the parent compound is modified by converting an existing acid or base moiety to its salt form (e.g., by reacting the free base group with a suitable organic acid). Examples of pharmaceutically acceptable salts include, but are not limited to, mineral or organic acid salts of basic residues such as amines; alkali or organic salts of acidic residues such as carboxylic acids; and the like. Representative acid addition salts include acetate, acetic acid, adipate, alginate, ascorbate, aspartate, benzenesulfonate, benzene sulfonic acid, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. The pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985, p. 1418, Pharmaceutical Salts: Properties, Selection, and Use, P. H. Stahl and C. G. Wermuth (eds.), Wiley-VCH, 2008, and Berge et al., Journal of Pharmaceutical Science, 66, 1-19 (1977), each of which is incorporated herein by reference in its entirety.

Polypeptide: As used herein, the term “polypeptide” or “polypeptide of interest” refers to a polymer of amino acid residues typically joined by peptide bonds that can be produced naturally (e.g., isolated or purified) or synthetically.

RNA: As used herein, an “RNA” refers to a ribonucleic acid that may be naturally or non-naturally occurring. For example, an RNA may include modified and/or non-naturally occurring components such as one or more nucleobases, nucleosides, nucleotides, or linkers. An RNA may include a cap structure, a chain terminating nucleoside, a stem loop, a polyA sequence, and/or a polyadenylation signal. An RNA may have a nucleotide sequence encoding a polypeptide of interest. For example, an RNA may be a messenger RNA (mRNA). Translation of an mRNA encoding a particular polypeptide, for example, in vivo translation of an mRNA inside a mammalian cell, may produce the encoded polypeptide. RNAs may be selected from the non-liming group consisting of small interfering RNA (siRNA), asymmetrical interfering RNA (aiRNA), microRNA (miRNA), Dicer-substrate RNA (dsRNA), small hairpin RNA (shRNA), mRNA, long non-coding RNA (lncRNA) and mixtures thereof.

RNA element: As used herein, the term “RNA element” refers to a portion, fragment, or segment of an RNA molecule that provides a biological function and/or has biological activity (e.g., translational regulatory activity). Modification of a polynucleotide by the incorporation of one or more RNA elements, such as those described herein, provides one or more desirable functional properties to the modified polynucleotide. RNA elements, as described herein, can be naturally-occurring, non-naturally occurring, synthetic, engineered, or any combination thereof. For example, naturally-occurring RNA elements that provide a regulatory activity include elements found throughout the transcriptomes of viruses, prokaryotic and eukaryotic organisms (e.g., humans). RNA elements in particular eukaryotic mRNAs and translated viral RNAs have been shown to be involved in mediating many functions in cells. Exemplary natural RNA elements include, but are not limited to, translation initiation elements (e.g., internal ribosome entry site (IRES), see Kieft et al., (2001) RNA 7(2):194-206), translation enhancer elements (e.g., the APP mRNA translation enhancer element, see Rogers et al., (1999) J Biol Chem 274(10):6421-6431), mRNA stability elements (e.g., AU-rich elements (AREs), see Garneau et al., (2007) Nat Rev Mol Cell Biol 8(2):113-126), translational repression element (see e.g., Blumer et al., (2002) Mech Dev 110(1-2):97-112), protein-binding RNA elements (e.g., iron-responsive element, see Selezneva et al., (2013) J Mol Biol 425(18):3301-3310), cytoplasmic polyadenylation elements (Villalba et al., (2011) Curr Opin Genet Dev 21(4):452-457), and catalytic RNA elements (e.g., ribozymes, see Scott et al., (2009) Biochim Biophys Acta 1789(9-10):634-641).

Specific delivery: As used herein, the term “specific delivery,” “specifically deliver,” or “specifically delivering” means delivery of more (e.g., at least 10% more, at least 20% more, at least 30% more, at least 40% more, at least 50% more, at least 1.5 fold more, at least 2-fold more, at least 3-fold more, at least 4-fold more, at least 5-fold more, at least 6-fold more, at least 7-fold more, at least 8-fold more, at least 9-fold more, at least 10-fold more) of a therapeutic and/or prophylactic by a nanoparticle to a target cell of interest (e.g., mammalian target cell) compared to an off-target cell (e.g., non-target cells). The level of delivery of a nanoparticle to a particular cell may be measured by comparing the amount of protein produced in target cells versus non-target cells (e.g., by mean fluorescence intensity using flow cytometry, comparing the % of target cells versus non-target cells expressing the protein (e.g., by quantitative flow cytometry), comparing the amount of protein produced in a target cell versus non-target cell to the amount of total protein in said target cells versus non-target cell, or comparing the amount of therapeutic and/or prophylactic in a target cell versus non-target cell to the amount of total therapeutic and/or prophylactic in said target cell versus non-target cell. It will be understood that the ability of a nanoparticle to specifically deliver to a target cell need not be determined in a subject being treated, it may be determined in a surrogate such as an animal model (e.g., a mouse or NHP model).

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Suffering from: An individual who is “suffering from” a disease, disorder, and/or condition has been diagnosed with or displays one or more symptoms of a disease, disorder, and/or condition.

Targeting moiety: As used herein, a “targeting moiety” is a compound or agent that may target a nanoparticle to a particular cell, tissue, and/or organ type. In some embodiments, an LNP of the disclosure does not include an additional targeting moiety, e.g., it transfects (e.g., at least 10%, 20%, 30%, 40%, 50%, 60%, 70%, 80%, 90%, or 95%) of stem or progenitor cells (e.g., HSPCs) without an additional targeting moiety.

Therapeutic Agent: The term “therapeutic agent” refers to any agent that, when administered to a subject, has a therapeutic, diagnostic, and/or prophylactic effect and/or elicits a desired biological and/or pharmacological effect. In some embodiments, the therapeutic agent comprises or is a therapeutic payload. In some embodiments, the therapeutic agent comprises or is a small molecule or a biologic (e.g., an antibody molecule).

Transfection: As used herein, the term “transfection” refers to methods to introduce a species (e.g., a polynucleotide, such as a mRNA) into a cell.

Translational Regulatory Activity: As used herein, the term “translational regulatory activity” (used interchangeably with “translational regulatory function”) refers to a biological function, mechanism, or process that modulates (e.g., regulates, influences, controls, varies) the activity of the translational apparatus, including the activity of the PIC and/or ribosome. In some aspects, the desired translation regulatory activity promotes and/or enhances the translational fidelity of mRNA translation. In some aspects, the desired translational regulatory activity reduces and/or inhibits leaky scanning.

Subject: As used herein, the term “subject” refers to any organism to which a composition in accordance with the disclosure may be administered, e.g., for experimental, diagnostic, prophylactic, and/or therapeutic purposes. Typical subjects include animals (e.g., mammals such as mice, rats, rabbits, non-human primates, and humans) and/or plants. In some embodiments, a subject may be a patient.

Treating: As used herein, the term “treating” refers to partially or completely alleviating, ameliorating, improving, relieving, delaying onset of, inhibiting progression of, reducing severity of, and/or reducing incidence of one or more symptoms or features of a particular infection, disease, disorder, and/or condition. Treatment may be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition.

Preventing: As used herein, the term “preventing” refers to partially or completely inhibiting the onset of one or more symptoms or features of a particular infection, disease, disorder, and/or condition.

Unmodified: As used herein, “unmodified” refers to any substance, compound or molecule prior to being changed in any way. Unmodified may, but does not always, refer to the wild type or native form of a biomolecule. Molecules may undergo a series of modifications whereby each modified molecule may serve as the “unmodified” starting molecule for a subsequent modification.

Variant: As used herein, the term “variant” refers to a molecule having at least 50%, 60%, 70%, 80%, 85%, 90%, 95%, 96%, 97%, 98%, 99% or 100% activity of, or structural similarity to, the wild type molecule, e.g., as measured by an art-recognized assay.

### In Vivo Methods of Modifying a Cell or Tissue and Related Methods

In an aspect, disclosed herein is a method of modifying a cell (e.g., stem or progenitor cell or a lineage of cells), e.g., modifying a parameter associated with the cell or a component associated with the cell, comprising contacting the cell with a lipid nanoparticle (LNP) composition comprising a payload, thereby modifying the cell. In an embodiment, contacting the cell with the LNP (e.g., administration of the LNP composition) modifies a parameter associated with the cell, e.g., as described herein. In an embodiment, contacting the cell with the LNP (e.g., administration) of the LNP composition modifies a component associated with the cell, e.g., as described herein. In an embodiment, the LNP composition does not comprise an additional targeting moiety.

In another aspect, disclosed herein is a method of modifying a tissue, e.g., modifying a parameter associated with the tissue or a component associated with the tissue, comprising contacting the cell with a lipid nanoparticle (LNP) composition comprising a payload. In an embodiment, contacting the cell with the LNP (e.g., administration of the LNP composition) modifies a parameter associated with the tissue, e.g., as described herein. In an embodiment, contacting the cell with the LNP (e.g., administration of the LNP composition) modifies a component associated with the tissue, e.g., as described herein. In an embodiment, the LNP composition does not comprise an additional targeting moiety.

In yet another aspect, provided herein is a method of treating a subject having a disease, a disorder, a mutation, or a single nucleotide polymorphism (SNP), comprising administering to the subject an effective amount of an LNP composition comprising a payload, wherein said LNP composition results in a modification of a cell (e.g., stem or progenitor cell) in the subject, e.g., modification of a component associated with the cell or a parameter associated with the cell, thereby treating the subject. In an embodiment, the LNP composition does not comprise an additional targeting moiety. In an embodiment, administration of the LNP composition modifies a parameter associated with the cell, e.g., as described herein. In an embodiment, administration of the LNP composition modifies a component associated with the cell, e.g., as described herein.

In an aspect, the disclosure provides a method of ameliorating a symptom of a subject having a disease, a disorder, a mutation, or a single nucleotide polymorphism (SNP), comprising administering to the subject an effective amount of an LNP composition comprising a payload, wherein said LNP composition results in a modification of a cell (e.g., stem or progenitor cell) in the subject, e.g., modification of a component associated with the cell or a parameter associated with the cell, thereby ameliorating the symptom of the subject. In an embodiment, the LNP composition does not comprise an additional targeting moiety. In an embodiment, administration of the LNP composition modifies a parameter associated with the cell, e.g., as described herein. In an embodiment, administration of the LNP composition modifies a component associated with the cell, e.g., as described herein.

### Hematopoietic Stem and Progenitor Cells

Hematopoietic stem and progenitor cells (HSPCs) are the stem and progenitor cells that give rise to other blood cells via a process called hematopoiesis. Hematopoiesis occurs in the bone marrow and/or in other immune sites, e.g., spleen, liver, thymus, lymph nodes. Without wishing to be bound by theory, it is believed that during hematopoiesis, HSCs which are multipotent and capable of self-renewal, differentiate into progenitor cells which give rise to mature blood cells in the myeloid lineage and the lymphoid lineage. Myeloid cells include monocytes, macrophages, neutrophils, basophils, eosinophils, erythrocytes, megakaryocytes, and platelets. Lymphoid cells include T cells, B cells, natural killer cells, and innate lymphoid cells. As used herein, the term “HSPC” encompasses both hematopoietic stem cell (HSC) and hematopoietic progenitor cell (HPC).

In an embodiment, any of the methods disclosed herein comprise in vivo modification of a stem or progenitor cell, e.g., a hematopoietic stem and progenitor cell (HSPC). In an embodiment, any of the methods disclosed herein comprise in vivo gene editing of a stem or progenitor cell, e.g., a hematopoietic stem and progenitor cell (HSPC). In an embodiment, the stem or progenitor cell comprises a HSPC or a population of HSPCs. In an embodiment, the HSPC comprises a HSPC derived from an embryonic stem cell or a HSPC derived from an induced pluripotent stem cell.

In an embodiment of any of the methods, compositions, or cells disclosed herein, the cell is a HSPC, e.g., a multipotent HSC or multipotent HPC. In an embodiment, the HSPC is an HSC. In an embodiment, the HSPC is an HPC.

In an embodiment of any of the methods, compositions, or cells disclosed herein, the HSPC has one, two, three, four, five or all of the following functional characteristics: (i) ability to self-renew; (ii) unlimited proliferative potential; (iii) ability to enter and/or exit a quiescent state, e.g., a cell state where no proliferation occurs, e.g., GO phase of the cell cycle; (iv) ability to differentiate into any hematopoietic lineage, e.g., myeloid and/or lymphoid lineages, e.g., common lymphoid progenitor (CLP) or a differentiated cell thereof, and/or common myeloid progenitor (CMP) or a differentiated cell thereof, (v) ability to repopulate any hematopoietic lineage, e.g., myeloid and/or lymphoid lineages, e.g., common lymphoid progenitor (CLP) or a differentiated cell thereof; and/or common myeloid progenitor (CMP) or a differentiated cell thereof, e.g., in an organism; and/or (vi) ability to form colony forming units (CFU). In an embodiment, the HSPC has (i) the ability to self-renew. In an embodiment, the HSPC has (ii) unlimited proliferative potential. In an embodiment, the HSPC has (iii) the ability to enter and/or exit a quiescent state, e.g., a cell state where no proliferation occurs, e.g., GO phase of the cell cycle. In an embodiment, the HSPC has (iv) the ability to differentiate into any hematopoietic lineage, e.g., myeloid and/or lymphoid lineages, e.g., common lymphoid progenitor (CLP) or a differentiated cell thereof; and/or common myeloid progenitor (CMP) or a differentiated cell thereof. In an embodiment, the HSPC has (v) ability to repopulate any hematopoietic lineage, e.g., myeloid and/or lymphoid lineages, e.g., common lymphoid progenitor (CLP) or a differentiated cell thereof; and/or common myeloid progenitor (CMP) or a differentiated cell thereof, e.g., in an organism. In an embodiment, the HSPC has (vi) the ability to form colony forming units (CFU).

In an embodiment of any of the methods or compositions disclosed herein, the HSPC is a human HSPC, and has one, two, three, four, five, six, seven, eight, or all of the following expression characteristics: (i) expression of CD45, e.g., detectable expression of CD45, e.g., cell surface expression of CD45; (ii) expression of CD34, e.g., detectable expression of CD34, e.g., cell surface expression of CD34; (iii) expression of CD38, e.g., detectable expression of CD38, e.g., cell surface expression of CD38; (iv) expression of CD90 e.g., detectable expression of CD90, e.g., cell surface expression of CD90; (v) expression of CD133 e.g., detectable expression of CD133, e.g., cell surface expression of CD133; (vi) expression of CD45RA, e.g., detectable expression of CD45RA, e.g., cell surface expression of CD45RA; (vii) no detectable or low expression of markers associated with primitive progenitor cells, e.g., CMP, MEP, GMP and/or CLP; (viii) no detectable or low expression of markers associated with lineage committed cells, e.g., TCP, NKP, GP, MP, EP and/or MkP; or (ix) no detectable or low expression of markers associated with one, two or all cell lineage markers of (vii)-(viii), e.g., lineage negative (Lin-). In an embodiment, the HSPC is a human HSPC and has (i) expression of CD45, e.g., detectable expression of CD45, e.g., cell surface expression of CD45. In an embodiment, the HSPC is a human HSPC and has (ii) expression of CD34, e.g., detectable expression of CD34, e.g., cell surface expression of CD34. In an embodiment, the HSPC is a human HSPC and has (iii) expression of CD38, e.g., detectable expression of CD38, e.g., cell surface expression of CD38. In an embodiment, the HSPC is a human HSPC and has (iv) expression of CD90 e.g., detectable expression of CD90, e.g., cell surface expression of CD90. In an embodiment, the HSPC is a human HSPC and has (v) expression of CD133 e.g., detectable expression of CD133, e.g., cell surface expression of CD133. In an embodiment, the HSPC is a human HSPC and has (vi) expression of CD45RA, e.g., detectable expression of CD45RA, e.g., cell surface expression of CD45RA. In an embodiment, the HSPC is a human HSPC and has (vii) no detectable or low expression of markers associated with primitive progenitor cells, e.g., CMP, MEP, GMP and/or CLP. In an embodiment, the HSPC is a human HSPC and has (viii) no detectable or low expression of markers associated with lineage committed cells, e.g., TCP, NKP, GP, MP, EP and/or MkP. In an embodiment, the modified cell is a modified human HSPC and has (ix) no detectable or low expression of markers associated with one, two or all cell lineage markers of (vii)-(viii), e.g., lineage negative (Lin-).

In an embodiment, the human HSPC expresses any one of (i)-(vi). In an embodiment, the modified human HSPC expresses any two of (i)-(vi). In an embodiment, the human HSPC expresses any three of (i)-(vi). In an embodiment, the human HSPC expresses all of (i)-(vi).

In an embodiment, the human HSPC has no detectable or low expression of (vii) or (viii). In an embodiment, the human HSPC has no detectable or low expression of both (vii) and (viii), e.g., wherein the human HSPC is a lineage negative HSPC.

In an embodiment of any of the methods and compositions disclosed herein, the HSPC is an NHP HSPC and has one, two, three, four, five, six, seven, eight, or all of the following expression characteristics: (i) expression of CD45, e.g., detectable expression of CD45, e.g., cell surface expression of CD45; (ii) expression of CD34, e.g., detectable expression of CD34, e.g., cell surface expression of CD34; (iii) expression of c-Kit (CD117), e.g., detectable expression of c-Kit (CD117), e.g., cell surface expression of c-Kit (CD117); (iv) expression of CD90 e.g., detectable expression of CD90, e.g., cell surface expression of CD90; (v) expression of CD123 e.g., detectable expression of CD123, e.g., cell surface expression of CD123; (vi) expression of CD45RA, e.g., detectable expression of CD45RA, e.g., cell surface expression of CD45RA; (vii) no detectable or low expression of markers associated with primitive progenitor cells, e.g., CMP, MEP, GMP and/or CLP; (viii) no detectable or low expression of markers associated with lineage committed cells, e.g., TCP, NKP, GP, MP, EP and/or MkP; or (ix) no detectable or low expression of markers associated with one, two or all cell lineage markers of (vii)-(viii), e.g., lineage negative (Lin-). In an embodiment, the HSPC is an NHP HSPC and has (i) expression of CD45, e.g., detectable expression of CD45, e.g., cell surface expression of CD45. In an embodiment, the HSPC is an NHP HSPC and has (ii) expression of CD34, e.g., detectable expression of CD34, e.g., cell surface expression of CD34. In an embodiment, the HSPC is an NHP HSPC and has (iii) expression of c-Kit (CD117), e.g., detectable expression of c-Kit (CD117), e.g., cell surface expression of c-Kit (CD117). In an embodiment, the HSPC is an NHP HSPC and has (iv) expression of CD90 e.g., detectable expression of CD90, e.g., cell surface expression of CD90. In an embodiment, the HSPC is an NHP HSPC and has (v) expression of CD123 e.g., detectable expression of CD123, e.g., cell surface expression of CD123. In an embodiment, the HSPC is an NHP HSPC and has (vi) expression of CD45RA, e.g., detectable expression of CD45RA, e.g., cell surface expression of CD45RA. In an embodiment, the HSPC is an NHP HSPC and has (vii) no detectable or low expression of markers associated with primitive progenitor cells, e.g., CMP, MEP, GMP and/or CLP. In an embodiment, the HSPC is an NHP HSPC and has (viii) no detectable or low expression of markers associated with lineage committed cells, e.g., TCP, NKP, GP, MP, EP and/or MkP. In an embodiment, the HSPC is an NHP HSPC and has (ix) no detectable or low expression of markers associated with one, two or all cell lineage markers of (vii)-(viii), e.g., lineage negative (Lin-).

In an embodiment, the NHP HSPC expresses any one of (i)-(vi). In an embodiment, the NHP HSPC expresses any two of (i)-(vi). In an embodiment, the NHP HSPC expresses any three of (i)-(vi). In an embodiment, the NHP HSPC expresses all of (i)-(vi).

In an embodiment, the NHP HSPC has no detectable or low expression of (vii) or (viii). In an embodiment, the NHP HSPC has no detectable or low expression of both (vii) and (viii), e.g., wherein the NHP HSPC is a lineage negative HSPC.

In an embodiment of any of the methods and compositions disclosed herein, the HSPC is a mouse HSPC and has one, two, three, four, five, six, seven or all of the following expression characteristics: (i) expression of CD34, e.g., detectable expression of CD34, e.g., cell surface expression of CD34; (ii) expression of CD150 e.g., detectable expression of CD150, e.g., cell surface expression of CD150; (iii) expression of Sca-1 e.g., detectable expression of Sca-1, e.g., cell surface expression of Sca-1; (iv) expression of c-kit e.g., detectable expression of c-KIT, e.g., cell surface expression of c-kit; (v) no detectable or low expression of markers associated with primitive progenitor cells, e.g., CMP and/or CLP; (vi) no detectable or low expression of markers associated with committed precursor cells, e.g., MEP, GM, TNK and/or BCP; (vii) no detectable or low expression of markers associated with lineage committed cells, e.g., TCP, NKP, GP, MP, EP and/or MkP; or (viii) no detectable or low expression of markers associated with one, two or all cell lineage markers of (viii)-(x), e.g., lineage negative (Lin-). In an embodiment, the HSPC is a mouse HSPC and has (i) expression of CD34, e.g., detectable expression of CD34, e.g., cell surface expression of CD34. In an embodiment, the HSPC is a mouse HSPC and has (ii) expression of CD150 e.g., detectable expression of CD150, e.g., cell surface expression of CD150. In an embodiment, the HSPC is a mouse HSPC and has (iii) expression of Sca-1 e.g., detectable expression of Sca-1, e.g., cell surface expression of Sca-1. In an embodiment, the HSPC is a mouse HSPC and has (iv) expression of c-kit e.g., detectable expression of c-KIT, e.g., cell surface expression of c-kit. In an embodiment, the HSPC is a mouse HSPC and has (v) no detectable or low expression of markers associated with primitive progenitor cells, e.g., CMP and/or CLP. In an embodiment, the HSPC is a mouse HSPC and has (vi) no detectable or low expression of markers associated with committed precursor cells, e.g., MEP, GM, TNK and/or BCP. In an embodiment, the HSPC is a mouse HSPC and has (vii) no detectable or low expression of markers associated with lineage committed cells, e.g., TCP, NKP, GP, MP, EP and/or MkP. In an embodiment, the HSPC is a mouse HSPC and has (viii) no detectable or low expression of markers associated with one, two or all cell lineage markers of (v)-(vii), e.g., lineage negative (Lin-).

In an embodiment of any of the methods, compositions, or cells disclosed herein, the mouse HSPC has no detectable expression or low expression of any one of (v)-(vii). In an embodiment of any of the methods, compositions, or cells disclosed herein, the mouse HSPC has no detectable expression or low expression of any two of (v)-(vii). In an embodiment of any of the methods, compositions, or cells disclosed herein, the mouse HSPC has no detectable expression or low expression of all of (v)-(vii), e.g., wherein the mouse HSPC is a lineage negative HSPC.

In an embodiment of any of the methods, compositions, or cells disclosed herein, the mouse HSPC expresses c-Kit and Scal, e.g., a c-KIT+ and Sca-1+HSC. In an embodiment of any of the methods, compositions, or cells disclosed herein, the mouse HSPC expresses c-Kit and Scal, e.g., a c-KIT+ and Sca-1+HSC, and the mouse HSPC has no detectable expression or low expression of any one, any two or all of (v)-(vii).

In an embodiment of any of the methods, compositions, or cells disclosed herein, the mouse HSPC has any one, or all, or a combination of the functional characteristics disclosed herein and the HSPC has any one, or all, or a combination of the expression characteristics disclosed herein. In an embodiment, the functional characteristics comprise: (i) ability to self-renew; (ii) unlimited proliferative potential; (iii) ability to enter and/or exit a quiescent state, e.g., a cell state where no proliferation occurs, e.g., GO phase of the cell cycle; (iv) ability to differentiate into any hematopoietic lineage, e.g., myeloid and/or lymphoid lineages, e.g., common lymphoid progenitor (CLP) or a differentiated cell thereof, and/or common myeloid progenitor (CMP) or a differentiated cell thereof, (v) ability to repopulate any hematopoietic lineage, e.g., myeloid and/or lymphoid lineages, e.g., common lymphoid progenitor (CLP) or a differentiated cell thereof; and/or common myeloid progenitor (CMP) or a differentiated cell thereof, e.g., in an organism; and/or (vi) ability to form colony forming units (CFU). In an embodiment, the expression characteristics comprise: (i) expression of CD34, e.g., detectable expression of CD34, e.g., cell surface expression of CD34; (ii) expression of CD150 e.g., detectable expression of CD150, e.g., cell surface expression of CD150; (iii) expression of Sca-1 e.g., detectable expression of Sca-1, e.g., cell surface expression of Sca-1; (iv) expression of c-kit e.g., detectable expression of c-KIT, e.g., cell surface expression of c-kit; (v) no detectable or low expression of markers associated with primitive progenitor cells, e.g., CMP and/or CLP; (vi) no detectable or low expression of markers associated with committed precursor cells, e.g., MEP, GM, TNK and/or BCP; (vii) no detectable or low expression of markers associated with lineage committed cells, e.g., TCP, NKP, GP, MP, EP and/or MkP; or (viii) no detectable or low expression of markers associated with one, two or all cell lineage markers of (v)-(vii), e.g., lineage negative (Lin-).

In one embodiment, it will be understood that the exemplary markers described herein encompass other mammalian (e.g., human) orthologs or equivalents of the exemplary NHP or mouse markers described herein.

In an embodiment of any of the methods, compositions, or cells disclosed herein, the modified cell (e.g., modified stem or progenitor cell, e.g., modified HSPC is a modified human cell and has one, two, three, four, five, six, seven, eight, or all of the following expression characteristics: (i) expression (e.g., detectable expression, e.g., cell surface expression) of a human ortholog or equivalent of NHP CD45; (ii) expression (e.g., detectable expression, e.g., cell surface expression) of a human ortholog or equivalent of NHP CD34; (iii) expression (e.g., detectable expression, e.g., cell surface expression) of a human ortholog or equivalent of NHP c-Kit (CD117); (iv) expression (e.g., detectable expression, e.g., cell surface expression) of a human ortholog or equivalent of NHP CD90; (v) expression (e.g., detectable expression, e.g., cell surface expression) of a human ortholog or equivalent of NHP CD123; (vi) expression (e.g., detectable expression, e.g., cell surface expression) of a human ortholog or equivalent of NHP CD45RA; (vii) no detectable or low expression of markers associated with primitive progenitor cells, e.g., a human ortholog or equivalent of NHP CMP, MEP, GMP and/or CLP; (viii) no detectable or low expression of markers associated with lineage committed cells, e.g., a human ortholog or equivalent of NHP TCP, NKP, GP, MP, EP and/or MkP; or (ix) no detectable or low expression of markers associated with one, two or all cell lineage markers of (vii)-(viii), e.g., lineage negative (Lin-), or a human ortholog or equivalent thereof.

In an embodiment of any of the methods, compositions, or cells disclosed herein, the modified cell, (e.g., modified stem or progenitor cell, e.g., modified HSPC) is a modified human cell and has one, two, three, four, five, six, seven or all of the following expression characteristics: (i) expression (e.g., detectable expression, e.g., cell surface expression) of a human ortholog or equivalent of mouse CD34; (ii) expression (e.g., detectable expression, e.g., cell surface expression) of a human ortholog or equivalent of mouse CD150; (iii) expression (e.g., detectable expression, e.g., cell surface expression) of a human ortholog or equivalent of mouse Sca-1; (iv) expression (e.g., detectable expression, e.g., cell surface expression) of a human ortholog or equivalent of mouse c-kit; (v) no detectable or low expression of markers associated with primitive progenitor cells, e.g., a human ortholog or equivalent of mouse CMP and/or CLP; (vi) no detectable or low expression of markers associated with committed precursor cells, e.g., a human ortholog or equivalent of mouse MEP, GM, TNK and/or BCP; (vii) no detectable or low expression of markers associated with lineage committed cells, e.g., a human ortholog or equivalent of mouse TCP, NKP, GP, MP, EP and/or MkP; or (viii) no detectable or low expression of markers associated with one, two or all cell lineage markers of (v)-(vii), e.g., lineage negative (Lin-), or a human ortholog or equivalent thereof. In an embodiment, the modified human HSPC expresses human orthologs or equivalents of mouse c-Kit and Scal. In an embodiment, the modified human HSPC expresses human orthologs or equivalents of mouse c-Kit and Scal, and has no detectable expression or low expression of any one, two or all of (v)-(vii).

In an embodiment of any of the methods, compositions, or cells disclosed herein, the HSPC has any one, or all, or a combination of the functional characteristics disclosed herein and the HSPC has any one, or all, or a combination of the expression characteristics disclosed herein. In an embodiment, the functional characteristics comprise: (i) ability to self-renew; (ii) unlimited proliferative potential; (iii) ability to enter and/or exit a quiescent state, e.g., a cell state where no proliferation occurs, e.g., GO phase of the cell cycle; (iv) ability to differentiate into any hematopoietic lineage, e.g., myeloid and/or lymphoid lineages, e.g., common lymphoid progenitor (CLP) or a differentiated cell thereof, and/or common myeloid progenitor (CMP) or a differentiated cell thereof; (v) ability to repopulate any hematopoietic lineage, e.g., myeloid and/or lymphoid lineages, e.g., common lymphoid progenitor (CLP) or a differentiated cell thereof, and/or common myeloid progenitor (CMP) or a differentiated cell thereof, e.g., in an organism; and/or (vi) ability to form colony forming units (CFU). In an embodiment, the expression characteristics comprise: (i) expression of CD45, e.g., detectable expression of CD45, e.g., cell surface expression of CD45; (ii) expression of CD34, e.g., detectable expression of CD34, e.g., cell surface expression of CD34; (iii) expression of CD38, e.g., detectable expression of CD38, e.g., cell surface expression of CD38; (iv) expression of CD90 e.g., detectable expression of CD90, e.g., cell surface expression of CD90; (v) expression of CD133 e.g., detectable expression of CD133, e.g., cell surface expression of CD133; (vi) expression of CD45RA, e.g., detectable expression of CD45RA, e.g., cell surface expression of CD45RA; (vii) no detectable or low expression of markers associated with primitive progenitor cells, e.g., CMP, MEP, GMP and/or CLP; (viii) no detectable or low expression of markers associated with lineage committed cells, e.g., TCP, NKP, GP, MP, EP and/or MkP; or (ix) no detectable or low expression of markers associated with one, two or all cell lineage markers of (vii)-(viii), e.g., lineage negative (Lin-).

In an embodiment of any of the methods, compositions, or cells disclosed herein, prior to contacting the cell with the LNP composition, the cell (e.g., population of cells) is isolated from a subject and expanded, enriched and/or cultured in vitro. In an embodiment of any of the methods, compositions, or cells disclosed herein, the expanded, enriched and/or cultured cell, e.g., population of cells, is administered into a host, e.g., an autologous or allogeneic host.

Modification of a Component or Parameter Associated with a Cell or Tissue

In an embodiment of any of the methods, compositions, or cells disclosed herein, administration or delivery of the LNP composition results in a modification of the cell, or tissue, e.g., a component associated with the cell or tissue, or a parameter associated with the cell or tissue. In an embodiment, administration or delivery of the LNP composition modifies a parameter associated with the cell, e.g., as described herein. In an embodiment, administration or delivery of the LNP composition modifies a component associated with the cell, e.g., as described herein. In an embodiment, administration or delivery of the LNP composition modifies a genotype, a phenotype, and/or a function of a cell, e.g., a common myeloid progenitor cell, a common lymphoid progenitor cell, a multipotent progenitor cell, or a multipotent stem cell. In an embodiment, the cell is an HSPC.

In an embodiment, the component associated with the cell or tissue comprises: (1) a nucleic acid associated with the cell or fragment thereof, e.g., DNA (e.g., exonic, intronic, intergenic, telomeric, promoter, enhancer, insulator, repressor, coding, or non-coding) or RNA (e.g., mRNA, rRNA, tRNA, regulatory RNA, non-coding RNA, long non-coding RNA (lncRNA), guide RNA (gRNA), piwi-interacting RNA (piRNA), small nucleolar RNA (snoRNA), small nuclear RNA (snRNA), extracellular RNA (exRNA), small Cajal body-specific RNA (scaRNA), micro RNA (miRNA), circular RNA, or an RNAi molecule, e.g., small interfering RNA (siRNA) or small hairpin RNA (shRNA)); (2) a peptide or protein associated with the cell or fragment thereof; (3) a lipid component associated with the cell or fragment thereof, or a combination thereof. In an embodiment, the component comprises: (1) a nucleic acid associated with the cell or fragment thereof, e.g., DNA (e.g., exonic, intronic, intergenic, telomeric, promoter, enhancer, insulator, repressor, coding, or non-coding) or RNA (e.g., mRNA, rRNA, tRNA, regulatory RNA, non-coding RNA, long non-coding RNA (lncRNA), guide RNA (gRNA), piwi-interacting RNA (piRNA), small nucleolar RNA (snoRNA), small nuclear RNA (snRNA), extracellular RNA (exRNA), small Cajal body-specific RNA (scaRNA), micro RNA (miRNA), circular RNA, or an RNAi molecule, e.g., small interfering RNA (siRNA) or small hairpin RNA (shRNA)). In an embodiment, the component comprises DNA. In an embodiment, the component comprises RNA. In an embodiment, the component comprises (2) a peptide or protein associated with the cell or fragment thereof. In an embodiment, the component comprises (3) a lipid component associated with the cell or fragment thereof.

In an embodiment of any of the methods, compositions, or cells disclosed herein, the component is endogenous to the cell.

In an embodiment of any of the methods, compositions, or cells disclosed herein, the component is exogenous to the cell, e.g., has been introduced into the cell by a method known in the art, e.g., electroporation, transformation, vector-based delivery, viral delivery or lipid-based delivery.

In an embodiment of any of the methods, compositions, or cells disclosed herein, the parameter associated with the cell or tissue comprises an expression parameter, a phenotypic parameter or a signaling parameter. In an embodiment, the parameter associated with the cell or tissue comprises an expression parameter. In an embodiment, the parameter associated with the cell or tissue comprises a phenotypic parameter. In an embodiment, the parameter associated with the cell or tissue comprises a signaling parameter.

In an embodiment of any of the methods, compositions, or cells disclosed herein, the expression parameter comprises one, two, three, four or all of the following: (a) expression level (e.g., of polypeptide or protein, or polynucleotide or nucleic acid, e.g., mRNA); (b) activity (e.g., of polypeptide or protein, or polynucleotide or nucleic acid, e.g., mRNA), (c) post-translational modification of polypeptide or protein; (d) folding (e.g., of polypeptide or protein, or polynucleotide or nucleic acid, e.g., mRNA), and/or (e) stability (e.g., of polypeptide or protein, or polynucleotide or nucleic acid, e.g., mRNA). In an embodiment, the expression parameter comprises(a) expression level (e.g., of polypeptide or protein, or polynucleotide or nucleic acid, e.g., mRNA). In an embodiment, the expression parameter comprises, (b) activity (e.g., of polypeptide or protein, or polynucleotide or nucleic acid, e.g., mRNA). In an embodiment, the expression parameter comprises, (c) post-translational modification of polypeptide or protein. In an embodiment, the expression parameter comprises, (d) folding (e.g., of polypeptide or protein, or polynucleotide or nucleic acid, e.g., mRNA). In an embodiment, the expression parameter comprises, (e) stability (e.g., of polypeptide or protein, or polynucleotide or nucleic acid, e.g., mRNA).

In an embodiment of any of the methods, compositions, or cells disclosed herein, the signaling parameter comprises one, two, three, four or all of the following: (1) modulation of a signaling pathway, e.g., a cellular signaling pathway; (2) cell fate modulation; (3) modulation of expression level (e.g., of polypeptide or protein, or polynucleotide or nucleic acid, e.g., mRNA); (4) modulation of activity (e.g., of polypeptide or protein, or polynucleotide or nucleic acid, e.g., mRNA), and/or (5) modulation of stability e.g., of polypeptide or protein, or polynucleotide or nucleic acid, e.g., mRNA). In an embodiment, the signaling parameter comprises (1) modulation of a signaling pathway, e.g., a cellular signaling pathway. In an embodiment, the signaling parameter comprises (2) cell fate modulation. In an embodiment, the signaling parameter comprises (3) modulation of expression level (e.g., of polypeptide or protein, or polynucleotide or nucleic acid, e.g., mRNA). In an embodiment, the signaling parameter comprises (4) modulation of activity (e.g., of polypeptide or protein, or polynucleotide or nucleic acid, e.g., mRNA). In an embodiment, the signaling parameter comprises (5) modulation of stability e.g., of polypeptide or protein, or polynucleotide or nucleic acid, e.g., mRNA).

In an embodiment of any of the methods, compositions, or cells disclosed herein, the phenotypic parameter comprises expression and/or activity of a molecule, e.g., cell surface protein, lipid or adhesion molecule, on the surface of the cell.

Effect of Modifying an HSC In Vivo with an LNP Composition

In an embodiment of any of the methods, compositions, or cells disclosed herein, the cell or tissue modified with an LNP composition disclosed herein, e.g., modified cell, e.g., modified stem or progenitor cell, e.g., modified HSPC, has a characteristic disclosed herein.

In an embodiment of any of the methods, compositions, or cells disclosed herein, the modified cell, e.g., modified stem or progenitor cell, e.g., modified HSPC, has one, two, three, four, five or all of the following functional characteristics: (i) ability to self-renew; (ii) unlimited proliferative potential; (iii) ability to enter and/or exit a quiescent state, e.g., a cell state where no proliferation occurs, e.g., GO phase of the cell cycle; (iv) ability to differentiate into any hematopoietic lineage, e.g., myeloid and/or lymphoid lineages, e.g., common lymphoid progenitor (CLP) or a differentiated cell thereof, and/or common myeloid progenitor (CMP) or a differentiated cell thereof; (v) ability to repopulate any hematopoietic lineage, e.g., myeloid and/or lymphoid lineages, e.g., common lymphoid progenitor (CLP) or a differentiated cell thereof, and/or common myeloid progenitor (CMP) or a differentiated cell thereof, e.g., in an organism; and/or (vi) ability to form colony forming units (CFU). In an embodiment, the modified cell, e.g., modified stem cell, e.g., modified HSPC, has (i) the ability to self-renew. In an embodiment, the modified cell, e.g., modified stem cell, e.g., modified HSPC, has (ii) unlimited proliferative potential. In an embodiment, the modified cell, e.g., modified stem or progenitor cell, e.g., modified HSPC, has (iii) the ability to enter and/or exit a quiescent state, e.g., a cell state where no proliferation occurs, e.g., GO phase of the cell cycle. In an embodiment, the modified cell, e.g., modified stem cell, e.g., modified HSPC, has (iv) the ability to differentiate into any hematopoietic lineage, e.g., myeloid and/or lymphoid lineages, e.g., common lymphoid progenitor (CLP) or a differentiated cell thereof, and/or common myeloid progenitor (CMP) or a differentiated cell thereof. In an embodiment, the modified cell, e.g., modified stem or progenitor cell, e.g., modified HSPC, has (v) ability to repopulate any hematopoietic lineage, e.g., myeloid and/or lymphoid lineages, e.g., common lymphoid progenitor (CLP) or a differentiated cell thereof, and/or common myeloid progenitor (CMP) or a differentiated cell thereof; e.g., in an organism. In an embodiment, the modified cell, e.g., modified stem or progenitor cell, e.g., modified HSPC, has (vi) the ability to form colony forming units (CFU).

In an embodiment, the modified HSPC has the ability to form CFU, e.g., as measured in an ex-vivo colony-forming unit (CFU) assay, e.g., as described in Example 2. In an embodiment, the CFU ability is compared to an otherwise similar HSPC which has not been contacted with an LNP, or has been contacted with a different LNP.

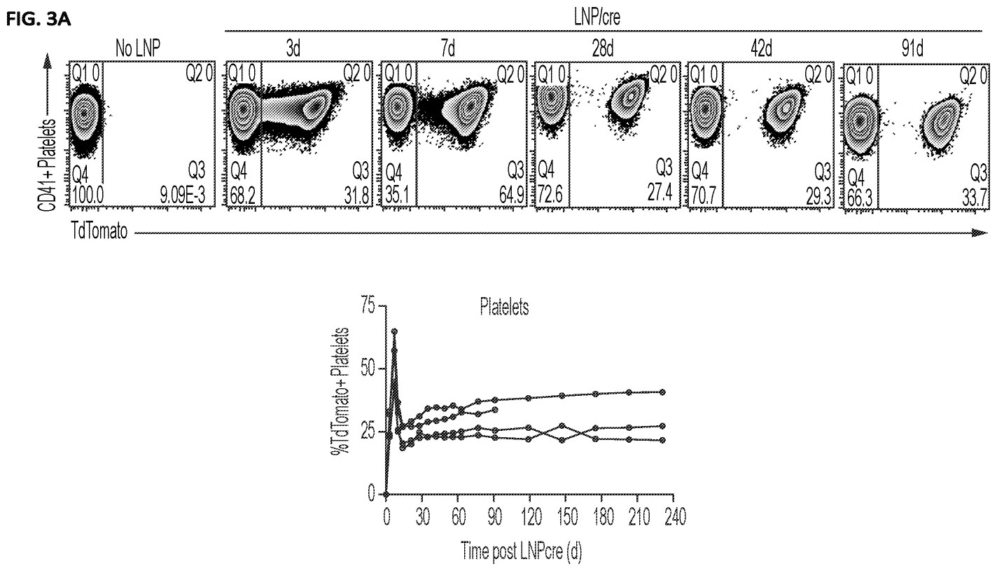

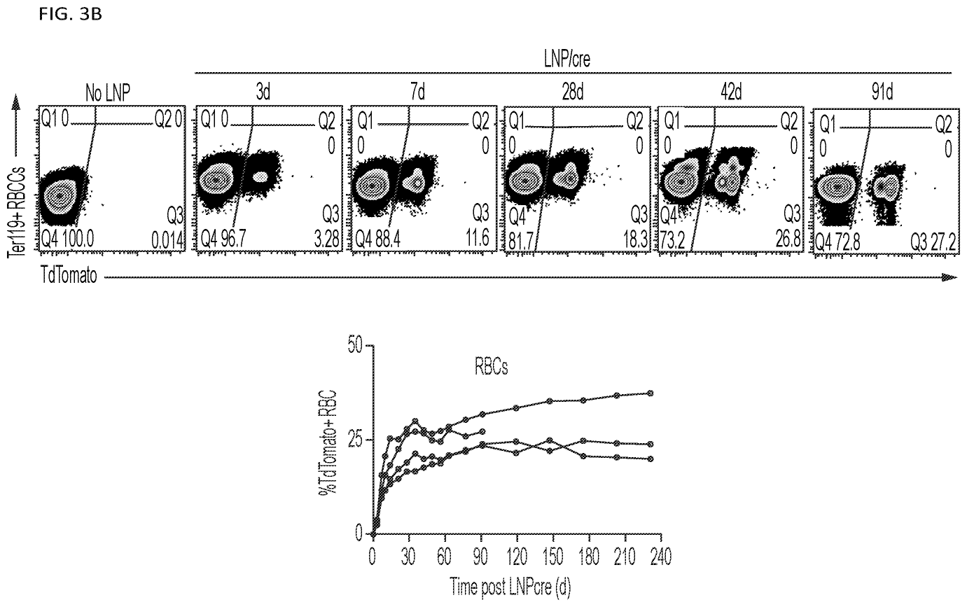

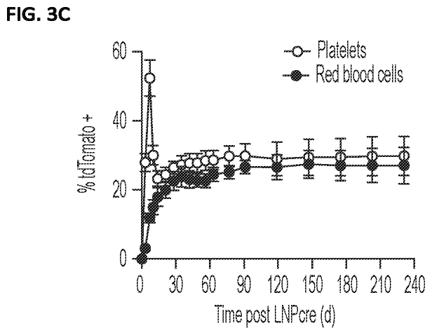

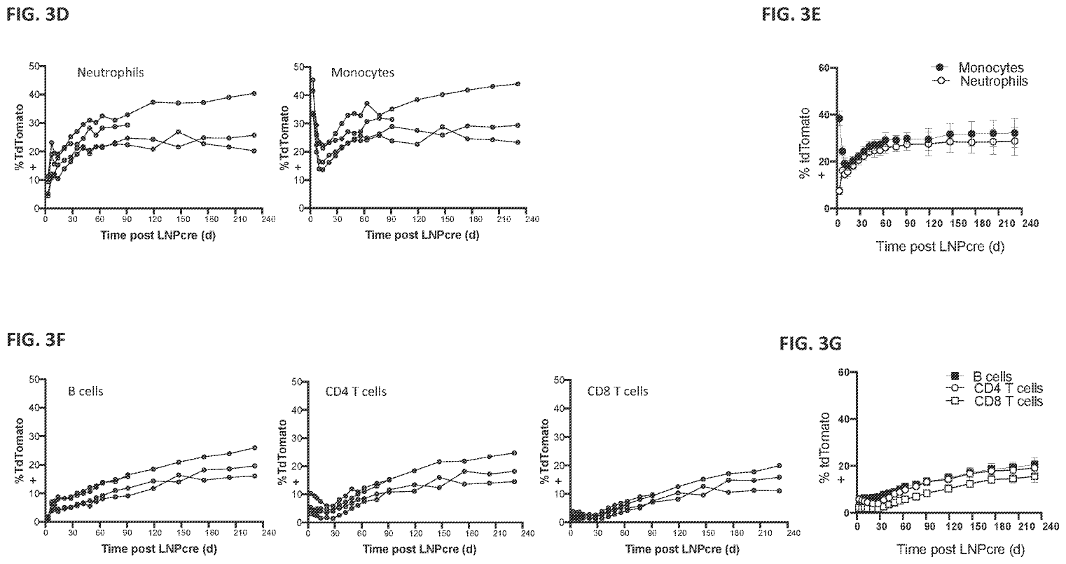

In an embodiment, the modified HSPC has the ability to differentiate into myeloid cells, e.g., as measured in an ex-vivo colony-forming unit (CFU) assay, e.g., as described in Example 2, or as measured in a lineage tracing experiment, e.g., as described in Example 3 (e.g., FIG. 3D). In an embodiment the ability of the modified HSPC to differentiate into myeloid cells is compared to an otherwise similar HSPC which has not been contacted with an LNP, or has been contacted with a different LNP.

In an embodiment, the modified HSPC has the ability to differentiate into lymphoid cells, e.g., as measured in a lineage tracing experiment, e.g., as described in Example 3 (e.g., FIG. 3C). In an embodiment, the ability of the modified HSPC to differentiate into lymphoid cells is compared to an otherwise similar HSPC which has not been contacted with an LNP, or has been contacted with a different LNP.

In an embodiment, the modified HSPC has the ability to differentiate into an erythrocyte cell or a platelet, e.g., as described in Example 3 (e.g., FIGS. 3A-3B). In an embodiment, the ability of the modified HSPC to differentiate into an erythrocyte cell or a platelet is compared to an otherwise similar HSPC which has not been contacted with an LNP, or has been contacted with a different LNP. In an embodiment, the modified HSPC differentiates into an erythrocyte cell or a platelet in vivo. In an embodiment, the modified HSPC differentiates into an erythrocyte cell or a platelet in vitro.

In an embodiment, the modified HSPC persists, e.g., in vivo, for at least 1, 2, 3, 4, 5, 6, 7, 10, 15, 20, 25 or 30 days. In an embodiment, the modified HSPC persists, e.g., in vivo, for 1-30, 2-30, 3-30, 4-30, 5-30, 6-30, 7-30, 10-30, 15-30, 20-30, 25-30, 1-25, 1-20, 1-15, 1-10, 1-7, 1-6, 1-5, 1-4, 1-3 or 1-2 days. In an embodiment, the in vivo persistence of the modified HSPC results in differentiation into one or more cells, e.g., cells in the myeloid and/or cells in the lymphoid lineage, e.g., as shown in Example 3.

In an embodiment of any of the methods, compositions, or cells disclosed herein, the modified cell (e.g., modified stem or progenitor cell, e.g., modified HSPC) is a human cell, and has one, two, three, four, five, six, seven, eight, or all of the following expression characteristics: (i) expression of CD45, e.g., detectable expression of CD45, e.g., cell surface expression of CD45; (ii) expression of CD34, e.g., detectable expression of CD34, e.g., cell surface expression of CD34; (iii) expression of CD38, e.g., detectable expression of CD38, e.g., cell surface expression of CD38; (iv) expression of CD90 e.g., detectable expression of CD90, e.g., cell surface expression of CD90; (v) expression of CD133 e.g., detectable expression of CD133, e.g., cell surface expression of CD133; (vi) expression of CD45RA, e.g., detectable expression of CD45RA, e.g., cell surface expression of CD45RA; (vii) no detectable or low expression of markers associated with primitive progenitor cells, e.g., CMP, MEP, GMP and/or CLP; (viii) no detectable or low expression of markers associated with lineage committed cells, e.g., TCP, NKP, GP, MP, EP and/or MkP; or (ix) no detectable or low expression of markers associated with one, two or all cell lineage markers of (vii)-(viii), e.g., lineage negative (Lin-). In an embodiment, the modified cell is a modified human HSPC and has (i) expression of CD45, e.g., detectable expression of CD45, e.g., cell surface expression of CD45. In an embodiment, the modified cell is a modified human HSPC and has (ii) expression of CD34, e.g., detectable expression of CD34, e.g., cell surface expression of CD34. In an embodiment, the modified cell is a modified human HSPC and has (iii) expression of CD38, e.g., detectable expression of CD38, e.g., cell surface expression of CD38. In an embodiment, the modified cell is a modified human HSPC and has (iv) expression of CD90 e.g., detectable expression of CD90, e.g., cell surface expression of CD90. In an embodiment, the modified cell is a modified human HSPC and has (v) expression of CD133 e.g., detectable expression of CD133, e.g., cell surface expression of CD133. In an embodiment, the modified cell is a modified human HSPC and has (vi) expression of CD45RA, e.g., detectable expression of CD45RA, e.g., cell surface expression of CD45RA. In an embodiment, the modified cell is a modified human HSPC and has (vii) no detectable or low expression of markers associated with primitive progenitor cells, e.g., CMP, MEP, GMP and/or CLP. In an embodiment, the modified cell is a modified human HSPC and has (viii) no detectable or low expression of markers associated with lineage committed cells, e.g., TCP, NKP, GP, MP, EP and/or MkP. In an embodiment, the modified cell is a modified human HSPC and has (ix) no detectable or low expression of markers associated with one, two or all cell lineage markers of (vii)-(viii), e.g., lineage negative (Lin-).

In an embodiment, the modified human HSPC expresses any one of (i)-(vi). In an embodiment, the modified human HSPC expresses any two of (i)-(vi). In an embodiment, the modified human HSPC expresses any three of (i)-(vi). In an embodiment, the modified human HSPC expresses all of (i)-(vi).

In an embodiment, the modified human HSPC has no detectable or low expression of (vii) or (viii). In an embodiment, the modified human HSPC has no detectable or low expression of both (vii) and (viii), e.g., wherein the human HSPC is a lineage negative HSPC.

In an embodiment of any of the methods, compositions, or cells disclosed herein, the modified cell (e.g., modified stem or progenitor cell, e.g., modified HSPC) is a non-human primate (NHP) cell and has one, two, three, four, five, six, seven, eight, or all of the following expression characteristics: (i) expression of CD45, e.g., detectable expression of CD45, e.g., cell surface expression of CD45; (ii) expression of CD34, e.g., detectable expression of CD34, e.g., cell surface expression of CD34; (iii) expression of c-Kit (CD117), e.g., detectable expression of c-Kit (CD117), e.g., cell surface expression of c-Kit (CD117); (iv) expression of CD90 e.g., detectable expression of CD90, e.g., cell surface expression of CD90; (v) expression of CD123 e.g., detectable expression of CD123, e.g., cell surface expression of CD123; (vi) expression of CD45RA, e.g., detectable expression of CD45RA, e.g., cell surface expression of CD45RA; (vii) no detectable or low expression of markers associated with primitive progenitor cells, e.g., CMP, MEP, GMP and/or CLP; (viii) no detectable or low expression of markers associated with lineage committed cells, e.g., TCP, NKP, GP, MP, EP and/or MkP; or (ix) no detectable or low expression of markers associated with one, two or all cell lineage markers of (vii)-(viii), e.g., lineage negative (Lin-). In an embodiment, the modified cell is a modified NHP HSPC and has (i) expression of CD45, e.g., detectable expression of CD45, e.g., cell surface expression of CD45. In an embodiment, the modified cell is a modified NHP HSPC and has (ii) expression of CD34, e.g., detectable expression of CD34, e.g., cell surface expression of CD34. In an embodiment, the modified cell is a modified NHP HSPC and has (iii) expression of c-Kit (CD117), e.g., detectable expression of c-Kit (CD117), e.g., cell surface expression of c-Kit (CD117). In an embodiment, the modified cell is a modified NHP HSPC and has (iv) expression of CD90 e.g., detectable expression of CD90, e.g., cell surface expression of CD90. In an embodiment, the modified cell is a modified NHP HSPC and has (v) expression of CD123 e.g., detectable expression of CD123, e.g., cell surface expression of CD123. In an embodiment, the modified cell is a modified NHP HSPC and has (vi) expression of CD45RA, e.g., detectable expression of CD45RA, e.g., cell surface expression of CD45RA. In an embodiment, the modified cell is a modified NHP HSPC and has (vii) no detectable or low expression of markers associated with primitive progenitor cells, e.g., CMP, MEP, GMP and/or CLP. In an embodiment, the modified cell is a modified NHP HSPC and has (viii) no detectable or low expression of markers associated with lineage committed cells, e.g., TCP, NKP, GP, MP, EP and/or MkP. In an embodiment, the modified cell is a modified NHP HSPC and has (ix) no detectable or low expression of markers associated with one, two or all cell lineage markers of (vii)-(viii), e.g., lineage negative (Lin-).

In an embodiment, the modified NHP HSPC expresses any one of (i)-(vi). In an embodiment, the modified NHP HSPC expresses any two of (i)-(vi). In an embodiment, the modified NHP HSPC expresses any three of (i)-(vi). In an embodiment, the modified NHP HSPC expresses all of (i)-(vi).

In an embodiment, the modified NHP HSPC has no detectable or low expression of (vii) or (viii). In an embodiment, the modified NHP HSPC has no detectable or low expression of both (vii) and (viii), e.g., wherein the NHP HSPC is a lineage negative HSPC.

In an embodiment of any of the methods, compositions, or cells disclosed herein, the modified cell, (e.g., modified stem or progenitor cell, e.g., modified HSPC) is a modified mouse cell and has one, two, three, four, five, six, seven or all of the following expression characteristics: (i) expression of CD34, e.g., detectable expression of CD34, e.g., cell surface expression of CD34; (ii) expression of CD150, e.g., detectable expression of CD150, e.g., cell surface expression of CD150; (iii) expression of Sca-1 e.g., detectable expression of Sca-1, e.g., cell surface expression of Sca-1; (iv) expression of c-kit e.g., detectable expression of c-KIT, e.g., cell surface expression of c-kit; (v) no detectable or low expression of markers associated with primitive progenitor cells, e.g., CMP and/or CLP; (vi) no detectable or low expression of markers associated with committed precursor cells, e.g., MEP, GM, TNK and/or BCP; (vii) no detectable or low expression of markers associated with lineage committed cells, e.g., TCP, NKP, GP, MP, EP and/or MkP; or (viii) no detectable or low expression of markers associated with one, two or all cell lineage markers of (v)-(vii), e.g., lineage negative (Lin-). In an embodiment, the modified cell is a modified mouse HSPC and has (i) expression of CD34, e.g., detectable expression of CD34, e.g., cell surface expression of CD34. In an embodiment, the modified cell is a modified mouse HSPC and has (ii) expression of CD150 e.g., detectable expression of CD150, e.g., cell surface expression of CD150. In an embodiment, the modified cell is a modified mouse HSPC and has (iii) expression of Sca-1 e.g., detectable expression of Sca-1, e.g., cell surface expression of Sca-1. In an embodiment, the modified cell is a modified mouse HSPC and has (iv) expression of c-kit e.g., detectable expression of c-KIT, e.g., cell surface expression of c-kit. In an embodiment, the modified cell is a modified mouse HSPC and has (v) no detectable or low expression of markers associated with primitive progenitor cells, e.g., CMP and/or CLP. In an embodiment, the modified cell is a modified mouse HSPC and has (vi) no detectable or low expression of markers associated with committed precursor cells, e.g., MEP, GM, TNK and/or BCP. In an embodiment, the modified cell is a modified mouse HSPC and has (vii) no detectable or low expression of markers associated with lineage committed cells, e.g., TCP, NKP, GP, MP, EP and/or MkP. In an embodiment, the modified cell is a modified mouse HSPC and has (viii) no detectable or low expression of markers associated with one, two or all cell lineage markers of (v)-(vii), e.g., lineage negative (Lin-).

In an embodiment, the modified mouse HSPC expresses any one of (i)-(iv). In an embodiment, the modified mouse HSPC expresses any two of (i)-(iv). In an embodiment, the modified mouse HSPC expresses any three of (i)-(iv). In an embodiment, the modified mouse HSPC expresses all of (i)-(iv).

In an embodiment, the modified mouse HSPC has no detectable or low expression of any one of (v)-(vii). In an embodiment, the modified mouse HSPC has no detectable or low expression of any two of (v)-(vii). In an embodiment, the modified mouse HSPC has no detectable or low expression of all of (v)-(vii), e.g., wherein the mouse HSPC is a lineage negative HSPC.

In an embodiment, the modified mouse HSPC expresses c-Kit and Scal, e.g., a c-KIT+ and Sca-1+HSC. In an embodiment, the modified mouse HSPC expresses c-Kit and Scal, e.g., a c-KIT+ and Sca-1+HSC, and has no detectable expression or low expression of any one, two or all of (v)-(vii).

In an embodiment of any of the methods, compositions, or cells disclosed herein, the modified cell (e.g., modified stem or progenitor cell, e.g., modified HSPC is a modified human cell and has one, two, three, four, five, six, seven, eight, or all of the following expression characteristics: (i) expression (e.g., detectable expression, e.g., cell surface expression) of a human ortholog or equivalent of NHP CD45; (ii) expression (e.g., detectable expression, e.g., cell surface expression) of a human ortholog or equivalent of NHP CD34; (iii) expression (e.g., detectable expression, e.g., cell surface expression) of a human ortholog or equivalent of NHP c-Kit (CD117); (iv) expression (e.g., detectable expression, e.g., cell surface expression) of a human ortholog or equivalent of NHP CD90; (v) expression (e.g., detectable expression, e.g., cell surface expression) of a human ortholog or equivalent of NHP CD123; (vi) expression (e.g., detectable expression, e.g., cell surface expression) of a human ortholog or equivalent of NHP CD45RA; (vii) no detectable or low expression of markers associated with primitive progenitor cells, e.g., a human ortholog or equivalent of NHP CMP, MEP, GMP and/or CLP; (viii) no detectable or low expression of markers associated with lineage committed cells, e.g., a human ortholog or equivalent of NHP TCP, NKP, GP, MP, EP and/or MkP; or (ix) no detectable or low expression of markers associated with one, two or all cell lineage markers of (vii)-(viii), e.g., lineage negative (Lin-), or a human ortholog or equivalent thereof.

In an embodiment of any of the methods, compositions, or cells disclosed herein, the modified cell, (e.g., modified stem or progenitor cell, e.g., modified HSPC) is a modified human cell and has one, two, three, four, five, six, seven or all of the following expression characteristics: (i) expression (e.g., detectable expression, e.g., cell surface expression) of a human ortholog or equivalent of mouse CD34; (ii) expression (e.g., detectable expression, e.g., cell surface expression) of a human ortholog or equivalent of mouse CD150; (iii) expression (e.g., detectable expression, e.g., cell surface expression) of a human ortholog or equivalent of mouse Sca-1; (iv) expression (e.g., detectable expression, e.g., cell surface expression) of a human ortholog or equivalent of mouse c-kit; (v) no detectable or low expression of markers associated with primitive progenitor cells, e.g., a human ortholog or equivalent of mouse CMP and/or CLP; (vi) no detectable or low expression of markers associated with committed precursor cells, e.g., a human ortholog or equivalent of mouse MEP, GM, TNK and/or BCP; (vii) no detectable or low expression of markers associated with lineage committed cells, e.g., a human ortholog or equivalent of mouse TCP, NKP, GP, MP, EP and/or MkP; or (viii) no detectable or low expression of markers associated with one, two or all cell lineage markers of (v)-(vii), e.g., lineage negative (Lin-), or a human ortholog or equivalent thereof. In an embodiment, the modified human HSPC expresses human orthologs or equivalents of mouse c-Kit and Scal. In an embodiment, the modified human HSPC expresses human orthologs or equivalents of mouse c-Kit and Scal, and has no detectable expression or low expression of any one, two or all of (v)-(vii).

### Payload

In an embodiment of any of the methods, compositions, or cells disclosed herein, the LNP composition comprises a payload, e.g., as described herein. In an embodiment, the payload modifies, e.g., increases or decreases, the component or parameter associated with the cell or tissue, resulting in a modified cell, e.g., modified HSPC, or tissue. In an embodiment, the payload comprises a nucleic-acid molecule, a peptide molecule, a lipid molecule, a low molecular weight molecule, or a combination thereof. In an embodiment, the payload affects a parameter or component of a stem or progenitor cell, e.g., a common myeloid progenitor cell, a common lymphoid progenitor cell, a multipotent progenitor cell, or a multipotent stem cell. In an embodiment, the progenitor cell is an HSPC, e.g., an HSC or HPC. In a preferred embodiment, the payload produces a change in a hemoglobinopathy, a clotting factor disorder, a blood cell disorder, or an immune cell disorder in a subject.

In an embodiment, the payload comprises a nucleic acid molecule comprising a DNA molecule, e.g., double stranded DNA; single stranded DNA; or plasmid DNA. In an embodiment, the payload comprises a nucleic acid molecule comprising an RNA molecule, e.g., mRNA, rRNA, tRNA regulatory RNA, non-coding RNA, long non-coding RNA (lncRNA), guide RNA (gRNA), piwi-interacting RNA (piRNA), small nucleolar RNA (snoRNA), small nuclear RNA (snRNA), extracellular RNA (exRNA), small Cajal body-specific RNA (scaRNA), microRNA (miRNA), microRNA (miRNA), circular RNA, or an RNAi molecule, e.g., small interfering (siRNA) or small hairpin RNA (shRNA). In an embodiment, the payload comprises the payload comprises mRNA. In an embodiment, the mRNA comprises at least one chemical modification. In an embodiment, the chemical modification is selected from the group consisting of pseudouridine, N1-methylpseudouridine, 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methyluridine, 5-methoxyuridine, and 2′-0-methyl uridine. In an embodiment, the chemical modification is selected from the group consisting of pseudouridine, N1-methylpseudouridine, 5-methylcytosine, 5-methoxyuridine, and a combination thereof. In an embodiment, the chemical modification is N1-methylpseudouridine.

In an embodiment, the mRNA comprises fully modified N1-methylpseudouridine.

In an embodiment, the payload comprises a protein, polypeptide, or peptide molecule.

In an embodiment, the payload comprises a lipid molecule, e.g., as described herein.

In an embodiment, the payload comprises a low molecular weight molecule, e.g., as described herein.

In an embodiment of any of the methods, compositions, or cells disclosed herein, the payload comprises a genetic modulator (e.g., a modulator that genetically alters the cell or tissue); an epigenetic modulator (e.g., a modulator that epigenetically alters the cell or tissue); an RNA modulator (e.g., a modulator that alters an RNA molecule in the cell or tissue); a peptide modulator (e.g., a modulator that alters a peptide molecule in the cell or tissue); a lipid modulator (e.g., a modulator that alters a lipid molecule in the cell or tissue); or a combination thereof.

In an embodiment of any of the methods disclosed herein the payload comprises a peptide modulator (e.g., a modulator that alters a peptide molecule in the cell or tissue).

In an embodiment, the payload comprises a lipid modulator (e.g., a modulator that alters a lipid molecule in the cell or tissue); or a combination thereof.

### Genetic Modulators

In an embodiment, the payload comprises a genetic modulator (e.g., a modulator that genetically alters the cell or tissue). In an embodiment the genetic modulator comprises a system which modifies a nucleic acid sequence in a DNA molecule, e.g., by altering a nucleobase, e.g., introducing an insertion, a deletion, a mutation (e.g., a missense mutation, a silent mutation or a nonsense mutation), a duplication, or an inversion, or any combination thereof. In an embodiment, the genetic modulator comprises a DNA base editor, CRISPR/Cas gene editing system, a zinc finger nuclease (ZFN) system, a Transcription activator-like effector nuclease (TALEN) system, a meganuclease system, or a transposase system, or any combination thereof.

In an embodiment, the genetic modulator comprises a template DNA. In an embodiment, the genetic modulator does not comprise a template DNA. In an embodiment, the genetic modulator comprises a template RNA. In an embodiment, the genetic modulator does not comprise a template RNA.

In an embodiment, the genetic modulator is a CRISPR/Cas gene editing system. In an embodiment, the CRISPR/Cas gene editing system comprises a guide RNA (gRNA) molecule comprising a targeting sequence specific to a sequence of a target gene and a peptide having nuclease activity, e.g., endonuclease activity, e.g., a Cas protein or a fragment (e.g., biologically active fragment) or a variant thereof, e.g., a Cas9 protein, a fragment (e.g., biologically active fragment) or a variant thereof; a Cas3 protein, a fragment (e.g., biologically active fragment) or a variant thereof; a Cas12a protein, a fragment (e.g., biologically active fragment) or a variant thereof, a Cas 12e protein, a fragment (e.g., biologically active fragment) or a variant thereof; a Cas 13 protein, a fragment (e.g., biologically active fragment) or a variant thereof; or a Cas14 protein, a fragment (e.g., biologically active fragment) or a variant thereof.

In an embodiment, the CRISPR/Cas gene editing system comprises a gRNA molecule comprising a targeting sequence specific to a sequence of a target gene, and a nucleic acid encoding a peptide having nuclease activity, e.g., endonuclease activity, e.g., a Cas protein or a fragment (e.g., biologically active fragment) or variant thereof, e.g., a Cas9 protein, a fragment (e.g., biologically active fragment) or a variant thereof, a Cas3 protein, a fragment (e.g., biologically active fragment) or a variant thereof, a Cas12a protein, a fragment (e.g., biologically active fragment) or a variant thereof; a Cas12e protein, a fragment (e.g., biologically active fragment) or a variant thereof; a Cas13 protein, a fragment (e.g., biologically active fragment) or a variant thereof, or a Cas14 protein, a fragment (e.g., biologically active fragment) or a variant thereof.

In an embodiment, the CRISPR/Cas gene editing system comprises a nucleic acid encoding a gRNA molecule comprising a targeting sequence specific to a sequence of a target gene, and a Cas9 protein, a fragment (e.g., biologically active fragment) or a variant thereof.

In an embodiment, the CRISPR/Cas gene editing system comprises a nucleic acid encoding a gRNA molecule comprising a targeting sequence specific to a sequence of a target gene, and a nucleic acid encoding a Cas9 protein, a fragment (e.g., biologically active fragment) or a variant thereof.

In an embodiment, the CRISPR/Cas gene editing system further comprises a template DNA. In an embodiment, the CRISPR/Cas gene editing system further comprises a template RNA. In an embodiment, the CRISPR/Cas gene editing system further comprises a Reverse transcriptase.

In an embodiment of any of the methods, compositions, or cells disclosed herein, the genetic modulator is a zinc finger nuclease (ZFN) system. In an embodiment, the ZFN system comprises a peptide having: a Zinc finger DNA binding domain, a fragment (e.g., biologically active fragment) or a variant thereof; and/or nuclease activity, e.g., endonuclease activity. In an embodiment, the ZFN system comprises a peptide having a Zn finger DNA binding domain. In an embodiment, the Zn finger binding domain comprises 1, 2, 3, 4, 5, 6, 7, 8 or more Zinc fingers. In an embodiment, the ZFN system comprises a peptide having nuclease activity e.g., endonuclease activity. In an embodiment, the peptide having nuclease activity is a type-II restriction 1-like endonuclease, e.g., a FokI endonuclease. In an embodiment, the ZFN system comprises a nucleic acid encoding a peptide having: a Zinc finger DNA binding domain, a fragment (e.g., biologically active fragment) or a variant thereof; and/or nuclease activity, e.g., endonuclease activity.

In an embodiment, the ZFN system comprises a nucleic acid encoding a peptide having a Zn finger DNA binding domain. In an embodiment, the Zn finger binding domain comprises 1, 2, 3, 4, 5, 6, 7, 8 or more Zinc fingers. In an embodiment, the ZFN system comprises a nucleic acid encoding a peptide having nuclease activity e.g., endonuclease activity. In an embodiment, the peptide having nuclease activity is a type-II restriction 1-like endonuclease, e.g., a FokI endonuclease.

In an embodiment, the system further comprises a template, e.g., template DNA.

In an embodiment of any of the methods, compositions, or cells disclosed herein, the genetic modulator is a Transcription activator-like effector nuclease (TALEN) system. In an embodiment, the system comprises a peptide having: a Transcription activator-like (TAL) effector DNA binding domain, a fragment (e.g., biologically active fragment) or a variant thereof, and/or nuclease activity, e.g., endonuclease activity. In an embodiment, the system comprises a peptide having a TAL effector DNA binding domain, a fragment (e.g., biologically active fragment) or a variant thereof. In an embodiment, the system comprises a peptide having nuclease activity, e.g., endonuclease activity. In an embodiment, the peptide having nuclease activity is a type-II restriction 1-like endonuclease, e.g., a FokI endonuclease.

In an embodiment, the system comprises a nucleic acid encoding a peptide having: a Transcription activator-like (TAL) effector DNA binding domain, a fragment (e.g., biologically active fragment) or a variant thereof; and/or nuclease activity, e.g., endonuclease activity. In an embodiment, the system comprises a nucleic acid encoding a peptide having a Transcription activator-like (TAL) effector DNA binding domain, a fragment (e.g., biologically active fragment) or a variant thereof. In an embodiment, the system comprises a nucleic acid encoding a peptide having nuclease activity, e.g., endonuclease activity. In an embodiment, the peptide having nuclease activity is a type-II restriction 1-like endonuclease, e.g., a FokI endonuclease.

In an embodiment, the system further comprises a template, e.g., a template DNA.

In an embodiment of any of the methods, compositions, or cells disclosed herein, the genetic modulator is a meganuclease system. In an embodiment, the meganuclease system comprises a peptide having a DNA binding domain and nuclease activity, e.g., a homing endonuclease. In an embodiment, the homing endonuclease comprises a LAGLIDADG endonuclease (SEQ ID NO: 270), GIY-YIG endonuclease, HNH endonuclease, His-Cys box endonuclease or a PD-(D/E)XK endonuclease, or a fragment (e.g., biologically active fragment) or variant thereof, e.g., as described in Silva G. et al, (2011) Curr Gene Therapy 11(1): 11-27.

In an embodiment, the meganuclease system comprises a nucleic acid encoding a peptide having a DNA binding domain and nuclease activity, e.g., a homing endonuclease. In an embodiment, the homing endonuclease comprises a LAGLIDADG endonuclease (SEQ ID NO: 270), GIY-YIG endonuclease, HNH endonuclease, His-Cys box endonuclease or a PD-(D/E)XK endonuclease, or a fragment (e.g., biologically active fragment) or variant thereof, e.g., as described in Silva G. et al, (2011) Curr Gene Therapy 11(1): 11-27.

In an embodiment, the system further comprises a template, e.g., a template DNA.

In an embodiment of any of the methods, compositions, or cells disclosed herein, the genetic modulator is a transposase system. In an embodiment, the transposase system comprises a nucleic acid sequence encoding a peptide having reverse transcriptase and/or nuclease activity, e.g., a retrotransposon, e.g., an LTR retrotransposon or a non-LTR retrotransposon. In an embodiment, the transposase system comprises a template, e.g., an RNA template.

### Epigenetic Modulators

In an embodiment of any of the methods, compositions, or cells disclosed herein, the payload comprises an epigenetic modulator (e.g., a modulator that epigenetically alters the cell or tissue). In an embodiment, the epigenetic modulator comprises a molecule that modifies chromatin architecture, methylates DNA, and/or modifies a histone. In an embodiment, the epigenetic modulator is a molecule that modifies chromatin architecture, e.g., a SWI/SNF remodeling complex or a component thereof. In an embodiment, the epigenetic modulator is a molecule that methylates DNA, e.g., a DNA methyltransferase, a fragment (e.g., biologically active fragment) or variant thereof (e.g., DNMT1, DNMT2 DNMT3A, DNMT3B, DNMT3L, or CpG methyltransferase (M. SssI)); a polycomb repressive complex or a component thereof, e.g., PRC1 or PRC2, or PR-DUB, or a fragment (e.g., biologically active fragment) or a variant thereof, a demethylase, or a fragment (e.g., biologically active fragment) or a variant thereof (e.g., Tet1, Tet2 or Tet3). In an embodiment, the epigenetic modulator is a molecule that modifies a histone, e.g., methylates and/or acetylates a histone, e.g., a histone modifying enzyme or a fragment (e.g., biologically active fragment) or a variant thereof, e.g., HMT, HDM, HAT, or HDAC.

### RNA Modulators

In an embodiment of any of the methods, compositions, or cells disclosed herein, the payload comprises an RNA modulator (e.g., a modulator that alters an RNA molecule in the cell or tissue). In an embodiment, the RNA modulator comprises a molecule that alters the expression and/or activity; stability or compartmentalization of an RNA molecule. In an embodiment, the RNA modulator comprises an RNA molecule, e.g., mRNA, rRNA, tRNA, regulatory RNA, non-coding RNA, long non-coding RNA (lncRNA), guide RNA (gRNA), piwi-interacting RNA (piRNA), small nucleolar RNA (snoRNA), small nuclear RNA (snRNA), extracellular RNA (exRNA), small Cajal body-specific RNA (scaRNA), microRNA (miRNA), circular RNA, or an RNAi molecule, e.g., small interfering RNA (siRNA) or small hairpin RNA (shRNA). In an embodiment, the RNA modulator comprises a DNA molecule. In an embodiment, the RNA modulator comprises a low molecular weight molecule. In an embodiment, the RNA modulator comprises a peptide, e.g., an RNA binding protein, a fragment (e.g., biologically active fragment), or a variant thereof, or an enzyme, or a fragment (e.g., biologically active fragment) or variant thereof. In an embodiment, the RNA modulator comprises an RNA base editor system. In an embodiment, the RNA base editor system comprises: a deaminase, e.g., an RNA-specific adenosine deaminase (ADAR); a Cas protein, a fragment (e.g., biologically active fragment) or a variant thereof; and/or a guide RNA. In an embodiment, the RNA base editor system further comprises a template, e.g., a DNA or RNA template.

### Therapeutic Payload or Prophylactic Payload

In an embodiment, an LNP composition disclosed herein comprises a payload, e.g., a polynucleotide, e.g., mRNA, encoding a payload or a peptide payload. In an embodiment, the LNP composition comprises one payload. In an embodiment, the LNP composition comprises more than one payload, e.g., 2, 3, 4, 5, 6, or more payloads, e.g., same or different payloads. In an embodiment, the payload is a therapeutic payload. In an embodiment, the payload is a prophylactic payload.

In some embodiments, the therapeutic payload or prophylactic payload comprises an mRNA encoding: a secreted protein; a membrane-bound protein; or an intercellular protein, or peptides, polypeptides or biologically active fragments thereof.

In some embodiments, the therapeutic payload or prophylactic payload comprises an mRNA encoding a secreted protein, or a peptide, a polypeptide or a biologically active fragment thereof. In some embodiments, the therapeutic payload or prophylactic payload comprises an mRNA encoding a membrane-bound protein, or a peptide, a polypeptide or a biologically active fragment thereof. In some embodiments, the therapeutic payload or prophylactic payload comprises an mRNA encoding an intracellular protein, or a peptide, a polypeptide or a biologically active fragment thereof. In some embodiments, the therapeutic payload or prophylactic payload comprises a protein, polypeptide, or peptide.

### Disease Disorder

In an embodiment of any of the methods, compositions, or cells disclosed herein, the disease or disorder is selected from the group consisting of a hemoglobinopathy, a clotting factor disorder, a blood cell disorder, and an immune cell disorder. In an embodiment of any of the methods, compositions, or cells disclosed herein, the subject has a mutation or SNP that is associated with, or causes, a disease or disorder selected from the group consisting of a hemoglobinopathy, a clotting factor disorder, a blood cell disorder, and an immune cell disorder.

In an embodiment of any of the methods disclosed herein the subject is a mammal, e.g., human.

### Lipid Content of LNPs

As set forth above, with respect to lipids, LNPs disclosed herein comprise an (i) ionizable lipid; (ii) sterol or other structural lipid; (iii) a non-cationic helper lipid or phospholipid; and, optionally a (iv) PEG lipid. These categories of lipids are set forth in more detail below.

In some embodiments, nucleic acids of the invention are formulated as lipid nanoparticle (LNP) compositions. Lipid nanoparticles typically comprise amino lipid, phospholipid, structural lipid and PEG lipid components along with the nucleic acid cargo of interest. The lipid nanoparticles of the invention can be generated using components, compositions, and methods as are generally known in the art, see for example PCT/US2016/052352; PCT/US2016/068300; PCT/US2018/022717; PCT/US2017/037551; PCT/US2015/027400; PCT/US2016/047406; PCT/US2016000129; PCT/US2016/014280; PCT/US2016/014280; PCT/US2017/038426; PCT/US2014/027077; PCT/US2014/055394; PCT/US2016/52117; PCT/US2012/069610; PCT/US2017/027492; PCT/US2016/059575; PCT/US2016/069491; PCT/US2016/069493; and PCT/US2014/66242, all of which are incorporated by reference herein in their entirety.

In some embodiments, the lipid nanoparticle comprises a molar ratio of 20-60% amino lipid relative to the other lipid components. For example, the lipid nanoparticle may comprise a molar ratio of 20-50%, 20-40%, 20-30%, 30-60%, 30-50%, 30-40%, 40-60%, 40-50%, or 50-60% amino lipid. In some embodiments, the lipid nanoparticle comprises a molar ratio of 20%, 30%, 40%, 50, or 60% amino lipid.

In some embodiments, the lipid nanoparticle comprises a molar ratio of 5-25% phospholipid relative to the other lipid components. For example, the lipid nanoparticle may comprise a molar ratio of 5-30%, 5-15%, 5-10%, 10-25%, 10-20%, 10-25%, 15-25%, 15-20%, 20-25%, or 25-30% phospholipid. In some embodiments, the lipid nanoparticle comprises a molar ratio of 5%, 10%, 15%, 20%, 25%, or 30% non-cationic lipid.

In some embodiments, the lipid nanoparticle comprises a molar ratio of 25-55% structural lipid relative to the other lipid components. For example, the lipid nanoparticle may comprise a molar ratio of 10-55%, 25-50%, 25-45%, 25-40%, 25-35%, 25-30%, 30-55%, 30-50%, 30-45%, 30-40%, 30-35%, 35-55%, 35-50%, 35-45%, 35-40%, 40-55%, 40-50%, 40-45%, 45-55%, 45-50%, or 50-55% structural lipid. In some embodiments, the lipid nanoparticle comprises a molar ratio of 10%, 15%, 20%, 25%, 30%, 35%, 40%, 45%, 50%, or 55% structural lipid.

In some embodiments, the lipid nanoparticle comprises a molar ratio of 0.5-15% PEG lipid relative to the other lipid components. For example, the lipid nanoparticle may comprise a molar ratio of 0.5-10%, 0.5-5%, 1-15%, 1-10%, 1-5%, 2-15%, 2-10%, 2-5%, 5-15%, 5-10%, or 10-15% PEG lipid. In some embodiments, the lipid nanoparticle comprises a molar ratio of 0.5%, 1%, 2%, 3%, 4%, 5%, 6%, 7%, 8%, 9%, 10%, 11%, 12%, 13%, 14%, or 15% PEG-lipid.

In some embodiments, the lipid nanoparticle comprises a molar ratio of 20-60% amino lipid, 5-25% phospholipid, 25-55% structural lipid, and 0.5-15% PEG lipid.

In some embodiments, the lipid nanoparticle comprises a molar ratio of 20-60% amino lipid, 5-30% phospholipid, 10-55% structural lipid, and 0.5-15% PEG lipid.

### Amino Lipids

In some aspects, the ionizable lipids (e.g., amino lipids) of the present disclosure may be one or more of a compound of Formula (I):

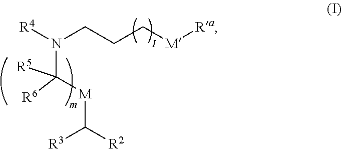

or its N-oxide, or a salt or isomer thereof, wherein:

R′ a is R′^{branched}; R′^{branched} is:

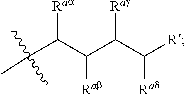

wherein

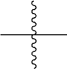

denotes a point of attachment; R^{aα}, R^{aβ}, R^{aγ}, and R^{aδ} are each independently selected from the group consisting of H, C₂₋₁₂ alkyl, and C₂₋₁₂ alkenyl; and R′ is C1-12 alkyl or C₂₋₁₂ alkenyl;

R² and R³ are each independently selected from the group consisting of C₁₋₁₄ alkyl and C₂₋₁₄ alkenyl;

R⁴ is selected from the group consisting of —(CH₂)ₙOH and

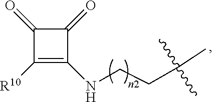

wherein

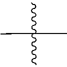

denotes a point of attachment; R¹⁰ is N(R)₂; each R is independently selected from the group consisting of C₁₋₆ alkyl, C₂₋₃ alkenyl, and H; n2 is selected from the group consisting of 1, 2, 3, 4, 5, 6, 7, 8, 9, and 10; and n is selected from the group consisting of 1, 2, 3, 4, and 5;

each R⁵ is independently selected from the group consisting of C₁₋₃ alkyl, C₂₋₃ alkenyl, and H;

each R⁶ is independently selected from the group consisting of C₁₋₃ alkyl, C₂₋₃ alkenyl, and H;

M and M′ are each independently selected from the group consisting of —C(O)O— and —OC(O)—;

1 is selected from the group consisting of 1, 2, 3, 4, and 5; and

m is selected from the group consisting of 5, 6, 7, 8, 9, 10, 11, 12, and 13.

In some embodiments, R′ a is R′branched; R′^{branched} is

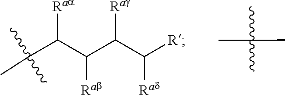

denotes a point of attachment; R^{aα}, R^{aβ}, R^{aγ}, and R^{aδ} are each independently H; R² and R³ are each independently C₁₋₁₄ alkyl; R⁴ is —(CH₂)ₙOH; n is 2; each R⁵ is independently H; each R⁶ is independently H; M and M′ are each independently —C(O)O—; R′ is C₁₋₁₂ alkyl; 1 is 5; and m is 7.

In some embodiments, R′ a is R′^{branched}; R′^{branched} is

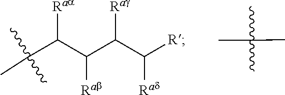

denotes a point of attachment; R^{aα}, R^{aβ}, R^{aγ}, and R^{aδ} are each independently H; R² and R³ are each independently C₁₋₁₄ alkyl; R⁴ is —(CH₂)ₙOH; n is 2; each R⁵ is independently H; each R⁶ is independently H; M and M′ are each independently —C(O)O—; R′ is C₁₋₁₂ alkyl; 1 is 3; and m is 7.

In some embodiments, R′ a is R′^{branched}; R′^{branched} is

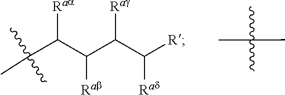

denotes a point of attachment; R^{aα} is C₂₋₁₂ alkyl; R^{aβ}, R^{aγ}, and R^{aδ} are each independently H; R² and R³ are each independently C₁₋₁₄ alkyl; R⁴ is

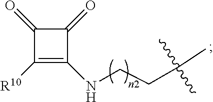

R¹⁰ is N(R)₂; one R is H and the other R is C₁₋₆ alkyl; n2 is 2; R⁵ is H; each R⁶ is independently H; M and M′ are each independently —C(O)O—; R′ is C₁₋₁₂ alkyl; 1 is 5; and m is 7.

In some embodiments, R′ a is R′^{branched}; R′^{branched} is

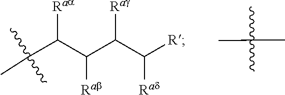

denotes a point of attachment; R^{aα}, R^{aβ}, and R^{aδ} are each independently H; R^{aγ} is C₂₋₁₂ alkyl; R² and R³ are each independently C₁₋₁₄ alkyl; R⁴ is —(CH₂)ₙOH; n is 2; each R⁵ is independently H; each R⁶ is independently H; M and M′ are each independently —C(O)O—; R′ is C₁₋₁₂ alkyl; 1 is 5; and m is 7.

In some embodiments, the compound of Formula (I) is selected from:

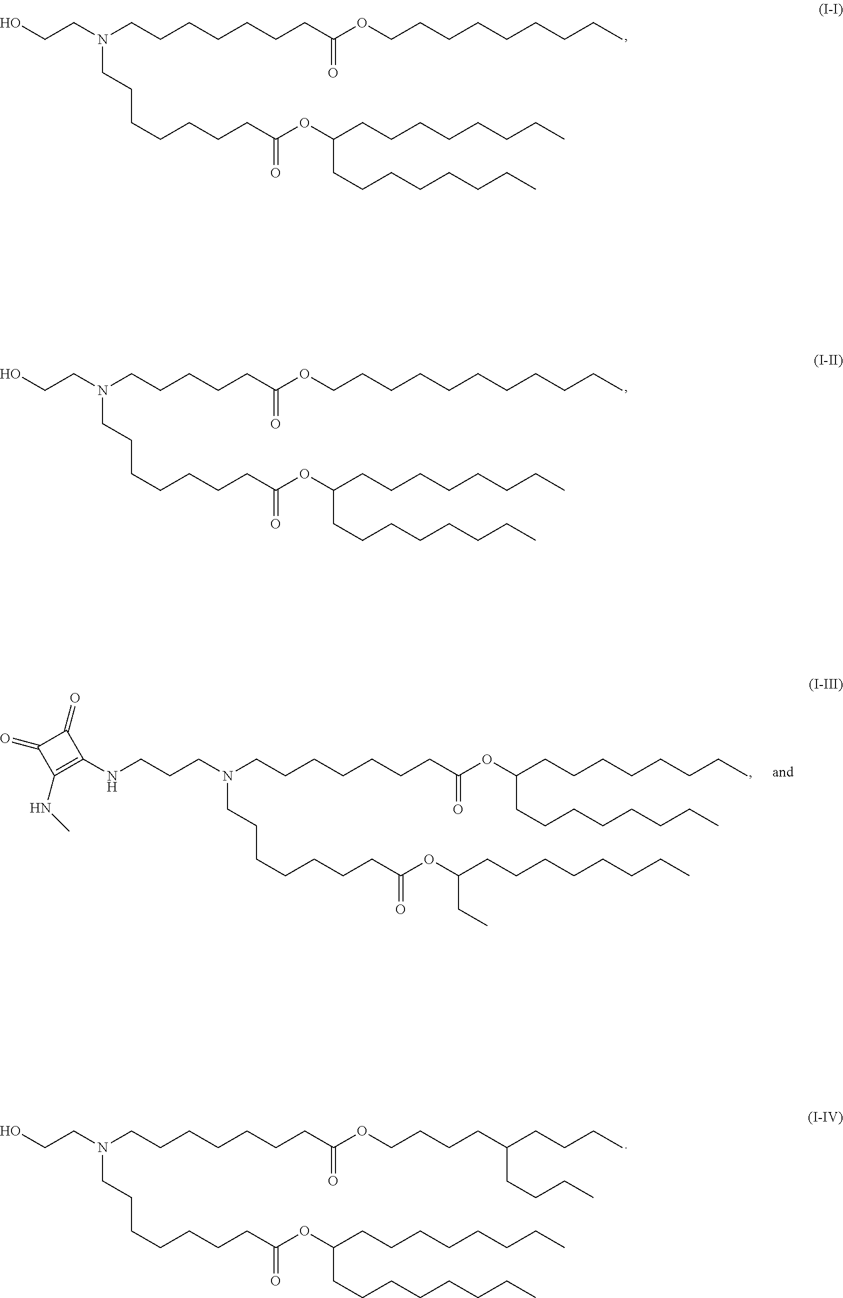 [SMILES: CCCCCCCCCOC(=O)CCCCCCCN(CCO)CCCCCCCC(=O)OC(CCCCCCCC)CCCCCCCC ; CCCCCCCCCCCOC(=O)CCCCCN(CCO)CCCCCCCC(=O)OC(CCCCCCCC)CCCCCCCC ; CCCCCCCCC(CC)OC(=O)CCCCCCCN(CCCCCCCC(=O)OC(CCCCCCCC)CCCCCCCC)CCCNc1c(NC)c(=O)c1=O ; CCCCCCCCC(CCCCCCCC)OC(=O)CCCCCCCN(CCO)CCCCCCCC(=O)OCCCCC(CCCC)CCCC]

In some embodiments, the compound of Formula (I) is Compound (I-II):

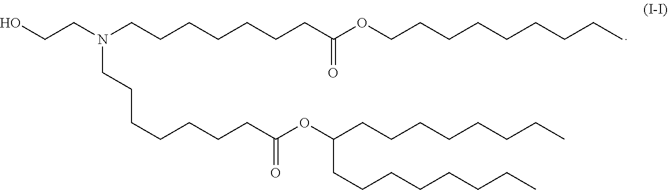 [SMILES: CCCCCCCCCOC(=O)CCCCCCCN(CCO)CCCCCCCC(=O)OC(CCCCCCCC)CCCCCCCC]

In some embodiments, the compound of Formula (I) is Compound (I-I):

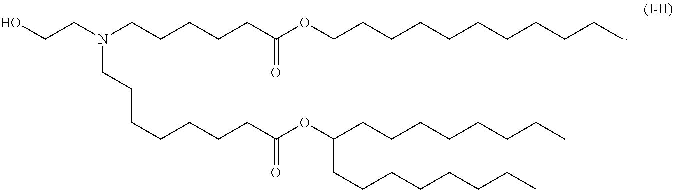 [SMILES: CCCCCCCCCCCOC(=O)CCCCCN(CCO)CCCCCCCC(=O)OC(CCCCCCCC)CCCCCCCC]

In some embodiments, the compound of Formula (I) is Compound (I-III):

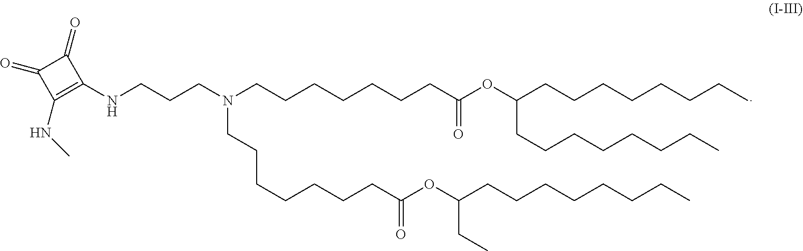 [SMILES: CCCCCCCCC(CC)OC(=O)CCCCCCCN(CCCCCCCC(=O)OC(CCCCCCCC)CCCCCCCC)CCCNc1c(NC)c(=O)c1=O]

In some embodiments, the compound of Formula (I) is Compound (I-IV):

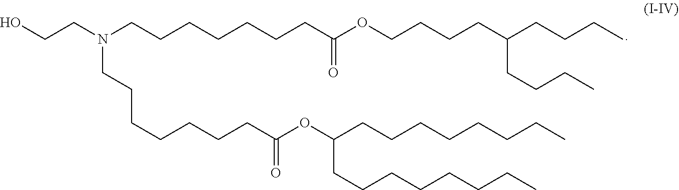 [SMILES: CCCCCCCCC(CCCCCCCC)OC(=O)CCCCCCCN(CCO)CCCCCCCC(=O)OCCCCC(CCCC)CCCC]

In some aspects, the disclosure relates to a compound of Formula (Ia):

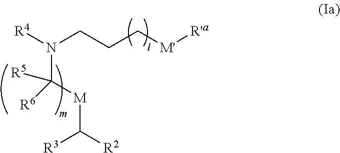

or its N-oxide, or a salt or isomer thereof, wherein:

R′ a is R′^{branched}; wherein R′^{branched} is:

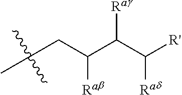

wherein

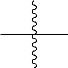

denotes a point of attachment; wherein R^{aβ}, R^{aγ}, and R^{aδ} are each independently selected from the group consisting of H, C₂₋₁₂ alkyl, and C₂₋₁₂ alkenyl; and R′ is C₁₋₁₂ alkyl or C₂₋₁₂ alkenyl;

R² and R³ are each independently selected from the group consisting of C₁₋₁₄ alkyl and C₂₋₁₄ alkenyl;

R⁴ is selected from the group consisting of —(CH₂)ₙOH and

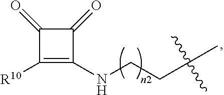

wherein

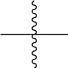

denotes a point of attachment; wherein R¹⁰ is N(R)₂; each R is independently selected from the group consisting of C₁₋₆ alkyl, C₂₋₃ alkenyl, and H; n2 is selected from the group consisting of 1, 2, 3, 4, 5, 6, 7, 8, 9, and 10; and n is selected from the group consisting of 1, 2, 3, 4, and 5;

each R⁵ is independently selected from the group consisting of C₁₋₃ alkyl, C₂₋₃ alkenyl, and H;

each R⁶ is independently selected from the group consisting of C₁₋₃ alkyl, C₂₋₃ alkenyl, and H;

M and M′ are each independently selected from the group consisting of —C(O)O— and —OC(O)—;

l is selected from the group consisting of 1, 2, 3, 4, and 5; and

m is selected from the group consisting of 5, 6, 7, 8, 9, 10, 11, 12, and 13.

In some aspects, the disclosure relates to a compound of Formula (Ib):

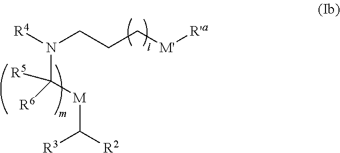

or its N-oxide, or a salt or isomer thereof, wherein:

R′ a is R′^{branched}; wherein R′^{branched} is:

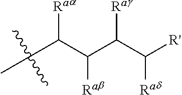

wherein

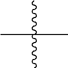

denotes a point of attachment; wherein R^{aα}, R^{aβ}, R^{aγ}, and R^{aδ} are each independently selected from the group consisting of H, C₂₋₁₂ alkyl, and C₂₋₁₂ alkenyl; and R′ is C₁₋₁₂ alkyl or C₂₋₁₂ alkenyl;

R² and R³ are each independently selected from the group consisting of C₁₋₁₄ alkyl and C₂₋₁₄ alkenyl;

R⁴ is —(CH₂)ₙOH, wherein n is selected from the group consisting of 1, 2, 3, 4, and 5;

each R⁵ is independently selected from the group consisting of C₁₋₃ alkyl, C₂₋₃ alkenyl, and H;

each R⁶ is independently selected from the group consisting of C13 alkyl, C₂₋₃ alkenyl, and H;

M and M′ are each independently selected from the group consisting of —C(O)O— and —OC(O)—;

l is selected from the group consisting of 1, 2, 3, 4, and 5; and

m is selected from the group consisting of 5, 6, 7, 8, 9, 10, 11, 12, and 13.

In some embodiments, R′ a is R′^{branched}; R′^{branched} is

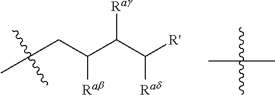

denotes a point of attachment; R^{aβ}, R^{aγ}, and R^{aδ} are each independently H; R² and R³ are each independently C₁₋₁₄ alkyl; R⁴ is —(CH₂)ₙOH; n is 2; each R⁵ is independently H; each R⁶ is independently H; M and M′ are each independently —C(O)O—; R′ is C₁₋₁₂ alkyl; 1 is 5; and m is 7.

In some embodiments, R′ a is R′^{branched}; R′^{branched} is

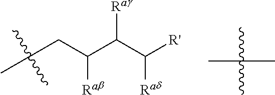

denotes a point of attachment; R^{aβ}, R^{aγ}, and R^{aδ} are each independently H; R² and R³ are each independently C₁₋₁₄ alkyl; R⁴ is —(CH₂)ₙOH; n is 2; each R⁵ is independently H; each R⁶ is independently H; M and M′ are each independently —C(O)O—; R′ is C₁₋₁₂ alkyl; 1 is 3; and m is 7.

In some embodiments, R′ a is R′^{branched}; R′^{branched} is

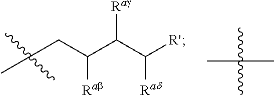

denotes a point of attachment; R^{aβ} and R^{aδ} are each independently H; Ray is C₂₋₁₂ alkyl; R² and R³ are each independently C₁₋₁₄ alkyl; R⁴ is —(CH₂)ₙOH; n is 2; each R⁵ is independently H; each R⁶ is independently H; M and M′ are each independently —C(O)O—; R′ is C₁₋₁₂ alkyl; 1 is 5; and m is 7.

In some aspects, the disclosure relates to a compound of Formula (Ic):

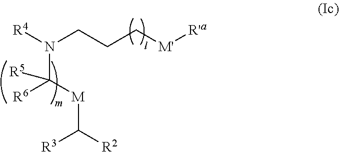

or its N-oxide, or a salt or isomer thereof, wherein:

R′ a is R′^{branched}; wherein R′^{branched} is:

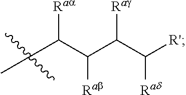

wherein

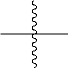

denotes a point of attachment; R^{aα}, R^{aβ}, R^{aγ}, and R^{aδ} are each independently selected from the group consisting of H, C₂₋₁₂ alkyl, and C₂₋₁₂ alkenyl; and R′ is a C₁₋₁₂ alkyl or C₂₋₁₂ alkenyl;

R² and R³ are each independently selected from the group consisting of C₁₋₁₄ alkyl and C₂₋₁₄ alkenyl;

R⁴ is

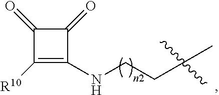

wherein

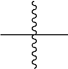

denotes a point of attachment; R¹⁰ is N(R)₂; each R is independently selected from the group consisting of C₁₋₆ alkyl, C₂₋₃ alkenyl, and H; n2 is selected from the group consisting of 1, 2, 3, 4, 5, 6, 7, 8, 9, and 10;

each R⁵ is independently selected from the group consisting of C₁₋₃ alkyl, C₂₋₃ alkenyl, and H;

each R⁶ is independently selected from the group consisting of C₁₋₃ alkyl, C₂₋₃ alkenyl, and H;

M and M′ are each independently selected from the group consisting of —C(O)O— and —OC(O)—;

l is selected from the group consisting of 1, 2, 3, 4, and 5; and

m is selected from the group consisting of 5, 6, 7, 8, 9, 10, 11, 12, and 13.

In some embodiments, R′ a is R′^{branched}; R′^{branched} is

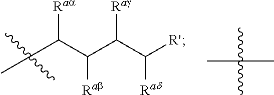

denotes a point of attachment; R^{aβ}, R^{aγ}, and R^{aδ} are each independently H; R^{aα} is C₂₋₁₂ alkyl; R′ is C₁₋₁₂ alkyl; R² and R³ are each independently C₁₋₁₄ alkyl; R⁴ is

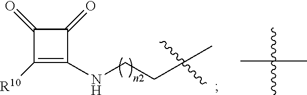

denotes a point of attachment; R¹⁰ is N(R)₂; one R is H and the other R is C₁₋₆ alkyl; n2 is 2; each R⁵ is independently H; each R⁶ is independently H; M and M′ are each independently-C(O)O—; 1 is 5; and m is 7.

In some embodiments, the compound of Formula (Ic) is Compound (I-III):

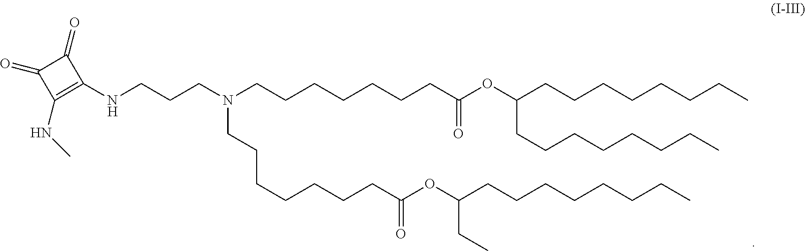

In some aspects, the ionizable lipids (e.g., amino lipids) of the present disclosure may be one or more of a compound of Formula (II),

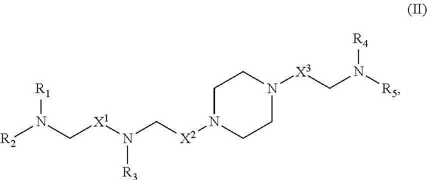

or a salt or isomer thereof, wherein:

R₁, R₂, R₃, R₄, and R₅ are each independently selected from the group consisting of C₅₋₂₀ alkyl, C₅₋₂₀ alkenyl, —R″MR′, —R*YR″, —YR″, and —R*OR″;

each M is independently selected from the group consisting of —C(O)O—, —OC(O)—, —OC(O)O—, —C(O)N(R′)—, —N(R′)C(O)—, —C(O)—, —C(S)—, —C(S)S—, —SC(S)—, —CH(OH)—, —P(O)(OR′)O—, —S(O)₂—, an aryl group, and a heteroaryl group;

X¹, X², and X³ are each independently selected from the group consisting of a bond, —CH₂—, —(CH₂)₂—, —CHR—, —CHY—, —C(O)—, —C(O)O—, —OC(O)—, —C(O)—CH₂—, —CH₂—C(O)—, —C(O)O—CH₂—, —OC(O)—CH₂—, —CH₂—C(O)O—, —CH₂—OC(O)—, —CH(OH)—, —C(S)—, and —CH(SH)—;

each Y is independently a C₃₋₆ carbocycle;

each R* is independently selected from the group consisting of C₁₋₁₂ alkyl and C₂₋₁₂ alkenyl;

each R is independently selected from the group consisting of C₁₋₃ alkyl and a C₃₋₆ carbocycle;

each R′ is independently selected from the group consisting of C₁₋₁₂ alkyl, C₂₋₁₂ alkenyl, and H; and each R″ is independently selected from the group consisting of C₃₋₁₂ alkyl and C₃₋₁₂ alkenyl, and wherein:

i) at least one of X¹, X², and X³ is not —CH₂—; and/or

ii) at least one of R₁, R₂, R₃, R₄, and R₅ is —R″MR′.

In some embodiments, R₁, R₂, R₃, R₄, and R₅ are each C₅₋₂₀ alkyl; X¹ is —CH₂—; and X² and X³ are each independently —C(O)—.

In some embodiments, the compound of Formula (II) is Compound (II-I):

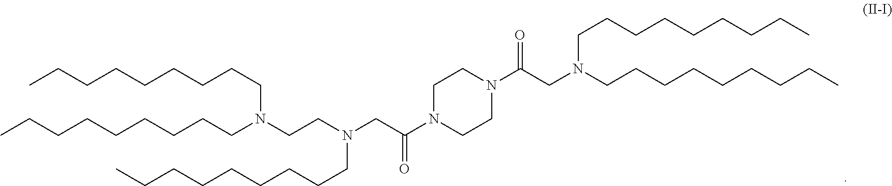 [SMILES: CCCCCCCCCN(CCCCCCCCC)CCN(CCCCCCCCC)CC(=O)N1CCN(C(=O)CN(CCCCCCCCC)CCCCCCCCC)CC1]

An amine moiety (e.g., a central amine moiety) of an ionizable lipid (e.g., an amino lipid) according to any one of Formulas (I), (I-I), (I-II), (I-III), (I-IV), (Ia), (Ib), (Ic), (II), or (II-I) may be protonated at a physiological pH. Thus, a lipid may have a positive or partial positive charge at physiological pH.

### Phospholipids

The lipid composition of the lipid nanoparticle composition disclosed herein can comprise one or more phospholipids, for example, one or more saturated or (poly)unsaturated phospholipids or a combination thereof. In general, phospholipids comprise a phospholipid moiety and one or more fatty acid moieties.

A phospholipid moiety can be selected, for example, from the non-limiting group consisting of phosphatidyl choline, phosphatidyl ethanolamine, phosphatidyl glycerol, phosphatidyl serine, phosphatidic acid, 2-lysophosphatidyl choline, and a sphingomyelin.

A fatty acid moiety can be selected, for example, from the non-limiting group consisting of lauric acid, myristic acid, myristoleic acid, palmitic acid, palmitoleic acid, stearic acid, oleic acid, linoleic acid, alpha-linolenic acid, erucic acid, phytanoic acid, arachidic acid, arachidonic acid, eicosapentaenoic acid, behenic acid, docosapentaenoic acid, and docosahexaenoic acid.

Particular phospholipids can facilitate fusion to a membrane. For example, a cationic phospholipid can interact with one or more negatively charged phospholipids of a membrane (e.g., a cellular or intracellular membrane). Fusion of a phospholipid to a membrane can allow one or more elements (e.g., a therapeutic agent) of a lipid-containing composition (e.g., LNPs) to pass through the membrane permitting, e.g., delivery of the one or more elements to a target tissue.

Non-natural phospholipid species including natural species with modifications and substitutions including branching, oxidation, cyclization, and alkynes are also contemplated. For example, a phospholipid can be functionalized with or cross-linked to one or more alkynes (e.g., an alkenyl group in which one or more double bonds is replaced with a triple bond). Under appropriate reaction conditions, an alkyne group can undergo a copper-catalyzed cycloaddition upon exposure to an azide. Such reactions can be useful in functionalizing a lipid bilayer of a nanoparticle composition to facilitate membrane permeation or cellular recognition or in conjugating a nanoparticle composition to a useful component such as a targeting or imaging moiety (e.g., a dye).

Phospholipids include, but are not limited to, glycerophospholipids such as phosphatidylcholines, phosphatidylethanolamines, phosphatidylserines, phosphatidylinositols, phosphatidy glycerols, and phosphatidic acids. Phospholipids also include phosphosphingolipid, such as sphingomyelin.

In some embodiments, a phospholipid of the invention comprises 1,2-distearoyl-sn-glycero-3-phosphocholine (DSPC), 1,2-Distearoyl-sn-glycero-3-phosphoethanolamine (DSPE), 1,2-dioleoyl-sn-glycero-3-phosphoethanolamine (DOPE), 1,2-dilinoleoyl-sn-glycero-3-phosphocholine (DLPC), 1,2-dimyristoyl-sn-gly cero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2 cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine,1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin, and mixtures thereof.

In certain embodiments, a phospholipid useful or potentially useful in the present invention is an analog or variant of DSPC. In certain embodiments, a phospholipid useful or potentially useful in the present invention is a compound of Formula (IV):

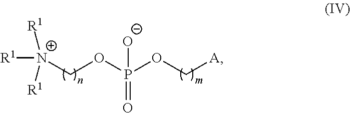

or a salt thereof, wherein:

each R¹ is independently optionally substituted alkyl; or optionally two R¹ are joined together with the intervening atoms to form optionally substituted monocyclic carbocyclyl or optionally substituted monocyclic heterocyclyl; or optionally three R¹ are joined together with the intervening atoms to form optionally substituted bicyclic carbocyclyl or optionally substitute bicyclic heterocyclyl;

n is 1, 2, 3, 4, 5, 6, 7, 8, 9, or 10;

m is 0, 1, 2, 3, 4, 5, 6, 7, 8, 9, or 10;

A is of the formula:

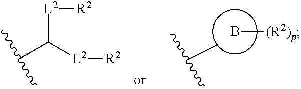

each instance of L² is independently a bond or optionally substituted C₁₋₆ alkylene, wherein one methylene unit of the optionally substituted C₁₋₆ alkylene is optionally replaced with O, N(R^{N}), S, C(O), C(O)N(R^{N}), NR^{N}C(O), C(O)O, OC(O), OC(O)O, OC(O)N(R^{N}), NR^{N}C(O)O or NR^{N}C(O)N(R^{N});

each instance of R² is independently optionally substituted C₁₋₃₀ alkyl, optionally substituted C₁₋₃₀ alkenyl, or optionally substituted C₁₋₃₀ alkynyl; optionally wherein one or more methylene units of R² are independently replaced with optionally substituted carbocyclylene, optionally substituted heterocyclylene, optionally substituted arylene, optionally substituted heteroarylene, N(R^{N}), O, S, C(O), C(O)N(R^{N}), NR^{N}C(O), NR^{N}C(O)N(R^{N}) C(O)O, OC(O), —OC(O)O, OC(O)N(R^{N}), NR^{N}C(O)O, C(O)S, SC(O), C(═NR^{N}), C(═NR^{N})N(R^{N}), NR^{N}C(═NR^{N}) NR^{N}C(═NR^{N})N(R^{N}), C(S), C(S)N(R^{N}), NR^{N}C(S), NR^{N}C(S)N(R^{N}) S(O), OS(O), S(O)O, —OS(O)O, OS(O)₂, S(O)₂O, OS(O)₂O, N(R^{N})S(O), S(O)N(R^{N}), N(R^{N})S(O)N(R^{N}), OS(O)N(R^{N}), N(R^{N})S(O)O, S(O)₂, N(R^{N})S(O)₂, S(O)₂N(R^{N}), N(R^{N})S(O)₂N(R^{N}), OS(O)₂N(R^{N}), or —N(R^{N})S(O)₂O;

each instance of R^{N} is independently hydrogen, optionally substituted alkyl, or a nitrogen protecting group;

Ring B is optionally substituted carbocyclyl, optionally substituted heterocyclyl, optionally substituted aryl, or optionally substituted heteroaryl; and

p is 1 or 2;

provided that the compound is not of the formula:

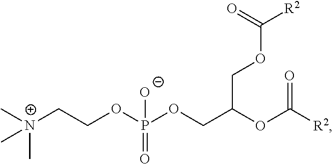

wherein each instance of R² is independently unsubstituted alkyl, unsubstituted alkenyl, or unsubstituted alkynyl.

In some embodiments, the phospholipids may be one or more of the phospholipids described in U.S. Application No. 62/520,530, or in International Application PCT/US2018/037922 filed on 15 Jun. 2018, the entire contents of each of which is hereby incorporated by reference in its entirety.

### Structural Lipids

The lipid composition of a pharmaceutical composition disclosed herein can comprise one or more structural lipids. As used herein, the term “structural lipid” refers to sterols and also to lipids containing sterol moieties.

Incorporation of structural lipids in the lipid nanoparticle may help mitigate aggregation of other lipids in the particle. Structural lipids can be selected from the group including but not limited to, cholesterol, fecosterol, sitosterol, ergosterol, campesterol, stigmasterol, brassicasterol, tomatidine, tomatine, ursolic acid, alpha-tocopherol, hopanoids, phytosterols, steroids, and mixtures thereof. In some embodiments, the structural lipid is a sterol. As defined herein, “sterols” are a subgroup of steroids consisting of steroid alcohols. In certain embodiments, the structural lipid is a steroid. In certain embodiments, the structural lipid is cholesterol. In certain embodiments, the structural lipid is an analog of cholesterol. In certain embodiments, the structural lipid is alpha-tocopherol.

In some embodiments, the structural lipids may be one or more of the structural lipids described in U.S. application Ser. No. 16/493,814.

### Polyethylene Glycol (PEG)-Lipids

The lipid composition of a pharmaceutical composition disclosed herein can comprise one or more polyethylene glycol (PEG) lipids.

As used herein, the term “PEG-lipid” refers to polyethylene glycol (PEG)-modified lipids. Non-limiting examples of PEG-lipids include PEG-modified phosphatidylethanolamine and phosphatidic acid, PEG-ceramide conjugates (e.g., PEG-CerC14 or PEG-CerC20), PEG-modified dialkylamines and PEG-modified 1,2-diacyloxypropan-3-amines. Such lipids are also referred to as PEGylated lipids. For example, a PEG lipid can be PEG-c-DOMG, PEG-DMG, PEG-DLPE, PEG-DMPE, PEG-DPPC, or a PEG-DSPE lipid.

In some embodiments, the PEG-lipid includes, but not limited to 1,2-dimyristoyl-sn-glycerol methoxypolyethylene glycol (PEG-DMG), 1,2-distearoyl-sn-glycero-3-phosphoethanolamine-N-[amino(polyethylene glycol)] (PEG-DSPE), PEG-disteryl glycerol (PEG-DSG), PEG-dipalmetoleyl, PEG-dioleyl, PEG-distearyl, PEG-diacylglycamide (PEG-DAG), PEG-dipalmitoyl phosphatidylethanolamine (PEG-DPPE), or PEG-1,2-dimyristyloxlpropyl-3-amine (PEG-c-DMA).

In one embodiment, the PEG-lipid is selected from the group consisting of a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof. In some embodiments, the PEG-modified lipid is PEG-DMG, PEG-c-DOMG (also referred to as PEG-DOMG), PEG-DSG and/or PEG-DPG.

In some embodiments, the lipid moiety of the PEG-lipids includes those having lengths of from about C₁₄ to about C₂₂, preferably from about C₁₄ to about C₁₆. In some embodiments, a PEG moiety, for example an mPEG-NH₂, has a size of about 1000, 2000, 5000, 10,000, 15,000 or 20,000 daltons. In one embodiment, the PEG-lipid is PEG₂ₖ-DMG.

In one embodiment, the lipid nanoparticles described herein can comprise a PEG lipid which is a non-diffusible PEG. Non-limiting examples of non-diffusible PEGs include PEG-DSG and PEG-DSPE.

PEG-lipids are known in the art, such as those described in U.S. Pat. No. 8,158,601 and International Publ. No. WO 2015/130584 A2, which are incorporated herein by reference in their entirety.

In general, some of the other lipid components (e.g., PEG lipids) of various formulae, described herein may be synthesized as described International Patent Application No. PCT/US2016/000129, filed Dec. 10, 2016, entitled “Compositions and Methods for Delivery of Therapeutic Agents,” which is incorporated by reference in its entirety.

The lipid component of a lipid nanoparticle composition may include one or more molecules comprising polyethylene glycol, such as PEG or PEG-modified lipids. Such species may be alternately referred to as PEGylated lipids. A PEG lipid is a lipid modified with polyethylene glycol. A PEG lipid may be selected from the non-limiting group including PEG-modified phosphatidylethanolamines, PEG-modified phosphatidic acids, PEG-modified ceramides, PEG-modified dialkylamines, PEG-modified diacylglycerols, PEG-modified dialkylglycerols, and mixtures thereof. For example, a PEG lipid may be PEG-c-DOMG, PEG-DMG, PEG-DLPE, PEG-DMPE, PEG-DPPC, or a PEG-DSPE lipid.

In some embodiments the PEG-modified lipids are a modified form of PEG DMG. PEG-DMG has the following structure:

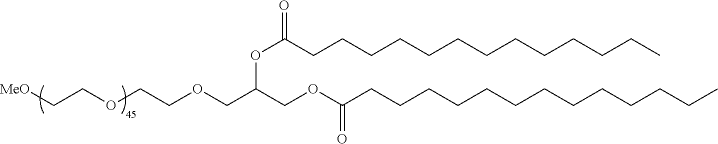

In one embodiment, PEG lipids useful in the present invention can be PEGylated lipids described in International Publication No. WO2012099755, the contents of which is herein incorporated by reference in its entirety. Any of these exemplary PEG lipids described herein may be modified to comprise a hydroxyl group on the PEG chain. In certain embodiments, the PEG lipid is a PEG-OH lipid. As generally defined herein, a “PEG-OH lipid” (also referred to herein as “hydroxy-PEGylated lipid”) is a PEGylated lipid having one or more hydroxyl (—OH) groups on the lipid. In certain embodiments, the PEG-OH lipid includes one or more hydroxyl groups on the PEG chain. In certain embodiments, a PEG-OH or hydroxy-PEGylated lipid comprises an —OH group at the terminus of the PEG chain. Each possibility represents a separate embodiment of the present invention.

In certain embodiments, a PEG lipid useful in the present invention is a compound of Formula (V). Provided herein are compounds of Formula (V):

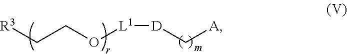

or salts thereof, wherein:

R³ is —OR^{O};

R^{O} is hydrogen, optionally substituted alkyl, or an oxygen protecting group;

r is an integer between 1 and 100, inclusive;

L¹ is optionally substituted C₁₋₁₀ alkylene, wherein at least one methylene of the optionally substituted C₁₋₁₀ alkylene is independently replaced with optionally substituted carbocyclylene, optionally substituted heterocyclylene, optionally substituted arylene, optionally substituted heteroarylene, 0, N(R^{N}), S, C(O), C(O)N(R^{N}), NR^{N}C(O) C(O)O, OC(O), OC(O)O, OC(O)N(R^{N}), NR^{N}C(O)O, or NR^{N}C(O)N(R^{N});

D is a moiety obtained by click chemistry or a moiety cleavable under physiological conditions;

m is 0, 1, 2, 3, 4, 5, 6, 7, 8, 9, or 10;

A is of the formula:

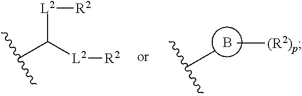

each instance of L² is independently a bond or optionally substituted C₁₋₆ alkylene, wherein one methylene unit of the optionally substituted C₁₋₆ alkylene is optionally replaced with O, N(R^{N}), S, C(O), C(O)N(R^{N}), NR^{N}C(O), C(O)O, OC(O), OC(O)O, OC(O)N(R^{N}), NR^{N}C(O)O or NR^{N}C(O)N(R^{N});

each instance of R² is independently optionally substituted C₁₋₃₀ alkyl, optionally substituted C₁₋₃₀ alkenyl, or optionally substituted C₁₋₃₀ alkynyl; optionally wherein one or more methylene units of R² are independently replaced with optionally substituted carbocyclylene, optionally substituted heterocyclylene, optionally substituted arylene, optionally substituted heteroarylene, N(R^{N}), O, S, C(O), C(O)N(R^{N}), NR^{N}C(O), NR^{N}C(O)N(R^{N}) C(O)O, OC(O), —OC(O)O, OC(O)N(R^{N}), NR^{N}C(O)O, C(O)S, SC(O), C(═NR^{N}), C(═NR^{N})N(R^{N}), NR^{N}C(═NR^{N}) NR^{N}C(═NR^{N})N(R^{N}), C(S), C(S)N(R^{N}), NR^{N}C(S), NR^{N}C(S)N(R^{N}) S(O), OS(O), S(O)O, —OS(O)O, OS(O)₂, S(O)₂O, OS(O)₂O, N(R^{N})S(O), S(O)N(R^{N}), N(R^{N})S(O)N(R^{N}), OS(O)N(R^{N}), N(R^{N})S(O)O, S(O)₂, N(R^{N})S(O)₂, S(O)₂N(R^{N}), N(R^{N})S(O)₂N(R^{N}), OS(O)₂N(R^{N}), or —N(R^{N})S(O)₂O;

each instance of R^{N} is independently hydrogen, optionally substituted alkyl, or a nitrogen protecting group;

Ring B is optionally substituted carbocyclyl, optionally substituted heterocyclyl, optionally substituted aryl, or optionally substituted heteroaryl; and

p is 1 or 2.

In certain embodiments, the compound of Formula (V) is a PEG-OH lipid (i.e., R³ is —OR^{O}, and R^{O} is hydrogen). In certain embodiments, the compound of Formula (V) is of Formula (V-OH):

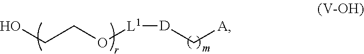

or a salt thereof.

In certain embodiments, a PEG lipid useful in the present invention is a PEGylated fatty acid. In certain embodiments, a PEG lipid useful in the present invention is a compound of Formula (VI). Provided herein are compounds of Formula (VI-A):

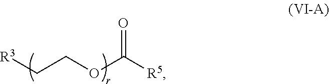

or a salts thereof, wherein:

R³ is-OR^{O};

R^{O} is hydrogen, optionally substituted alkyl or an oxygen protecting group;

r is an integer between 1 and 100, inclusive;

R⁵ is optionally substituted C₁₀₋₄₀ alkyl, optionally substituted C₁₀₋₄₀ alkenyl, or optionally substituted C₁₀₋₄₀ alkynyl; and optionally one or more methylene groups of R⁵ are replaced with optionally substituted carbocyclylene, optionally substituted heterocyclylene, optionally substituted arylene, optionally substituted heteroarylene, N(R^{N}), 0, S, C(O), C(O)N(R^{N}), —NR^{N}C(O), NR^{N}C(O)N(R^{N}), C(O)O, OC(O), OC(O)O, OC(O)N(R^{N}), NR^{N}C(O)O C(O)S, SC(O), C(═NR^{N}), C(═NR^{N})N(R^{N}), NR^{N}C(NR^{N}) NR^{N}C(═NR^{N})N(R^{N}), C(S), C(S)N(R^{N}), NR^{N}C(S)—NR^{N}C(S)N(R^{N}), S(O), OS(O), S(O)O, OS(O)O, OS(O)₂, S(O)₂O, OS(O)₂O, N(R^{N})S(O), —S(O)N(R^{N}), N(R^{N})S(O)N(R^{N}), OS(O)N(R^{N}), N(R^{N})S(O)O, S(O)₂, N(R^{N})S(O)₂, S(O)₂N(R^{N}), —N(R^{N})S(O)₂N(R^{N}), OS(O)₂N(R^{N}), or N(R^{N})S(O)₂O; and

each instance of R^{N} is independently hydrogen, optionally substituted alkyl, or a nitrogen protecting group.

In certain embodiments, the compound of Formula (VI) is of Formula (VI-OH):

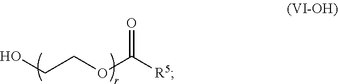

or a salt thereof. In some embodiments, r is 40-50.

In yet other embodiments the compound of Formula (VI-C) is:

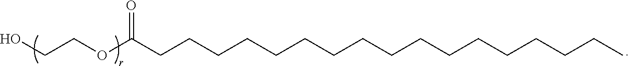

or a salt thereof.

In one embodiment, the compound of Formula (VI-D) is

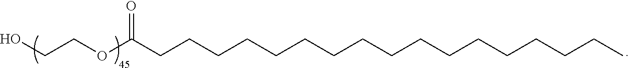

In some aspects, the lipid composition of the pharmaceutical compositions disclosed herein does not comprise a PEG-lipid.

In some embodiments, the PEG-lipids may be one or more of the PEG lipids described in U.S. Application No. U.S. Ser. No. 15/674,872.

In some embodiments, an LNP of the invention comprises an amino lipid of any of Formula I, II or III, a phospholipid comprising DSPC, a structural lipid, and a PEG lipid comprising PEG-DMG.

In some embodiments, an LNP of the invention comprises an amino lipid of any of Formula I, II or III, a phospholipid comprising DSPC, a structural lipid, and a PEG lipid comprising a compound having Formula VI.

In some embodiments, an LNP of the invention comprises an amino lipid of Formula I, II or III, a phospholipid comprising a compound having Formula IV, a structural lipid, and the PEG lipid comprising a compound having Formula V or VI.

In some embodiments, an LNP of the invention comprises an amino lipid of Formula I, II or III, a phospholipid comprising a compound having Formula IV, a structural lipid, and the PEG lipid comprising a compound having Formula V or VI.

In some embodiments, an LNP of the invention comprises an amino lipid of Formula I, II or III, a phospholipid having Formula IV, a structural lipid, and a PEG lipid comprising a compound having Formula VI.

In some embodiments, an LNP of the invention comprises an amino lipid comprising a compound of Formula (I-I), a phospholipid comprising DSPC, a structural lipid comprising cholesterol, and a PEG lipid comprising a compound of Formula (VI-D).

In some embodiments, an LNP of the invention comprises an N:P ratio of from about 2:1 to about 30:1. In some embodiments, an LNP of the invention comprises an N:P ratio of about 6:1. In some embodiments, an LNP of the invention comprises an N:P ratio of about 3:1, 4:1, or 5:1. In some embodiments, an LNP of the invention comprises a wt/wt ratio of the amino lipid component to the RNA of from about 10:1 to about 100:1. In some embodiments, an LNP of the invention comprises a wt/wt ratio of the amino lipid component to the RNA of about 20:1. In some embodiments, an LNP of the invention comprises a wt/wt ratio of the amino lipid component to the RNA of about 10:1.

In some embodiments, an LNP of the invention has a mean diameter from about 30 nm to about 150 nm. In some embodiments, an LNP of the invention has a mean diameter from about 60 nm to about 120 nm.

### Exemplary Additional LNP Components

### Surfactants

In certain embodiments, the lipid nanoparticles of the disclosure optionally includes one or more surfactants.

In certain embodiments, the surfactant is an amphiphilic polymer. As used herein, an amphiphilic “polymer” is an amphiphilic compound that comprises an oligomer or a polymer.

For example, an amphiphilic polymer can comprise an oligomer fragment, such as two or more PEG monomer units. For example, an amphiphilic polymer described herein can be PS 20.

For example, the amphiphilic polymer is a block copolymer. For example, the amphiphilic polymer is a lyoprotectant.

For example, amphiphilic polymer has a critical micelle concentration (CMC) of less than 2×10−4 M in water at about 30° C. and atmospheric pressure.

For example, amphiphilic polymer has a critical micelle concentration (CMC) ranging between about 0.1×10−4 M and about 1.3×10−4 M in water at about 30° C. and atmospheric pressure.

For example, the concentration of the amphiphilic polymer ranges between about its CMC and about 30 times of CMC (e.g., up to about 25 times, about 20 times, about 15 times, about 10 times, about 5 times, or about 3 times of its CMC) in the formulation, e.g., prior to freezing or lyophilization.

For example, the amphiphilic polymer is selected from poloxamers (Pluronic®), poloxamines (Tetronic®), polyoxyethylene glycol sorbitan alkyl esters (polysorbates) and polyvinyl pyrrolidones (PVPs).

For example, the amphiphilic polymer is a poloxamer. For example, the amphiphilic polymer is of the following structure:

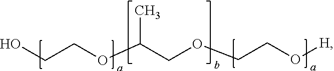

wherein a is an integer between 10 and 150 and b is an integer between 20 and 60. For example, a is about 12 and b is about 20, or a is about 80 and b is about 27, or a is about 64 and b is about 37, or a is about 141 and b is about 44, or a is about 101 and b is about 56.

For example, the amphiphilic polymer is P124, P188, P237, P338, or P407.

For example, the amphiphilic polymer is P188 (e.g., Poloxamer 188, CAS Number 9003-11-6, also known as Kolliphor P188).

For example, the amphiphilic polymer is a poloxamine, e.g., tetronic 304 or tetronic 904.

For example, the amphiphilic polymer is a polyvinylpyrrolidone (PVP), such as PVP with molecular weight of 3 kDa, 10 kDa, or 29 kDa.

For example, the amphiphilic polymer is a polysorbate, such as PS 20.

In certain embodiments, the surfactant is a non-ionic surfactant.

In some embodiments, the lipid nanoparticle comprises a surfactant. In some embodiments, the surfactant is an amphiphilic polymer. In some embodiments, the surfactant is a non-ionic surfactant.

For example, the non-ionic surfactant is selected from the group consisting of polyethylene glycol ether (Brij), poloxamer, polysorbate, sorbitan, and derivatives thereof. For example, the polyethylene glycol ether is a compound of Formula (VIII):

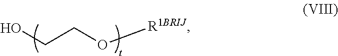

or a salt or isomer thereof, wherein:

t is an integer between 1 and 100;

R¹BRIJ independently is C10-40 alkyl, C10-40 alkenyl, or C10-40 alkynyl; and optionally one or more methylene groups of R⁵PEG are independently replaced with C3-10 carbocyclylene, 4 to 10 membered heterocyclylene, C6-10 arylene, 4 to 10 membered heteroarylene, —N(R^{N})—, —O—, —S—, —C(O)—, —C(O)N(R^{N})—, —NR^{N}C(O)—, —NR^{N}C(O)N(R^{N})—, —C(O)O—, —OC(O)—, —OC(O)O—, —OC(O)N(R^{N})—, —NR^{N}C(O)O—, —C(O)S—, —SC(O)—, —C(═NR^{N})—, —C(═NR^{N})N(R^{N})—, —NR^{N}C(═NR^{N})—, —NR^{N}C(═NR^{N})N(R^{N})—, —C(S)—, —C(S)N(R^{N})—, —NR^{N}C(S)—, —NR^{N}C(S)N(R^{N})—, —S(O)—, —OS(O)—, —S(O)O—, —OS(O)O—, —OS(O)2-, —S(O)2O—S(O)2O—, —N(R^{N})S(O)—, —S(O)N(R^{N})—, —N(R^{N})S(O)N(R^{N})—, —OS(O)N(R^{N})—, —N(R^{N})S(O)O—, —S(O)2-, —N(R^{N})S(O)2-, —S(O)2N(R^{N})—, —N(R^{N})S(O)2N(R^{N})—, —OS(O)2N(R^{N})—, or —N(R^{N})S(O)2O—; and

each instance of R^{N} is independently hydrogen, C₁₋₆ alkyl, or a nitrogen protecting group

In some embodiment, R1BRIJ is C18 alkyl. For example, the polyethylene glycol ether is a compound of Formula (VIII-a):

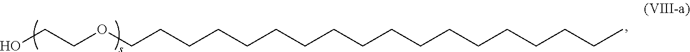

or a salt or isomer thereof.

In some embodiments, R1BRIJ is C18 alkenyl. For example, the polyethylene glycol ether is a compound of Formula (VIII-b):

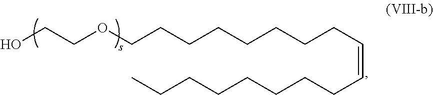

or a salt or isomer thereof

In some embodiments, the poloxamer is selected from the group consisting of poloxamer 101, poloxamer 105, poloxamer 108, poloxamer 122, poloxamer 123, poloxamer 124, poloxamer 181, poloxamer 182, poloxamer 183, poloxamer 184, poloxamer 185, poloxamer 188, poloxamer 212, poloxamer 215, poloxamer 217, poloxamer 231, poloxamer 234, poloxamer 235, poloxamer 237, poloxamer 238, poloxamer 282, poloxamer 284, poloxamer 288, poloxamer 331, poloxamer 333, poloxamer 334, poloxamer 335, poloxamer 338, poloxamer 401, poloxamer 402, poloxamer 403, and poloxamer 407.

In some embodiments, the polysorbate is Tween® 20, Tween® 40, Tween®, 60, or Tween® 80.

In some embodiments, the derivative of sorbitan is Span® 20, Span® 60, Span® 65, Span® 80, or Span® 85.

In some embodiments, the concentration of the non-ionic surfactant in the lipid nanoparticle ranges from about 0.00001% w/v to about 1% w/v, e.g., from about 0.00005% w/v to about 0.5% w/v, or from about 0.0001% w/v to about 0.1% w/v.

In some embodiments, the concentration of the non-ionic surfactant in lipid nanoparticle ranges from about 0.000001 wt % to about 1 wt %, e.g., from about 0.000002 wt % to about 0.8 wt %, or from about 0.000005 wt % to about 0.5 wt %.

In some embodiments, the concentration of the PEG lipid in the lipid nanoparticle ranges from about 0.01% by molar to about 50% by molar, e.g., from about 0.05% by molar to about 20% by molar, from about 0.07% by molar to about 10% by molar, from about 0.1% by molar to about 8% by molar, from about 0.2% by molar to about 5% by molar, or from about 0.25% by molar to about 3% by molar.

### Adjuvants

In some embodiments, an LNP of the invention optionally includes one or more adjuvants, e.g., Glucopyranosyl Lipid Adjuvant (GLA), CpG oligodeoxynucleotides (e.g., Class A or B), poly(I:C), aluminum hydroxide, and Pam3CSK4.

### Other Components

An LNP of the invention may optionally include one or more components in addition to those described in the preceding sections. For example, a lipid nanoparticle may include one or more small hydrophobic molecules such as a vitamin (e.g., vitamin A or vitamin E) or a sterol. Lipid nanoparticles may also include one or more permeability enhancer molecules, carbohydrates, polymers, surface altering agents, or other components. A permeability enhancer molecule may be a molecule described by U.S. patent application publication No. 2005/0222064, for example. Carbohydrates may include simple sugars (e.g., glucose) and polysaccharides (e.g., glycogen and derivatives and analogs thereof).

A polymer may be included in and/or used to encapsulate or partially encapsulate a lipid nanoparticle. A polymer may be biodegradable and/or biocompatible. A polymer may be selected from, but is not limited to, polyamines, polyethers, polyamides, polyesters, polycarbamates, polyureas, polycarbonates, polystyrenes, polyimides, polysulfones, polyurethanes, polyacetylenes, polyethylenes, polyethyleneimines, polyisocyanates, polyacrylates, polymethacrylates, polyacrylonitriles, and polyarylates. For example, a polymer may include poly(caprolactone) (PCL), ethylene vinyl acetate polymer (EVA), poly(lactic acid) (PLA), poly(L-lactic acid) (PLLA), poly(glycolic acid) (PGA), poly(lactic acid-co-glycolic acid) (PLGA), poly(L-lactic acid-co-glycolic acid) (PLLGA), poly(D,L-lactide) (PDLA), poly(L-lactide) (PLLA), poly(D,L-lactide-co-caprolactone), poly(D,L-lactide-co-caprolactone-co-glycolide), poly(D,L-lactide-co-PEO-co-D,L-lactide), poly(D,L-lactide-co-PPO-co-D,L-lactide), polyalkyl cyanoacrylate, polyurethane, poly-L-lysine (PLL), hydroxypropyl methacrylate (HPMA), polyethyleneglycol, poly-L-glutamic acid, poly(hydroxy acids), polyanhydrides, polyorthoesters, poly(ester amides), polyamides, poly(ester ethers), polycarbonates, polyalkylenes such as polyethylene and polypropylene, polyalkylene glycols such as poly(ethylene glycol) (PEG), polyalkylene oxides (PEO), polyalkylene terephthalates such as poly(ethylene terephthalate), polyvinyl alcohols (PVA), polyvinyl ethers, polyvinyl esters such as poly(vinyl acetate), polyvinyl halides such as poly(vinyl chloride) (PVC), polyvinylpyrrolidone (PVP), polysiloxanes, polystyrene, polyurethanes, derivatized celluloses such as alkyl celluloses, hydroxyalkyl celluloses, cellulose ethers, cellulose esters, nitro celluloses, hydroxypropylcellulose, carboxymethylcellulose, polymers of acrylic acids, such as poly(methyl(meth)acrylate) (PMMA), poly(ethyl(meth)acrylate), poly(butyl(meth)acrylate), poly(isobutyl(meth)acrylate), poly(hexyl(meth)acrylate), poly(isodecyl(meth)acrylate), poly(lauryl(meth)acrylate), poly(phenyl(meth)acrylate), poly(methyl acrylate), poly(isopropyl acrylate), poly(isobutyl acrylate), poly(octadecyl acrylate) and copolymers and mixtures thereof, polydioxanone and its copolymers, polyhydroxyalkanoates, polypropylene fumarate, polyoxymethylene, poloxamers, poloxamines, poly(ortho)esters, poly(butyric acid), poly(valeric acid), poly(lactide-co-caprolactone), trimethylene carbonate, poly(N-acryloylmorpholine) (PAcM), poly(2-methyl-2-oxazoline) (PMOX), poly(2-ethyl-2-oxazoline) (PEOZ), and polyglycerol.

Surface altering agents may include, but are not limited to, anionic proteins (e.g., bovine serum albumin), surfactants (e.g., cationic surfactants such as dimethyldioctadecyl-ammonium bromide), sugars or sugar derivatives (e.g., cyclodextrin), nucleic acids, polymers (e.g., heparin, polyethylene glycol, and poloxamer), mucolytic agents (e.g., acetylcysteine, mugwort, bromelain, papain, clerodendrum, bromhexine, carbocisteine, eprazinone, mesna, ambroxol, sobrerol, domiodol, letosteine, stepronin, tiopronin, gelsolin, thymosin β4, dornase alfa, neltenexine, and erdosteine), and DNases (e.g., rhDNase). A surface altering agent may be disposed within a nanoparticle and/or on the surface of a LNP (e.g., by coating, adsorption, covalent linkage, or other process).

A lipid nanoparticle may also comprise one or more functionalized lipids. For example, a lipid may be functionalized with an alkyne group that, when exposed to an azide under appropriate reaction conditions, may undergo a cycloaddition reaction. In particular, a lipid bilayer may be functionalized in this fashion with one or more groups useful in facilitating membrane permeation, cellular recognition, or imaging. The surface of a LNP may also be conjugated with one or more useful antibodies. Functional groups and conjugates useful in targeted cell delivery, imaging, and membrane permeation are well known in the art.

In addition to these components, lipid nanoparticles may include any substance useful in pharmaceutical compositions. For example, the lipid nanoparticle may include one or more pharmaceutically acceptable excipients or accessory ingredients such as, but not limited to, one or more solvents, dispersion media, diluents, dispersion aids, suspension aids, granulating aids, disintegrants, fillers, glidants, liquid vehicles, binders, surface active agents, isotonic agents, thickening or emulsifying agents, buffering agents, lubricating agents, oils, preservatives, and other species. Excipients such as waxes, butters, coloring agents, coating agents, flavorings, and perfuming agents may also be included. Pharmaceutically acceptable excipients are well known in the art (see for example Remington's The Science and Practice of Pharmacy, 21st Edition, A. R. Gennaro; Lippincott, Williams & Wilkins, Baltimore, M D, 2006).

Examples of diluents may include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and/or combinations thereof. Granulating and dispersing agents may be selected from the non-limiting list consisting of potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (VEEGUM®), sodium lauryl sulfate, quaternary ammonium compounds, and/or combinations thereof.

Surface active agents and/or emulsifiers may include, but are not limited to, natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite [aluminum silicate] and VEEGUM® [magnesium aluminum silicate]), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate [TWEEN®20], polyoxyethylene sorbitan [TWEEN® 60], polyoxyethylene sorbitan monooleate [TWEEN®80], sorbitan monopalmitate [SPAN®40], sorbitan monostearate [SPAN®60], sorbitan tristearate [SPAN®65], glyceryl monooleate, sorbitan monooleate [SPAN®80]), polyoxyethylene esters (e.g., polyoxyethylene monostearate [MYRJ® 45], polyoxyethylene hydrogenated castor oil, polyethoxylated castor oil, polyoxymethylene stearate, and SOLUTOL®), sucrose fatty acid esters, polyethylene glycol fatty acid esters (e.g., CREMOPHOR®), polyoxyethylene ethers, (e.g., polyoxyethylene lauryl ether [BRIJ® 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, PLURONIC®F 68, POLOXAMER® 188, cetrimonium bromide, cetylpyridinium chloride, benzalkonium chloride, docusate sodium, and/or combinations thereof. A binding agent may be starch (e.g., cornstarch and starch paste); gelatin; sugars (e.g., sucrose, glucose, dextrose, dextrin, molasses, lactose, lactitol, mannitol); natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (VEEGUM®), and larch arabogalactan); alginates; polyethylene oxide; polyethylene glycol; inorganic calcium salts; silicic acid; polymethacrylates; waxes; water; alcohol; and combinations thereof, or any other suitable binding agent.

Examples of preservatives may include, but are not limited to, antioxidants, chelating agents, antimicrobial preservatives, antifungal preservatives, alcohol preservatives, acidic preservatives, and/or other preservatives. Examples of antioxidants include, but are not limited to, alpha tocopherol, ascorbic acid, ascorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and/or sodium sulfite. Examples of chelating agents include ethylenediaminetetraacetic acid (EDTA), citric acid monohydrate, disodium edetate, dipotassium edetate, edetic acid, fumaric acid, malic acid, phosphoric acid, sodium edetate, tartaric acid, and/or trisodium edetate. Examples of antimicrobial preservatives include, but are not limited to, benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and/or thimerosal. Examples of antifungal preservatives include, but are not limited to, butyl paraben, methyl paraben, ethyl paraben, propyl paraben, benzoic acid, hydroxybenzoic acid, potassium benzoate, potassium sorbate, sodium benzoate, sodium propionate, and/or sorbic acid. Examples of alcohol preservatives include, but are not limited to, ethanol, polyethylene glycol, benzyl alcohol, phenol, phenolic compounds, bisphenol, chlorobutanol, hydroxybenzoate, and/or phenylethyl alcohol. Examples of acidic preservatives include, but are not limited to, vitamin A, vitamin C, vitamin E, beta-carotene, citric acid, acetic acid, dehydroascorbic acid, ascorbic acid, sorbic acid, and/or phytic acid. Other preservatives include, but are not limited to, tocopherol, tocopherol acetate, deteroxime mesylate, cetrimide, butylated hydroxyanisole (BHA), butylated hydroxytoluene (BHT), ethylenediamine, sodium lauryl sulfate (SLS), sodium lauryl ether sulfate (SLES), sodium bisulfite, sodium metabisulfite, potassium sulfite, potassium metabisulfite, GLYDANT PLUS®, PHENONIP®, methylparaben, GERMALL® 115, GERMABEN®II, NEOLONE™, KATHON™, and/or EUXYL®.

Examples of buffering agents include, but are not limited to, citrate buffer solutions, acetate buffer solutions, phosphate buffer solutions, ammonium chloride, calcium carbonate, calcium chloride, calcium citrate, calcium glubionate, calcium gluceptate, calcium gluconate, d-gluconic acid, calcium glycerophosphate, calcium lactate, calcium lactobionate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, amino-sulfonate buffers (e.g., HEPES), magnesium hydroxide, aluminum hydroxide, alginic acid, pyrogen-free water, isotonic saline, Ringer's solution, ethyl alcohol, and/or combinations thereof. Lubricating agents may selected from the non-limiting group consisting of magnesium stearate, calcium stearate, stearic acid, silica, talc, malt, glyceryl behenate, hydrogenated vegetable oils, polyethylene glycol, sodium benzoate, sodium acetate, sodium chloride, leucine, magnesium lauryl sulfate, sodium lauryl sulfate, and combinations thereof.

Examples of oils include, but are not limited to, almond, apricot kernel, avocado, babassu, bergamot, black current seed, borage, cade, chamomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macadamia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils as well as butyl stearate, caprylic triglyceride, capric triglyceride, cyclomethicone, diethyl sebacate, dimethicone 360, simethicone, isopropyl myristate, mineral oil, octyldodecanol, oleyl alcohol, silicone oil, and/or combinations thereof.

In one embodiment, an LNP of the disclosure does not include an additional targeting moiety, e.g., it transfects (e.g., at least 10%, 20%, 30%, 40%, 50%, 60%, 70%, 80%, 90%, or 95%) of stem or progenitor cells (e.g., HSPCs) without an additional targeting moiety.

### Methods of Using the LNP Compositions

The present disclosure provides LNP compositions, which can be delivered to cells, e.g., target cells, e.g., in vitro or in vivo. For in vivo protein expression, the cell is contacted with the LNP by administering the LNP to a subject to thereby increase or induce protein expression in or on the cells within the subject. For example, in one embodiment, the LNP is administered intravenously. In another embodiment, the LNP is administered intramuscularly. In yet other embodiment, the LNP is administered by a route selected from the group consisting of subcutaneously, intranodally and intratumorally.

In one embodiment, the cell is contacted with the LNP for a single treatment/transfection. In another embodiment, the cell is contacted with the LNP for multiple treatments/transfections (e.g., two, three, four or more treatments/transfections of the same cells).

In another embodiment, for in vivo delivery, the cell is contacted with the LNP by administering the LNP to a subject to thereby deliver the payload to cells within the subject. For example, in one embodiment, the LNP is administered intravenously. In another embodiment, the LNP is administered intramuscularly. In yet other embodiment, the LNP is administered by a route selected from the group consisting of subcutaneously, intranodally and intratumorally.

In a related aspect, provided herein is an LNP composition (e.g., an LNP composition described herein) for use in a method of modifying a cell or tissue in a subject.

In yet another aspect, provided herein is a method of delivering an LNP composition disclosed herein.

In a related aspect, provided herein is an LNP composition (e.g., an LNP composition described herein) for use in a method of delivering the LNP composition to a cell or tissue, e.g., in vivo.

In an embodiment, the method or use, comprises contacting the cell in vitro, in vivo or ex vivo with the LNP composition.

In an embodiment, the LNP composition of the present disclosure is contacted with cells, e.g., ex vivo or in vivo and can be used to deliver a payload, e.g., a secreted polypeptide, an intracellular polypeptide or a transmembrane polypeptide to a subject.

In an embodiment, the LNP composition of the present disclosure is formulated for a single administration to a subject. In another embodiment, the LNP composition of the present disclosure is formulated for repeat administration to a subject.

### Combination Therapies

In some embodiments, the methods of treatment or compositions for use disclosed herein, comprise administering an LNP disclosed herein in combination with an additional agent. In an embodiment, the additional agent is a standard of care for the disease or disorder. In an embodiment, the additional agent is a nucleic acid, e.g., an mRNA.

In some aspects, the subject for the present methods or compositions has been treated with one or more standard of care therapies. In other aspects, the subject for the present methods or compositions has not been responsive to one or more standard of care therapies.

### Sequence Optimization and Methods Thereof

In some embodiments, a polynucleotide of the disclosure comprises a sequence-optimized nucleotide sequence encoding a polypeptide disclosed herein, e.g., a polynucleotide encoding a therapeutic payload or prophylactic payload. In some embodiments, the polynucleotide of the disclosure comprises an open reading frame (ORF) encoding a therapeutic payload or prophylactic payload, wherein the ORF has been sequence optimized.

The sequence-optimized nucleotide sequences disclosed herein are distinct from the corresponding wild type nucleotide acid sequences and from other known sequence-optimized nucleotide sequences, e.g., these sequence-optimized nucleic acids have unique compositional characteristics.

In some embodiments, the polynucleotide of the disclosure comprises a uracil-modified sequence. In some embodiments, the uracil-modified sequence comprises at least one chemically modified nucleobase, e.g., 5-methoxyuracil. In some embodiments, at least 95% of a nucleobase (e.g., uracil) in a uracil-modified sequence of the disclosure are modified nucleobases. In some embodiments, at least 95% of uracil in a uracil-modified sequence is 5-methoxyuracil.

In some embodiments, a polynucleotide of the disclosure is sequence optimized.

A sequence optimized nucleotide sequence (nucleotide sequence is also referred to as “nucleic acid” herein) comprises at least one codon modification with respect to a reference sequence (e.g., a wild-type sequence encoding a therapeutic payload or prophylactic payload). Thus, in a sequence optimized nucleic acid, at least one codon is different from a corresponding codon in a reference sequence (e.g., a wild-type sequence).

In general, sequence optimized nucleic acids are generated by at least a step comprising substituting codons in a reference sequence with synonymous codons (i.e., codons that encode the same amino acid). Such substitutions can be effected, for example, by applying a codon substitution map (i.e., a table providing the codons that will encode each amino acid in the codon optimized sequence), or by applying a set of rules (e.g., if glycine is next to neutral amino acid, glycine would be encoded by a certain codon, but if it is next to a polar amino acid, it would be encoded by another codon). In addition to codon substitutions (i.e., “codon optimization”) the sequence optimization methods disclosed herein comprise additional optimization steps which are not strictly directed to codon optimization such as the removal of deleterious motifs (destabilizing motif substitution). Compositions and formulations comprising these sequence optimized nucleic acids (e.g., a RNA, e.g., an mRNA) can be administered to a subject in need thereof to facilitate in vivo expression of functionally active encoding a therapeutic payload or prophylactic payload.

Additional and exemplary methods of sequence optimization are disclosed in International Application Publication No. WO 2017/201325, filed on 18 May 2017, the entire contents of which are hereby incorporated by reference.

Micro RNA (miRNA) Binding Sites

Nucleic acid molecules (e.g., RNA, e.g., mRNA) of the disclosure can include regulatory elements, for example, microRNA (miRNA) binding sites, transcription factor binding sites, structured mRNA sequences and/or motifs, artificial binding sites engineered to act as pseudo-receptors for endogenous nucleic acid binding molecules, and combinations thereof. In some embodiments, nucleic acid molecules (e.g., RNA, e.g., mRNA) including such regulatory elements are referred to as including “sensor sequences.”

In some embodiments, a nucleic acid molecule (e.g., RNA, e.g., mRNA) of the disclosure comprises an open reading frame (ORF) encoding a polypeptide of interest and further comprises one or more miRNA binding site(s). Inclusion or incorporation of miRNA binding site(s) provides for regulation of nucleic acid molecules (e.g., RNA, e.g., mRNA) of the disclosure, and in turn, of the polypeptides encoded therefrom, based on tissue-specific and/or cell-type specific expression of naturally occurring miRNAs.

The present invention also provides pharmaceutical compositions and formulations that comprise any of the nucleic acid molecules (e.g., RNA, e.g., mRNA) described above. In some embodiments, the composition or formulation further comprises a delivery agent.

In some embodiments, the composition or formulation can contain a nucleic acid molecules (e.g., RNA, e.g., mRNA) comprising a sequence optimized nucleic acid sequence disclosed herein which encodes a polypeptide of interest. In some embodiments, the composition or formulation can contain a polynucleotide (e.g., a RNA, e.g., an mRNA) comprising a polynucleotide (e.g., an ORF) having significant sequence identity to a sequence optimized nucleic acid sequence disclosed herein which encodes a polypeptide of interest. In some embodiments, the polynucleotide further comprises a miRNA binding site, e.g., a miRNA binding site that binds a miRNA.

A miRNA, e.g., a natural-occurring miRNA, is a 19-25 nucleotide long noncoding RNA that binds to a nucleic acid molecule (e.g., RNA, e.g., mRNA) and down-regulates gene expression either by reducing stability or by inhibiting translation of the polynucleotide. A miRNA sequence comprises a “seed” region, i.e., a sequence in the region of positions 2-8 of the mature miRNA. A miRNA seed can comprise positions 2-8 or 2-7 of the mature miRNA. In some embodiments, a miRNA seed can comprise 7 nucleotides (e.g., nucleotides 2-8 of the mature miRNA), wherein the seed-complementary site in the corresponding miRNA binding site is flanked by an adenosine (A) opposed to miRNA position 1. In some embodiments, a miRNA seed can comprise 6 nucleotides (e.g., nucleotides 2-7 of the mature miRNA), wherein the seed-complementary site in the corresponding miRNA binding site is flanked by an adenosine (A) opposed to miRNA position 1. See, for example, Grimson A, Farh K K, Johnston W K, Garrett-Engele P, Lim L P, Bartel D P; Mol Cell. 2007 Jul. 6; 27(1):91-105. miRNA profiling of the target cells or tissues can be conducted to determine the presence or absence of miRNA in the cells or tissues. In some embodiments, a nucleic acid molecule (e.g., RNA, e.g., mRNA) of the disclosure comprises one or more microRNA binding sites, microRNA target sequences, microRNA complementary sequences, or microRNA seed complementary sequences. Such sequences can correspond to, e.g., have complementarity to, any known microRNA such as those taught in US Publication US2005/0261218 and US Publication US2005/0059005, the contents of each of which are incorporated herein by reference in their entirety.

microRNAs derive enzymatically from regions of RNA transcripts that fold back on themselves to form short hairpin structures often termed a pre-miRNA (precursor-miRNA). A pre-miRNA typically has a two-nucleotide overhang at its 3′ end and has 3′ hydroxyl and 5′ phosphate groups. This precursor-mRNA is processed in the nucleus and subsequently transported to the cytoplasm where it is further processed by DICER (a RNase III enzyme), to form a mature microRNA of approximately 22 nucleotides. The mature microRNA is then incorporated into a ribonuclear particle to form the RNA-induced silencing complex, RISC, which mediates gene silencing. Art-recognized nomenclature for mature miRNAs typically designates the arm of the pre-miRNA from which the mature miRNA derives; “5p” means the microRNA is from the 5-prime arm of the pre-miRNA hairpin and “3p” means the microRNA is from the 3-prime end of the pre-miRNA hairpin. A miR referred to by number herein can refer to either of the two mature microRNAs originating from opposite arms of the same pre-miRNA (e.g., either the 3p or 5p microRNA). All miRs referred to herein are intended to include both the 3p and 5p arms/sequences, unless particularly specified by the 3p or 5p designation.

As used herein, the term “microRNA (miRNA or miR) binding site” refers to a sequence within a nucleic acid molecule, e.g., within a DNA or within an RNA transcript, including in the 5′UTR and/or 3′UTR, that has sufficient complementarity to all or a region of a miRNA to interact with, associated with or bind to the miRNA. In some embodiments, a nucleic acid molecule (e.g., RNA, e.g., mRNA) of the disclosure comprising an ORF encoding a polypeptide of interest and further comprises one or more miRNA binding site(s). In exemplary embodiments, a 5′UTR and/or 3′UTR of the nucleic acid molecule (e.g., RNA, e.g., mRNA) comprises the one or more miRNA binding site(s).

A miRNA binding site having sufficient complementarity to a miRNA refers to a degree of complementarity sufficient to facilitate miRNA-mediated regulation of a nucleic acid molecule (e.g., RNA, e.g., mRNA), e.g., miRNA-mediated translational repression or degradation of the nucleic acid molecule (e.g., RNA, e.g., mRNA). In exemplary aspects of the disclosure, a miRNA binding site having sufficient complementarity to the miRNA refers to a degree of complementarity sufficient to facilitate miRNA-mediated degradation of the nucleic acid molecule (e.g., RNA, e.g., mRNA), e.g., miRNA-guided RNA-induced silencing complex (RISC)-mediated cleavage of mRNA. The miRNA binding site can have complementarity to, for example, a 19-25 nucleotide long miRNA sequence, to a 19-23 long nucleotide miRNA sequence, or to a 22-nucleotide long miRNA sequence. A miRNA binding site can be complementary to only a portion of a miRNA, e.g., to a portion less than 1, 2, 3, or 4 nucleotides of the full length of a naturally occurring miRNA sequence, or to a portion less than 1, 2, 3, or 4 nucleotides shorter than a naturally occurring miRNA sequence. Full or complete complementarity (e.g., full complementarity or complete complementarity over all or a significant portion of the length of a naturally occurring miRNA) is preferred when the desired regulation is mRNA degradation.

In some embodiments, a miRNA binding site includes a sequence that has complementarity (e.g., partial or complete complementarity) with a miRNA seed sequence. In some embodiments, the miRNA binding site includes a sequence that has complete complementarity with a miRNA seed sequence. In some embodiments, a miRNA binding site includes a sequence that has complementarity (e.g., partial or complete complementarity) with a miRNA sequence. In some embodiments, the miRNA binding site includes a sequence that has complete complementarity with a miRNA sequence. In some embodiments, a miRNA binding site has complete complementarity with a miRNA sequence but for 1, 2, or 3 nucleotide substitutions, terminal additions, and/or truncations.

In some embodiments, the miRNA binding site is the same length as the corresponding miRNA. In other embodiments, the miRNA binding site is one, two, three, four, five, six, seven, eight, nine, ten, eleven or twelve nucleotide(s) shorter than the corresponding miRNA at the 5′ terminus, the 3′ terminus, or both. In still other embodiments, the microRNA binding site is two nucleotides shorter than the corresponding microRNA at the 5′ terminus, the 3′ terminus, or both. The miRNA binding sites that are shorter than the corresponding miRNAs are still capable of degrading the mRNA incorporating one or more of the miRNA binding sites or preventing the mRNA from translation.

In some embodiments, the miRNA binding site binds the corresponding mature miRNA that is part of an active RISC containing Dicer. In another embodiment, binding of the miRNA binding site to the corresponding miRNA in RISC degrades the mRNA containing the miRNA binding site or prevents the mRNA from being translated. In some embodiments, the miRNA binding site has sufficient complementarity to miRNA so that a RISC complex comprising the miRNA cleaves the nucleic acid molecule (e.g., RNA, e.g., mRNA) comprising the miRNA binding site. In other embodiments, the miRNA binding site has imperfect complementarity so that a RISC complex comprising the miRNA induces instability in the nucleic acid molecule (e.g., RNA, e.g., mRNA) comprising the miRNA binding site. In another embodiment, the miRNA binding site has imperfect complementarity so that a RISC complex comprising the miRNA represses transcription of the nucleic acid molecule (e.g., RNA, e.g., mRNA) comprising the miRNA binding site.

In some embodiments, the miRNA binding site has one, two, three, four, five, six, seven, eight, nine, ten, eleven or twelve mismatch(es) from the corresponding miRNA.

In some embodiments, the miRNA binding site has at least about ten, at least about eleven, at least about twelve, at least about thirteen, at least about fourteen, at least about fifteen, at least about sixteen, at least about seventeen, at least about eighteen, at least about nineteen, at least about twenty, or at least about twenty-one contiguous nucleotides complementary to at least about ten, at least about eleven, at least about twelve, at least about thirteen, at least about fourteen, at least about fifteen, at least about sixteen, at least about seventeen, at least about eighteen, at least about nineteen, at least about twenty, or at least about twenty-one, respectively, contiguous nucleotides of the corresponding miRNA.

By engineering one or more miRNA binding sites into a nucleic acid molecule (e.g., RNA, e.g., mRNA) of the disclosure, the nucleic acid molecule (e.g., RNA, e.g., mRNA) can be targeted for degradation or reduced translation, provided the miRNA in question is available. This can reduce off-target effects upon delivery of the nucleic acid molecule (e.g., RNA, e.g., mRNA). For example, if a nucleic acid molecule (e.g., RNA, e.g., mRNA) of the disclosure is not intended to be delivered to a tissue or cell but ends up is said tissue or cell, then a miRNA abundant in the tissue or cell can inhibit the expression of the gene of interest if one or multiple binding sites of the miRNA are engineered into the 5′UTR and/or 3′UTR of the nucleic acid molecule (e.g., RNA, e.g., mRNA). Thus, in some embodiments, incorporation of one or more miRNA binding sites into an mRNA of the disclosure may reduce the hazard of off-target effects upon nucleic acid molecule delivery and/or enable tissue-specific regulation of expression of a polypeptide encoded by the mRNA. In yet other embodiments, incorporation of one or more miRNA binding sites into an mRNA of the disclosure can modulate immune responses upon nucleic acid delivery in vivo. In further embodiments, incorporation of one or more miRNA binding sites into an mRNA of the disclosure can modulate accelerated blood clearance (ABC) of lipid-comprising compounds and compositions described herein.

For example, one of skill in the art would understand that one or more miR binding sites can be included in a nucleic acid molecule (e.g., an RNA, e.g., mRNA) to minimize expression in cell types other than lymphoid cells. In one embodiment, a miR122 binding site can be used. In another embodiment, a miR126 binding site can be used. In still another embodiment, multiple copies of these miR binding sites or combinations may be used.

Conversely, miRNA binding sites can be removed from nucleic acid molecule (e.g., RNA, e.g., mRNA) sequences in which they naturally occur in order to increase protein expression in specific tissues. For example, a binding site for a specific miRNA can be removed from a nucleic acid molecule (e.g., RNA, e.g., mRNA) to improve protein expression in tissues or cells containing the miRNA.

Regulation of expression in multiple tissues can be accomplished through introduction or removal of one or more miRNA binding sites, e.g., one or more distinct miRNA binding sites. The decision whether to remove or insert a miRNA binding site can be made based on miRNA expression patterns and/or their profiling in tissues and/or cells in development and/or disease. Identification of miRNAs, miRNA binding sites, and their expression patterns and role in biology have been reported (e.g., Bonauer et al., Curr Drug Targets 2010 11:943-949; Anand and Cheresh Curr Opin Hematol 2011 18:171-176; Contreras and Rao Leukemia 2012 26:404-413 (2011 Dec 20. doi: 10.1038/leu.2011.356); Bartel Cell 2009 136:215-233; Landgraf et al, Cell, 2007 129:1401-1414; Gentner and Naldini, Tissue Antigens. 2012 80:393-403 and all references therein; each of which is incorporated herein by reference in its entirety).

miRNAs and miRNA binding sites can correspond to any known sequence, including non-limiting examples described in U.S. Publication Nos. 2014/0200261, 2005/0261218, and 2005/0059005, each of which are incorporated herein by reference in their entirety.

Examples of tissues where miRNA are known to regulate mRNA, and thereby protein expression, include, but are not limited to, liver (miR-122), muscle (miR-133, miR-206, miR-208), endothelial cells (miR-17-92, miR-126), myeloid cells (miR-142-3p, miR-142-5p, miR-16, miR-21, miR-223, miR-24, miR-27), adipose tissue (let-7, miR-30c), heart (miR-1d, miR-149), kidney (miR-192, miR-194, miR-204), and lung epithelial cells (let-7, miR-133, miR-126).

Specifically, miRNAs are known to be differentially expressed in immune cells (also called hematopoietic cells), such as antigen presenting cells (APCs) (e.g., dendritic cells and monocytes), monocytes, monocytes, B lymphocytes, T lymphocytes, granulocytes, natural killer cells, etc. Immune cell specific miRNAs are involved in immunogenicity, autoimmunity, the immune response to infection, inflammation, as well as unwanted immune response after gene therapy and tissue/organ transplantation. Immune cell specific miRNAs also regulate many aspects of development, proliferation, differentiation and apoptosis of hematopoietic cells (e.g., immune cells). For example, miR-142 and miR-146 are exclusively expressed in immune cells, particularly abundant in myeloid dendritic cells. It has been demonstrated that the immune response to a nucleic acid molecule (e.g., RNA, e.g., mRNA) can be shut-off by adding miR-142 binding sites to the 3′-UTR of the polynucleotide, enabling more stable gene transfer in tissues and cells. miR-142 efficiently degrades exogenous nucleic acid molecules (e.g., RNA, e.g., mRNA) in antigen presenting cells and suppresses cytotoxic elimination of transduced cells (e.g., Annoni A et al., blood, 2009, 114, 5152-5161; Brown B D, et al., Nat med. 2006, 12(5), 585-591; Brown B D, et al., blood, 2007, 110(13): 4144-4152, each of which is incorporated herein by reference in its entirety).

An antigen-mediated immune response can refer to an immune response triggered by foreign antigens, which, when entering an organism, are processed by the antigen presenting cells and displayed on the surface of the antigen presenting cells. T cells can recognize the presented antigen and induce a cytotoxic elimination of cells that express the antigen.

Introducing a miR-142 binding site into the 5′UTR and/or 3′UTR of a nucleic acid molecule of the disclosure can selectively repress gene expression in antigen presenting cells through miR-142 mediated degradation, limiting antigen presentation in antigen presenting cells (e.g., dendritic cells) and thereby preventing antigen-mediated immune response after the delivery of the nucleic acid molecule (e.g., RNA, e.g., mRNA). The nucleic acid molecule (e.g., RNA, e.g., mRNA) is then stably expressed in target tissues or cells without triggering cytotoxic elimination.

In one embodiment, binding sites for miRNAs that are known to be expressed in immune cells, in particular, antigen presenting cells, can be engineered into a nucleic acid molecule (e.g., RNA, e.g., mRNA) of the disclosure to suppress the expression of the nucleic acid molecule (e.g., RNA, e.g., mRNA) in antigen presenting cells through miRNA mediated RNA degradation, subduing the antigen-mediated immune response. Expression of the nucleic acid molecule (e.g., RNA, e.g., mRNA) is maintained in non-immune cells where the immune cell specific miRNAs are not expressed. For example, in some embodiments, to prevent an immunogenic reaction against a liver specific protein, any miR-122 binding site can be removed and a miR-142 (and/or mirR-146) binding site can be engineered into the 5′UTR and/or 3′UTR of a nucleic acid molecule of the disclosure.

To further drive the selective degradation and suppression in APCs and macrophage, a nucleic acid molecule (e.g., RNA, e.g., mRNA) of the disclosure can include a further negative regulatory element in the 5′UTR and/or 3′UTR, either alone or in combination with miR-142 and/or miR-146 binding sites. As a non-limiting example, the further negative regulatory element is a Constitutive Decay Element (CDE).

Immune cell specific miRNAs include, but are not limited to, hsa-let-7a-2-3p, hsa-let-7a-3p, hsa-7a-5p, hsa-let-7c, hsa-let-7e-3p, hsa-let-7e-5p, hsa-let-7g-3p, hsa-let-7g-5p, hsa-let-7i-3p, hsa-let-7i-5p, miR-10a-3p, miR-10a-5p, miR-1184, hsa-let-7f-1-3p, hsa-let-7f-2-5p, hsa-let-7f-5p, miR-125b-1-3p, miR-125b-2-3p, miR-125b-5p, miR-1279, miR-130a-3p, miR-130a-5p, miR-132-3p, miR-132-5p, miR-142-3p, miR-142-5p, miR-143-3p, miR-143-5p, miR-146a-3p, miR-146a-5p, miR-146b-3p, miR-146b-5p, miR-147a, miR-147b, miR-148a-5p, miR-148a-3p, miR-150-3p, miR-150-5p, miR-151b, miR-155-3p, miR-155-5p, miR-15a-3p, miR-15a-5p, miR-15b-5p, miR-15b-3p, miR-16-1-3p, miR-16-2-3p, miR-16-5p, miR-17-5p, miR-181a-3p, miR-181a-5p, miR-181a-2-3p, miR-182-3p, miR-182-5p, miR-197-3p, miR-197-5p, miR-21-5p, miR-21-3p, miR-214-3p, miR-214-5p, miR-223-3p, miR-223-5p, miR-221-3p, miR-221-5p, miR-23b-3p, miR-23b-5p, miR-24-1-5p,miR-24-2-5p, miR-24-3p, miR-26a-1-3p, miR-26a-2-3p, miR-26a-5p, miR-26b-3p, miR-26b-5p, miR-27a-3p, miR-27a-5p, miR-27b-3p,miR-27b-5p, miR-28-3p, miR-28-5p, miR-2909, miR-29a-3p, miR-29a-5p, miR-29b-1-5p, miR-29b-2-5p, miR-29c-3p, miR-29c-5p, miR-30e-3p, miR-30e-5p, miR-331-5p, miR-339-3p, miR-339-5p, miR-345-3p, miR-345-5p, miR-346, miR-34a-3p, miR-34a-5p, miR-363-3p, miR-363-5p, miR-372, miR-377-3p, miR-377-5p, miR-493-3p, miR-493-5p, miR-542, miR-548b-5p, miR548c-5p, miR-548i, miR-548j, miR-548n, miR-574-3p, miR-598, miR-718, miR-935, miR-99a-3p, miR-99a-5p, miR-99b-3p, and miR-99b-5p. Furthermore, novel miRNAs can be identified in immune cell through micro-array hybridization and microtome analysis (e.g., Jima D D et al, Blood, 2010, 116:e118-e127; Vaz C et al., BMC Genomics, 2010, 11, 288, the content of each of which is incorporated herein by reference in its entirety.)

In some embodiments, a nucleic acid molecule (e.g., RNA, e.g., mRNA) of the disclosure comprises a miRNA binding site, wherein the miRNA binding site comprises one or more nucleotide sequences selected from Table 3C or Table 4A, including one or more copies of any one or more of the miRNA binding site sequences. In some embodiments, a nucleic acid molecule (e.g., RNA, e.g., mRNA) of the disclosure further comprises at least one, two, three, four, five, six, seven, eight, nine, ten, or more of the same or different miRNA binding sites selected from Table 3C or Table 4A, including any combination thereof.

In some embodiments, the miRNA binding site binds to miR-142 or is complementary to miR-142. In some embodiments, the miR-142 comprises SEQ ID NO:114. In some embodiments, the miRNA binding site binds to miR-142-3p or miR-142-5p. In some embodiments, the miR-142-3p binding site comprises SEQ ID NO:202. In some embodiments, the miR-142-5p binding site comprises SEQ ID NO:204. In some embodiments, the miRNA binding site comprises a nucleotide sequence at least 80%, at least 85%, at least 90%, at least 95%, or 100% identical to SEQ ID NO:202 or SEQ ID NO:204.

In some embodiments, the miRNA binding site binds to miR-126 or is complementary to miR-126. In some embodiments, the miR-126 comprises SEQ ID NO: 205. In some embodiments, the miRNA binding site binds to miR-126-3p or miR-126-5p. In some embodiments, the miR-126-3p binding site comprises SEQ ID NO: 207. In some embodiments, the miR-126-5p binding site comprises SEQ ID NO: 209. In some embodiments, the miRNA binding site comprises a nucleotide sequence at least 80%, at least 85%, at least 90%, at least 95%, or 100% identical to SEQ ID NO: 121 or SEQ ID NO: 123.

In one embodiment, the 3′ UTR comprises two miRNA binding sites, wherein a first miRNA binding site binds to miR-142 and a second miRNA binding site binds to miR-126. In a specific embodiment, the 3′ UTR binding to miR-142 and miR-126 comprises, consists, or consists essentially of the sequence of SEQ ID NO: 249.

TABLE 3C
miR-142, miR-126, and miR-142 and
miR-126 binding sites

| SEQ | | |
|---|---|---|
| ID NO. | Description | Sequence |
| 114 | miR-142 | GACAGUGCAGUCACCCAUAAAGUAGAAAGCA |
| | | CUACUAACAGCACUGGAGGGUGUAGUGUUUC |
| | | CUACUUUAUGGAUGAGUGUACUGUG |
| 201 | miR-142-3p | UGUAGUGUUUCCUACUUUAUGGA |
| 202 | miR-142-3p | UCCAUAAAGUAGGAAACACUACA |
| | binding site | |
| 203 | miR-142-5p | CAUAAAGUAGAAAGCACUACU |
| 204 | miR-142-5p | AGUAGUGCUUUCUACUUUAUG |
| | binding site | |
| 205 | miR-126 | CGCUGGCGACGGGACAUUAUUACUUUUGGUA |
| | | CGCGCUGUGACACUUCAAACUCGUACCGUGA |
| | | GUAAUAAUGCGCCGUCCACGGCA |
| 206 | miR-126-3p | UCGUACCGUGAGUAAUAAUGCG |
| 207 | miR-126-3p | CGCAUUAUUACUCACGGUACGA |
| | binding site | |
| 208 | miR-126-5p | CAUUAUUACUUUUGGUACGCG |
| 209 | miR-126-5p | CGCGUACCAAAAGUAAUAAUG |
| | binding site | |

In some embodiments, a miRNA binding site is inserted in the nucleic acid molecule (e.g., RNA, e.g., mRNA) of the disclosure in any position of the nucleic acid molecule (e.g., RNA, e.g., mRNA) (e.g., the 5′UTR and/or 3′UTR). In some embodiments, the 5′UTR comprises a miRNA binding site. In some embodiments, the 3′UTR comprises a miRNA binding site. In some embodiments, the 5′UTR and the 3′UTR comprise a miRNA binding site. The insertion site in the nucleic acid molecule (e.g., RNA, e.g., mRNA) can be anywhere in the nucleic acid molecule (e.g., RNA, e.g., mRNA) as long as the insertion of the miRNA binding site in the nucleic acid molecule (e.g., RNA, e.g., mRNA) does not interfere with the translation of a functional polypeptide in the absence of the corresponding miRNA; and in the presence of the miRNA, the insertion of the miRNA binding site in the nucleic acid molecule (e.g., RNA, e.g., mRNA) and the binding of the miRNA binding site to the corresponding miRNA are capable of degrading the polynucleotide or preventing the translation of the nucleic acid molecule (e.g., RNA, e.g., mRNA).

In some embodiments, a miRNA binding site is inserted in at least about 30 nucleotides downstream from the stop codon of an ORF in a nucleic acid molecule (e.g., RNA, e.g., mRNA) of the disclosure comprising the ORF. In some embodiments, a miRNA binding site is inserted in at least about 10 nucleotides, at least about 15 nucleotides, at least about 20 nucleotides, at least about 25 nucleotides, at least about 30 nucleotides, at least about 35 nucleotides, at least about 40 nucleotides, at least about 45 nucleotides, at least about 50 nucleotides, at least about 55 nucleotides, at least about 60 nucleotides, at least about 65 nucleotides, at least about 70 nucleotides, at least about 75 nucleotides, at least about 80 nucleotides, at least about 85 nucleotides, at least about 90 nucleotides, at least about 95 nucleotides, or at least about 100 nucleotides downstream from the stop codon of an ORF in a polynucleotide of the disclosure. In some embodiments, a miRNA binding site is inserted in about 10 nucleotides to about 100 nucleotides, about 20 nucleotides to about 90 nucleotides, about 30 nucleotides to about 80 nucleotides, about 40 nucleotides to about 70 nucleotides, about 50 nucleotides to about 60 nucleotides, about 45 nucleotides to about 65 nucleotides downstream from the stop codon of an ORF in a nucleic acid molecule (e.g., RNA, e.g., mRNA) of the disclosure.

In some embodiments, a miRNA binding site is inserted within the 3′ UTR immediately following the stop codon of the coding region within the nucleic acid molecule (e.g., RNA, e.g., mRNA) of the disclosure. In some embodiments, if there are multiple copies of a stop codon in the construct, a miRNA binding site is inserted immediately following the final stop codon. In some embodiments, a miRNA binding site is inserted further downstream of the stop codon, in which case there are 3′ UTR bases between the stop codon and the miR binding site(s). In some embodiments, three non-limiting examples of possible insertion sites for a miR in a 3′ UTR are shown in SEQ ID NOs: 248, 249, and 250, which show a 3′ UTR sequence with a miR-142-3p site inserted in one of three different possible insertion sites, respectively, within the 3′ UTR.

In some embodiments, one or more miRNA binding sites can be positioned within the 5′ UTR at one or more possible insertion sites. For example, three non-limiting examples of possible insertion sites for a miR in a 5′ UTR are shown in SEQ ID NOs: 251, 252, or 253, which show a 5′ UTR sequence with a miR-142-3p site inserted into one of three different possible insertion sites, respectively, within the 5′ UTR.

In one embodiment, a codon optimized open reading frame encoding a polypeptide of interest comprises a stop codon and the at least one microRNA binding site is located within the 3′ UTR 1-100 nucleotides after the stop codon. In one embodiment, the codon optimized open reading frame encoding the polypeptide of interest comprises a stop codon and the at least one microRNA binding site for a miR expressed in immune cells is located within the 3′ UTR 30-50 nucleotides after the stop codon. In another embodiment, the codon optimized open reading frame encoding the polypeptide of interest comprises a stop codon and the at least one microRNA binding site for a miR expressed in immune cells is located within the 3′ UTR at least 50 nucleotides after the stop codon. In other embodiments, the codon optimized open reading frame encoding the polypeptide of interest comprises a stop codon and the at least one microRNA binding site for a miR expressed in immune cells is located within the 3′ UTR immediately after the stop codon, or within the 3′ UTR 15-20 nucleotides after the stop codon or within the 3′ UTR 70-80 nucleotides after the stop codon. In other embodiments, the 3′ UTR comprises more than one miRNA binding site (e.g., 2-4 miRNA binding sites), wherein there can be a spacer region (e.g., of 10-100, 20-70 or 30-50 nucleotides in length) between each miRNA binding site. In another embodiment, the 3′ UTR comprises a spacer region between the end of the miRNA binding site(s) and the poly A tail nucleotides. For example, a spacer region of 10-100, 20-70 or 30-50 nucleotides in length can be situated between the end of the miRNA binding site(s) and the beginning of the poly A tail.

In one embodiment, a codon optimized open reading frame encoding a polypeptide of interest comprises a start codon and the at least one microRNA binding site is located within the 5′ UTR 1-100 nucleotides before (upstream of) the start codon. In one embodiment, the codon optimized open reading frame encoding the polypeptide of interest comprises a start codon and the at least one microRNA binding site for a miR expressed in immune cells is located within the 5′ UTR 10-50 nucleotides before (upstream of) the start codon. In another embodiment, the codon optimized open reading frame encoding the polypeptide of interest comprises a start codon and the at least one microRNA binding site for a miR expressed in immune cells is located within the 5′ UTR at least 25 nucleotides before (upstream of) the start codon. In other embodiments, the codon optimized open reading frame encoding the polypeptide of interest comprises a start codon and the at least one microRNA binding site for a miR expressed in immune cells is located within the 5′ UTR immediately before the start codon, or within the 5′ UTR 15-20 nucleotides before the start codon or within the 5′ UTR 70-80 nucleotides before the start codon. In other embodiments, the 5′ UTR comprises more than one miRNA binding site (e.g., 2-4 miRNA binding sites), wherein there can be a spacer region (e.g., of 10-100, 20-70 or 30-50 nucleotides in length) between each miRNA binding site.

In one embodiment, the 3′ UTR comprises more than one stop codon, wherein at least one miRNA binding site is positioned downstream of the stop codons. For example, a 3′ UTR can comprise 1, 2 or 3 stop codons. Non-limiting examples of triple stop codons that can be used include: UGAUAAUAG (SEQ ID NO:210), UGAUAGUAA (SEQ ID NO:211), UAAUGAUAG (SEQ ID NO:277), UGAUAAUAA (SEQ ID NO:213), UGAUAGUAG (SEQ ID NO:214), UAAUGAUGA (SEQ ID NO:215), UAAUAGUAG (SEQ ID NO:216), UGAUGAUGA (SEQ ID NO:217), UAAUAAUAA (SEQ ID NO:218), and UAGUAGUAG (SEQ ID NO:219). Within a 3′ UTR, for example, 1, 2, 3 or 4 miRNA binding sites, e.g., miR-142-3p binding sites, can be positioned immediately adjacent to the stop codon(s) or at any number of nucleotides downstream of the final stop codon. When the 3′ UTR comprises multiple miRNA binding sites, these binding sites can be positioned directly next to each other in the construct (i.e., one after the other) or, alternatively, spacer nucleotides can be positioned between each binding site.

In one embodiment, the 3′ UTR comprises three stop codons with a single miR-142-3p binding site located downstream of the 3rd stop codon. Non-limiting examples of sequences of 3′ UTR having three stop codons and a single miR-142-3p binding site located at different positions downstream of the final stop codon are shown in SEQ TD NOs: 237, 248, 249, and 250.

TABLE 4A
5′ UTRs, 3′UTRs, miR sequences, and miR binding sites

| SEQ ID NO: | Sequence |
|---|---|
| 220 | GCUGGAGCCUCGGUGGCCAUGCUUCUUGCCCCUUGGGCCUCCCCCCAGCCCC |
| | UCCUCCCCUUCCUGCACCCGUACCCCCUCCAUAAAGUAGGAAACACUACAGU |
| | GGUCUUUGAAUAAAGUCUGAGUGGGCGGC |
| | (3′ UTR with miR 142-3p binding site) |
| 202 | UCCAUAAAGUAGGAAACACUACA |
| | (miR 142-3p binding site) |
| 201 | UGUAGUGUUUCCUACUUUAUGGA |
| | (miR 142-3p sequence) |
| 203 | CAUAAAGUAGAAAGCACUACU |
| | (miR 142-5p sequence) |
| 221 | CCUCUGAAAUUCAGUUCUUCAG |
| | (miR 146-3p sequence) |
| 222 | UGAGAACUGAAUUCCAUGGGUU |
| | (miR 146-5p sequence) |
| 223 | CUCCUACAUAUUAGCAUUAACA |
| | (miR 155-3p sequence) |
| 224 | UUAAUGCUAAUCGUGAUAGGGGU |
| | (miR 155-5p sequence) |
| 206 | UCGUACCGUGAGUAAUAAUGCG |
| | (miR 126-3p sequence) |
| 208 | CAUUAUUACUUUUGGUACGCG |
| | (miR 126-5p sequence) |
| 225 | CCAGUAUUAACUGUGCUGCUGA |
| | (miR 16-3p sequence) |
| 226 | UAGCAGCACGUAAAUAUUGGCG |
| | (miR 16-5p sequence) |
| 227 | CAACACCAGUCGAUGGGCUGU |
| | (miR 21-3p sequence) |
| 228 | UAGCUUAUCAGACUGAUGUUGA |
| | (miR 21-5p sequence) |
| 143 | UGUCAGUUUGUCAAAUACCCCA |
| | (miR 223-3p sequence) |
| 230 | CGUGUAUUUGACAAGCUGAGUU |
| | (miR 223-5p sequence) |
| 231 | UGGCUCAGUUCAGCAGGAACAG |
| | (miR 24-3p sequence) |
| 232 | UGCCUACUGAGCUGAUAUCAGU |
| | (miR 24-5p sequence) |
| 233 | UUCACAGUGGCUAAGUUCCGC |
| | (miR 27-3p sequence) |
| 234 | AGGGCUUAGCUGCUUGUGAGCA |
| | (miR 27-5p sequence) |
| 207 | CGCAUUAUUACUCACGGUACGA |
| | (miR 126-3p binding site) |
| 235 | UGAUAAUAGGCUGGAGCCUCGGUGGCCAUGCUUCUUGCCCCUUGGGCCUCCC |
| | CCCAGCCCCUCCUCCCCUUCCUGCACCCGUACCCCC [symbol] |
| | [symbol] GUGGUCUUUGAAUAAAGUCUGAGUGGGCGGC |
| | (3′ UTR with miR 126-3p binding site) |
| 236 | UGAUAAUAGGCUGGAGCCUCGGUGGCCAUGCUUCUUGCCCCUUGGGCCUCCC |
| | CCCAGCCCCUCCUCCCCUUCCUGCACCCGUACCCCCGUGGUCUUUGAAUAAA |
| | GUCUGAGUGGGCGGC |
| | (3′ UTR, no miR binding sites) |
| 237 | UGAUAAUAGGCUGGAGCCUCGGUGGCCAUGCUUCUUGCCCCUUGGGCCUCCC |
| | CCCAGCCCCUCCUCCCCUUCCUGCACCCGUACCCCCUCCAUAAAGUAGGAAA |
| | CACUACAGUGGUCUUUGAAUAAAGUCUGAGUGGGCGGC |
| | (3′ UTR with miR 142-3p binding site) |
| 199 | UGAUAAUAGUCCAUAAAGUAGGAAACACUACAGCUGGAGCCUCGGUGGCCAU |
| | GCUUCUUGCCCCUUGGGCCUCCCCCCAGCCCCUCCUCCCCUUCCUGCACCCG |
| | UACCCCC [symbol] GUGGUCUUUGAAUAAAGUCUGAG |
| | UGGGCGGC |
| | (3′ UTR with miR 142-3p and miR 126-3p binding sites |
| | variant 1) |
| 239 | UUAAUGCUAAUUGUGAUAGGGGU |
| | (miR 155-5p sequence) |
| 240 | ACCCCUAUCACAAUUAGCAUUAA |
| | (miR 155-5p binding site) |
| 241 | UGAUAAUAGUCCAUAAAGUAGGAAACACUACAGCUGGAGCCUCGGUGGCCAU |
| | GCUUCUUGCCCCUUGGGCCUCCAUAAAGUAGGAAACACUACAUCCCCCCAGC |
| | CCCUCCUCCCCUUCCUGCACCCGUACCCCCUCCAUAAAGUAGGAAACACUAC |
| | AGUGGUCUUUGAAUAAAGUCUGAGUGGGCGGC |
| | (3′ UTR with 3 miR 142-3p binding sites) |
| 242 | UGAUAAUAGGCUGGAGCCUCGGUGGCCAUGCUUCUUGCCCCUUGGGCCUCCC |
| | CCCAGCCCCUCCUCCCCUUCCUGCACCCGUACCCCC [symbol] |
| | [symbol] GUGGUCUUUGAAUAAAGUCUGAGUGGGCGGC |
| | (3′ UTR with miR 142-5p binding site) |
| 243 | UGAUAAUAG [symbol] GCUGGAGCCUCGGUGGCCAUGC |
| | UUCUUGCCCCUUGGGCC [symbol] UCCCCCCAGCCCCU |
| | CCUCCCCUUCCUGCACCCGUACCCCC [symbol] GUGGU |
| | CUUUGAAUAAAGUCUGAGUGGGCGGC |
| | (3′ UTR with 3 miR 142-5p binding sites) |
| 244 | UGAUAAUAGAGUAGUGCUUUCUACUUUAUGGCUGGAGCCUCGGUGGCCAUGC |
| | UUCUUGCCCCUUGGGCCUCCAUAAAGUAGGAAACACUACAUCCCCCCAGCCC |
| | CUCCUCCCCUUCCUGCACCCGUACCCCC [symbol] GUG |
| | GUCUUUGAAUAAAGUCUGAGUGGGCGGC |
| | (3′ UTR with 2 miR 142-5p binding sites and 1 miR |
| | 142-3p binding site) |
| 245 | UGAUAAUAGGCUGGAGCCUCGGUGGCCAUGCUUCUUGCCCCUUGGGCCUCCC |
| | CCCAGCCCCUCCUCCCCUUCCUGCACCCGUACCCCCACCCCUAUCACAAUUA |
| | GCAUUAAGUGGUCUUUGAAUAAAGUCUGAGUGGGCGGC |
| | (3′ UTR with miR 155-5p binding site) |
| 246 | UGAUAAUAGACCCCUAUCACAAUUAGCAUUAAGCUGGAGCCUCGGUGGCCAU |
| | GCUUCUUGCCCCUUGGGCCACCCCUAUCACAAUUAGCAUUAAUCCCCCCAGC |
| | CCCUCCUCCCCUUCCUGCACCCGUACCCCCACCCCUAUCACAAUUAGCAUUA |
| | AGUGGUCUUUGAAUAAAGUCUGAGUGGGCGGC |
| | (3′ UTR with 3 miR 155-5p binding sites) |
| 247 | UGAUAAUAGACCCCUAUCACAAUUAGCAUUAAGCUGGAGCCUCGGUGGCCAU |
| | GCUUCUUGCCCCUUGGGCCUCCAUAAAGUAGGAAACACUACAUCCCCCCAGC |
| | CCCUCCUCCCCUUCCUGCACCCGUACCCCCACCCCUAUCACAAUUAGCAUUA |
| | AGUGGUCUUUGAAUAAAGUCUGAGUGGGCGGC |
| | (3′ UTR with 2 miR 155-5p binding sites and 1 miR |
| | 142-3p binding site) |
| 248 | UGAUAAUAGUCCAUAAAGUAGGAAACACUACAGCUGGAGCCUCGGUGGCCAU |
| | GCUUCUUGCCCCUUGGGCCUCCCCCCAGCCCCUCCUCCCCUUCCUGCACCCG |
| | UACCCCCGUGGUCUUUGAAUAAAGUCUGAGUGGGCGGC |
| | (3′ UTR with miR 142-3p binding site, P1 insertion) |
| 249 | UGAUAAUAGGCUGGAGCCUCGGUGGCUCCAUAAAGUAGGAAACACUACACAU |
| | GCUUCUUGCCCCUUGGGCCUCCCCCCAGCCCCUCCUCCCCUUCCUGCACCCG |
| | UACCCCCGUGGUCUUUGAAUAAAGUCUGAGUGGGCGGC |
| | (3′ UTR with miR 142-3p binding site, P2 insertion) |
| 250 | UGAUAAUAGGCUGGAGCCUCGGUGGCCAUGCUUCUUGCCCCUUGGGCCUCCA |
| | UAAAGUAGGAAACACUACAUCCCCCCAGCCCCUCCUCCCCUUCCUGCACCCG |
| | UACCCCCGUGGUCUUUGAAUAAAGUCUGAGUGGGCGGC |
| | (3′ UTR with miR 142-3p binding site, P3 insertion) |
| 204 | AGUAGUGCUUUCUACUUUAUG |
| | (miR-142-5p binding site) |
| 200 | GACAGUGCAGUCACCCAUAAAGUAGAAAGCACUACUAACAGCACUGGAGGGU |
| | GUAGUGUUUCCUACUUUAUGGAUGAGUGUACUGUG |
| | (miR-142) |
| 197 | GGGAAAUAAGAGAGAAAAGAAGAGUAAGAAGAAAUAUAAGAGCCACC |
| | (5′ UTR) |
| 251 | GGGAAAUAAGAGUCCAUAAAGUAGGAAACACUACAAGAAAAGAAGAGUAAGA |
| | AGAAAUAUAAGAGCCACC |
| | (5′ UTR with miR142-3p binding site at position p1) |
| 252 | GGGAAAUAAGAGAGAAAAGAAGAGUAAUCCAUAAAGUAGGAAACACUACAGA |
| | AGAAAUAUAAGAGCCACC |
| | (5′ UTR with miR142-3p binding site at position p2) |
| 253 | GGGAAAUAAGAGAGAAAAGAAGAGUAAGAAGAAAUAUAAUCCAUAAAGUAGG |
| | AAACACUACAGAGCCACC |
| | (5′ UTR with miR142-3p binding site at position p3) |
| 254 | ACCCCUAUCACAAUUAGCAUUAA |
| | (miR 155-5p binding site) |
| 255 | UGAUAAUAG [symbol] GCUGGAGCCUCGGUGGCCAUGC |
| | UUCUUGCCCCUUGGGCC [symbol] UCCCCCCAGCCCCU |
| | CUCCCCUUCCUGCACCCGUACCCCC [symbol] GUGGUC |
| | UUUGAAUAAAGUCUGAGUGGGCGGC |
| | (3′ UTR with 3 miR 142-5p binding sites) |
| 256 | UGAUAAUAGGCUGGAGCCUCGGUGGCCAUGCUUCUUGCCCCUUCCAUAAAGU |
| | AGGAAACACUACAUGGGCCUCCCCCCAGCCCCUCCUCCCCUUCCUGCACCCG |
| | UACCCCCGUGGUCUUUGAAUAAAGUCUGAGUGGGCGGC |
| | (3′UTR including miR142-3p binding site) |
| 257 | UGAUAAUAGGCUGGAGCCUCGGUGGCCAUGCUUCUUGCCCCUUGGGCCUCCC |
| | CCCAGUCCAUAAAGUAGGAAACACUACACCCCUCCUCCCCUUCCUGCACCCG |
| | UACCCCCGUGGUCUUUGAAUAAAGUCUGAGUGGGCGGC |
| | (3′ UTR including miR142-3p binding site) |
| 258 | UGAUAAUAGGCUGGAGCCUCGGUGGCCAUGCUUCUUGCCCCUUGGGCCUCCC |
| | CCCAGCCCCUCCUCCCCUUCUCCAUAAAGUAGGAAACACUACACUGCACCCG |
| | UACCCCCGUGGUCUUUGAAUAAAGUCUGAGUGGGCGGC |
| | (3′ UTR including miR142-3p binding site) |
| 259 | UGAUAAUAGGCUGGAGCCUCGGUGGCCAUGCUUCUUGCCCCUUGGGCCUCCC |
| | CCCAGCCCCUCCUCCCCUUCCUGCACCCGUACCCCCGUGGUCUUUGAAUAAA |
| | GUUCCAUAAAGUAGGAAACACUACACUGAGUGGGCGGC |
| | (3′ UTR including miR142-3p binding site) |
| 260 | UGAUAAUAGUCCAUAAAGUAGGAAACACUACAGCUGGAGCCUCGGUGGCCUA |
| | GCUUCUUGCCCCUUGGGCCUCCCCCCAGCCCCUCCUCCCCUUCCUGCACCCG |
| | UACCCCC [symbol] GUGGUCUUUGAAUAAAGUCUGAG |
| | UGGGCGGC |
| | (3′ UTR with miR 142-3p and miR 126-3p binding sites |
| | variant 2) |
| 261 | UGAUAAUAGGCUGGAGCCUCGGUGGCCUAGCUUCUUGCCCCUUGGGCCUCCC |
| | CCCAGCCCCUCCUCCCCUUCCUGCACCCGUACCCCCGUGGUCUUUGAAUAAA |
| | GUCUGAGUGGGCGGC |
| | (3′ UTR, no miR binding sites variant 2) |
| 198 | UGAUAAUAGGCUGGAGCCUCGGUGGCCUAGCUUCUUGCCCCUUGGGCCUCCC |
| | CCCAGCCCCUCCUCCCCUUCCUGCACCCGUACCCCCUCCAUAAAGUAGGAAA |
| | CACUACAGUGGUCUUUGAAUAAAGUCUGAGUGGGCGGC |
| | (3′ UTR with miR 142-3p binding site variant 3) |
| 262 | UGAUAAUAGGCUGGAGCCUCGGUGGCCUAGCUUCUUGCCCCUUGGGCCUCCC |
| | CCCAGCCCCUCCUCCCCUUCCUGCACCCGUACCCCC [symbol] |
| | [symbol] GUGGUCUUUGAAUAAAGUCUGAGUGGGCGGC |
| | (3′ UTR with miR 126-3p binding site variant 3) |
| 263 | UGAUAAUAGUCCAUAAAGUAGGAAACACUACAGCUGGAGCCUCGGUGGCCUA |
| | GCUUCUUGCCCCUUGGGCCUCCAUAAAGUAGGAAACACUACAUCCCCCCAGC |
| | CCCUCCUCCCCUUCCUGCACCCGUACCCCCUCCAUAAAGUAGGAAACACUAC |
| | AGUGGUCUUUGAAUAAAGUCUGAGUGGGCGGC |
| | (3′ UTR with 3 miR 142-3p binding sites variant 2) |
| 264 | UGAUAAUAGUCCAUAAAGUAGGAAACACUACAGCUGGAGCCUCGGUGGCCUA |
| | GCUUCUUGCCCCUUGGGCCUCCCCCCAGCCCCUCCUCCCCUUCCUGCACCCG |
| | UACCCCCGUGGUCUUUGAAUAAAGUCUGAGUGGGCGGC |
| | (3′ UTR with miR 142-3p binding site, P1 insertion |
| | variant 2) |
| 265 | UGAUAAUAGGCUGGAGCCUCGGUGGCUCCAUAAAGUAGGAAACACUACACUA |
| | GCUUCUUGCCCCUUGGGCCUCCCCCCAGCCCCUCCUCCCCUUCCUGCACCCG |
| | UACCCCCGUGGUCUUUGAAUAAAGUCUGAGUGGGCGGC |
| | (3′UTR with miR 142-3p binding site, P2 insertion |
| | variant 2) |
| 266 | UGAUAAUAGGCUGGAGCCUCGGUGGCCUAGCUUCUUGCCCCUUGGGCCUCCA |
| | UAAAGUAGGAAACACUACAUCCCCCCAGCCCCUCCUCCCCUUCCUGCACCCG |
| | UACCCCCGUGGUCUUUGAAUAAAGUCUGAGUGGGCGGC |
| | (3′ UTR with miR 142-3p binding site, P3 insertion |
| | variant 2) |
| 267 | UGAUAAUAGGCUGGAGCCUCGGUGGCCUAGCUUCUUGCCCCUUGGGCCUCCC |
| | CCCAGCCCCUCCUCCCCUUCCUGCACCCGUACCCCCACCCCUAUCACAAUUA |
| | GCAUUAAGUGGUCUUUGAAUAAAGUCUGAGUGGGCGGC |
| | (3′ UTR with miR 155-5p binding site variant 2) |
| 268 | UGAUAAUAGACCCCUAUCACAAUUAGCAUUAAGCUGGAGCCUCGGUGGCCUA |
| | GCUUCUUGCCCCUUGGGCCACCCCUAUCACAAUUAGCAUUAAUCCCCCCAGC |
| | CCCUCCUCCCCUUCCUGCACCCGUACCCCCACCCCUAUCACAAUUAGCAUUA |
| | AGUGGUCUUUGAAUAAAGUCUGAGUGGGCGGC |
| | (3′ UTR with 3 miR 155-5p binding sites variant 2) |
| 269 | UGAUAAUAGACCCCUAUCACAAUUAGCAUUAAGCUGGAGCCUCGGUGGCCUA |
| | GCUUCUUGCCCCUUGGGCCUCCAUAAAGUAGGAAACACUACAUCCCCCCAGC |
| | CCCUCCUCCCCUUCCUGCACCCGUACCCCCACCCCUAUCACAAUUAGCAUUA |
| | AGUGGUCUUUGAAUAAAGUCUGAGUGGGCGGC |
| | (3′ UTR with 2 miR 155-5p binding sites and 1 miR |
| | 142-3p binding site variant 2) |
| 271 | AGGAAAUAAGAGAGAAAAGAAGAGUAAGAAGAAAUAUAAGAGCCACC |
| | 5′-UTR (v1 plus A-start) |
| 272 | GGGAAAUAAGAGAGAAAAGAAGAGUAAGAAGAAAUAUAAGACCCCGGCGCCG |
| | CCACC |
| | (5′ UTR v1.1 plus G-start) |
| 273 | AGGAAAUAAGAGAGAAAAGAAGAGUAAGAAGAAAUAUAAGACCCCGGCGCCG |
| | CCACC |
| | (5′ UTR v1.1 plus A-start) |
| 274 | GGGAGAUCAGAGAGAAAAGAAGAGUAAGAAGAAAUAUAAGAGCCACC |
| | 5′ UTR 002 (upstream UTR plus G-start) |
| 80 | GGGAGACAAGCUUGGCAUUCCGGUACUGUUGGUAAAGCCACC |
| | 5′ UTR-004 (Upstream UTR plus G-start) |
| 81 | GGGAAUUAACAGAGAAAAGAAGAGUAAGAAGAAAUAUAAGAGCCACC |
| | 5′ UTR-008 (Upstream UTR plus G-start) |
| 82 | GGGAAAUUAGACAGAAAAGAAGAGUAAGAAGAAAUAUAAGAGCCACC |
| | 5′ UTR-009 (Upstream UTR plus G-start) |
| 83 | GGGAAAUAAGAGAGUAAAGAACAGUAAGAAGAAAUAUAAGAGCCACC |
| | 5′ UTR-010, (Upstream UTR plus G-start) |
| 84 | GGGAAAAAAGAGAGAAAAGAAGACUAAGAAGAAAUAUAAGAGCCACC |
| | 5′ UTR-011 (Upstream UTR plus G-start) |
| 85 | GGGAAAUAAGAGAGAAAAGAAGAGUAAGAAGAUAUAUAAGAGCCACC |
| | 5′ UTR-012 (Upstream UTR plus G-start) |
| 86 | GGGAAAUAAGAGACAAAACAAGAGUAAGAAGAAAUAUAAGAGCCACC |
| | 5′ UTR-013 (Upstream UTR plus G-start) |
| 87 | GGGAAAUUAGAGAGUAAAGAACAGUAAGUAGAAUUAAAAGAGCCACC |
| | 5′ UTR-014 (Upstream UTR plus G-start) |
| 88 | GGGAAAUAAGAGAGAAUAGAAGAGUAAGAAGAAAUAUAAGAGCCACC |
| | 5′ UTR-015 (Upstream UTR plus G-start) |
| 89 | GGGAAAUAAGAGAGAAAAGAAGAGUAAGAAGAAAAUUAAGAGCCACC |
| | 5′ UTR-016 (Upstream UTR plus G-start) |
| 90 | GGGAAAUAAGAGAGAAAAGAAGAGUAAGAAGAAAUUUAAGAGCCACC |
| | 5′ UTR-017 (Upstream UTR plus G-start) |
| 91 | UCAAGCUUUUGGACCCUCGUACAGAAGCUAAUACGACUCACUAUAGGGAAAU |
| | AAGAGAGAAAAGAAGAGUAAGAAGAAAUAUAAGAGCCACC |
| | 5′ UTR-018 (Upstream UTR) |

Stop codon = bold
miR 142-3p binding site = underline
miR 126-3p binding site = bold underline
miR 155-5p binding site = italicized
miR 142-5p binding site = italicized and bold underline

In one embodiment, the nucleic acid molecule (e.g., RNA, e.g., mRNA) of the disclosure comprises a 5′ UTR, a codon optimized open reading frame encoding a polypeptide of interest, a 3′ UTR comprising the at least one miRNA binding site for a miR expressed in immune cells, and a 3′ tailing region of linked nucleosides. In various embodiments, the 3′ UTR comprises 1-4, at least two, one, two, three or four miRNA binding sites for miRs expressed in immune cells, preferably abundantly or preferentially expressed in immune cells.

In one embodiment, the at least one miRNA expressed in immune cells is a miR-142-3p microRNA binding site. In one embodiment, the miR-142-3p microRNA binding site comprises the sequence shown in SEQ ID NO: 202. In one embodiment, the 3′ UTR of the mRNA comprising the miR-142-3p microRNA binding site comprises the sequence shown in SEQ ID NO: 220.

In one embodiment, the at least one miRNA expressed in immune cells is a miR-126 microRNA binding site. In one embodiment, the miR-126 binding site is a miR-126-3p binding site. In one embodiment, the miR-126-3p microRNA binding site comprises the sequence shown in SEQ ID NO: 207. In one embodiment, the 3′ UTR of the mRNA of the invention comprising the miR-126-3p microRNA binding site comprises the sequence shown in SEQ ID NO: 235.

Non-limiting exemplary sequences for miRs to which a microRNA binding site(s) of the disclosure can bind include the following: miR-142-3p (SEQ ID NO: 201), miR-142-5p (SEQ ID NO: 203), miR-146-3p (SEQ ID NO: 221), miR-146-5p (SEQ ID NO: 222), miR-155-3p (SEQ ID NO: 223), miR-155-5p (SEQ ID NO: 224), miR-126-3p (SEQ ID NO: 206), miR-126-5p (SEQ ID NO: 208), miR-16-3p (SEQ ID NO: 225), miR-16-5p (SEQ ID NO: 226), miR-21-3p (SEQ ID NO: 227), miR-21-5p (SEQ ID NO: 228), miR-223-3p (SEQ ID NO: 143), miR-223-5p (SEQ ID NO: 230), miR-24-3p (SEQ ID NO: 231), miR-24-5p (SEQ ID NO: 232), miR-27-3p (SEQ ID NO: 233) and miR-27-5p (SEQ ID NO: 234). Other suitable miR sequences expressed in immune cells (e.g., abundantly or preferentially expressed in immune cells) are known and available in the art, for example at the University of Manchester's microRNA database, miRBase. Sites that bind any of the aforementioned miRs can be designed based on Watson-Crick complementarity to the miR, typically 100% complementarity to the miR, and inserted into an mRNA construct of the disclosure as described herein.

In another embodiment, a nucleic acid molecule (e.g., RNA, e.g., mRNA, e.g., the 3′ UTR thereof) of the disclosure can comprise at least one miRNA binding site to thereby reduce or inhibit accelerated blood clearance, for example by reducing or inhibiting production of IgMs, e.g., against PEG, by B cells and/or reducing or inhibiting proliferation and/or activation of pDCs, and can comprise at least one miRNA binding site for modulating tissue expression of an encoded protein of interest.

miRNA gene regulation can be influenced by the sequence surrounding the miRNA such as, but not limited to, the species of the surrounding sequence, the type of sequence (e.g., heterologous, homologous, exogenous, endogenous, or artificial), regulatory elements in the surrounding sequence and/or structural elements in the surrounding sequence. The miRNA can be influenced by the 5′UTR and/or 3′UTR. As a non-limiting example, a non-human 3′UTR can increase the regulatory effect of the miRNA sequence on the expression of a polypeptide of interest compared to a human 3′UTR of the same sequence type.

In one embodiment, other regulatory elements and/or structural elements of the 5′UTR can influence miRNA mediated gene regulation. One example of a regulatory element and/or structural element is a structured IRES (Internal Ribosome Entry Site) in the 5′UTR, which is necessary for the binding of translational elongation factors to initiate protein translation. EIF4A2 binding to this secondarily structured element in the 5′-UTR is necessary for miRNA mediated gene expression (Meijer H A et al., Science, 2013, 340, 82-85, herein incorporated by reference in its entirety). The nucleic acid molecules (e.g., RNA, e.g., mRNA) of the disclosure can further include this structured 5′UTR in order to enhance microRNA mediated gene regulation.

At least one miRNA binding site can be engineered into the 3′UTR of a polynucleotide of the disclosure. In this context, at least two, at least three, at least four, at least five, at least six, at least seven, at least eight, at least nine, at least ten, or more miRNA binding sites can be engineered into a 3′UTR of a nucleic acid molecule (e.g., RNA, e.g., mRNA) of the disclosure. For example, 1 to 10, 1 to 9, 1 to 8, 1 to 7, 1 to 6, 1 to 5, 1 to 4, 1 to 3, 2, or 1 miRNA binding sites can be engineered into the 3′UTR of a nucleic acid molecule (e.g., RNA, e.g., mRNA) of the disclosure. In one embodiment, miRNA binding sites incorporated into a nucleic acid molecule (e.g., RNA, e.g., mRNA) of the disclosure can be the same or can be different miRNA sites. A combination of different miRNA binding sites incorporated into a nucleic acid molecule (e.g., RNA, e.g., mRNA) of the disclosure can include combinations in which more than one copy of any of the different miRNA sites are incorporated. In another embodiment, miRNA binding sites incorporated into a nucleic acid molecule (e.g., RNA, e.g., mRNA) of the disclosure can target the same or different tissues in the body. As a non-limiting example, through the introduction of tissue-, cell-type-, or disease-specific miRNA binding sites in the 3′-UTR of a nucleic acid molecule (e.g., RNA, e.g., mRNA) of the disclosure, the degree of expression in specific cell types (e.g., myeloid cells, lymphoid cells, immune cells, blood cells, etc.) can be reduced.

In one embodiment, a miRNA binding site can be engineered near the 5′ terminus of the 3′UTR, about halfway between the 5′ terminus and 3′ terminus of the 3′UTR and/or near the 3′ terminus of the 3′UTR in a nucleic acid molecule (e.g., RNA, e.g., mRNA) of the disclosure. As a non-limiting example, a miRNA binding site can be engineered near the 5′ terminus of the 3′UTR and about halfway between the 5′ terminus and 3′ terminus of the 3′UTR. As another non-limiting example, a miRNA binding site can be engineered near the 3′ terminus of the 3′UTR and about halfway between the 5′ terminus and 3′ terminus of the 3′UTR. As yet another non-limiting example, a miRNA binding site can be engineered near the 5′ terminus of the 3′UTR and near the 3′ terminus of the 3′UTR.

In another embodiment, a 3′UTR can comprise 1, 2, 3, 4, 5, 6, 7, 8, 9, or 10 miRNA binding sites. The miRNA binding sites can be complementary to a miRNA, miRNA seed sequence, and/or miRNA sequences flanking the seed sequence.

In some embodiments, the expression of a nucleic acid molecule (e.g., RNA, e.g., mRNA) of the disclosure can be controlled by incorporating at least one sensor sequence in the polynucleotide and formulating the polynucleotide for administration. As a non-limiting example, a nucleic acid molecule (e.g., RNA, e.g., mRNA) of the disclosure can be targeted to a tissue or cell by incorporating a miRNA binding site and formulating the polynucleotide in a lipid nanoparticle comprising an ionizable lipid, including any of the lipids described herein.

A nucleic acid molecule (e.g., RNA, e.g., mRNA) of the disclosure can be engineered for more targeted expression in specific tissues, cell types, or biological conditions based on the expression patterns of miRNAs in the different tissues, cell types, or biological conditions. Through introduction of tissue-specific miRNA binding sites, a nucleic acid molecule (e.g., RNA, e.g., mRNA) of the disclosure can be designed for optimal protein expression in a tissue or cell, or in the context of a biological condition.

In some embodiments, a nucleic acid molecule (e.g., RNA, e.g., mRNA) of the disclosure can be designed to incorporate miRNA binding sites that either have 100% identity to known miRNA seed sequences or have less than 100% identity to miRNA seed sequences. In some embodiments, a nucleic acid molecule (e.g., RNA, e.g., mRNA) of the disclosure can be designed to incorporate miRNA binding sites that have at least: 60%, 65%, 70%, 75%, 80%, 85%, 90%, 95%, 96%, 97%, 98%, or 99% identity to known miRNA seed sequences. The miRNA seed sequence can be partially mutated to decrease miRNA binding affinity and as such result in reduced downmodulation of the nucleic acid molecule. In essence, the degree of match or mismatch between the miRNA binding site and the miRNA seed can act as a rheostat to more finely tune the ability of the miRNA to modulate protein expression. In addition, mutation in the non-seed region of a miRNA binding site can also impact the ability of a miRNA to modulate protein expression.

In one embodiment, a miRNA sequence can be incorporated into the loop of a stem loop.

In another embodiment, a miRNA seed sequence can be incorporated in the loop of a stem loop and a miRNA binding site can be incorporated into the 5′ or 3′ stem of the stem loop.

In one embodiment the miRNA sequence in the 5′ UTR can be used to stabilize a nucleic acid molecule (e.g., RNA, e.g., mRNA) of the disclosure described herein.

In another embodiment, a miRNA sequence in the 5′ UTR of a nucleic acid molecule (e.g., RNA, e.g., mRNA) of the disclosure can be used to decrease the accessibility of the site of translation initiation such as, but not limited to a start codon. See, e.g., Matsuda et al., PLoS One. 2010 11(5):e15057; incorporated herein by reference in its entirety, which used antisense locked nucleic acid (LNA) oligonucleotides and exon-junction complexes (EJCs) around a start codon (−4 to +37 where the A of the AUG codons is +1) to decrease the accessibility to the first start codon (AUG). Matsuda showed that altering the sequence around the start codon with an LNA or EJC affected the efficiency, length and structural stability of a polynucleotide. A nucleic acid molecule (e.g., RNA, e.g., mRNA) of the disclosure can comprise a miRNA sequence, instead of the LNA or EJC sequence described by Matsuda et al, near the site of translation initiation to decrease the accessibility to the site of translation initiation. The site of translation initiation can be prior to, after or within the miRNA sequence. As a non-limiting example, the site of translation initiation can be located within a miRNA sequence such as a seed sequence or binding site.

In some embodiments, a nucleic acid molecule (e.g., RNA, e.g., mRNA) of the disclosure can include at least one miRNA to dampen the antigen presentation by antigen presenting cells. The miRNA can be the complete miRNA sequence, the miRNA seed sequence, the miRNA sequence without the seed, or a combination thereof. As a non-limiting example, a miRNA incorporated into a nucleic acid molecule (e.g., RNA, e.g., mRNA) of the disclosure can be specific to the hematopoietic system. As another non-limiting example, a miRNA incorporated into a nucleic acid molecule (e.g., RNA, e.g., mRNA) of the disclosure on to dampen antigen presentation is miR-142-3p.

In some embodiments, a nucleic acid molecule (e.g., RNA, e.g., mRNA) of the disclosure can include at least one miRNA to dampen expression of the encoded polypeptide in a tissue or cell of interest. As a non-limiting example, a nucleic acid molecule (e.g., RNA, e.g., mRNA) of the disclosure can include at least one miR-142-3p binding site, miR-142-3p seed sequence, miR-142-3p binding site without the seed, miR-142-5p binding site, miR-142-5p seed sequence, miR-142-5p binding site without the seed, miR-146 binding site, miR-146 seed sequence and/or miR-146 binding site without the seed sequence.

In some embodiments, a nucleic acid molecule (e.g., RNA, e.g., mRNA) of the disclosure can comprise at least one miRNA binding site in the 3′UTR in order to selectively degrade mRNA therapeutics in the immune cells to subdue unwanted immunogenic reactions caused by therapeutic delivery. As a non-limiting example, the miRNA binding site can make a nucleic acid molecule (e.g., RNA, e.g., mRNA) of the disclosure more unstable in antigen presenting cells. Non-limiting examples of these miRNAs include mir-142-5p, mir-142-3p, mir-146a-5p, and mir-146-3p.

In one embodiment, a nucleic acid molecule (e.g., RNA, e.g., mRNA) of the disclosure comprises at least one miRNA sequence in a region of the nucleic acid molecule that can interact with an RNA binding protein.

In some embodiments, the nucleic acid molecule (e.g., RNA, e.g., mRNA) of the disclosure comprises (i) a sequence-optimized nucleotide sequence (e.g., an ORF) encoding a polypeptide of interest and (ii) a miRNA binding site (e.g., a miRNA binding site that binds to miR-142) and/or a miRNA binding site that binds to miR-126.

### IVT Polynucleotide Architecture

In some embodiments, the polynucleotide of the present disclosure comprising an mRNA encoding a therapeutic payload or prophylactic payload, an effector molecule and/or a tether molecule is an IVT polynucleotide. Traditionally, the basic components of an mRNA molecule include at least a coding region, a 5′UTR, a 3′UTR, a 5′ cap and a poly-A tail. The IVT polynucleotides of the present disclosure can function as mRNA but are distinguished from wild-type mRNA in their functional and/or structural design features which serve, e.g., to overcome existing problems of effective polypeptide production using nucleic-acid based therapeutics.

The primary construct of an IVT polynucleotide comprises a first region of linked nucleotides that is flanked by a first flanking region and a second flaking region. This first region can include, but is not limited to, the encoded therapeutic payload or prophylactic payload, an effector molecule and/or a tether molecule. The first flanking region can include a sequence of linked nucleosides which function as a 5′ untranslated region (UTR) such as the 5′ UTR of any of the nucleic acids encoding the native 5′ UTR of the polypeptide or a non-native 5′UTR such as, but not limited to, a heterologous 5′ UTR or a synthetic 5′ UTR. The IVT encoding a therapeutic payload or prophylactic payload, an effector molecule and/or a tether molecule can comprise at its 5 terminus a signal sequence region encoding one or more signal sequences. The flanking region can comprise a region of linked nucleotides comprising one or more complete or incomplete 5′ UTRs sequences. The flanking region can also comprise a 5′ terminal cap. The second flanking region can comprise a region of linked nucleotides comprising one or more complete or incomplete 3′ UTRs which can encode the native 3′ UTR of a therapeutic payload or prophylactic payload, an effector molecule and/or a tether molecule or a non-native 3′ UTR such as, but not limited to, a heterologous 3′ UTR or a synthetic 3′ UTR. The flanking region can also comprise a 3′ tailing sequence. The 3′ tailing sequence can be, but is not limited to, a polyA tail, a polyA-G quartet and/or a stem loop sequence.

Additional and exemplary features of IVT polynucleotide architecture are disclosed in International PCT application WO 2017/201325, filed on 18 May 2017, the entire contents of which are hereby incorporated by reference.

### 5′UTR and 3′ UTR

A UTR can be homologous or heterologous to the coding region in a polynucleotide. In some embodiments, the UTR is homologous to the ORF encoding the therapeutic payload or prophylactic payload, an effector molecule and/or a tether molecule. In some embodiments, the UTR is heterologous to the ORF encoding the therapeutic payload or prophylactic payload, an effector molecule and/or a tether molecule.

In some embodiments, the polynucleotide comprises two or more 5′ UTRs or functional fragments thereof, each of which has the same or different nucleotide sequences. In some embodiments, the polynucleotide comprises two or more 3′ UTRs or functional fragments thereof, each of which has the same or different nucleotide sequences.

In some embodiments, the 5′ UTR or functional fragment thereof, 3′ UTR or functional fragment thereof, or any combination thereof is sequence optimized.

In some embodiments, the 5′UTR or functional fragment thereof, 3′ UTR or functional fragment thereof, or any combination thereof comprises at least one chemically modified nucleobase, e.g., N1-methylpseudouracil or 5-methoxyuracil.

UTRs can have features that provide a regulatory role, e.g., increased or decreased stability, localization and/or translation efficiency. A polynucleotide comprising a UTR can be administered to a cell, tissue, or organism, and one or more regulatory features can be measured using routine methods. In some embodiments, a functional fragment of a 5′ UTR or 3′ UTR comprises one or more regulatory features of a full length 5′ or 3′ UTR, respectively.

Natural 5′UTRs bear features that play roles in translation initiation. They harbor signatures like Kozak sequences that are commonly known to be involved in the process by which the ribosome initiates translation of many genes. Kozak sequences have the consensus CCR(A/G)CCAUGG (SEQ ID NO:275), where R is a purine (adenine or guanine) three bases upstream of the start codon (AUG), which is followed by another ‘G’. 5′ UTRs also have been known to form secondary structures that are involved in elongation factor binding.

By engineering the features typically found in abundantly expressed genes of specific target organs, one can enhance the stability and protein production of a polynucleotide. For example, introduction of 5′ UTR of liver-expressed mRNA, such as albumin, serum amyloid A, Apolipoprotein A/B/E, transferrin, alpha fetoprotein, erythropoietin, or Factor VIII, can enhance expression of polynucleotides in hepatic cell lines or liver. Likewise, use of 5′UTR from other tissue-specific mRNA to improve expression in that tissue is possible for muscle (e.g., MyoD, Myosin, Myoglobin, Myogenin, Herculin), for endothelial cells (e.g., Tie-1, CD36), for myeloid cells (e.g., C/EBP, AML1, G-CSF, GM-CSF, CD11b, MSR, Fr-1, i-NOS), for leukocytes (e.g., CD45, CD18), for adipose tissue (e.g., CD36, GLUT4, ACRP30, adiponectin) and for lung epithelial cells (e.g., SP-A/B/C/D).

In some embodiments, UTRs are selected from a family of transcripts whose proteins share a common function, structure, feature or property. For example, an encoded polypeptide can belong to a family of proteins (i.e., that share at least one function, structure, feature, localization, origin, or expression pattern), which are expressed in a particular cell, tissue or at some time during development. The UTRs from any of the genes or mRNA can be swapped for any other UTR of the same or different family of proteins to create a new polynucleotide.

In some embodiments, the 5′ UTR and the 3′ UTR can be heterologous. In some embodiments, the 5′ UTR can be derived from a different species than the 3′ UTR. In some embodiments, the 3′ UTR can be derived from a different species than the 5′ UTR.

Co-owned International Patent Application No. PCT/US2014/021522 (Publ. No. WO/2014/164253, incorporated herein by reference in its entirety) provides a listing of exemplary UTRs that can be utilized in the polynucleotide of the present invention as flanking regions to an ORF.

Additional exemplary UTRs of the application include, but are not limited to, one or more 5′UTR and/or 3′UTR derived from the nucleic acid sequence of: a globin, such as an α- or β-globin (e.g., a Xenopus, mouse, rabbit, or human globin); a strong Kozak translational initiation signal; a CYBA (e.g., human cytochrome b-245 a polypeptide); an albumin (e.g., human albumin7); a HSD17B4 (hydroxysteroid (17-β) dehydrogenase); a virus (e.g., a tobacco etch virus (TEV), a Venezuelan equine encephalitis virus (VEEV), a Dengue virus, a cytomegalovirus (CMV) (e.g., CMV immediate early 1 (IE1)), a hepatitis virus (e.g., hepatitis B virus), a sindbis virus, or a PAV barley yellow dwarf virus); a heat shock protein (e.g., hsp70); a translation initiation factor (e.g., elF4G); a glucose transporter (e.g., hGLUT1 (human glucose transporter 1)); an actin (e.g., human α or β actin); a GAPDH; a tubulin; a histone; a citric acid cycle enzyme; a topoisomerase (e.g., a 5′UTR of a TOP gene lacking the 5′ TOP motif (the oligopyrimidine tract)); a ribosomal protein Large 32 (L32); a ribosomal protein (e.g., human or mouse ribosomal protein, such as, for example, rps9); an ATP synthase (e.g., ATP5A1 or the R subunit of mitochondrial H⁺-ATP synthase); a growth hormone e (e.g., bovine (bGH) or human (hGH)); an elongation factor (e.g., elongation factor 1 al (EEF1A1)); a manganese superoxide dismutase (MnSOD); a myocyte enhancer factor 2A (MEF2A); a 3-F1-ATPase, a creatine kinase, a myoglobin, a granulocyte-colony stimulating factor (G-CSF); a collagen (e.g., collagen type I, alpha 2 (Col1A2), collagen type I, alpha 1 (Col1A1), collagen type VI, alpha 2 (Col6A2), collagen type VI, alpha 1 (Col6A1)); a ribophorin (e.g., ribophorin I (RPNI)); a low density lipoprotein receptor-related protein (e.g., LRP1); a cardiotrophin-like cytokine factor (e.g., Nntl); calreticulin (Calr); a procollagen-lysine, 2-oxoglutarate 5-dioxygenase 1 (Plodl); and a nucleobindin (e.g., Nucb1).

In some embodiments, the 5′ UTR is selected from the group consisting of a β-globin 5′ UTR; a 5′UTR containing a strong Kozak translational initiation signal; a cytochrome b-245 a polypeptide (CYBA) 5′ UTR; a hydroxysteroid (17-β) dehydrogenase (HSD17B4) 5′ UTR; a Tobacco etch virus (TEV) 5′ UTR; a Venezuelan equine encephalitis virus (TEEV) 5′ UTR; a 5′ proximal open reading frame of rubella virus (RV) RNA encoding nonstructural proteins; a Dengue virus (DEN) 5′ UTR; a heat shock protein 70 (Hsp70) 5′ UTR; a eIF4G 5′ UTR; a GLUT1 5′ UTR; functional fragments thereof and any combination thereof.

In some embodiments, the 3′ UTR is selected from the group consisting of a β-globin 3′ UTR; a CYBA 3′ UTR; an albumin 3′ UTR; a growth hormone (GH) 3′ UTR; a VEEV 3′ UTR; a hepatitis B virus (HBV) 3′ UTR; α-globin 3′UTR; a DEN 3′ UTR; a PAV barley yellow dwarf virus (BYDV-PAV) 3′ UTR; an elongation factor 1 al (EEF1A1) 3′ UTR; a manganese superoxide dismutase (MnSOD) 3′ UTR; a R subunit of mitochondrial H(+)-ATP synthase ((3-mRNA) 3′ UTR; a GLUT1 3′ UTR; a MEF2A 3′ UTR; a 3-F1-ATPase 3′ UTR; functional fragments thereof and combinations thereof.

Wild-type UTRs derived from any gene or mRNA can be incorporated into the polynucleotides of the invention. In some embodiments, a UTR can be altered relative to a wild type or native UTR to produce a variant UTR, e.g., by changing the orientation or location of the UTR relative to the ORF; or by inclusion of additional nucleotides, deletion of nucleotides, swapping or transposition of nucleotides. In some embodiments, variants of 5′ or 3′ UTRs can be utilized, for example, mutants of wild type UTRs, or variants wherein one or more nucleotides are added to or removed from a terminus of the UTR.

Additionally, one or more synthetic UTRs can be used in combination with one or more non-synthetic UTRs. See, e.g., Mandal and Rossi, Nat. Protoc. 2013 8(3):568-82, the contents of which are incorporated herein by reference in their entirety.

UTRs or portions thereof can be placed in the same orientation as in the transcript from which they were selected or can be altered in orientation or location. Hence, a 5′ and/or 3′ UTR can be inverted, shortened, lengthened, or combined with one or more other 5′ UTRs or 3′ UTRs.

In some embodiments, the polynucleotide comprises multiple UTRs, e.g., a double, a triple or a quadruple 5′ UTR or 3′ UTR. For example, a double UTR comprises two copies of the same UTR either in series or substantially in series. For example, a double beta-globin 3′UTR can be used (see US2010/0129877, the contents of which are incorporated herein by reference in its entirety).

In certain embodiments, the 5′ UTR and/or 3′ UTR sequence of the invention comprises a nucleotide sequence at least about 60%, at least about 70%, at least about 80%, at least about 90%, at least about 95%, at least about 96%, at least about 97%, at least about 98%, at least about 99%, or about 100% identical to a sequence provided in selected from the group consisting of 5′ UTR sequences comprising any of the 5′ UTR or 3′ UTR sequences disclosed herein (e.g., in Table A or Table B), and any combination thereof.

The polynucleotides of the invention can comprise combinations of features. For example, the ORF can be flanked by a 5′UTR that comprises a strong Kozak translational initiation signal and/or a 3′UTR comprising an oligo(dT) sequence for templated addition of a poly-A tail. A 5′UTR can comprise a first polynucleotide fragment and a second polynucleotide fragment from the same and/or different UTRs (see, e.g., US2010/0293625, herein incorporated by reference in its entirety).

Other non-UTR sequences can be used as regions or subregions within the polynucleotides of the invention. For example, introns or portions of intron sequences can be incorporated into the polynucleotides of the invention. Incorporation of intronic sequences can increase protein production as well as polynucleotide expression levels. In some embodiments, the polynucleotide of the invention comprises an internal ribosome entry site (IRES) instead of or in addition to a UTR (see, e.g., Yakubov et al., Biochem. Biophys. Res. Commun. 2010 394(1):189-193, the contents of which are incorporated herein by reference in their entirety). In some embodiments, the polynucleotide comprises an IRES instead of a 5′ UTR sequence. In some embodiments, the polynucleotide comprises an ORF and a viral capsid sequence. In some embodiments, the polynucleotide comprises a synthetic 5′ UTR in combination with a non-synthetic 3′ UTR.

In some embodiments, the UTR can also include at least one translation enhancer polynucleotide, translation enhancer element, or translational enhancer elements (collectively, “TEE,” which refers to nucleic acid sequences that increase the amount of polypeptide or protein produced from a polynucleotide. As a non-limiting example, the TEE can be located between the transcription promoter and the start codon. In some embodiments, the 5′ UTR comprises a TEE.

In one aspect, a TEE is a conserved element in a UTR that can promote translational activity of a nucleic acid such as, but not limited to, cap-dependent or cap-independent translation.

a. 5′ UTR Sequences

5′ UTR sequences are important for ribosome recruitment to the mRNA and have been reported to play a role in translation (Hinnebusch A, et al., (2016) Science, 352:6292: 1413-6).

Disclosed herein, inter alia, is a polynucleotide, e.g., mRNA, comprising an open reading frame encoding a therapeutic payload or prophylactic payload, an effector molecule and/or a tether molecule (e.g., as described herein), wherein the polynucleotide has a 5′ UTR that confers an increased half-life, increased expression and/or increased activity of the polypeptide encoded by said polynucleotide, or of the polynucleotide itself. In an embodiment, a polynucleotide disclosed herein comprises: (a) a 5′-UTR (e.g., as provided in Table A or a variant or fragment thereof); (b) a coding region comprising a stop element (e.g., as described herein); and (c) a 3′-UTR (e.g., as described herein), and LNP compositions comprising the same. In an embodiment, the polynucleotide comprises a 5′-UTR comprising a sequence provided in Table A or a variant or fragment thereof (e.g., a functional variant or fragment thereof).

In an embodiment, the polynucleotide having a 5′ UTR sequence provided in Table A or a variant or fragment thereof, has an increase in the half-life of the polynucleotide, e.g., about 1.5-20-fold increase in half-life of the polynucleotide. In an embodiment, the increase in half-life is about 1.5, 2, 3, 4, 5, 6, 7, 8, 9, 10, 11, 12, 13, 14, 15, 16, 17, 18, 19, or 20-fold, or more. In an embodiment, the increase in half life is about 1.5-fold or more. In an embodiment, the increase in half life is about 2-fold or more. In an embodiment, the increase in half life is about 3-fold or more. In an embodiment, the increase in half life is about 4-fold or more. In an embodiment, the increase in half life is about 5-fold or more.

In an embodiment, the polynucleotide having a 5′ UTR sequence provided in Table A or a variant or fragment thereof, results in an increased level and/or activity, e.g., output, of the polypeptide encoded by the polynucleotide. In an embodiment, the 5′ UTR results in about 1.5-20-fold increase in level and/or activity, e.g., output, of the polypeptide encoded by the polynucleotide. In an embodiment, the increase in level and/or activity is about 1.5, 2, 3, 4, 5, 6, 7, 8, 9, 10, 11, 12, 13, 14, 15, 16, 17, 18, 19, or 20-fold, or more. In an embodiment, the increase in level and/or activity is about 1.5-fold or more. In an embodiment, the increase in level and/or activity is about 2-fold or more. In an embodiment, the increase in level and/or activity is about 3-fold or more. In an embodiment, the increase in level and/or activity is about 4-fold or more. In an embodiment, the increase in level and/or activity is about 5-fold or more.

In an embodiment, the increase is compared to an otherwise similar polynucleotide which does not have a 5′ UTR, has a different 5′ UTR, or does not have a 5′ UTR described in Table A or a variant or fragment thereof.

In an embodiment, the increase in half-life of the polynucleotide is measured according to an assay that measures the half-life of a polynucleotide, e.g., an assay described herein.

In an embodiment, the increase in level and/or activity, e.g., output, of the polypeptide encoded by the polynucleotide is measured according to an assay that measures the level and/or activity of a polypeptide, e.g., an assay described herein.

In an embodiment, the 5′ UTR comprises a sequence provided in Table A or a sequence with at least 80%, 85%, 90%, 95%, 96%, 97%, 98%, 99% or 100% identity to a 5′ UTR sequence provided in Table A, or a variant or a fragment thereof. In an embodiment, the 5′ UTR comprises a sequence with at least 80%, 85%, 90%, 95%, 96%, 97%, 98%, 99% or 100% identity to SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, or SEQ ID NO: 78.

In an embodiment, the 5′ UTR comprises a sequence with at least 80%, 85%, 90%, 95%, 96%, 97%, 98%, 99% or 100% identity to SEQ ID NO: 150. In an embodiment, the 5′ UTR comprises a sequence with at least 80%, 85%, 90%, 95%, 96%, 97%, 98%, 99% or 100% identity to SEQ ID NO: 51. In an embodiment, the 5′ UTR comprises a sequence with at least 80%, 85%, 90%, 95%, 96%, 97%, 98%, 99% or 100% identity to SEQ ID NO: 52. In an embodiment, the 5′ UTR comprises a sequence with at least 80%, 85%, 90%, 95%, 96%, 97%, 98%, 99% or 100% identity to SEQ ID NO: 53. In an embodiment, the 5′ UTR comprises a sequence with at least 80%, 85%, 90%, 95%, 96%, 97%, 98%, 99% or 100% identity to SEQ ID NO: 54. In an embodiment, the 5′ UTR comprises a sequence with at least 80%, 85%, 90%, 95%, 96%, 97%, 98%, 99% or 100% identity to SEQ ID NO: 55. In an embodiment, the 5′ UTR comprises a sequence with at least 80%, 85%, 90%, 95%, 96%, 97%, 98%, 99% or 100% identity to SEQ ID NO: 56. In an embodiment, the 5′ UTR comprises a sequence with at least 80%, 85%, 90%, 95%, 96%, 97%, 98%, 99% or 100% identity to SEQ ID NO: 57. In an embodiment, the 5′ UTR comprises a sequence with at least 80%, 85%, 90%, 95%, 96%, 97%, 98%, 99% or 100% identity to SEQ ID NO: 58. In an embodiment, the 5′ UTR comprises the sequence of SEQ ID NO: 78. In an embodiment, the 5′ UTR consists of the sequence of SEQ ID NO: 78.

In an embodiment, a 5′ UTR sequence provided in Table A has a first nucleotide which is an A. In an embodiment, a 5′ UTR sequence provided in Table A has a first nucleotide which is a G.

TABLE 3A
5′ UTR sequences

| SEQ | Sequence | |
|---|---|---|
| ID NO: | Name | Sequence |
| 50 | A1 | GGAAAUCGCAAAAUUUGCUCUUCGCGUUAGAUUUCUUUUAGU |
| | | UUUCUCGCAACUAGCAAGCUUUUUGUUCUCGCC |
| 51 | A5 | GGAAAUCCCCACAACCGCCUCAUAUCCAGGCUCAAGAAUAGA |
| | | GCUCAGUGUUUUGUUGUUUAAUCAUUCCGACGUGUUUUGCGA |
| | | UAUUCGCGCAAAGCAGCCAGUCGCGCGCUUGCUUUUAAGUAG |
| | | AGUUGUUUUUCCACCCGUUUGCCAGGCAUCUUUAAUUUAACA |
| | | UAUUUUUAUUUUUCAGGCUAACCUACGCCGCCACC |
| 52 | A6 | GGAAAUAAGAGAGAAAAGAAGAGUAAGAAGAAAUAUAAGAU |
| | | CUCCCUGAGCUUCAGGGAGCCCCGGCGCCGCCACC |
| 53 | A7 | GGAAACCCCCCACCCCCGUAAGAGAGAAAAGAAGAGUAAGAA |
| | | GAAAUAUAAGAUCUCCCUGAGCUUCAGGGAGCCCCGGCGCCG |
| | | CCACC |
| 54 | A8 | GGAGAACUUCCGCUUCCGUUGGCGCAAGCGCUUUCAUUUUUU |
| | | CUGCUACCGUGACUAAG |
| 55 | A9 | GGAAAUAAGAGAGAAAAGAAGAGUAAGAAGAAAUAUAAGAG |
| | | CCACC |
| 56 | A11 | GGAAAUAAGAGAGAAAAGAAGAGUAAGAAGAAAUAUAAGAC |
| | (Reference) | CCCGGCGCCGCCACC |
| 57 | A2 | GGAAAUCGCAAAAUUUGCUCUUCGCGUUAGAUUUCUUUUAGU |
| | | UUUCUCGCAACUAGCAAGCUUUUUGUUCUCGCCGCCGCC |
| 58 | A3 | GGAAAUCGCAAAAUUUUCUUUUCGCGUUAGAUUUCUUUUAGU |
| | | UUUCUUUCAACUAGCAAGCUUUUUGUUCUCGCCGCCGCC |
| 59 | A4 | G G A A A U C G C A A A A (N₂)ₓ(N₃)ₓ CU (N₄)ₓ(N₅)ₓ C G C G U U |
| | | A G A U U U C U U U U A G U U U U C U N₆ N₇ C A A C U A G C A A |
| | | G C U U U U U G U U C U C G C C (N₈ C C)x |
| | | (N₂)ₓ is a uracil and x is an integer from 0 to 5, e.g., wherein x = 3 or 4; |
| | | (N₃)ₓ is a guanine and x is an integer from 0 to 1; |
| | | (N₄)ₓ is a cytosine and x is an integer from 0 to 1; |
| | | (N₅)ₓ is a uracil and x is an integer from 0 to 5, e.g., wherein x = 2 or 3; |
| | | N₆ is a uracil or cytosine; |
| | | N₇ is a uracil or guanine; |
| | | N₈ is adenine or guanine and x is an integer from 0 to 1. |
| 60 | A27 | GGAAAAUUUUAGCCUGGAACGUUAGAUAACUGUCCUGUUGUC |
| | | UUUAUAUACUUGGUCCCCAAGUAGUUUGUCUUCCAAA |
| 61 | A12 | GGAAACUUUAUUUAGUGUUACUUUAUUUUCUGUUUAUUUGU |
| | | GUUUCUUCAGUGGGUUUGUUCUAAUUUCCUUGGCCGCC |
| 62 | A13 | GGAAAAUCUGUAUUAGGUUGGCGUGUUCUUUGGUCGGUUGU |
| | | UAGUAUUGUUGUUGAUUCGUUUGUGGUCGGUUGCCGCC |
| 63 | A14 | GGAAAAUUAUUAACAUCUUGGUAUUCUCGAUAACCAUUCGUU |
| | | GGAUUUUAUUGUAUUCGUAGUUUGGGUUCCUGCCGCC |
| 64 | A15 | GGAAAUUAUUAUUAUUUCUAGCUACAAUUUAUCAUUGUAUU |
| | | AUUUUAGCUAUUCAUCAUUAUUUACUUGGUGAUCAACA |
| 65 | A16 | GGAAAUAGGUUGUUAACCAAGUUCAAGCCUAAUAAGCUUGGA |
| | | UUCUGGUGACUUGCUUCACCGUUGGCGGGCACCGAUC |
| 66 | A17 | GGAAAUCGUAGAGAGUCGUACUUAGUACAUAUCGACUAUCGG |
| | | UGGACACCAUCAAGAUUAUAAACCAGGCCAGA |
| 67 | A18 | GGAAACCCGCCCAAGCGACCCCAACAUAUCAGCAGUUGCCCA |
| | | AUCCCAACUCCCAACACAAUCCCCAAGCAACGCCGCC |
| 68 | A19 | GGAAAGCGAUUGAAGGCGUCUUUUCAACUACUCGAUUAAGGU |
| | | UGGGUAUCGUCGUGGGACUUGGAAAUUUGUUGUUUCC |
| 69 | A20 | GGAAACUAAUCGAAAUAAAAGAGCCCCGUACUCUUUUAUUUC |
| | | UAUUAGGUUAGGAGCCUUAGCAUUUGUAUCUUAGGUA |
| 70 | A21 | GGAAAUGUGAUUUCCAGCAACUUCUUUUGAAUAUAUUGAAUU |
| | | CCUAAUUCAAAGCGAACAAAUCUACAAGCCAUAUACC |
| 71 | A22 | GGAAAUCGUAGAGAGUCGUACUUACGUGGUCGCCAUUGCAUA |
| | | GCGCGCGAAAGCAACAGGAACAAGAACGCGCC |
| 72 | A23 | GGAAAUCGUAGAGAGUCGUACUUAGAAUAAACAGAGUCGGGU |
| | | CGACUUGUCUCUGAUACUACGACGUCACAAUC |
| 73 | A24 | GGAAAAUUUGCCUUCGGAGUUGCGUAUCCUGAACUGCCCAGC |
| | | CUCCUGAUAUACAACUGUUCCGCUUAUUCGGGCCGCC |
| 74 | A25 | GGAAAUCUGAGCAGGAAUCCUUUGUGCAUUGAAGACUUUAGA |
| | | UUCCUCUCUGCGGUAGACGUGCACUUAUAAGUAUUUG |
| 75 | A26 | GGAAAGCGAUUGAAGGCGUCUUUUCAACUACUCGAUUAAGGU |
| | | UGGGUAUCGUCGUGGGACUUGGAAAUUUGUUGCCACC |
| 76 | A28 | GGAAAUUUUUUUUUGAUAUUAUAAGAGUUUUUUUUUGAUAU |
| | | UAAGAAAAUUUUUUUUUGAUAUUAGAAGAGUAAGAAGAAAU |
| | | AUAAGACCCCGGCGCCGCCACC |
| 77 | A29 | GGAAAUAAGAGAGAAAAGAAGAGUAAGAAGAAAUAUAAGAG |
| | | CCAAAAAAAAAAAACC |
| 78 | A30 | GGAAAUCUCCCUGAGCUUCAGGGAGUAAGAGAGAAAAGAAGA |
| | | GUAAGAAGAAAUAUAAGACCCCGGCGCCGCCACC |
| 79 | A31 | GCCRCC, wherein R = A or G |

In an embodiment, the 5′ UTR comprises a variant of SEQ ID NO: 50. In an embodiment, the variant of SEQ ID NO: 50 comprises a nucleic acid sequence of Formula A: G G A A A U C G C A A A A (N2)X (N3)X C U (N4)X (N5)X C G C G U U A G A U U U C UUUUAGUUUUCUN6N7CAACUAGCAAGCUUUUUGUUCUC GC C (N8 C C)x (SEQ ID NO: 59),
wherein:
   (N2)x is a uracil and x is an integer from 0 to 5, e.g., wherein x=3 or 4;
   (N3)x is a guanine and x is an integer from 0 to 1;
   (N4)x is a cytosine and x is an integer from 0 to 1;
   (N5)x is a uracil and x is an integer from 0 to 5, e.g., wherein x=2 or 3;
   N6 is a uracil or cytosine;
   N7 is a uracil or guanine;
   N8 is adenine or guanine and x is an integer from 0 to 1.

In an embodiment (N2)x is a uracil and x is 0. In an embodiment (N2)x is a uracil and x is 1. In an embodiment (N2)x is a uracil and x is 2. In an embodiment (N2)x is a uracil and x is 3.

In an embodiment, (N2)x is a uracil and x is 4. In an embodiment (N2)x is a uracil and x is 5.

In an embodiment, (N3)x is a guanine and x is 0. In an embodiment, (N3)x is a guanine and x is 1.

In an embodiment, (N4)x is a cytosine and x is 0. In an embodiment, (N4)x is a cytosine and x is 1.

In an embodiment (N5)x is a uracil and x is 0. In an embodiment (N5)x is a uracil and x is 1. In an embodiment (N5)x is a uracil and x is 2. In an embodiment (N5)x is a uracil and x is 3.

In an embodiment, (N5)x is a uracil and x is 4. In an embodiment (N5)x is a uracil and x is 5.

In an embodiment, N6 is a uracil. In an embodiment, N6 is a cytosine.

In an embodiment, N7 is a uracil. In an embodiment, N7 is a guanine.

In an embodiment, N8 is an adenine and x is 0. In an embodiment, N8 is an adenine and x is 1.

In an embodiment, N8 is a guanine and x is 0. In an embodiment, N8 is a guanine and x is 1.

In an embodiment, the 5′ UTR comprises a variant of SEQ ID NO: 50. In an embodiment, the variant of SEQ ID NO: 50 comprises a sequence with at least 50%, 60%, 70%, 80%, 85%, 90%, 95%, 96%, 97%, 98%, or 99% identity to SEQ ID NO: 50. In an embodiment, the variant of SEQ ID NO: 50 comprises a sequence with at least 50% identity to SEQ ID NO: 50. In an embodiment, the variant of SEQ ID NO: 50 comprises a sequence with at least 60% identity to SEQ ID NO: 50. In an embodiment, the variant of SEQ ID NO: 50 comprises a sequence with at least 70% identity to SEQ ID NO: 50. In an embodiment, the variant of SEQ ID NO: 50 comprises a sequence with at least 80% identity to SEQ ID NO: 50. In an embodiment, the variant of SEQ ID NO: 50 comprises a sequence with at least 90% identity to SEQ ID NO: 50. In an embodiment, the variant of SEQ ID NO: 50 comprises a sequence with at least 95% identity to SEQ ID NO: 50. In an embodiment, the variant of SEQ ID NO: 50 comprises a sequence with at least 96% identity to SEQ ID NO: 50. In an embodiment, the variant of SEQ ID NO: 50 comprises a sequence with at least 97% identity to SEQ ID NO: 50. In an embodiment, the variant of SEQ ID NO: 50 comprises a sequence with at least 98% identity to SEQ ID NO: 50. In an embodiment, the variant of SEQ ID NO: 50 comprises a sequence with at least 99% identity to SEQ ID NO: 50.

In an embodiment, the variant of SEQ ID NO: 50 comprises a uridine content of at least 5%, 10%, 20%, 30%, 40%, 50%, 60%, 70%, or 80%. In an embodiment, the variant of SEQ ID NO: 50 comprises a uridine content of at least 5%. In an embodiment, the variant of SEQ ID NO: 50 comprises a uridine content of at least 10%. In an embodiment, the variant of SEQ ID NO: 50 comprises a uridine content of at least 20%. In an embodiment, the variant of SEQ ID NO: 50 comprises a uridine content of at least 30%. In an embodiment, the variant of SEQ ID NO: 50 comprises a uridine content of at least 40%. In an embodiment, the variant of SEQ ID NO: 50 comprises a uridine content of at least 50%. In an embodiment, the variant of SEQ ID NO: 50 comprises a uridine content of at least 60%. In an embodiment, the variant of SEQ ID NO: 50 comprises a uridine content of at least 70%. In an embodiment, the variant of SEQ ID NO: 50 comprises a uridine content of at least 80%.

In an embodiment, the variant of SEQ ID NO: 50 comprises at least 2, 3, 4, 5, 6 or 7 consecutive uridines (e.g., a polyuridine tract). In an embodiment, the polyuridine tract in the variant of SEQ ID NO: 50 comprises at least 1-7, 2-7, 3-7, 4-7, 5-7, 6-7, 1-6, 1-5, 1-4, 1-3, 1-2, 2-6, or 3-5 consecutive uridines. In an embodiment, the polyuridine tract in the variant of SEQ ID NO: 50 comprises 4 consecutive uridines. In an embodiment, the polyuridine tract in the variant of SEQ ID NO: 50 comprises 5 consecutive uridines.

In an embodiment, the variant of SEQ ID NO: 50 comprises 1, 2, 3, 4, 5, 6, 7, 8, 9, 10, 11, 12, 13, 14, or 15 polyuridine tracts. In an embodiment, the variant of SEQ ID NO: 50 comprises 3 polyuridine tracts. In an embodiment, the variant of SEQ ID NO: 50 comprises 4 polyuridine tracts. In an embodiment, the variant of SEQ ID NO: 50 comprises 5 polyuridine tracts.

In an embodiment, one or more of the polyuridine tracts are adjacent to a different polyuridine tract. In an embodiment, each of, e.g., all, the polyuridine tracts are adjacent to each other, e.g., all of the polyuridine tracts are contiguous.

In an embodiment, one or more of the polyuridine tracts are separated by 1, 2, 3, 4, 5, 6, 7, 8, 9, 10, 11, 2, 13, 14, 15, 16, 17, 18, 19, 20, 30, 40, 50, or 60 nucleotides. In an embodiment, each of, e.g., all of, the polyuridine tracts are separated by 1, 2, 3, 4, 5, 6, 7, 8, 9, 10, 11, 2, 13, 14, 15, 16, 17, 18, 19, 20, 30, 40, 50, or 60 nucleotides.

In an embodiment, a first polyuridine tract and a second polyuridine tract are adjacent to each other.

In an embodiment, a subsequent, e.g., third, fourth, fifth, sixth, seventh, eighth, ninth, or tenth, polyuridine tract is separated by 1, 2, 3, 4, 5, 6, 7, 8, 9, 10, 11, 2, 13, 14, 15, 16, 17, 18, 19, 20, 30, 40, 50, or 60 nucleotides from the first polyuridine tract, the second polyuridine tract, or any one of the subsequent polyuridine tracts.

In an embodiment, a first polyuridine tract is separated by 1, 2, 3, 4, 5, 6, 7, 8, 9, 10, 11, 2, 13, 14, 15, 16, 17, 18, 19, 20, 30, 40, 50 or 60 nucleotides from a subsequent polyuridine tract, e.g., a second, third, fourth, fifth, sixth or seventh, eighth, ninth, or tenth polyuridine tract. In an embodiment, one or more of the subsequent polyuridine tracts are adjacent to a different polyuridine tract.

In an embodiment, the 5′ UTR comprises a Kozak sequence, e.g., a GCCRCC nucleotide sequence (SEQ ID NO: 79) wherein R is an adenine or guanine. In an embodiment, the Kozak sequence is disposed at the 3′ end of the 5‘ ’UTR sequence.

In an aspect, the polynucleotide (e.g., mRNA) comprising an open reading frame encoding a therapeutic payload or prophylactic payload, an effector molecule and/or a tether molecule (e.g., as disclosed herein) and comprising a 5′ UTR sequence disclosed herein is formulated as an LNP. In an embodiment, the LNP composition comprises: (i) an ionizable lipid, e.g., an amino lipid; (ii) a sterol or other structural lipid; (iii) a non-cationic helper lipid or phospholipid; and (iv) a PEG-lipid.

b. 3′ UTR Sequences

3′UTR sequences have been shown to influence translation, half-life, and subcellular localization of mRNAs (Mayr C., Cold Spring Harb Persp Biol 2019 Oct. 1;11(10):a034728).

Disclosed herein, inter alia, is a nucleic acid molecule (e.g., RNA, e.g., mRNA), comprising an open reading frame encoding a therapeutic payload or prophylactic payload, an effector molecule and/or a tether molecule (e.g., as described herein), which nucleic acid molecule has a 3′ UTR that confers an increased half-life, increased expression and/or increased activity of the polypeptide encoded by said nucleic acid molecule, or of the nucleic acid molecule itself. In an embodiment, a nucleic acid molecule (e.g., RNA, e.g., mRNA) disclosed herein comprises: (a) a 5′-UTR (e.g., as described herein); (b) a coding region comprising a stop element (e.g., as described herein); and (c) a 3′-UTR (e.g., as provided in Table B or a variant or fragment thereof), and LNP compositions comprising the same. In an embodiment, the nucleic acid molecule comprises a 3′-UTR comprising a sequence provided in Table B or a variant or fragment thereof.

In an embodiment, the nucleic acid molecule (e.g., RNA, e.g., mRNA) having a 3′ UTR sequence provided in Table B or a variant or fragment thereof, results in an increased half-life of the nucleic acid molecule, e.g., about 1.5-10-fold increase in half-life of the nucleic acid molecule. In an embodiment, the increase in half-life is about 1.5, 2, 3, 4, 5, 6, 7, 8, 9, or 10-fold, or more. In an embodiment, the increase in half-life is about 1.5-fold or more. In an embodiment, the increase in half-life is about 2-fold or more. In an embodiment, the increase in half-life is about 3-fold or more. In an embodiment, the increase in half-life is about 4-fold or more. In an embodiment, the increase in half-life is about 5-fold or more. In an embodiment, the increase in half-life is about 6-fold or more. In an embodiment, the increase in half-life is about 7-fold or more. In an embodiment, the increase in half-life is about 8-fold. In an embodiment, the increase in half-life is about 9-fold or more. In an embodiment, the increase in half-life is about 10-fold or more.

In an embodiment, the nucleic acid molecule having a 3′ UTR sequence provided in Table B or a variant or fragment thereof, results in a polynucleotide with a mean half-life score of greater than 10.

In an embodiment, the nucleic acid molecule having a 3′ UTR sequence provided in Table B or a variant or fragment thereof, results in an increased level and/or activity, e.g., output, of the polypeptide encoded by the nucleic acid molecule.

In an embodiment, the increase is compared to an otherwise similar nucleic acid molecule which does not have a 3′ UTR, has a different 3′ UTR, or does not have a 3′ UTR of Table B or a variant or fragment thereof.

In an embodiment, the nucleic acid molecule comprises a 3′ UTR sequence provided in Table B or a sequence with at least 80%, 85%, 90%, 95%, 96%, 97%, 98%, 99% or 100% identity to a 3′ UTR sequence provided in Table B, or a fragment thereof. In an embodiment, the 3′ UTR comprises a sequence with at least 80%, 85%, 90%, 95%, 96%, 97%, 98%, 99% or 100% identity to SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 276, SEQ ID NO:115, or SEQ ID NO: 136.

In an embodiment, the 3′ UTR comprises the sequence of SEQ ID NO: 100, or a sequence with at least 80%, 85%, 90%, 95%, 96%, 97%, 98%, 99% or 100% identity to SEQ ID NO: 100. In an embodiment, the 3′ UTR comprises the sequence of SEQ ID NO: 101, or a sequence with at least 80%, 85%, 90%, 95%, 96%, 97%, 98%, 99% or 100% identity to SEQ ID NO: 101. In an embodiment, the 3′ UTR comprises the sequence of SEQ ID NO: 102, or a sequence with at least 80%, 85%, 90%, 95%, 96%, 97%, 98%, 99% or 100% identity to SEQ ID NO: 102. In an embodiment, the 3′ UTR comprises the sequence of SEQ ID NO: 103, or a sequence with at least 80%, 85%, 90%, 95%, 96%, 97%, 98%, 99% or 100% identity to SEQ ID NO: 103. In an embodiment, the 3′ UTR comprises the sequence of SEQ ID NO: 104, or a sequence with at least 80%, 85%, 90%, 95%, 96%, 97%, 98%, 99% or 100% identity to SEQ ID NO: 104. In an embodiment, the 3′ UTR comprises the sequence of SEQ ID NO: 105, or a sequence with at least 80%, 85%, 90%, 95%, 96%, 97%, 98%, 99% or 100% identity to SEQ ID NO: 105. In an embodiment, the 3′ UTR comprises the sequence of SEQ ID NO: 106, or a sequence with at least 80%, 85%, 90%, 95%, 96%, 97%, 98%, 99% or 100% identity to SEQ ID NO: 106. In an embodiment, the 3′ UTR comprises the sequence of SEQ ID NO: 107, or a sequence with at least 80%, 85%, 90%, 95%, 96%, 97%, 98%, 99% or 100% identity to SEQ ID NO: 107. In an embodiment, the 3′ UTR comprises the sequence of SEQ ID NO: 108, or a sequence with at least 80%, 85%, 90%, 95%, 96%, 97%, 98%, 99% or 100% identity to SEQ ID NO: 108. In an embodiment, the 3′ UTR comprises the sequence of SEQ ID NO: 109, or a sequence with at least 80%, 85%, 90%, 95%, 96%, 97%, 98%, 99% or 100% identity to SEQ ID NO: 109. In an embodiment, the 3′ UTR comprises the sequence of SEQ ID NO: 110, or a sequence with at least 80%, 85%, 90%, 95%, 96%, 97%, 98%, 99% or 100% identity to SEQ ID NO: 110. In an embodiment, the 3′ UTR comprises the sequence of SEQ ID NO: 111, or a sequence with at least 80%, 85%, 90%, 95%, 96%, 97%, 98%, 99% or 100% identity to SEQ ID NO: 111. In an embodiment, the 3′ UTR comprises the sequence of SEQ ID NO: 112, or a sequence with at least 80%, 85%, 90%, 95%, 96%, 97%, 98%, 99% or 100% identity to SEQ ID NO: 112. In an embodiment, the 3′ UTR comprises the sequence of SEQ ID NO: 113, or a sequence with at least 80%, 85%, 90%, 95%, 96%, 97%, 98%, 99% or 100% identity to SEQ ID NO: 113. In an embodiment, the 3′ UTR comprises the sequence of SEQ ID NO: 276, or a sequence with at least 80%, 85%, 90%, 95%, 96%, 97%, 98%, 99% or 100% identity to SEQ ID NO: 276. In an embodiment, the 3′ UTR comprises the sequence of SEQ ID NO: 115, or a sequence with at least 80%, 85%, 90%, 95%, 96%, 97%, 98%, 99% or 100% identity to SEQ ID NO: 115. In an embodiment, the 3′ UTR comprises the sequence of SEQ ID NO: 136, or a sequence with at least 80%, 85%, 90%, 95%, 96%, 97%, 98%, 99% or 100% identity to SEQ ID NO: 136.

TABLE 3B
3′ UTR sequences

| SEQ | | |
|---|---|---|
| ID | Sequence | |
| NO | Name | Sequence |
| 100 | B1 | UAAUAGUAAGCUGGAGCCUCCUGAGAGACCUGUGUG |
| | | AACUAUUGAGAAGAUCGGAACAGCUCCUUACUCUGA |
| | | GGAAGUUGGUACCCCCGUGGUCUUUGAAUAAAGUCU |
| | | GAGUGGGCGGC |
| 101 | B3 | UAAUAGUAAGCUGGAGCCUCACUCUCCUCUCCAUCCC |
| | | GUAUCCAGGCUGUGAAUUUUUCAAGGAAUAUAAAGA |
| | | UCGGGAUGUACCCCCGUGGUCUUUGAAUAAAGUCUG |
| | | AGUGGGCGGC |
| 102 | B4 | UGAUAGUAAGCUGGAGCCUCUAGUGACGGCAACAGG |
| | | GCUUGGUUUUUCCUUGUUGUGAAAUCGACAUCUCUG |
| | | AAGACAGGGUACCCCCGUGGUCUUUGAAUAAAGUCU |
| | | GAGUGGGCGGC |
| 103 | B5 | UGAUAGUAAGCUGGAGCCUCCUUCCAUCUAGUCACA |
| | | AAGACUCCUUCGUCCCCAGUUGCCGUCUAGGAUUGG |
| | | GCCUCCCAGUACCCCCGUGGUCUUUGAAUAAAGUCUG |
| | | AGUGGGCGGC |
| 104 | B6 | UGAUAGUAAGCUGGAGCCUCCCAUAACAUGACAUAU |
| | | CUGGAUUUUGUGCUUAGAACCUUAAAUUGGAAGCAU |
| | | UCUUAAUUGUACCCCCGUGGUCUUUGAAUAAAGUCU |
| | | GAGUGGGCGGC |
| 105 | B7 | UAAUAGUAAGCUGGAGCCUCCGGAAAACUAAAAUAG |
| | | AGAUAUUUCAAGAUUUUAUAAUUUUCAAAGACCUUU |
| | | GAAAUAUUGUACCCCCGUGGUCUUUGAAUAAAGUCU |
| | | GAGUGGGCGGC |
| 106 | B8 | UAAUAGUAAGCUGGAGCCUCUACACAUUGCUUCUAG |
| | | UUGGCAGAAAUAAUUGAUUAAAAGACCAGAAACUGU |
| | | GAUAACUGGUACCCCCGUGGUCUUUAAAUAAAGUCU |
| | | AAGUGGGCGGC |
| 107 | B9 | UGAUAAUAGGCUGGAGCCUCGGUGGCCAUGCUUCUU |
| | | GCCCCUUGGGCCUCCCCCCAGCCCCUCCUCCCCUUCC |
| | | UGCACCCGUACCCCCGUGGUCUUUGAAUAAAGUCUG |
| | | AGUGGGGGC |
| 108 | B10 | UGAUAAUAGGCUGGAGCCUCGGUGGCCUAGCUUCUU |
| | | GCCCCUUGGGCCUCCCCCCAGCCCCUCCUCCCCUUCC |
| | | UGCACCCGUACCCCCGUGGUCUUUGAAUAAAGUCUG |
| | | AGUGGGCGGC |
| 109 | B11 | UGAUAAUAGGCUGGAGCCUCGGUGGCCAUGCUUCUU |
| | | GCCCCUUGGGCCUCCCCCCAGCCCCUCCUCCCCUUCC |
| | | UGCACCCGUACCCCCCAAACACCAUUGUCACACUCCA |
| | | GUGGUCUUUGAAUAAAGUCUGAGUGGGCGGC |
| 110 | B12 | UGAUAAUAGGCUGGAGCCUCGGUGGCCUAGCUUCUU |
| | | GCCCCUUGGGCCUCCCCCCAGCCCCUCCUCCCCUUCC |
| | | UGCACCCGUACCCCCCAAACACCAUUGUCACACUCCA |
| | | GUGGUCUUUGAAUAAAGUCUGAGUGGGCGGC |
| 111 | B13 | UGAUAAUAGGCUGGAGCCUCGGUGGCCUAGCUUCUU |
| | | GCCCCUUGGGCCUCCCCCCAGCCCCUCCUCCCCUUCC |
| | | UGCACCCGUACCCCCUCCAUAAAGUAGGAAACACUAC |
| | | AGUGGUCUUUGAAUAAAGUCUGAGUGGGCGGC |
| 112 | B14 | UGAUAAUAGGCUGGAGCCUCGGUGGCCUAGCUUCUU |
| | | GCCCCUUGGGCCUCCCCCCAGCCCCUCCUCCCCUUCC |
| | | UGCACCCGUACCCCCCGCAUUAUUACUCACGGUACGA |
| | | GUGGUCUUUGAAUAAAGUCUGAGUGGGCGGC |
| 113 | B15 | UGAUAAUAGUCCAUAAAGUAGGAAACACUACAGCUG |
| | | GAGCCUCGGUGGCCAUGCUUCUUGCCCCUUGGGCCUC |
| | | CCCCCAGCCCCUCCUCCCCUUCCUGCACCCGUACCCCC |
| | | CGCAUUAUUACUCACGGUACGAGUGGUCUUUGAAUA |
| | | AAGUCUGAGUGGGCGGC |
| 276 | B16 | UGAUAAUAGUCCAUAAAGUAGGAAACACUACAGCUG |
| | | GAGCCUCGGUGGCCUAGCUUCUUGCCCCUUGGGCCUC |
| | | CAUAAAGUAGGAAACACUACAUCCCCCCAGCCCCUCC |
| | | UCCCCUUCCUGCACCCGUACCCCCUCCAUAAAGUAGG |
| | | AAACACUACAGUGGUCUUUGAAUAAAGUCUGAGUGG |
| | | GCGGC |
| 115 | B2 | CUGAGAGACCUGUGUGAACUAUUGAGAAGAUCGGAA |
| | | CAGCUCCUUACUCUGAGGAAGUUG |
| 116 | B17 | UGAUAAUAGGCUGGAGCCUCUCACACACCUCUGCCCC |
| | | UUGGGCCUCCCACUCCCAUGGCUCUGGGCGGUCCAGA |
| | | AGGAGCGUACCCCCGUGGUCUUUGAAUAAAGUCUGA |
| | | GUGGGCGGC |
| 117 | B18 | UGAUAAUAGGCUGGAGCCUCCACCGCGUUAUCCGUU |
| | | CCUCGUAGGCUGGUCCUGGGGAACGGGUCGGGGGU |
| | | ACCCCCGUGGUCUUUGAAUAAAGUCUGAGUGGGGGG |
| | | C |
| 118 | B19 | UGAUAAUAGGCUGGAGCCUCUGCCCGGCAACGGCCA |
| | | GGUCUGUGCCAAGUGUUUGCUGACGCAACCCCCACUG |
| | | GCUGGGGCUUGGUCAUGGGCCAUCAGCGCGUGCGUG |
| | | GAACCUUUUCGGCUCCUCUGCCGAUCCAUACUGCGGA |
| | | ACUCCUAGCCGCUUGUUUUGCUCGCAGCAGGUCUGG |
| | | AGCAAACAUUAUCGGGACUGAUAACUCUGUUGUCCU |
| | | GUACCCCCGUGGUCUUUGAAUAAAGUCUGAGUGGGC |
| | | GGC |
| 119 | B20 | UGAUAAUAGGCUGGAGCCUCGGUGGCCUAGCUUCUU |
| | | GCCCCUUGGGCCUCCCCCCAGCCCCUCCUCCCCUUCC |
| | | UGCACCCGUACCCUUUUUUUUUUUUUUUUUUUCUUC |
| | | UUUUCUUUUUUUUCUUUUUUUUUUUUCUUUCUUUUU |
| | | UUCUUUUUUUUUCUUUUUUUUUUCUUUUUUUUUUU |
| | | UUUUUCCGUGGUCUUUGAAUAAAGUCUGAGUGGGCG |
| | | GC |
| 120 | B21 | UGAUAAUAGGCUGGAGCCUCGGUGGCCUAGCUUCUU |
| | | GCCCCUUGGGCCUCCCCCCAGCCCCUCCUCCCCUUCC |
| | | UGCACCCGUACCCUUUUUUUUUUUUUUUUUUUUUUU |
| | | UUUUUUUUUUUUUUUUUUUUUUUUUUUUUUUUUUUU |
| | | UUUUUUUUUUUUUUUUUUUUUUUUUUUUUUUUUUUU |
| | | UUUUUCCGUGGUCUUUGAAUAAAGUCUGAGUGGGCG |
| | | GC |
| 133 | B22 | UAAGUCUCCAUAAAGUAGGAAACACUACAGCUGGAG |
| | | CCUCGGUGGCCUAGCUUCUUGCCCCUUGGGCCUCCAU |
| | | AAAGUAGGAAACACUACAUCCCCCCAGCCCCUCCUCC |
| | | CCUUCCUGCACCCGUACCCCCUCCAUAAAGUAGGAAA |
| | | CACUACAGUGGUCUUUGAAUAAAGUCUGAGUGGGCG |
| | | GC |
| 134 | B23 | UAAAGCGCUGGAGCCUCGGUGGCCUAGCUUCUUGCCC |
| | | CUUGGGCCUCCCCCCAGCCCCUCCUCCCCUUCCUGCA |
| | | CCCGUACCCCCUCCAUAAAGUAGGAAACACUACAGUG |
| | | GUCUUUGAAUAAAGUCUGAGUGGGCGGC |
| 135 | B24 | UAAAGCUCCCCGGGGUCCAUAAAGUAGGAAACACUA |
| | | CAGCUGGAGCCUCCUGAGAGACCUGUGUGAACUAUU |
| | | GAGAAGAUCGGAACAGCUCCUUACUCUGAGGAAGUU |
| | | GUCCAUAAAGUAGGAAACACUACAGUACCCCCUCCAU |
| | | AAAGUAGGAAACACUACAGUGGUCUUUGAAUAAAGU |
| | | CUGAGUGGGCGGC |
| 136 | B25 | UAAUAGUAAACCUCACUCACGGCCACAUUGAGUGCC |
| | | AGGCUCCGGGCUGGUUUAUAGUAGUGUAGAGCAUUG |
| | | CAGCACUUAGACUGGGGUGCUGUAGUCUUUAUUGUA |
| | | GUCUUUCCACAUACCUGAUAAUUCUUAGAUAAUUUC |
| | | UUAUUUUAAUUCCAUAAAGUAGGAAACACUACAUAA |
| | | AUCUCCAUAAAGUAGGAAACACUACAUAUUCUUCCA |
| | | UAAAGUAGGAAACACUACAUAGGCU |
| 137 | B26 | GCCUCCACCGCGUUAUCCGUUCCUCGUAGGCUGGUCC |
| | | UGGGGAACGGGUCGGCGGGUACCCCCGUGGUCUUUG |
| | | AAUAAAGUCUGAGUGGGCGGC |
| 138 | B27 | CACCGCGUUAUCCGUUCCUCGUAGGCUGGUCCUGGGG |
| | | AACGGGUCGGCGGGCCUCGGUGGCCUAGCUUCUUGCC |
| | | CCUUGGGCCCACCGCGUUAUCCGUUCCUCGUAGGCUG |
| | | GUCCUGGGGAACGGGUCGGCGGUCCCCCCAGCCCCUC |
| | | CUCCCCUUCCUGCACCCGUACCCCCCACCGCGUUAUC |
| | | CGUUCCUCGUAGGCUGGUCCUGGGGAACGGGUCGGC |
| | | GGGUGGUCUUUGAAUAAAGUCUGAGUGGGCGGC |

In an embodiment, the 3′ UTR comprises a micro RNA (miRNA) binding site, e.g., as described herein, which binds to a miR present in a human cell. In an embodiment, the 3′ UTR comprises a miRNA binding site of SEQ ID NO: 212, SEQ ID NO: 174, SEQ ID NO: 152 or a combination thereof. In an embodiment, the 3′ UTR comprises a plurality of miRNA binding sites, e.g., 2, 3, 4, 5, 6, 7 or 8 miRNA binding sites. In an embodiment, the plurality of miRNA binding sites comprises the same or different miRNA binding sites.

(SEQ ID NO: 212)
miR 122 bs = CAAACACCAUUGUCACACUCCA
(SEQ ID NO: 174)
miR-142-3p bs = UCCAUAAAGUAGGAAACACUACA
(SEQ ID NO: 152)
miR-126 bs = CGCAUUAUUACUCACGGUACGA

In an aspect, disclosed herein is a nucleic acid molecule (e.g., RNA, e.g., mRNA) encoding a polypeptide, wherein the nucleic acid molecule comprises: (a) a 5′-UTR, e.g., as described herein; (b) a coding region comprising a stop element (e.g., as described herein); and (c) a 3′-UTR (e.g., as described herein).

In an aspect, an LNP composition comprising a nucleic acid molecule (e.g., RNA, e.g., mRNA) comprising an open reading frame encoding a therapeutic payload or prophylactic payload, an effector molecule and/or a tether molecule polypeptide and comprising a 3′ UTR disclosed herein comprises: (i) an ionizable lipid, e.g., an amino lipid; (ii) a sterol or other structural lipid; (iii) a non-cationic helper lipid or phospholipid; and (iv) a PEG-lipid.

### Regions Having a 5′ Cap

The disclosure also includes a polynucleotide (e.g., mRNA) that comprises both a 5′ Cap and a polynucleotide of the present invention (e.g., a polynucleotide comprising a nucleotide sequence encoding a therapeutic payload or prophylactic payload, an effector molecule and/or a tether molecule).

The 5′ cap structure of a natural mRNA is involved in nuclear export, increasing mRNA stability and binds the mRNA Cap Binding Protein (CBP), which is responsible for mRNA stability in the cell and translation competency through the association of CBP with poly(A) binding protein to form the mature cyclic mRNA species. The cap further assists the removal of 5′ proximal introns during mRNA splicing.

Endogenous mRNA molecules can be 5′-end capped generating a 5′-ppp-5′-triphosphate linkage between a terminal guanosine cap residue and the 5′-terminal transcribed sense nucleotide of the mRNA molecule. This 5′-guanylate cap can then be methylated to generate an N7-methyl-guanylate residue. The ribose sugars of the terminal and/or ante-terminal transcribed nucleotides of the 5′ end of the mRNA can optionally also be 2′-O-methylated. 5′-decapping through hydrolysis and cleavage of the guanylate cap structure can target a nucleic acid molecule, such as an mRNA molecule, for degradation.

In some embodiments, the polynucleotides of the present invention (e.g., a polynucleotide comprising a nucleotide sequence encoding a therapeutic payload or prophylactic payload, an effector molecule and/or a tether molecule) incorporate a cap moiety.

In some embodiments, polynucleotides of the present invention (e.g., a polynucleotide comprising a nucleotide sequence encoding a therapeutic payload or prophylactic payload, an effector molecule and/or a tether molecule) comprise a non-hydrolyzable cap structure preventing decapping and thus increasing mRNA half-life. Because cap structure hydrolysis requires cleavage of 5′-ppp-5′ phosphorodiester linkages, modified nucleotides can be used during the capping reaction. For example, a Vaccinia Capping Enzyme from New England Biolabs (Ipswich, MA) can be used with α-thio-guanosine nucleotides according to the manufacturer's instructions to create a phosphorothioate linkage in the 5′-ppp-5′ cap. Additional modified guanosine nucleotides can be used such as α-methyl-phosphonate and seleno-phosphate nucleotides.

Additional modifications include, but are not limited to, 2′-O-methylation of the ribose sugars of 5′-terminal and/or 5′-anteterminal nucleotides of the polynucleotide (as mentioned above) on the 2′-hydroxyl group of the sugar ring. Multiple distinct 5′-cap structures can be used to generate the 5′-cap of a nucleic acid molecule, such as a polynucleotide that functions as an mRNA molecule. Cap analogs, which herein are also referred to as synthetic cap analogs, chemical caps, chemical cap analogs, or structural or functional cap analogs, differ from natural (i.e., endogenous, wild-type or physiological) 5′-caps in their chemical structure, while retaining cap function. Cap analogs can be chemically (i.e., non-enzymatically) or enzymatically synthesized and/or linked to the polynucleotides of the invention.

For example, the Anti-Reverse Cap Analog (ARCA) cap contains two guanines linked by a 5′-5′-triphosphate group, wherein one guanine contains an N7 methyl group as well as a 3′-O-methyl group (i.e., N7,3′-O-dimethyl-guanosine-5′-triphosphate-5′-guanosine (m7G-3′mppp-G; which can equivalently be designated 3′ O-Me-m7G(5′)ppp(5′)G). The 3′-0 atom of the other, unmodified, guanine becomes linked to the 5′-terminal nucleotide of the capped polynucleotide.

The N7- and 3′-O-methlyated guanine provides the terminal moiety of the capped polynucleotide.

Another exemplary cap is mCAP, which is similar to ARCA but has a 2′-O-methyl group on guanosine (e.g., N7,2′-O-dimethyl-guanosine-5′-triphosphate-5′-guanosine, m7Gm-ppp-G).

Another exemplary cap is m?G-ppp-Gm-A (i.e., N7, guanosine-5′-triphosphate-2′-O-dimethyl-guanosine-adenosine).

In some embodiments, the cap is a dinucleotide cap analog. As a non-limiting example, the dinucleotide cap analog can be modified at different phosphate positions with a boranophosphate group or a phosphoroselenoate group such as the dinucleotide cap analogs described in U.S. Pat. No. 8,519,110, the contents of which are herein incorporated by reference in its entirety.

In another embodiment, the cap is a cap analog is a N7-(4-chlorophenoxyethyl) substituted dinucleotide form of a cap analog known in the art and/or described herein. Non-limiting examples of a N7-(4-chlorophenoxyethyl) substituted dinucleotide form of a cap analog include a N7-(4-chlorophenoxyethyl)-G(5′)ppp(5′)G and a N7-(4-chlorophenoxyethyl)-m3′-OG(5′)ppp(5′)G cap analog (See, e.g., the various cap analogs and the methods of synthesizing cap analogs described in Kore et al. Bioorganic & Medicinal Chemistry 2013 21:4570-4574; the contents of which are herein incorporated by reference in its entirety). In another embodiment, a cap analog of the present invention is a 4-chloro/bromophenoxyethyl analog.

While cap analogs allow for the concomitant capping of a polynucleotide or a region thereof, in an in vitro transcription reaction, up to 20% of transcripts can remain uncapped. This, as well as the structural differences of a cap analog from an endogenous 5′-cap structures of nucleic acids produced by the endogenous, cellular transcription machinery, can lead to reduced translational competency and reduced cellular stability.

Polynucleotides of the invention (e.g., a polynucleotide comprising a nucleotide sequence encoding a therapeutic payload or prophylactic payload, an effector molecule and/or a tether molecule) can also be capped post-manufacture (whether IVT or chemical synthesis), using enzymes, to generate more authentic 5′-cap structures. As used herein, the phrase “more authentic” refers to a feature that closely mirrors or mimics, either structurally or functionally, an endogenous or wild type feature. That is, a “more authentic” feature is better representative of an endogenous, wild-type, natural or physiological cellular function and/or structure as compared to synthetic features or analogs, etc., of the prior art, or which outperforms the corresponding endogenous, wild-type, natural or physiological feature in one or more respects. Non-limiting examples of more authentic 5′cap structures of the present invention are those that, among other things, have enhanced binding of cap binding proteins, increased half-life, reduced susceptibility to 5′ endonucleases and/or reduced 5′decapping, as compared to synthetic 5′cap structures known in the art (or to a wild-type, natural or physiological 5′cap structure). For example, recombinant Vaccinia Virus Capping Enzyme and recombinant 2′-O-methyltransferase enzyme can create a canonical 5′-5′-triphosphate linkage between the 5′-terminal nucleotide of a polynucleotide and a guanine cap nucleotide wherein the cap guanine contains an N7 methylation and the 5′-terminal nucleotide of the mRNA contains a 2′-O-methyl. Such a structure is termed the CapI structure. This cap results in a higher translational-competency and cellular stability and a reduced activation of cellular pro-inflammatory cytokines, as compared, e.g., to other 5′cap analog structures known in the art. Cap structures include, but are not limited to, 7mG(5′)ppp(5′)N,pN2p (cap 0), 7mG(5′)ppp(5′)N1mpNp (cap 1), and 7mG(5′)-ppp(5′)N1mpN2mp (cap 2). Cap 1 is sometimes referred to as Cap C1 herein. In some embodiments, Cap C1 can optionally include an additional G at the 3′ end of the cap. In some embodiments, in Cap C1, N2 may comprise the first nucleotide of a 5′ UTR.

As a non-limiting example, capping chimeric polynucleotides post-manufacture can be more efficient as nearly 100% of the chimeric polynucleotides can be capped. This is in contrast to ˜80% efficiency when a cap analog is linked to a chimeric polynucleotide during an in vitro transcription reaction.

According to the present invention, 5′ terminal caps can include endogenous caps or cap analogs. According to the present invention, a 5′ terminal cap can comprise a guanine analog. Useful guanine analogs include, but are not limited to, inosine, N1-methyl-guanosine, 2′fluoro-guanosine, 7-deaza-guanosine, 8-oxo-guanosine, 2-amino-guanosine, LNA-guanosine, and 2-azido-guanosine.

Also provided herein are exemplary caps including those that can be used in co-transcriptional capping methods for ribonucleic acid (RNA) synthesis, using RNA polymerase, e.g., wild type RNA polymerase or variants thereof, e.g., such as those variants described herein.

In one embodiment, caps can be added when RNA is produced in a “one-pot” reaction, without the need for a separate capping reaction. Thus, the methods, in some embodiments, comprise reacting a polynucleotide template with an RNA polymerase variant, nucleoside triphosphates, and a cap analog under in vitro transcription reaction conditions to produce RNA transcript.

A cap analog may be, for example, a dinucleotide cap, a trinucleotide cap, or a tetranucleotide cap. In some embodiments, a cap analog is a dinucleotide cap. In some embodiments, a cap analog is a trinucleotide cap. In some embodiments, a cap analog is a tetranucleotide cap. As used here the term “cap” includes the inverted G nucleotide and can comprise one or more additional nucleotides 3′ of the inverted G nucleotide, e.g., 1, 2, or more nucleotides 3′ of the inverted G nucleotide and 5′ to the 5′ UTR, e.g., a 5′ UTR described herein.

Exemplary caps comprise a sequence of GG, GA, or GGA, wherein the underlined, italicized G is an in inverted G nucleotide followed by a 5′-5′-triphosphate group. A trinucleotide cap, in some embodiments, comprises a compound of formula (I)

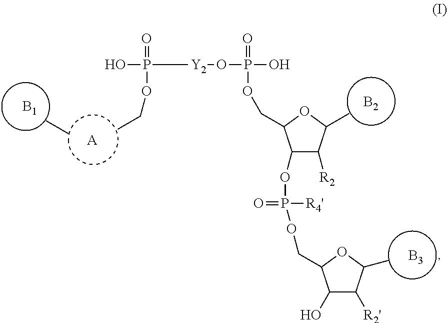

or a stereoisomer, tautomer or salt thereof, wherein

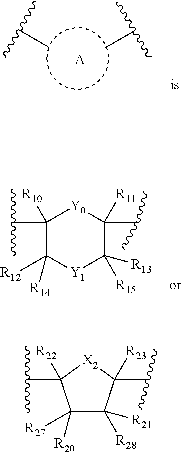

ring B1 is a modified or unmodified Guanine;

ring B2 and ring B3 each independently is a nucleobase or a modified nucleobase;

X2 is O, S(O)p, NR24 or CR25R26 in which p is 0, 1, or 2;

Y0 is O or CR6R7;

Y1 is O, S(O)n, CR6R7, or NR8, in which n is 0, 1, or 2;

each [symbol] is a single bond or absent, wherein when each [symbol] is a single bond, Y1 is O, S(O)n, CR6R7, or NR8; and when each [symbol] is absent, Y1 is void;

Y2 is (OP(O)R4)m in which m is 0, 1, or 2, or —O—(CR40R41)u-Q0-(CR42R43)v-, in which Q0 is a bond, O, S(O)r, NR44, or CR45R46, r is 0, 1, or 2, and each of u and v independently is 1, 2, 3 or 4;

each R2 and R2′ independently is halo, LNA, or OR3;

each R3 independently is H, C1-C6 alkyl, C2-C6 alkenyl, or C2-C6 alkynyl and R3, when being C1-C6 alkyl, C2-C6 alkenyl, or C2-C6 alkynyl, is optionally substituted with one or more of halo, OH and C1-C6 alkoxyl that is optionally substituted with one or more OH or OC(O)—C1-C6 alkyl;

each R4 and R4′ independently is H, halo, C1-C6 alkyl, OH, SH, SeH, or BH₃—;

each of R6, R7, and R8, independently, is -Q1-T1, in which Q1 is a bond or C1-C3 alkyl linker optionally substituted with one or more of halo, cyano, OH and C1-C6 alkoxy, and T1 is H, halo, OH, COOH, cyano, or Rs1, in which Rs1 is C1-C3 alkyl, C2-C6 alkenyl, C2-C6 alkynyl, C1-C6 alkoxyl, C(O)O-C1-C6 alkyl, C3-C8 cycloalkyl, C6-C10 aryl, NR31R32, (NR31R32R33)+, 4 to 12-membered heterocycloalkyl, or 5- or 6-membered heteroaryl, and Rs1 is optionally substituted with one or more substituents selected from the group consisting of halo, OH, oxo, C1-C6 alkyl, COOH, C(O)O-C1-C6 alkyl, cyano, C1-C6 alkoxyl, NR31R32, (NR31R32R33)+, C3-C8 cycloalkyl, C6-C10 aryl, 4 to 12-membered heterocycloalkyl, and 5- or 6-membered heteroaryl;

each of R10, R11, R12, R13 R14, and R15, independently, is -Q2-T2, in which Q2 is a bond or C1-C3 alkyl linker optionally substituted with one or more of halo, cyano, OH and C1-C6 alkoxy, and T2 is H, halo, OH, NH2, cyano, NO2, N3, Rs2, or ORs2, in which Rs2 is C1-C6 alkyl, C2-C6 alkenyl, C2-C6 alkynyl, C3-C8 cycloalkyl, C6-C10 aryl, NHC(O)—C1-C6 alkyl, NR31R32, (NR31R32R33)+, 4 to 12-membered heterocycloalkyl, or 5- or 6-membered heteroaryl, and Rs2 is optionally substituted with one or more substituents selected from the group consisting of halo, OH, oxo, C1-C6 alkyl, COOH, C(O)O-C1-C6 alkyl, cyano, C1-C6 alkoxyl, NR31R32, (NR31R32R33)+, C3-C8 cycloalkyl, C6-C10 aryl, 4 to 12-membered heterocycloalkyl, and 5- or 6-membered heteroaryl; or alternatively R12 together with R14 is oxo, or R13 together with R15 is oxo,

each of R20, R21, R22, and R23 independently is -Q3-T3, in which Q3 is a bond or C1-C3 alkyl linker optionally substituted with one or more of halo, cyano, OH and C1-C6 alkoxy, and T3 is H, halo, OH, NH2, cyano, NO2, N3, RS3, or ORS3, in which RS3 is C1-C6 alkyl, C2-C6 alkenyl, C2-C6 alkynyl, C3-C8 cycloalkyl, C6-C10 aryl, NHC(O)—C1-C6 alkyl, mono-C1-C6 alkylamino, di-C1-C6 alkylamino, 4 to 12-membered heterocycloalkyl, or 5- or 6-membered heteroaryl, and Rs3 is optionally substituted with one or more substituents selected from the group consisting of halo, OH, oxo, C1-C6 alkyl, COOH, C(O)O-C1-C6 alkyl, cyano, C1-C6 alkoxyl, amino, mono-C1-C6 alkylamino, di-C1-C6 alkylamino, C3-C8 cycloalkyl, C6-C10 aryl, 4 to 12-membered heterocycloalkyl, and 5- or 6-membered heteroaryl;

each of R24, R25, and R26 independently is H or C1-C6 alkyl;

each of R27 and R28 independently is H or OR29; or R27 and R28 together form O-R30-O; each R29 independently is H, C1-C6 alkyl, C2-C6 alkenyl, or C2-C6 alkynyl and R29, when being C1-C6 alkyl, C2-C6 alkenyl, or C2-C6 alkynyl, is optionally substituted with one or more of halo, OH and C1-C6 alkoxyl that is optionally substituted with one or more OH or OC(O)—C1-C6 alkyl;

R30 is C1-C6 alkylene optionally substituted with one or more of halo, OH and C1-C6 alkoxyl;

each of R31, R32, and R33, independently is H, C1-C6 alkyl, C3-C8 cycloalkyl, C6-C10 aryl, 4 to 12-membered heterocycloalkyl, or 5- or 6-membered heteroaryl;

each of R40, R41, R42, and R43 independently is H, halo, OH, cyano, N3, OP(O)R47R48, or C1-C6 alkyl optionally substituted with one or more OP(O)R47R48, or one R41 and one R43, together with the carbon atoms to which they are attached and Q0, form C4-C10 cycloalkyl, 4- to 14-membered heterocycloalkyl, C6-C10 aryl, or 5- to 14-membered heteroaryl, and each of the cycloalkyl, heterocycloalkyl, phenyl, or 5- to 6-membered heteroaryl is optionally substituted with one or more of OH, halo, cyano, N3, oxo, OP(O)R47R48, C1-C6 alkyl, C1-C6 haloalkyl, COOH, C(O)O-C1-C6 alkyl, C1-C6 alkoxyl, C1-C6 haloalkoxyl, amino, mono-C1-C6 alkylamino, and di-C1-C6 alkylamino; R44 is H, C1-C6 alkyl, or an amine protecting group;

each of R45 and R46 independently is H, OP(O)R47R48, or C1-C6 alkyl optionally substituted with one or more OP(O)R47R48, and

each of R47 and R48, independently is H, halo, C1-C6 alkyl, OH, SH, SeH, or BH₃.

It should be understood that a cap analog, as provided herein, may include any of the cap analogs described in international publication WO 2017/066797, published on 20 Apr. 2017, incorporated by reference herein in its entirety.

In some embodiments, the B2 middle position can be a non-ribose molecule, such as arabinose.

In some embodiments R2 is ethyl-based.

Thus, in some embodiments, a trinucleotide cap comprises the following structure:

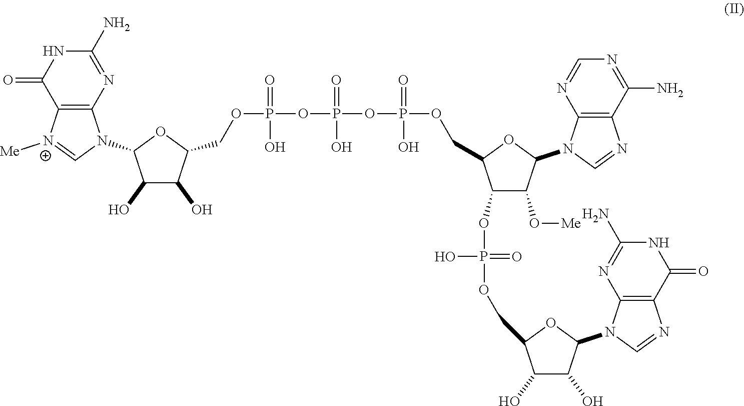

In other embodiments, a trinucleotide cap comprises the following structure:

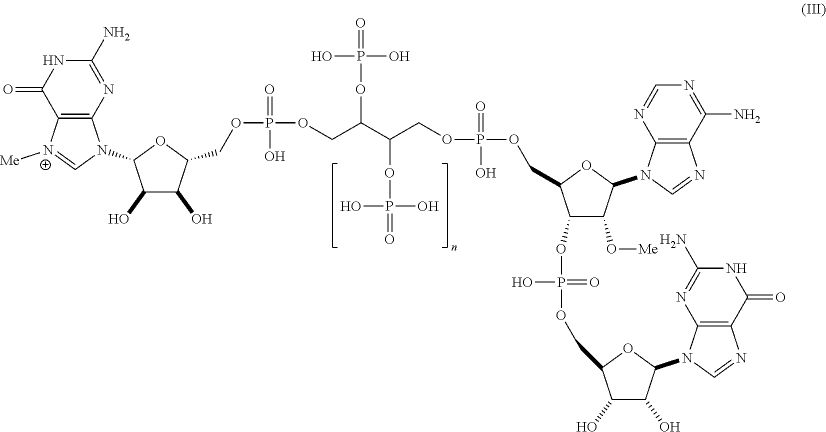

In yet other embodiments, a trinucleotide cap comprises the following structure:

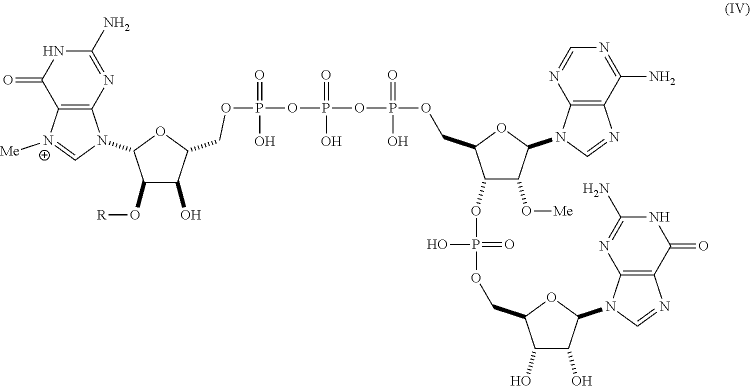

In still other embodiments, a trinucleotide cap comprises the following structure:

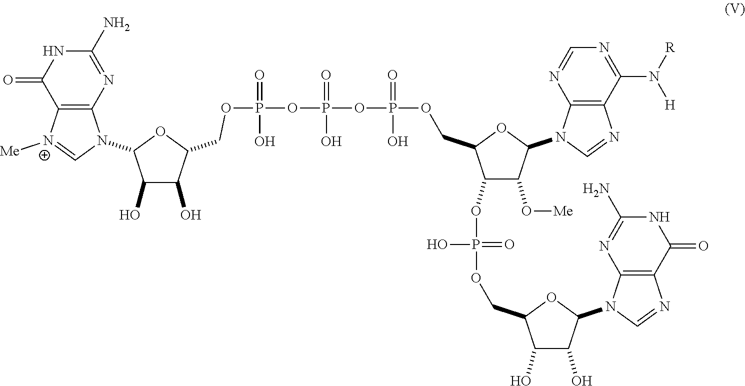

A dinucleotide cap, in some embodiments, comprises a compound of formula (I-b)

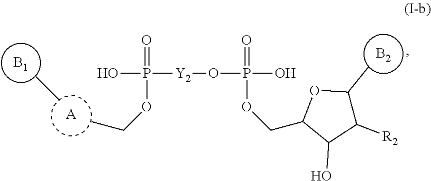

or a stereoisomer, tautomer or salt thereof, wherein

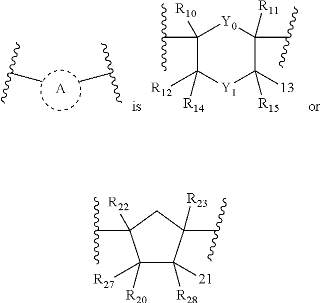

ring B1 is a modified or unmodified Guanine;

ring B2 is a nucleobase or a modified nucleobase;

X2 is O, S(O)p, NR24 or CR25R26 in which p is 0, 1, or 2; Y0 is O or CR6R7; Y1 is O, S(O)n, CR6R7, or NR8, in which n is 0, 1, or 2;

[symbol]

each [symbol] is a single bond or absent, wherein when each [symbol] is a single bond, Y1 is O, S(O)n, CR6R7, or NR8; and when each [symbol] is absent, Y1 is void;

Y2 is (OP(O)R4)m in which m is 0, 1, or 2, or —O—(CR40R41)u-Q0-(CR42R43)v-, in which Q0 is a bond, O, S(O)r, NR44, or CR45R46, r is 0, 1, or 2, and each of u and v independently is 1, 2, 3 or 4;

R2 is halo, LNA, or OR3;

each R3 independently is H, C1-C6 alkyl, C2-C6 alkenyl, or C2-C6 alkynyl and R3, when being C1-C6 alkyl, C2-C6 alkenyl, or C2-C6 alkynyl, is optionally substituted with one or more of halo, OH and C1-C6 alkoxyl that is optionally substituted with one or more OH or OC(O)—C1-C6 alkyl;

R4 is H, halo, C1-C6 alkyl, OH, SH, SeH, or BH₃—;

each of R6, R7, and R8, independently, is -Q1-T1, in which Q1 is a bond or C1-C3 alkyl linker optionally substituted with one or more of halo, cyano, OH and C1-C6 alkoxy, and T1 is H, halo, OH, COOH, cyano, or Rs1, in which Rs1 is C1-C3 alkyl, C2-C6 alkenyl, C2-C6 alkynyl, C1-C6 alkoxyl, C(O)O-C1-C6 alkyl, C3-C8 cycloalkyl, C6-C10 aryl, NR31R32, (NR31R32R33)+, 4 to 12-membered heterocycloalkyl, or 5- or 6-membered heteroaryl, and Rs1 is optionally substituted with one or more substituents selected from the group consisting of halo, OH, oxo, C1-C6 alkyl, COOH, C(O)O-C1-C6 alkyl, cyano, C1-C6 alkoxyl, NR31R32, (NR31R32R33)+, C3-C8 cycloalkyl, C6-C10 aryl, 4 to 12-membered heterocycloalkyl, and 5- or 6-membered heteroaryl;

each of R10, R11, R12, R13 R14, and R15, independently, is -Q2-T2, in which Q2 is a bond or C1-C3 alkyl linker optionally substituted with one or more of halo, cyano, OH and C1-C6 alkoxy, and T2 is H, halo, OH, NH₂, cyano, NO₂, N₃, Rs2, or ORs2, in which Rs2 is C1-C6 alkyl, C2-C6 alkenyl, C2-C6 alkynyl, C3-C8 cycloalkyl, C6-C10 aryl, NHC(O)—C1-C6 alkyl, NR31R32, (NR31R32R33)+, 4 to 12-membered heterocycloalkyl, or 5- or 6-membered heteroaryl, and Rs2 is optionally substituted with one or more substituents selected from the group consisting of halo, OH, oxo, C1-C6 alkyl, COOH, C(O)O-C1-C6 alkyl, cyano, C1-C6 alkoxyl, NR31R32, (NR31R32R33)+, C3-C8 cycloalkyl, C6-C10 aryl, 4 to 12-membered heterocycloalkyl, and 5- or 6-membered heteroaryl; or alternatively R12 together with R14 is oxo, or R13 together with R15 is oxo,

each of R20, R21, R22, and R23 independently is -Q3-T3, in which Q3 is a bond or C1-C3 alkyl linker optionally substituted with one or more of halo, cyano, OH and C1-C6 alkoxy, and T3 is H, halo, OH, NH2, cyano, NO2, N3, RS3, or ORS3, in which RS3 is C1-C6 alkyl, C2-C6 alkenyl, C2-C6 alkynyl, C3-C8 cycloalkyl, C6-C10 aryl, NHC(O)—C1-C6 alkyl, mono-C1-C6 alkylamino, di-C1-C6 alkylamino, 4 to 12-membered heterocycloalkyl, or 5- or 6-membered heteroaryl, and Rs3 is optionally substituted with one or more substituents selected from the group consisting of halo, OH, oxo, C1-C6 alkyl, COOH, C(O)O-C1-C6 alkyl, cyano, C1-C6 alkoxyl, amino, mono-C1-C6 alkylamino, di-C1-C6 alkylamino, C3-C8 cycloalkyl, C6-C10 aryl, 4 to 12-membered heterocycloalkyl, and 5- or 6-membered heteroaryl;

each of R24, R25, and R26 independently is H or C1-C6 alkyl;

each of R27 and R28 independently is H or OR29; or R27 and R28 together form O-R30-O; each R29 independently is H, C1-C6 alkyl, C2-C6 alkenyl, or C2-C6 alkynyl and R29, when being C1-C6 alkyl, C2-C6 alkenyl, or C2-C6 alkynyl, is optionally substituted with one or more of halo, OH and C1-C6 alkoxyl that is optionally substituted with one or more OH or OC(O)—C1-C6 alkyl;

R30 is C1-C6 alkylene optionally substituted with one or more of halo, OH and C1-C6 alkoxyl;

each of R31, R32, and R33, independently is H, C1-C6 alkyl, C3-C8 cycloalkyl, C6-C10 aryl, 4 to 12-membered heterocycloalkyl, or 5- or 6-membered heteroaryl;

each of R40, R41, R42, and R43 independently is H, halo, OH, cyano, N₃, OP(O)R47R48, or C1-C6 alkyl optionally substituted with one or more OP(O)R47R48, or one R41 and one R43, together with the carbon atoms to which they are attached and Q0, form C4-C10 cycloalkyl, 4- to 14-membered heterocycloalkyl, C6-C10 aryl, or 5- to 14-membered heteroaryl, and each of the cycloalkyl, heterocycloalkyl, phenyl, or 5- to 6-membered heteroaryl is optionally substituted with one or more of OH, halo, cyano, N₃, oxo, OP(O)R47R48, C1-C6 alkyl, C1-C6 haloalkyl, COOH, C(O)O-C1-C6 alkyl, C1-C6 alkoxyl, C1-C6 haloalkoxyl, amino, mono-C1-C6 alkylamino, and di-C1-C6 alkylamino;

R44 is H, C1-C6 alkyl, or an amine protecting group;

each of R45 and R46 independently is H, OP(O)R47R48, or C1-C6 alkyl optionally substituted with one or more OP(O)R47R48, and

each of R47 and R48, independently is H, halo, C1-C6 alkyl, OH, SH, SeH, or BH₃.

Thus, in some embodiments, a dinucleotide cap comprises the following structure:

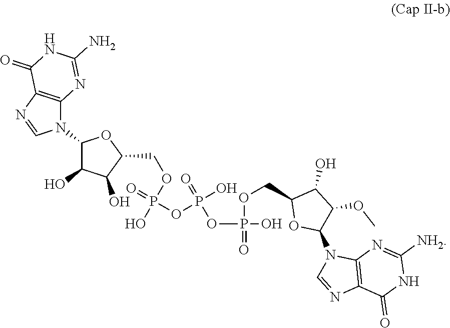 [SMILES: CO[C@H]1[C@@H](O)[C@H](COP(=O)(O)OP(=O)(O)OP(=O)(O)OC[C@H]2O[C@@H](n3cnc4c(=O)[nH]c(N)nc43)[C@H](O)[C@@H]2O)O[C@@H]1n1cnc2c(=O)[nH]c(N)nc21]

### 3′ Stabilizing Region

In some embodiments, the polynucleotides (e.g., mRNAs) of the present disclosure (e.g., a polynucleotide comprising a nucleotide sequence encoding a therapeutic payload or prophylactic payload, an effector molecule and/or a tether molecule) further comprise a 3′ stabilizing region, e.g., a stabilized tail e.g., as described herein. A polynucleotide containing a 3′-stabilizing region (e.g., a 3′-stabilizing region including an alternative nucleobase, sugar, and/or backbone) may be particularly effective for use in therapeutic compositions, because they may benefit from increased stability, high expression levels.

In an embodiment, the 3′ stabilizing region comprises a poly A tail, e.g., a poly A tail comprising 80-150, e.g., 120, adenines. In an embodiment, the poly A tail comprises a UCUAG sequence (SEQ ID NO: 92). In an embodiment, the poly A tail comprises about 80-120, e.g., 100, adenines upstream of SEQ ID NO: 92. In an embodiment, the poly A tail comprises about 1-40, e.g., 20, adenines downstream of SEQ ID NO: 92.

In an embodiment, the 3′ stabilizing region comprises at least one alternative nucleoside. In an embodiment, the alternative nucleoside is an inverted thymidine (idT). In an embodiment, the alternative nucleoside is disposed at the 3′ end of the 3′ stabilizing region.

In an embodiment, the 3′ stabilizing region comprises a structure of Formula VII:

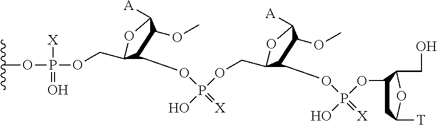

or a salt thereof, wherein each X is independently O or S, and A represents adenine and T represents Thymine.

In an aspect, an LNP composition comprising a polynucleotide comprising a stabilizing region disclosed herein comprises: (i) an ionizable lipid, e.g., an amino lipid; (ii) a sterol or other structural lipid; (iii) a non-cationic helper lipid or phospholipid; and (iv) a PEG-lipid.

In another aspect, the LNP compositions of the disclosure are used in a method of treating a disease or disorder.

In another aspect, the LNP compositions of the disclosure are used to modify a cell (e.g., stem cell), e.g., modify a parameter associated with the cell or a component associated with the cell,

In an aspect, an LNP composition comprising a polynucleotide disclosed herein encoding a therapeutic payload or prophylactic payload, e.g., as described herein, can be administered with an additional agent, e.g., as described herein.

Tails, e.g. Poly A Tails

In some embodiments, the polynucleotides (e.g., mRNAs) of the present disclosure (e.g., a polynucleotide comprising a nucleotide sequence encoding a therapeutic payload or prophylactic payload, an effector molecule and/or a tether molecule) further comprise a tail, e.g., a poly-A tail. In further embodiments, terminal groups on the poly-A tail can be incorporated for stabilization. In other embodiments, a poly-A tail comprises des-3′ hydroxyl tails.

During RNA processing, a long chain of adenine nucleotides (poly-A tail) can be added to a polynucleotide such as an mRNA molecule to increase stability. Immediately after transcription, the 3′ end of the transcript can be cleaved to free a 3′ hydroxyl. Then poly-A polymerase adds a chain of adenine nucleotides to the RNA. The process, called polyadenylation, adds a poly-A tail that can be between, for example, approximately 80 to approximately 250 residues long, including approximately 80, 90, 100, 110, 120, 130, 140, 150, 160, 170, 180, 190, 200, 210, 220, 230, 240 or 250 residues long. In one embodiment, the poly-A tail is 100 nucleotides in length (SEQ ID NO:25).

(SEQ ID NO: 25)
aaaaaaaaaa aaaaaaaaaa aaaaaaaaaa aaaaaaaaaa
aaaaaaaaaa aaaaaaaaaa aaaaaaaaaa aaaaaaaaaa
aaaaaaaaaa aaaaaaaaaa

PolyA tails can also be added after the construct is exported from the nucleus.

According to the present invention, terminal groups on the poly A tail can be incorporated for stabilization. Polynucleotides of the present invention can include des-3′ hydroxyl tails. They can also include structural moieties or 2′-Omethyl modifications as taught by Junjie Li, et al. (Current Biology, Vol. 15, 1501-1507, Aug. 23, 2005, the contents of which are incorporated herein by reference in its entirety).

The polynucleotides of the present invention can be designed to encode transcripts with alternative polyA tail structures including histone mRNA. According to Norbury, “Terminal uridylation has also been detected on human replication-dependent histone mRNAs. The turnover of these mRNAs is thought to be important for the prevention of potentially toxic histone accumulation following the completion or inhibition of chromosomal DNA replication. These mRNAs are distinguished by their lack of a 3′ poly(A) tail, the function of which is instead assumed by a stable stem-loop structure and its cognate stem-loop binding protein (SLBP); the latter carries out the same functions as those of PABP on polyadenylated mRNAs” (Norbury, “Cytoplasmic RNA: a case of the tail wagging the dog,” Nature Reviews Molecular Cell Biology; AOP, published online 29 Aug. 2013; doi:10.1038/nrm3645) the contents of which are incorporated herein by reference in its entirety.

Unique poly-A tail lengths provide certain advantages to the polynucleotides of the present invention. Generally, the length of a poly-A tail, when present, is greater than 30 nucleotides in length. In another embodiment, the poly-A tail is greater than 35 nucleotides in length (e.g., at least or greater than about 35, 40, 45, 50, 55, 60, 70, 80, 90, 100, 120, 140, 160, 180, 200, 250, 300, 350, 400, 450, 500, 600, 700, 800, 900, 1,000, 1,100, 1,200, 1,300, 1,400, 1,500, 1,600, 1,700, 1,800, 1,900, 2,000, 2,500, and 3,000 nucleotides).

In some embodiments, the polynucleotide or region thereof includes from about 30 to about 3,000 nucleotides (e.g., from 30 to 50, from 30 to 100, from 30 to 250, from 30 to 500, from 30 to 750, from 30 to 1,000, from 30 to 1,500, from 30 to 2,000, from 30 to 2,500, from 50 to 100, from 50 to 250, from 50 to 500, from 50 to 750, from 50 to 1,000, from 50 to 1,500, from 50 to 2,000, from 50 to 2,500, from 50 to 3,000, from 100 to 500, from 100 to 750, from 100 to 1,000, from 100 to 1,500, from 100 to 2,000, from 100 to 2,500, from 100 to 3,000, from 500 to 750, from 500 to 1,000, from 500 to 1,500, from 500 to 2,000, from 500 to 2,500, from 500 to 3,000, from 1,000 to 1,500, from 1,000 to 2,000, from 1,000 to 2,500, from 1,000 to 3,000, from 1,500 to 2,000, from 1,500 to 2,500, from 1,500 to 3,000, from 2,000 to 3,000, from 2,000 to 2,500, and from 2,500 to 3,000).

In some embodiments, the poly-A tail is designed relative to the length of the overall polynucleotide or the length of a particular region of the polynucleotide. This design can be based on the length of a coding region, the length of a particular feature or region or based on the length of the ultimate product expressed from the polynucleotides.

In this context, the poly-A tail can be 10, 20, 30, 40, 50, 60, 70, 80, 90, or 100% greater in length than the polynucleotide or feature thereof. The poly-A tail can also be designed as a fraction of the polynucleotides to which it belongs. In this context, the poly-A tail can be 10, 20, 30, 40, 50, 60, 70, 80, or 90% or more of the total length of the construct, a construct region or the total length of the construct minus the poly-A tail. Further, engineered binding sites and conjugation of polynucleotides for Poly-A binding protein can enhance expression.

Additionally, multiple distinct polynucleotides can be linked together via the PABP (Poly-A binding protein) through the 3′-end using modified nucleotides at the 3′-terminus of the poly-A tail. Transfection experiments can be conducted in relevant cell lines at and protein production can be assayed by ELISA at 12 hr, 24 hr, 48 hr, 72 hr and day 7 post-transfection.

In some embodiments, the polynucleotides of the present invention are designed to include a polyA-G Quartet region. The G-quartet is a cyclic hydrogen bonded array of four guanine nucleotides that can be formed by G-rich sequences in both DNA and RNA. In this embodiment, the G-quartet is incorporated at the end of the poly-A tail. The resultant polynucleotide is assayed for stability, protein production and other parameters including half-life at various time points. It has been discovered that the polyA-G quartet results in protein production from an mRNA equivalent to at least 75% of that seen using a poly-A tail of 120 nucleotides alone (SEQ ID NO:26).

(SEQ ID NO: 26)
aaaaaaaaaa aaaaaaaaaa aaaaaaaaaa aaaaaaaaaa
aaaaaaaaaa aaaaaaaaaa aaaaaaaaaa aaaaaaaaaa
aaaaaaaaaa aaaaaaaaaa aaaaaaaaaa aaaaaaaaaa

### Start Codon Region

The disclosure also includes a polynucleotide (e.g., mRMA) that comprises both a start codon region and the polynucleotide described herein (e.g., a polynucleotide comprising a nucleotide sequence encoding a therapeutic payload or prophylactic payload, an effector molecule and/or a tether molecule). In some embodiments, the polynucleotides of the present disclosure can have regions that are analogous to or function like a start codon region.

In some embodiments, the translation of a polynucleotide can initiate on a codon that is not the start codon AUG. Translation of the polynucleotide can initiate on an alternative start codon such as, but not limited to, ACG, AGG, AAG, CTG/CUG, GTG/GUG, ATA/AUA, ATT/AUU, TTG/UUG (see Touriol et al. Biology of the Cell 95 (2003) 169-178 and Matsuda and Mauro PLoS ONE, 2010 5:11; the contents of each of which are herein incorporated by reference in its entirety).

As a non-limiting example, the translation of a polynucleotide begins on the alternative start codon ACG. As another non-limiting example, polynucleotide translation begins on the alternative start codon CTG or CUG. As another non-limiting example, the translation of a polynucleotide begins on the alternative start codon GTG or GUG.

Nucleotides flanking a codon that initiates translation such as, but not limited to, a start codon or an alternative start codon, are known to affect the translation efficiency, the length and/or the structure of the polynucleotide. See, e.g., Matsuda and Mauro PLoS ONE, 2010 5:11; the contents of which are herein incorporated by reference in its entirety. Masking any of the nucleotides flanking a codon that initiates translation can be used to alter the position of translation initiation, translation efficiency, length and/or structure of a polynucleotide.

In some embodiments, a masking agent can be used near the start codon or alternative start codon to mask or hide the codon to reduce the probability of translation initiation at the masked start codon or alternative start codon. Non-limiting examples of masking agents include antisense locked nucleic acids (LNA) polynucleotides and exon-junction complexes (EJCs) (See, e.g., Matsuda and Mauro describing masking agents LNA polynucleotides and EJCs (PLoS ONE, 2010 5:11); the contents of which are herein incorporated by reference in its entirety).

In another embodiment, a masking agent can be used to mask a start codon of a polynucleotide to increase the likelihood that translation will initiate on an alternative start codon. In some embodiments, a masking agent can be used to mask a first start codon or alternative start codon to increase the chance that translation will initiate on a start codon or alternative start codon downstream to the masked start codon or alternative start codon.

In some embodiments, a start codon or alternative start codon can be located within a perfect complement for a miRNA binding site. The perfect complement of a miRNA binding site can help control the translation, length and/or structure of the polynucleotide similar to a masking agent. As a non-limiting example, the start codon or alternative start codon can be located in the middle of a perfect complement for a miRNA binding site. The start codon or alternative start codon can be located after the first nucleotide, second nucleotide, third nucleotide, fourth nucleotide, fifth nucleotide, sixth nucleotide, seventh nucleotide, eighth nucleotide, ninth nucleotide, tenth nucleotide, eleventh nucleotide, twelfth nucleotide, thirteenth nucleotide, fourteenth nucleotide, fifteenth nucleotide, sixteenth nucleotide, seventeenth nucleotide, eighteenth nucleotide, nineteenth nucleotide, twentieth nucleotide or twenty-first nucleotide.

In another embodiment, the start codon of a polynucleotide can be removed from the polynucleotide sequence to have the translation of the polynucleotide begin on a codon that is not the start codon. Translation of the polynucleotide can begin on the codon following the removed start codon or on a downstream start codon or an alternative start codon. In a non-limiting example, the start codon ATG or AUG is removed as the first 3 nucleotides of the polynucleotide sequence to have translation initiate on a downstream start codon or alternative start codon. The polynucleotide sequence where the start codon was removed can further comprise at least one masking agent for the downstream start codon and/or alternative start codons to control or attempt to control the initiation of translation, the length of the polynucleotide and/or the structure of the polynucleotide.

### Stop Codon Region

The disclosure also includes a polynucleotide (e.g., mRNA) that comprises both a stop codon region and the polynucleotide described herein (e.g., a polynucleotide comprising a nucleotide sequence encoding a therapeutic payload or prophylactic payload, an effector molecule and/or a tether molecule). In some embodiments, the polynucleotides of the present disclosure can include at least two stop codons before the 3′ untranslated region (UTR). The stop codon can be selected from TGA, TAA and TAG in the case of DNA, or from UGA, UAA and UAG in the case of RNA. In some embodiments, the polynucleotides of the present disclosure include the stop codon TGA in the case or DNA, or the stop codon UGA in the case of RNA, and one additional stop codon. In a further embodiment the addition stop codon can be TAA or UAA. In another embodiment, the polynucleotides of the present disclosure include three consecutive stop codons, four stop codons, or more.

### Methods of Making Polynucleotides

The present disclosure also provides methods for making a polynucleotide disclosed herein or a complement thereof. In some aspects, a polynucleotide (e.g., an mRNA) disclosed herein encoding a therapeutic payload or prophylactic payload, an effector molecule and/or a tether molecule can be constructed using in vitro transcription.

In other aspects, a polynucleotide (e.g., an mRNA) disclosed herein encoding a therapeutic payload or prophylactic payload, an effector molecule and/or a tether molecule can be constructed by chemical synthesis using an oligonucleotide synthesizer. In other aspects, a polynucleotide (e.g., an mRNA) disclosed herein encoding a therapeutic payload or prophylactic payload, an effector molecule and/or a tether molecule is made by using a host cell. In certain aspects, a polynucleotide (e.g., an mRNA) disclosed herein encoding a therapeutic payload or prophylactic payload, an effector molecule and/or a tether molecule is made by one or more combination of the IVT, chemical synthesis, host cell expression, or any other methods known in the art.

Naturally occurring nucleosides, non-naturally occurring nucleosides, or combinations thereof, can totally or partially naturally replace occurring nucleosides present in the candidate nucleotide sequence and can be incorporated into a sequence-optimized nucleotide sequence (e.g., an mRNA) encoding a therapeutic payload or prophylactic payload, an effector molecule and/or a tether molecule. The resultant mRNAs can then be examined for their ability to produce protein and/or produce a therapeutic outcome.

Exemplary methods of making a polynucleotide disclosed herein include: in vitro transcription enzymatic synthesis and chemical synthesis which are disclosed in International PCT application WO 2017/201325, filed on 18 May 2017, the entire contents of which are hereby incorporated by reference.

Purification

In other aspects, a polynucleotide (e.g., an mRNA) disclosed herein encoding a therapeutic payload or prophylactic payload, an effector molecule and/or a tether molecule can be purified. Purification of the polynucleotides (e.g., mRNA) encoding a therapeutic payload or prophylactic payload, an effector molecule and/or a tether molecule described herein can include, but is not limited to, polynucleotide clean-up, quality assurance and quality control. Clean-up can be performed by methods known in the arts such as, but not limited to, AGENCOURT® beads (Beckman Coulter Genomics, Danvers, MA), poly-T beads, LNA™ oligo-T capture probes (EXIQON® Inc, Vedbaek, Denmark) or HPLC based purification methods such as, but not limited to, strong anion exchange HPLC, weak anion exchange HPLC, reverse phase HPLC (RP-HPLC), and hydrophobic interaction HPLC (HIC-HPLC). The term “purified” when used in relation to a polynucleotide such as a “purified polynucleotide” refers to one that is separated from at least one contaminant. As used herein, a “contaminant” is any substance which makes another unfit, impure or inferior. Thus, a purified polynucleotide (e.g., DNA and RNA) is present in a form or setting different from that in which it is found in nature, or a form or setting different from that which existed prior to subjecting it to a treatment or purification method.

In some embodiments, purification of a polynucleotide (e.g., mRNA) encoding a therapeutic payload or prophylactic payload, an effector molecule and/or a tether molecule of the disclosure removes impurities that can reduce or remove an unwanted immune response, e.g., reducing cytokine activity.

In some embodiments, the polynucleotide (e.g., mRNA) encoding a therapeutic payload or prophylactic payload, an effector molecule and/or a tether molecule of the disclosure is purified prior to administration using column chromatography (e.g., strong anion exchange HPLC, weak anion exchange HPLC, reverse phase HPLC (RP-HPLC), and hydrophobic interaction HPLC (HIC-HPLC), or (LCMS)). In some embodiments, a column chromatography (e.g., strong anion exchange HPLC, weak anion exchange HPLC, reverse phase HPLC (RP-HPLC), and hydrophobic interaction HPLC (HIC-HPLC), or (LCMS)) purified polynucleotide, which encodes a therapeutic payload or prophylactic payload, an effector molecule and/or a tether molecule disclosed herein increases expression of the therapeutic payload or prophylactic payload, an effector molecule and/or a tether molecule compared to polynucleotides encoding the therapeutic payload or prophylactic payload, an effector molecule and/or a tether molecule purified by a different purification method.

In some embodiments, a column chromatography (e.g., strong anion exchange HPLC, weak anion exchange HPLC, reverse phase HPLC (RP-HPLC), and hydrophobic interaction HPLC (HIC-HPLC), or (LCMS)) purified polynucleotide encodes a therapeutic payload or prophylactic payload, an effector molecule and/or a tether molecule. In some embodiments, the purified polynucleotide encodes a therapeutic payload or prophylactic payload, an effector molecule and/or a tether molecule.

In some embodiments, the purified polynucleotide is at least about 80% pure, at least about 85% pure, at least about 90% pure, at least about 95% pure, at least about 96% pure, at least about 97% pure, at least about 98% pure, at least about 99% pure, or about 100% pure.

A quality assurance and/or quality control check can be conducted using methods such as, but not limited to, gel electrophoresis, UV absorbance, or analytical HPLC.

In another embodiment, the polynucleotides can be sequenced by methods including, but not limited to reverse-transcriptase-PCR.

### Chemical Modifications of Polynucleotides

The present disclosure provides for modified nucleosides and nucleotides of a nucleic acid (e.g., RNA nucleic acids, such as mRNA nucleic acids). A “nucleoside” refers to a compound containing a sugar molecule (e.g., a pentose or ribose) or a derivative thereof in combination with an organic base (e.g., a purine or pyrimidine) or a derivative thereof (also referred to herein as “nucleobase”). A “nucleotide” refers to a nucleoside, including a phosphate group. Modified nucleotides may by synthesized by any useful method, such as, for example, chemically, enzymatically, or recombinantly, to include one or more modified or non-natural nucleosides. Nucleic acids can comprise a region or regions of linked nucleosides. Such regions may have variable backbone linkages. The linkages can be standard phosphodiester linkages, in which case the nucleic acids would comprise regions of nucleotides.

Modified nucleotide base pairing encompasses not only the standard adenosine-thymine, adenosine-uracil, or guanosine-cytosine base pairs, but also base pairs formed between nucleotides and/or modified nucleotides comprising non-standard or modified bases, wherein the arrangement of hydrogen bond donors and hydrogen bond acceptors permits hydrogen bonding between a non-standard base and a standard base or between two complementary non-standard base structures, such as, for example, in those nucleic acids having at least one chemical modification. One example of such non-standard base pairing is the base pairing between the modified nucleotide inosine and adenine, cytosine or uracil. Any combination of base/sugar or linker may be incorporated into nucleic acids of the present disclosure.

In some embodiments, modified nucleobases in nucleic acids (e.g., RNA nucleic acids, such as mRNA nucleic acids) comprise N1-methyl-pseudouridine (m1ψ), 1-ethyl-pseudouridine (e1ψ), 5-methoxy-uridine (mo5U), 5-methyl-cytidine (m5C), and/or pseudouridine (ψ). In some embodiments, modified nucleobases in nucleic acids (e.g., RNA nucleic acids, such as mRNA nucleic acids) comprise 5-methoxymethyl uridine, 5-methylthio uridine, 1-methoxymethyl pseudouridine, 5-methyl cytidine, and/or 5-methoxy cytidine. In some embodiments, the polyribonucleotide includes a combination of at least two (e.g., 2, 3, 4 or more) of any of the aforementioned modified nucleobases, including but not limited to chemical modifications.

In some embodiments, an RNA nucleic acid of the disclosure comprises N1-methyl-pseudouridine (m1ψ) substitutions at one or more or all uridine positions of the nucleic acid.

In some embodiments, an RNA nucleic acid of the disclosure comprises N1-methyl-pseudouridine (m1ψ) substitutions at one or more or all uridine positions of the nucleic acid and 5-methyl cytidine substitutions at one or more or all cytidine positions of the nucleic acid.

In some embodiments, an RNA nucleic acid of the disclosure comprises pseudouridine (ψ) substitutions at one or more or all uridine positions of the nucleic acid.

In some embodiments, an RNA nucleic acid of the disclosure comprises pseudouridine (ψ) substitutions at one or more or all uridine positions of the nucleic acid and 5-methyl cytidine substitutions at one or more or all cytidine positions of the nucleic acid.

In some embodiments, an RNA nucleic acid of the disclosure comprises uridine at one or more or all uridine positions of the nucleic acid.

In some embodiments, nucleic acids (e.g., RNA nucleic acids, such as mRNA nucleic acids) are uniformly modified (e.g., fully modified, modified throughout the entire sequence) for a particular modification. For example, a nucleic acid can be uniformly modified with N1-methyl-pseudouridine, meaning that all uridine residues in the mRNA sequence are replaced with N1-methyl-pseudouridine. Similarly, a nucleic acid can be uniformly modified for any type of nucleoside residue present in the sequence by replacement with a modified residue such as those set forth above.

The nucleic acids of the present disclosure may be partially or fully modified along the entire length of the molecule. For example, one or more or all or a given type of nucleotide (e.g., purine or pyrimidine, or any one or more or all of A, G, U, C) may be uniformly modified in a nucleic acid of the disclosure, or in a predetermined sequence region thereof (e.g., in the mRNA including or excluding the polyA tail). In some embodiments, all nucleotides X in a nucleic acid of the present disclosure (or in a sequence region thereof) are modified nucleotides, wherein X may be any one of nucleotides A, G, U, C, or any one of the combinations A+G, A+U, A+C, G+U, G+C, U+C, A+G+U, A+G+C, G+U+C or A+G+C.

The nucleic acid may contain from about 1% to about 100% modified nucleotides (either in relation to overall nucleotide content, or in relation to one or more types of nucleotide, i.e., any one or more of A, G, U or C) or any intervening percentage (e.g., from 1% to 20%, from 1% to 25%, from 1% to 50%, from 1% to 60%, from 1% to 70%, from 1% to 80%, from 1% to 90%, from 1% to 95%, from 10% to 20%, from 10% to 25%, from 10% to 50%, from 10% to 60%, from 10% to 70%, from 10% to 80%, from 10% to 90%, from 10% to 95%, from 10% to 100%, from 20% to 25%, from 20% to 50%, from 20% to 60%, from 20% to 70%, from 20% to 80%, from 20% to 90%, from 20% to 95%, from 20% to 100%, from 50% to 60%, from 50% to 70%, from 50% to 80%, from 50% to 90%, from 50% to 95%, from 50% to 100%, from 70% to 80%, from 70% to 90%, from 70% to 95%, from 70% to 100%, from 80% to 90%, from 80% to 95%, from 80% to 100%, from 90% to 95%, from 90% to 100%, and from 95% to 100%). It will be understood that any remaining percentage is accounted for by the presence of unmodified A, G, U, or C.

The nucleic acids may contain at a minimum 1% and at maximum 100% modified nucleotides, or any intervening percentage, such as at least 5% modified nucleotides, at least 10% modified nucleotides, at least 25% modified nucleotides, at least 50% modified nucleotides, at least 80% modified nucleotides, or at least 90% modified nucleotides. For example, the nucleic acids may contain a modified pyrimidine such as a modified uracil or cytosine. In some embodiments, at least 5%, at least 10%, at least 25%, at least 50%, at least 80%, at least 90% or 100% of the uracil in the nucleic acid is replaced with a modified uracil (e.g., a 5-substituted uracil). The modified uracil can be replaced by a compound having a single unique structure, or can be replaced by a plurality of compounds having different structures (e.g., 2, 3, 4 or more unique structures). In some embodiments, at least 5%, at least 10%, at least 25%, at least 50%, at least 80%, at least 90% or 100% of the cytosine in the nucleic acid is replaced with a modified cytosine (e.g., a 5-substituted cytosine). The modified cytosine can be replaced by a compound having a single unique structure, or can be replaced by a plurality of compounds having different structures (e.g., 2, 3, 4 or more unique structures).

### Pharmaceutical Compositions

The present disclosure provides pharmaceutical formulations comprising any of the systems, or LNP compositions disclosed herein, e.g., a system or an LNP composition comprising: (a) a first polynucleotide (e.g., mRNA) comprising: (1) a sequence encoding a therapeutic payload or prophylactic payload, and (2) a binding element; and (b) a second polynucleotide (e.g., mRNA) comprising a sequence encoding: (1) an effector molecule, and (2) a polypeptide that recognizes the binding element (a tether molecule). In an embodiment, the LNP composition does not comprise an additional targeting moiety. In some embodiments of the disclosure, the polynucleotide is formulated in compositions and complexes in combination with one or more pharmaceutically acceptable excipients. Pharmaceutical compositions can optionally comprise one or more additional active substances, e.g. therapeutically and/or prophylactically active substances. Pharmaceutical compositions of the present disclosure can be sterile and/or pyrogen-free. General considerations in the formulation and/or manufacture of pharmaceutical agents can be found, for example, in Remington: The Science and Practice of Pharmacy 21st ed., Lippincott Williams & Wilkins, 2005.

In some embodiments, compositions are administered to humans, human patients or subjects. For the purposes of the present disclosure, the phrase “active ingredient” generally refers to polynucleotides to be delivered as described herein.

Although the descriptions of pharmaceutical compositions provided herein are principally directed to pharmaceutical compositions which are suitable for administration to humans, it will be understood by the skilled artisan that such compositions are generally suitable for administration to any other animal, e.g., to non-human animals, e.g. non-human mammals. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the pharmaceutical compositions is contemplated include, but are not limited to, humans and/or other primates; mammals.

In some embodiments, the polynucleotide of the present disclosure is formulated for subcutaneous, intravenous, intraperitoneal, intramuscular, intra-articular, intra-synovial, intrasternal, intrathecal, intrahepatic, intralesional, intracranial, intraventricular, oral, inhalation spray, pulmonary, topical, rectal, nasal, buccal, vaginal, or implanted reservoir intramuscular, subcutaneous, or intradermal delivery. In other embodiments, the polynucleotide is formulated for subcutaneous or intravenous delivery.

Formulations of the pharmaceutical compositions described herein can be prepared by any method known or hereafter developed in the art of pharmacology. In general, such preparatory methods include the step of bringing the active ingredient into association with an excipient and/or one or more other accessory ingredients, and then, if necessary and/or desirable, dividing, shaping and/or packaging the product into a desired single- or multi-dose unit.

Relative amounts of the active ingredient, the pharmaceutically acceptable excipient, and/or any additional ingredients in a pharmaceutical composition in accordance with the disclosure will vary, depending upon the identity, size, and/or condition of the subject treated and further depending upon the route by which the composition is to be administered. By way of example, the composition can comprise between 0.1% and 100%, e.g., between 0.5% and 50%, between 1% and 30%, between 5% and 80%, or at least 80% (w/w) active ingredient.

### Formulations and Delivery

The polynucleotide comprising an mRNA of the disclosure can be formulated using one or more excipients.

The function of the one or more excipients is, e.g., to: (1) increase stability; (2) increase cell transfection; (3) permit the sustained or delayed release (e.g., from a depot formulation of the polynucleotide); (4) alter the biodistribution (e.g., target the polynucleotide to specific tissues or cell types); (5) increase the translation of encoded protein in vivo; and/or (6) alter the release profile of encoded protein in vivo. In addition to traditional excipients such as any and all solvents, dispersion media, diluents, or other liquid vehicles, dispersion or suspension aids, surface active agents, isotonic agents, thickening or emulsifying agents, preservatives, excipients of the present disclosure can include, without limitation, lipidoids, liposomes, lipid nanoparticles, polymers, lipoplexes, core-shell nanoparticles, peptides, proteins, cells transfected with polynucleotides (e.g., for transplantation into a subject), hyaluronidase, nanoparticle mimics and combinations thereof. Accordingly, the formulations of the disclosure can include one or more excipients, each in an amount that together increases the stability of the polynucleotide, increases cell transfection by the polynucleotide, increases the expression of polynucleotides encoded protein, and/or alters the release profile of polynucleotide encoded proteins. Further, the polynucleotides of the present disclosure can be formulated using self-assembled nucleic acid nanoparticles.

Formulations of the pharmaceutical compositions described herein can be prepared by any method known or hereafter developed in the art of pharmacology. In general, such preparatory methods include the step of associating the active ingredient with an excipient and/or one or more other accessory ingredients.

A pharmaceutical composition in accordance with the present disclosure can be prepared, packaged, and/or sold in bulk, as a single unit dose, and/or as a plurality of single unit doses. As used herein, a “unit dose” refers to a discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

Relative amounts of the active ingredient, the pharmaceutically acceptable excipient, and/or any additional ingredients in a pharmaceutical composition in accordance with the present disclosure can vary, depending upon the identity, size, and/or condition of the subject being treated and further depending upon the route by which the composition is to be administered. For example, the composition can comprise between 0.1% and 99% (w/w) of the active ingredient. By way of example, the composition can comprise between 0.1% and 100%, e.g., between 0.5 and 50%, between 1-30%, between 5-80%, at least 80% (w/w) active ingredient.

In some embodiments, the formulations described herein contain at least one polynucleotide. As a non-limiting example, the formulations contain 1, 2, 3, 4 or 5 polynucleotides.

Pharmaceutical formulations can additionally comprise a pharmaceutically acceptable excipient, which, as used herein, includes, but is not limited to, any and all solvents, dispersion media, diluents, or other liquid vehicles, dispersion or suspension aids, surface active agents, isotonic agents, thickening or emulsifying agents, preservatives, and the like, as suited to the particular dosage form desired. Various excipients for formulating pharmaceutical compositions and techniques for preparing the composition are known in the art (see Remington: The Science and Practice of Pharmacy, 21st Edition, A. R. Gennaro, Lippincott, Williams & Wilkins, Baltimore, M D, 2006). The use of a conventional excipient medium can be contemplated within the scope of the present disclosure, except insofar as any conventional excipient medium can be incompatible with a substance or its derivatives, such as by producing any undesirable biological effect or otherwise interacting in a deleterious manner with any other component(s) of the pharmaceutical composition.

In some embodiments, the particle size of the lipid nanoparticle is increased and/or decreased. The change in particle size can be able to help counter biological reaction such as, but not limited to, inflammation or can increase the biological effect of the modified mRNA delivered to mammals.

Pharmaceutically acceptable excipients used in the manufacture of pharmaceutical compositions include, but are not limited to, inert diluents, surface active agents and/or emulsifiers, preservatives, buffering agents, lubricating agents, and/or oils. Such excipients can optionally be included in the pharmaceutical formulations of the disclosure.

In some embodiments, the polynucleotides are administered in or with, formulated in or delivered with nanostructures that can sequester molecules such as cholesterol. Non-limiting examples of these nanostructures and methods of making these nanostructures are described in U.S. Application Publication No. US 2013/0195759. Exemplary structures of these nanostructures are shown in U.S. Application Publication No. US 2013/0195759, and can include a core and a shell surrounding the core.

A polynucleotide comprising an mRNA of the disclosure can be delivered to a cell using any method known in the art. For example, the polynucleotide comprising an mRNA of the disclosure can be delivered to a cell by a lipid-based delivery, e.g., transfection, or by electroporation.

### EQUIVALENTS AND SCOPE

Those skilled in the art will recognize, or be able to ascertain using no more than routine experimentation, many equivalents to the specific embodiments in accordance with the disclosure described herein. The scope of the present disclosure is not intended to be limited to the Description below, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or all of the group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the disclosure, to the tenth of the unit of the lower limit of the range, unless the context clearly dictates otherwise.

All cited sources, for example, references, publications, databases, database entries, and art cited herein, are incorporated into this application by reference, even if not expressly stated in the citation. In case of conflicting statements of a cited source and the instant application, the statement in the instant application shall control.

### EXAMPLES

The disclosure will be more fully understood by reference to the following examples. They should not, however, be construed as limiting the scope of the disclosure. It is understood that the examples and embodiments described herein are for illustrative purposes only and that various modifications or changes in light thereof will be suggested to persons skilled in the art and are to be included within the spirit and purview of this application and scope of the appended claims.

### Example 1: In Vivo Gene Editing of HSPCs with LNP Comprising mRNA Encoding Cre Recombinase

This Example describes in vivo transfection and Cre-mediated gene editing of hematopoietic stem and progenitor cells (HSPCs) upon administration of LNP formulated with Cre-mRNA (LNPcre).

Briefly, Ai14 mice which contain a silent genetic locus encoding red fluorescent protein (Td-Tomato) were given an intravenous administration of 0.5 mg/kg LNPcre. As a control, a cohort of separate mice were injected with Tris/sucrose vehicle. There were three animals in each LNPcre group (n=3). 48 hours after administration of the LNP or vehicle control, mice were euthanized and bone marrow cells were harvested and processed for flow cytometry with the indicated markers.

In this experiment, cell lineage tracing was performed utilizing Ai14 mice, which contain a silent genetic locus encoding red fluorescent protein (Td-Tomato) that can be activated by ‘Cre’ recombinase. In these mice, cells only express Td-Tomato if they are successfully transfected with Cre mRNA. Thus, in addition to providing organism-wide information as to which cell types were successfully transfected by a particular formulation and route of administration (ROA), this approach also revealed which cells are gene editable.

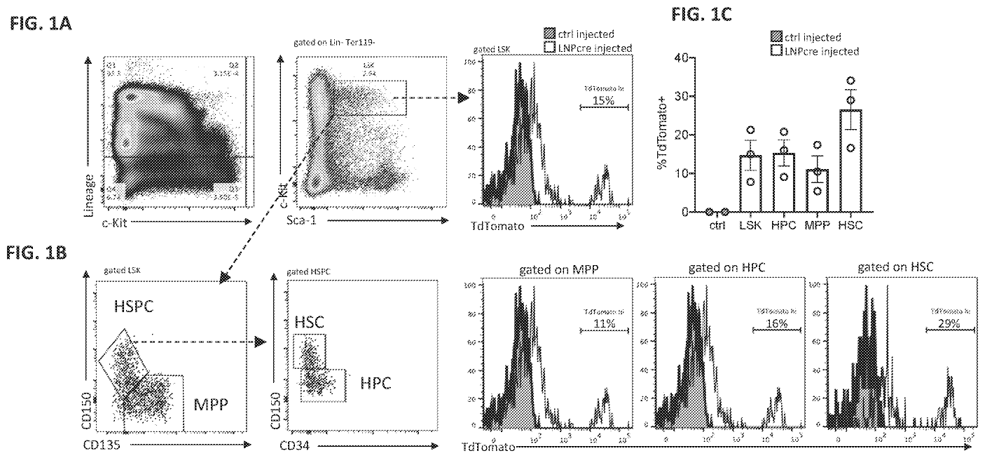

As shown in FIGS. 1A-1C, administration of Cre-mRNA LNP resulted in Cre-mediated gene editing in vivo as measured by an increase in TdTomato fluorescence. FIGS. 1A-1C depict flow cytometry plots/histograms that show TdTomato fluorescence/expression in bone marrow HSPC subsets, including LSK gate, multi-potent progenitor (MPP) cells, hematopoietic progenitors (HPC), and long-term HSC.

In summary, data using Ai14 mice and Cre-mRNA LNP showed that a Cre-mediated gene editing event could be triggered in HSPCs (LSK gate) and in the progenitor population enriched in HSCs, demonstrating efficient in vivo HSPC/HSC gene editing in mice.

### Example 2: Generation of HSPC-Derived Colony Forming Units (CFU) with Bone-Marrow Cells from Ai14 Mice Administered LNP Comprising mRNA Encoding Cre Recombinase

This Example describes the generation of HSPC-derived CFU upon ex vivo plating of bone marrow cells from mice administered LNP comprising mRNA encoding Cre recombinase (LNPcre).

The animal model used in this Example is similar to that used in Example 1. Ai14 mice which contain a silent genetic locus encoding red fluorescent protein (Td-Tomato) that can be activated by ‘Cre’ recombinase were used. In these mice, cells only express Td-Tomato if they are successfully transfected with Cre mRNA. Ai14 mice were given an intravenous administration of 0.5 mg/kg LNPcre. As a control, mice were injected with Tris/sucrose. 48 hours after administration of the LNP or control, mice were euthanized and bone marrow cells were harvested and plated in methylcellulose-based medium enriched with recombinant cytokines/growth factors (interleukin-3, interleukin-6, insulin, erythropoietin, stem cell factor, transferrin). Plates were imaged for the appearance of colonies using confocal microscopy on days 2, 5, 7, 9, 12 and 14 post plating.

In this Example, ex-vivo colony-forming unit (CFU) assays were used as a readout of functional HSPCs. In this assay, HSPC give rise to multicellular myeloid, granulocyte, and/or erythroid/megakaryocyte colonies that can be quantified and characterized by microscopy.

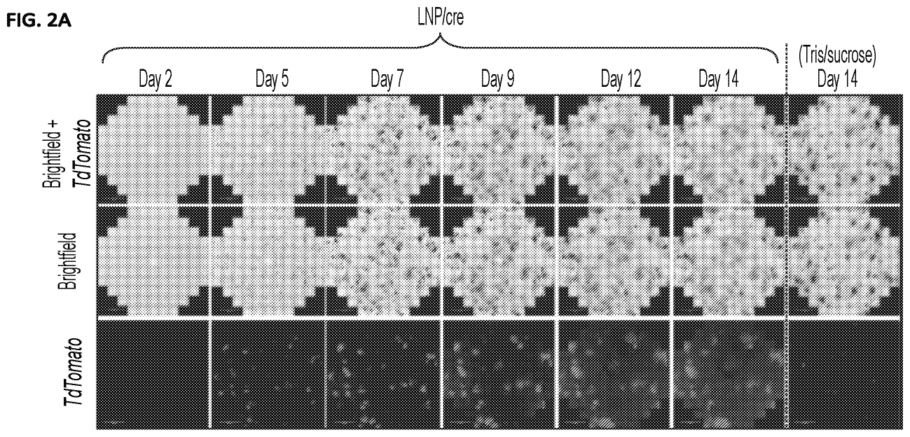

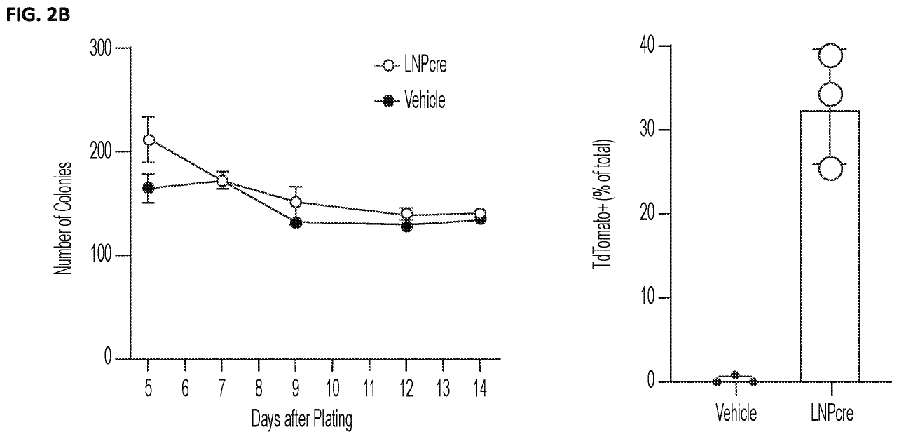

As shown in FIG. 2A, a progressive increase in the number of red fluorescent (TdTomato) CFU in bone marrow cells from mice administered LNPcre was observed starting on day 7 after plating. No TdTomato expressing cells or TdTomato expressing colonies were seen in the control. This data demonstrates that the HSPCs were successfully gene edited in vivo upon administration of LNPcre and led to formation of TdTomato positive colonies ex vivo. The colony forming unit (CFU) counts at different time points and % of TdTomato+cells are depicted in FIG. 2B.

### Example 3: In Vivo Gene Edited Hematopoietic Precursor Cells Give Rise to Myeloid Cells and Lymphoid Cells In Vivo

This Example describes a progressive increase in the frequency of fluorescent (TdTomato) platelets and red blood cells (RBC), and immune cell subsets (neutrophils, monocytes, B cells, CD4+ T cells, and CD8+ T cells) in vivo in the peripheral blood circulation of Ai14 mice administered LNP comprising mRNA encoding Cre recombinase (LNPcre).

A similar animal model as used in Examples 1 and 2 was used in this Example. Ai14 mice which contain a silent genetic locus encoding red fluorescent protein (Td-Tomato) were given an intravenous administration of 0.5 mg/kg LNPcre. Mice were serially bled at indicated days up to ˜240d or ˜8 months post administration and the blood was processed for flow cytometry.

FIGS. 3A-3C show the results of this experiment. In FIG. 3A, an increase in the percent of TdTomato fluorescent platelets from 0.01% at baseline (No LNP group) to ˜30% by day 91 was observed. A similar increase in the percent of TdTomato fluorescent red blood cells was observed: from 0.01% at baseline (No LNP group) to ˜27% by day 91 (FIG. 3B). The increase in TdTomato fluorescent platelets and red blood cells was maintained at least up to 8 months. In other examples the frequency of tdTomato fluorescent platelets and red blood cells were maintained up to 1 year post LNPcre dosing. Since red blood cells and platelets do not have a nucleus and Cre recombinase can only act if genomic DNA is present, this data shows that a progenitor cell type (upstream of red blood cells and platelets) was successfully transfected and gene edited with the LNPcre in vivo. Also, since the presence of TdTomato+platelets and red blood cells observed in blood (˜90d) exceeds the lifespan of platelets and red blood cells in mice (FIG. 3C), which is ˜5-10d and ˜45d, respectively, it further shows that a cell with progenitor activity was targeted by LNPcre and thus giving rise to TdTomato+platelets and red blood cells.

FIG. 3D-3E shows the frequency of TdTomato+myeloid cells, including neutrophils (27±5%, mean±SD) and monocytes (30±5%, mean±SD) up to ˜90d post LNPcre administration. Since circulating neutrophils and monocytes have relatively short lifespans in blood, the presence of up to ˜30% TdTomato+neutrophils and monocytes by 90d (similar to platelets and red blood cells) shows that newly produced myeloid cells are emerging in the periphery as TdTomato+, and that a HSPC upstream of these cells was targeted by the LNP. The level of TdTomato+cells was maintained up to 8 months. In other examples the frequency of tdTomato fluorescent myeloid cells were maintained up to 1 year post LNPcre dosing. FIG. 3F-3G shows progressive increases in the frequency of TdTomato+lymphoid cells, including B cells (13±4%, mean±SD), CD4+ T cells (13±2%, mean±SD), and CD8+ T cells (8±1%, mean±SD) up to ˜90d post LNPcre administration. The progressive increase was maintained at least up to 8 months post LNPcre administration. In other examples the frequency of tdTomato fluorescent lymphoid cells were maintained up to 1 year post LNPcre dosing. Unlike circulating myeloid cells, lymphocyte have a long lifespan and as they gradually turnover, these data show that newly produced lymphocytes are emerging in the periphery as TdTomato+ at an increasing rate. This data further supports in vivo gene editing of hematopoietic stem and precursor cells (HSPC) with LNPs.

### Example 4: Evaluation of Stemness Potential of In Vivo Gene Edited HSPCs

In this Example, the stemness potential of in vivo gene edited HSPCs can be determined.

For this experiment, primary and secondary bone marrow transplants were performed. For the primary BM transplants, Ai14 mice administered LNPcre were serially bled on days 7, 14, 21, 28, and 35. The mice are then to be sacrificed at 5 weeks (day 35) post LNPcre administration and various hematopoietic organs are be harvested, including bone marrow (BM). The isolated BM cells from the Ai14 mice are transplanted into CD45.1 hosts which were lethally irradiated to deplete the host hematopoietic compartment. The recipient CD45.1 mice were serially bled on days 14, 28, 42, 56, 84, 112, 140 and up to 20 wks to monitor hematopoietic cell repopulation from the donor Ai14 mice. In some examples the recipient CD45.1 mice were bled up to 1 year post BM transplantation. In some examples the frequency of tdTomato fluorescent platelets, red blood cells, myeloid and lymphoid cells were maintained up to 1 year post primary BM transplantation.

For the secondary transplants, a subset of the first cohort of CD45.1 recipient mice who received a primary BM transplant of Ai14 BM cells, were sacrificed at 8 weeks (or up to 20 wks) post administration and various hematopoietic organs were harvested, including bone marrow. The isolated BM cells from the CD45.1 primary transplant mice (which contain engrafted HSPC from the original Ai14 donor) were transplanted into secondary lethally irradiated CD45.1 hosts. The recipient CD45.1 mice were serially bled on days 14, 28, 42, and 56 to monitor hematopoietic cell repopulation from the donor CD45.1 primary transplant mice.

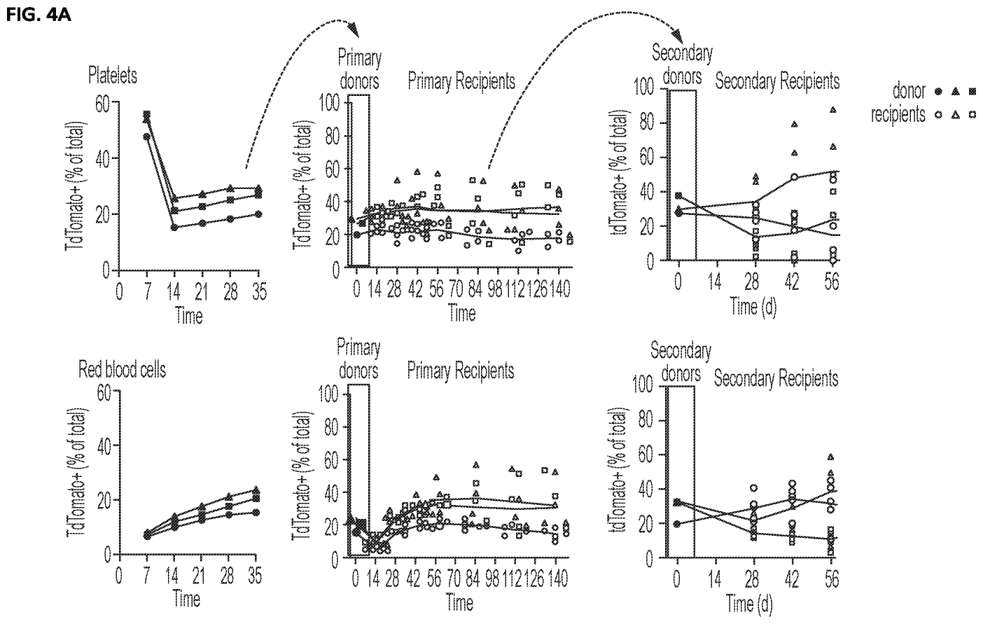

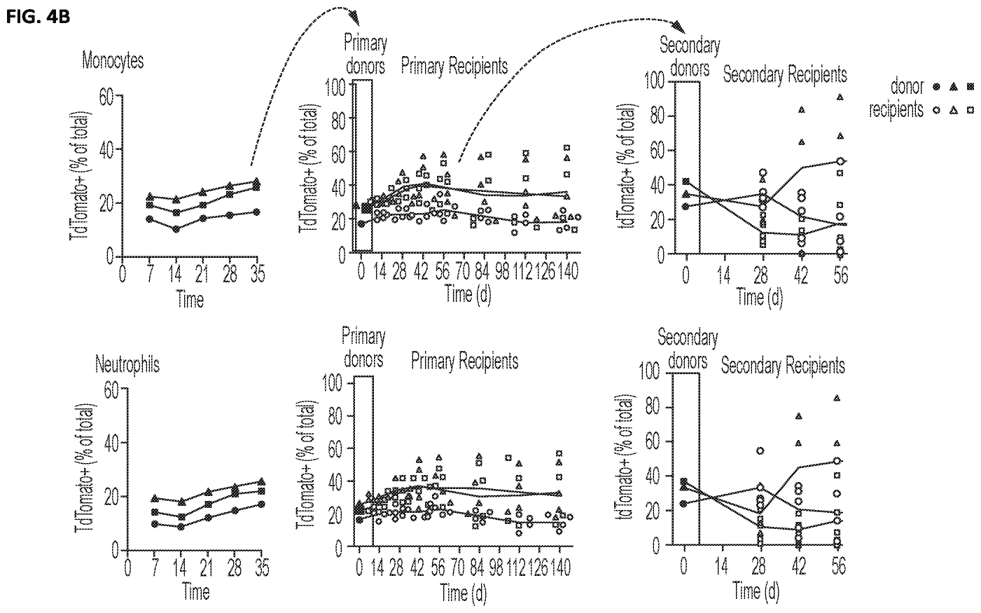

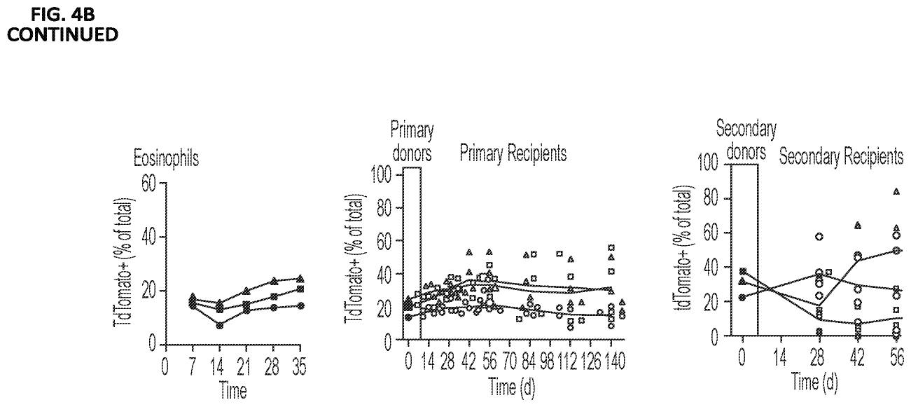

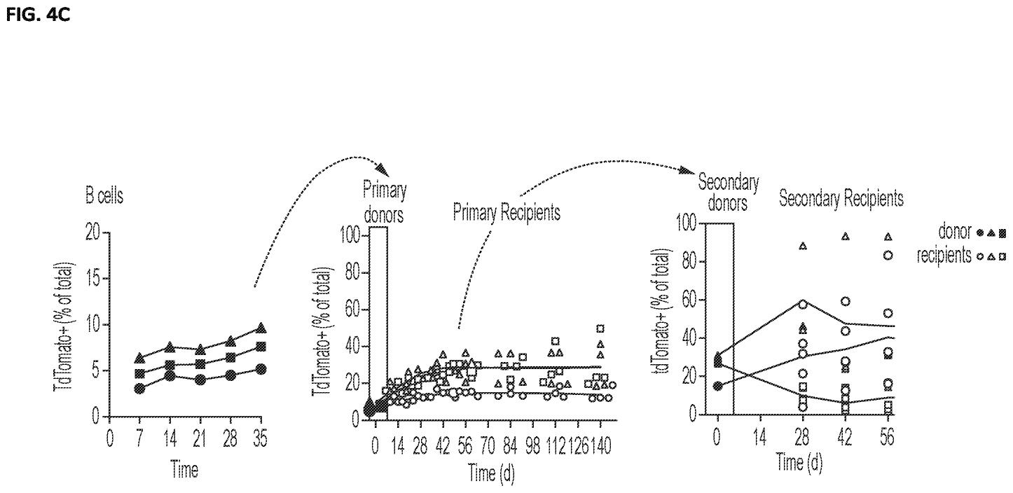

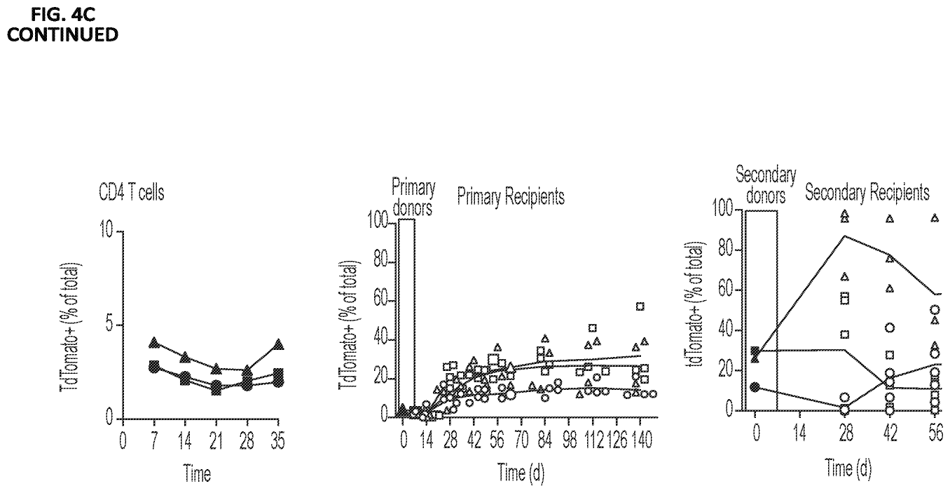

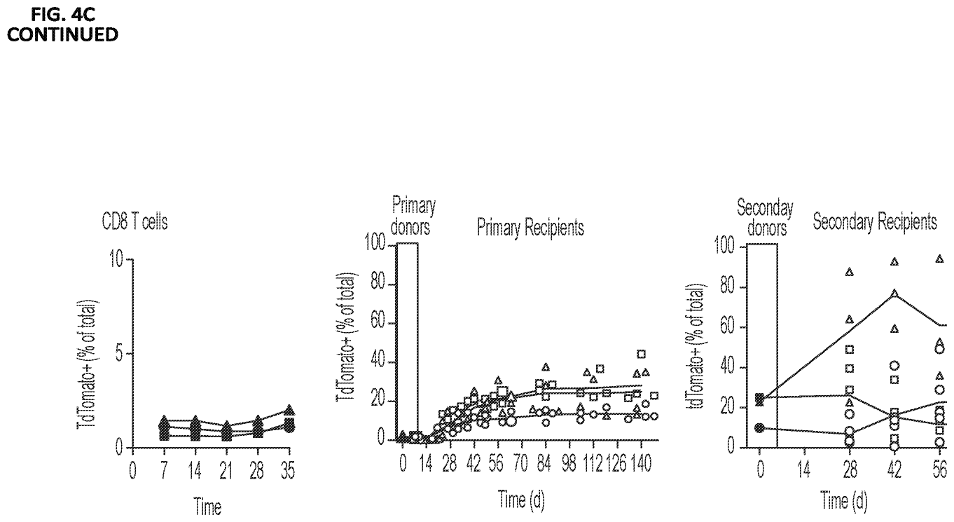

As shown in FIGS. 4A-4C, full hematopoietic reconstitution upon serial bone marrow transplantation provides evidence of in vivo LNPcre delivery to bona fide LT-HSC.

The methods described in this example can be used to evaluate the stemness potential of in vivo gene edited HSPCs.

### Example 5: Multiple Dosing of LNP Comprising mRNA Encoding Cre Recombinase Leads to an Additive Cumulative Effect on HSPC Delivery in Ai14 Mice

This Example describes in vivo transfection and Cre-mediated gene editing of hematopoietic stem and progenitor cells (HSPCs) by repeated dosing of LNP formulated with Cre-mRNA (LNPcre). Ai14 mice were given 1, 3, or 5 intravenous administrations of 0.5 mg/kg LNPcre.

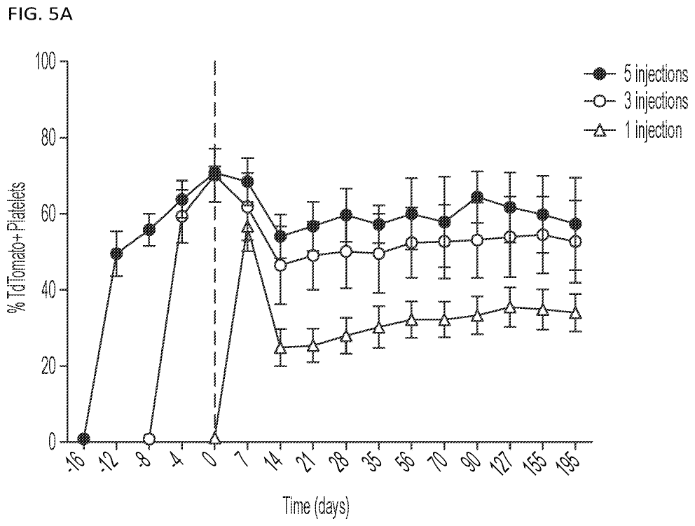

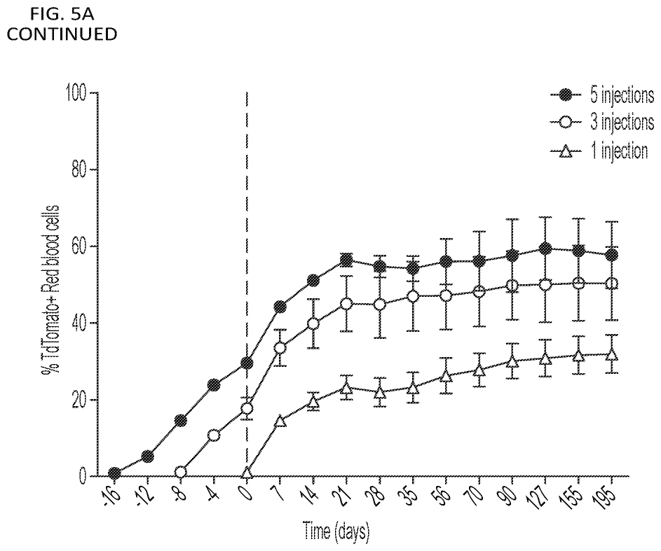

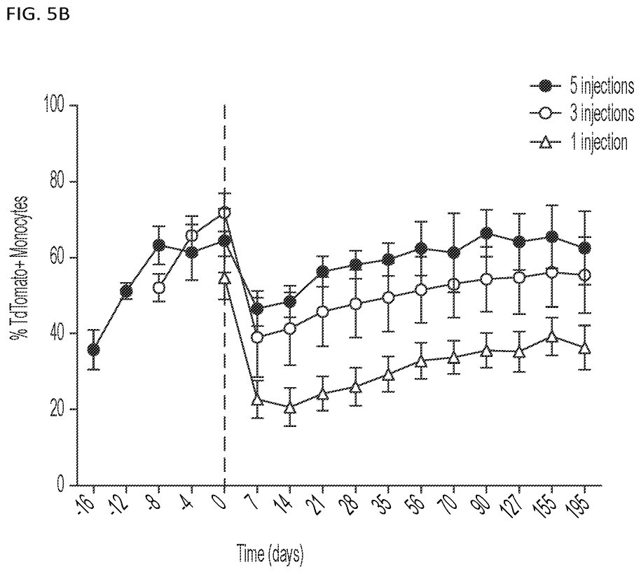

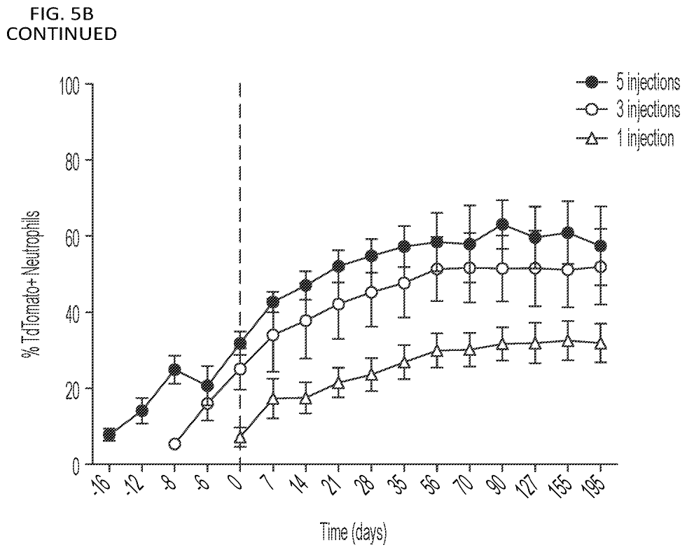

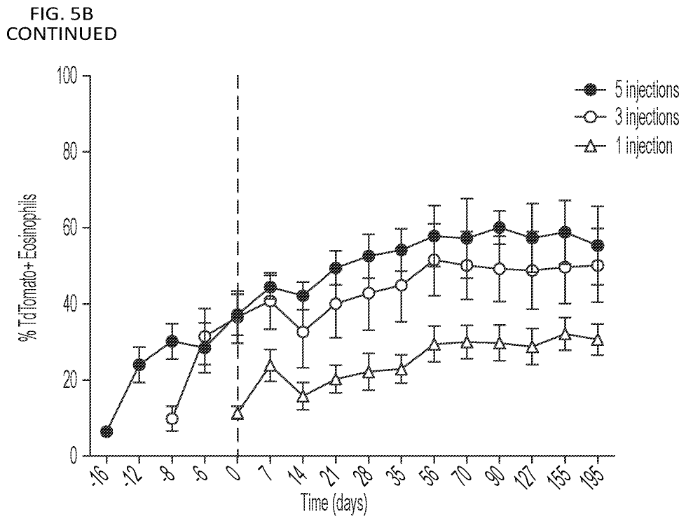

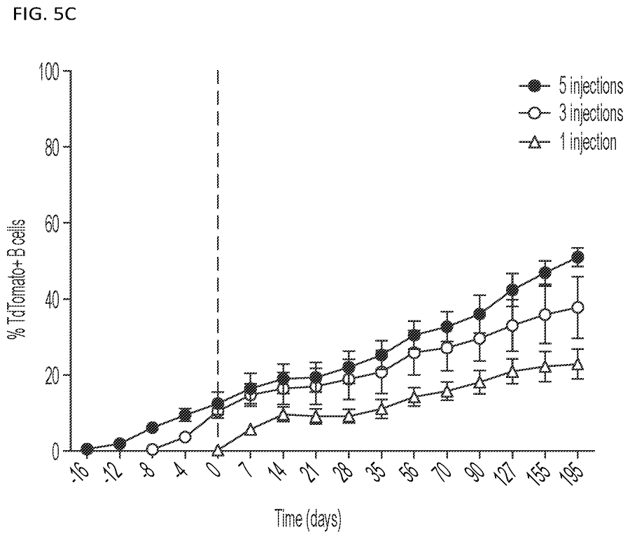

All mice were serially bled at 7d, 14d, 21d, 28d, 35d, 56d, 70d, 90d, 127d, 155d, up to ˜195d or 6 months following the last administration of the LNP and the blood was processed for flow cytometry. In some examples mice were bled at 1 year post multiple LNP dosing. Frequency plots in FIGS. 5A-5C depict the % tdT⁺ cells circulating among platelets and red blood cells (FIG. 5A), myeloid cells (FIG. 5B), including monocytes, neutrophils, and eosinophils, and lymphocytes (FIG. 5C), including B cells, CD4 T cells, and CD8 T cells. In some examples, the frequency of tdTomato fluorescent platelets, red blood cells, myeloid and lymphoid cells were maintained up to 1 year post multiple LNP dosing.

### Example 6: Delivery of LNP-mOX40L mRNA to Bone Marrow HSPC in Non-Human Primates

This Example describes in vivo transfection in non-human primates (NHP) by administration of LNP formulated with mOX40L mRNA.

Each non-human primate was administered an intravenous injection of 0.5 mg/kg LNP-mOX40L mRNA or OX40L vehicle alone. Bone marrow was collected 24 h post injection and processed for flow cytometry.

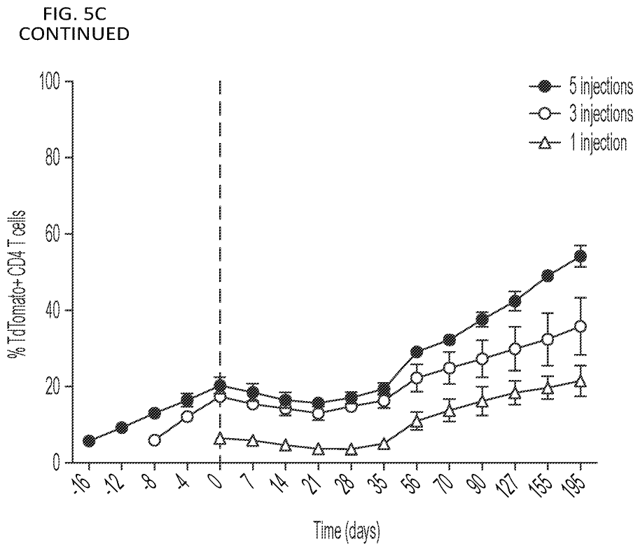

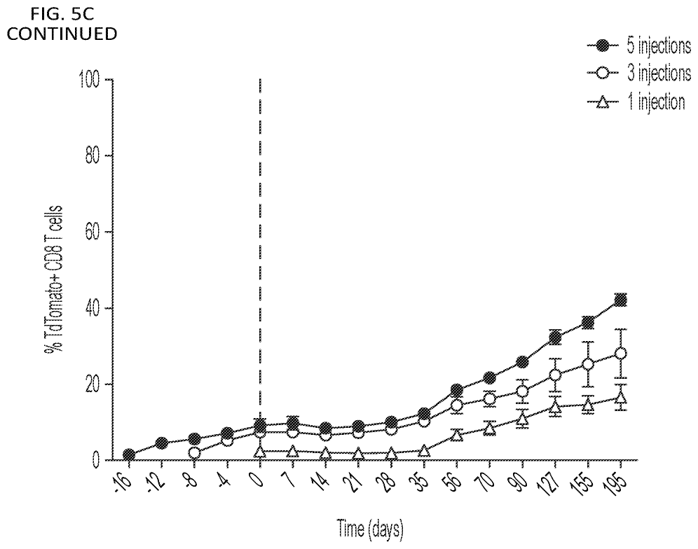

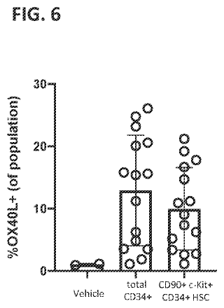

FIG. 6 summarizes the frequency (%) of mOX40L in HSPC subsets among bone marrow cells following LNP delivery.

### Example 7: Delivery of LNP-mOX40L mRNA to Human HSPC in Humanized Mice

This Example describes in vivo delivery of LNP formulated with mOX40L mRNA to human HSPC in humanized mice.

NOG-EXL mice were reconstituted with human CD34⁺ cord blood cells to produce humanized mice. Humanized mice were administered LNP-mOX40L mRNA by intravenous injection at 0.5 mg/kg 12-14 weeks post-engraftment. Mice were sacrificed 24 h post-administration for analyses.

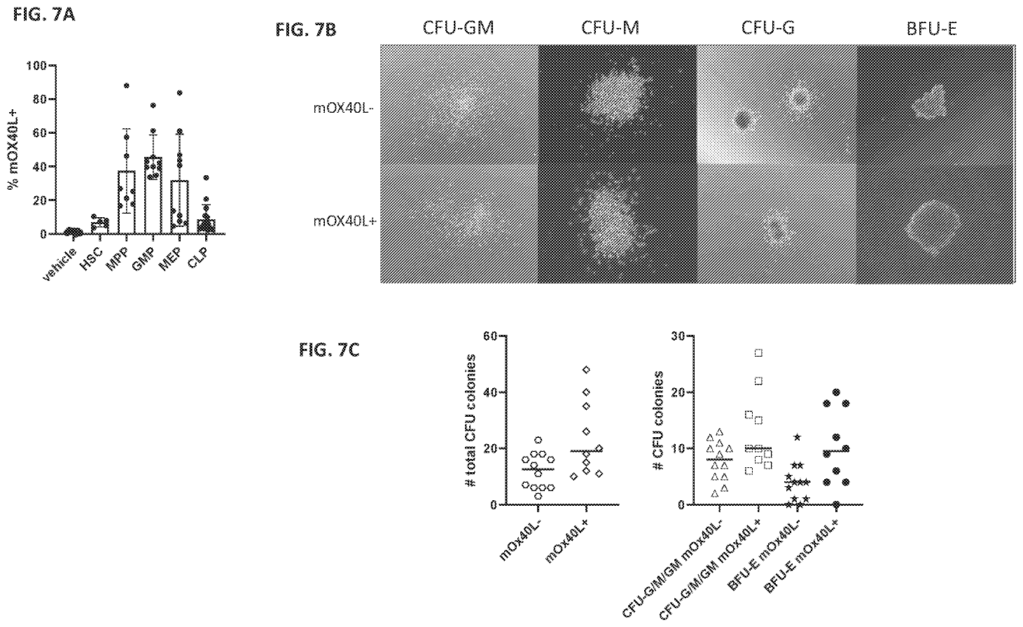

FIG. 7A summarizes the frequency (%) of OX40L expression in human HSPC subsets. Each point denotes data from a single humanized mouse. Representative images of CFU assays after plating FACS-sorted mOX40L⁺ and mOX40L⁻ human CD34⁺ progenitors from bone marrow of LNP-injected humanized mice are FIG. 7B. FIG. 7C summarizes the results of the CFU assays graphically.

### Example 8: Preparation of Ionizable Lipids

Ionizable lipids (e.g., amino lipids) of the present disclosure were prepared using known methods. For example, Compounds (I-I) and (I-II) were prepared according to methods described in WO 2017/049245 (see, e.g., paragraphs [00426], [00427], and [00434]), which is incorporated herein by reference in its entirety. Compound (I-III) was prepared according to methods described in WO 2018/170306 (see, e.g., paragraphs [0259], [0260] and [0387]), which is incorporated herein by reference in its entirety. Compound (II-I) was prepared according to methods described in WO 2017/112865 (see, e.g., paragraphs [0575]-[0583]), which is incorporated herein by reference in its entirety.

Compound (I-IV) was prepared according to the following methods and representative procedures.

### Representative Synthesis of Compound d1

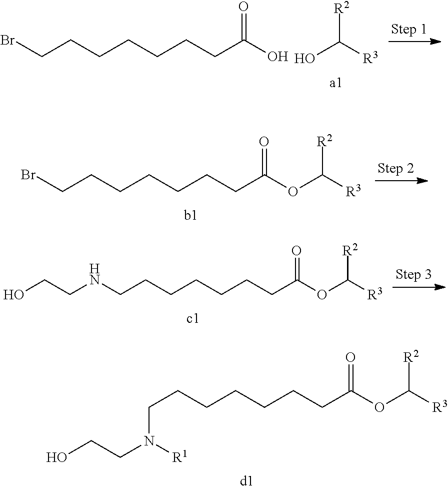

8-bromooctanoic acid was reacted with an alcohol al (e.g., heptadecan-9-ol) to afford an ester b1 (e.g., heptadecan-9-yl 8-bromooctanoate). Step 1 can take place in an organic solvent (e.g., dichloromethane) in the presence of, e.g., N-(3-dimethylaminopropyl)-N′-ethylcarbodiimide hydrochloride, N,N-diisopropylethylamine and DMAP. Step 1 can take place at room temperature for 18 h. Next, ester b1 can be reacted with 2-aminoethan-1-ol to afford amine cl (e.g., heptadecan-9-yl 8-((2-hydroxyethyl)amino)octanoate). Step 2 can take place in ethanol at, e.g., a temperature of about 60° C. Then amine cl can be reacted with a bromoalkyl R¹—Br (e.g., 1-bromotetradecane) to afford compound d1 (e.g., heptadecan-9-yl 8-((2-hydroxyethyl)(tetradecyl)amino)octanoate). Step 3 can take place in ethanol in the presence of a base (e.g., N,N-diisopropylethylamine).

### Synthesis of 2-octyldecanoic acid

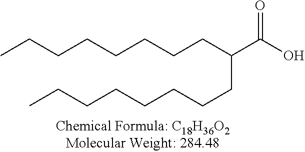 [SMILES: CCCCCCCCC(CCCCCCCC)C(=O)O]

A solution LDA was prepared by cooling diisopropylamine (2.92 mL, 20.8 mmol) in tetrahydrofuran (10 mL) to −78° C. and treating the solution with n-BuLi (7.5 mL, 18.9 mmol, 2.5 M in hexanes), then warming the mixture to 0° C. A separate solution of decanoic acid (2.96 g, 17.2 mmol) and NaH (754 mg, 18.9 mmol, 60% w/w) in THE (20 mL) at 0° C. was then added to the solution of LDA and the mixture was stirred at room temperature for 30 min. After this time 1-iodooctane (5 g, 20.8 mmol) was added and the reaction mixture was heated at 45° C. for 6 h. The reaction was quenched with 1N HCl (10 mL). The organic layer was dried over MgSO₄, filtered and evaporated under vacuum. The residue was purified by silica gel chromatography (0-20% ethyl acetate in hexanes) to afford 2-octyldecanoic acid (1.9 g, 6.6 mmol). ¹H NMR (300 MHz, CDCl₃) δ: ppm 2.38 (br. m, 1H); 1.74-1.03 (br. m, 28H); 0.91 (m, 6H).

### Synthesis of 2-octyldecanol

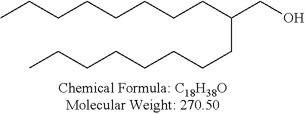 [SMILES: CCCCCCCCC(CO)CCCCCCCC]

A solution of 2-octyldecanoic acid (746 mg, 2.6 mmol) in dry THE (12 mL) was added to a stirred solution of LAH (5.2 mL, 5.2 mmol, 1M solution in THF) in dry THE (6 mL) under nitrogen at 0° C. The reaction was warmed to room temperature and stirred for 12 h. A solution of saturated Na₂SO₄*10H₂O solution (10 mL) was added. The solids were filtered through a plug of Celite. The filtrate was evaporated under vacuum and the residue was purified by silica gel chromatography (0-20% ethyl acetate in hexanes) to yield 2-octyldecan-1-ol (635 mg, 2.3 mmol). ¹H NMR (300 MHz, CDCl₃) δ: ppm 3.55 (d, 2H); 1.57-1.18 (m, 30H); 0.91 (m, 6H).

### Representative Synthesis of 3-butylnonyl 8-bromooctanoate

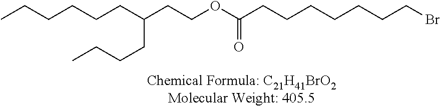 [SMILES: CCCCCCC(CCCC)CCOC(=O)CCCCCCCBr]

To a solution of 3-butylnonan-1-ol (458 mg, 2.28 mmol), 8-bromooctanoic acid (611.9 mg, 2.74 mmol) and DMAP (55.9 mg, 0.46 mmol) in dichloromethane (30 mL) at 0° C. was added EDCI (657.3 mg, 3.43 mmol) and the reaction mixture stirred at room temperature overnight. The reaction mixture was cooled to 0° C. and 1N hydrochloric acid (3 mL) was added slowly, then the mixture was extracted with diethyl ether (100 mL) and the layers were separated. The organic layer washed with saturated sodium bicarbonate (100 mL), water and brine. The organic layer was separated and concentrated. The crude was purified by flash chromatography (SiO₂: hexane/diethyl ether 0-100%) to afford 3-butylnonyl 8-bromooctanoate (680 mg. 73%). ¹H NMR (300 MHz, CDCl₃): δ ppm 4.07 (t, 2H, J=6.8 Hz); 3.39 (t, 2H, J=6.8 Hz); 2.28 (t, 2H, J=7.6 Hz); 1.88-1.79 (m, 2H); 1.70-1.42 (m, 6H); 1.38-1.17 (m, 21H); 0.88-0.82 (m, 6H).

### Representative Synthesis of 3-propylhexyl 8-((2-hydroxyethyl)amino)octanoate

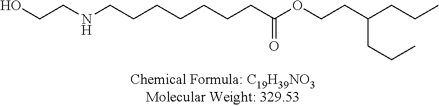 [SMILES: CCCC(CCC)CCOC(=O)CCCCCCCNCCO]

To a round bottom flask equipped with a stir bar was added 3-propylhexyl 8-bromooctanoate (2.82 g, 8.06 mmol), ethanolamine (14.6 mL, 242 mmol), and ethyl alcohol (6 mL). The resulting mixture was stirred at 40° C. for 16 h. The reaction was diluted with dichloromethane, washed with water (2×), and the layers were separated. The organic layer was dried (MgSO₄), filtered and concentrated. The crude material was purified by silica gel chromatography (0-5-10-25-50-100% (mixture of 1% NH40H, 20% MeOH in dichloromethane) in dichloromethane) to afford 3-propylhexyl 8-((2-hydroxyethyl)amino)octanoate (876 mg, 2.66 mmol, 33%) as an oil. ¹H NMR (300 MHz, CDCl₃) δ: ppm 4.08 (t, 2H, J=6.0 Hz); 3.63 (t, 2H, J=6.0 Hz); 2.77 (t, 2H, J=6.0 Hz); 2.61 (t, 2H, J=6.0 Hz); 2.28 (t, 2H, J=6.0 Hz); 1.91 (br. s, 2H); 1.68-1.39 (m, 7H); 1.38-1.18 (m, 14H); 0.88 (t, 6H, J=6.0 Hz).

### Synthesis of 3-butylheptan-1-ol

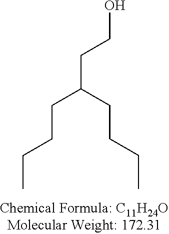

To a mixture of lithium aluminum hydride (850 mg, 22.4 mmol) in dry ether (23 mL) under N₂ at 0° C., was added dropwise ethyl 3-butylheptanoate (4.00 g, 18.7 mmol) in dry ether (15 mL). The mixture was stirred at room temperature for 2.5 h prior to being cooled to 0° C. Water (1 mL per g of LiAlH₄) was added to the solution dropwise, followed by the slow addition of 15% sodium hydroxide (1 mL per g of LiAlH₄) and water (3 mL per g of LiAlH₄). The solution was stirred for a few minutes at room temperature and filtered through a Celite pad. The Celite pad was washed with diethyl ether and the filtrate was concentrated. The crude material was purified by silica gel chromatography (0-40% EtOAc:hexanes) to afford 3-butylheptan-1-ol (3.19 g, 18.5 mmol, 99%) as an oil. ¹H NMR (300 MHz, CDCl₃) δ: ppm 3.66 (t, 2H, J=6.0 Hz); 1.53 (q, 2H, J=6.0 Hz); 1.46-1.36 (m, 1H); 1.35-1.21 (m, 12H); 1.18 (br. s, 1H); 0.89 (br. t, 6H, J=6.0 Hz).

### Synthesis of 3-butylheptyl 8-bromooctanoate

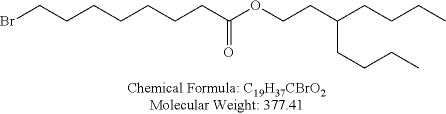 [SMILES: CCCCC(CCCC)CCOC(=O)CCCCCCCBr]

To a solution of 3-butylheptan-1-ol (3.19 g, 18.5 mmol), 8-bromooctanoic acid (4.96 g, 22.2 mmol), and DMAP (453 mg, 3.71 mmol) in methylene chloride (32 mL) at 0° C. was added EDCI (5.33 g, 27.8 mmol) and the reaction mixture stirred at room temperature overnight. The reaction mixture was then cooled to 0° C. and a solution of 10% hydrochloric acid (150 mL) was added slowly over 20 minutes. The layers were separated, and the organic layer was concentrated in vacuum to give a crude oil. The oil was dissolved in hexane (150 mL) and washed with a mixture of acetonitrile (150 mL) and 5% sodium bicarbonate (150 mL). The hexane layer was separated, dried (MgSO₄), and filtered. The solvent was removed under vacuum to give 3-butylheptyl 8-bromooctanoate (6.90 g, 18.3 mmol, 99%) as an oil. The compound was carried onto the next step without further purification. ¹H NMR (300 MHz, CDCl₃) δ: ppm 4.08 (t, 2H, J=6.0 Hz); 3.40 (t, 2H, J=6.0 Hz); 2.29 (t, 2H, J=6.0 Hz); 1.85 (pent., 2H, J=6.0 Hz); 1.69-1.52 (m, 4H); 1.49-1.20 (m, 19H); 0.89 (br. t, 6H, J=6.0 Hz).

### Representative Synthesis of 3-butylnonyl 8-((8-(heptadecan-9-yloxy)-8-oxooctyl)(2-hydroxyethyl)amino)octanoate

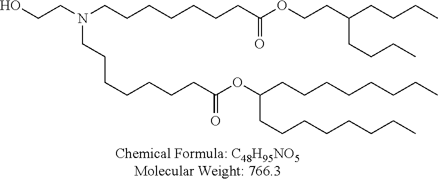 [SMILES: CCCCCCCCC(CCCCCCCC)OC(=O)CCCCCCCN(CCO)CCCCCCCC(=O)OCCC(CCCC)CCCC]

In a 500 mL round bottom flask connected with condenser, heptadecan-9-yl 8-((2-hydroxyethyl)amino)octanoate (601 mg, 1.36 mmol), 3-butylnonyl 8-bromooctanoate (606 mg, 1.49 mmol), potassium carbonate (676 mg, 4.9 mmol) and potassium iodide (248.4 mg, 1.49 mmol) were mixed in cyclopentylmethyl ether (30 mL) and acetonitrile (30 mL), and the reaction mixture was heated to 85° C. for 18 h. MS showed clean conversion, and the mixture was cooled to room temperature and diluted with hexanes. The mixture was filtered through pad of Celite. After washing with hexanes, the filtrate was concentrated to give brown oil which was purified by flash chromatography (SiO₂: hexane/diethyl ether 0-100%) to afford 3-butylnonyl 8-((8-(heptadecan-9-yloxy)-8-oxooctyl)(2-hydroxyethyl)amino)octanoate as an oil (588 mg. 56%). HPLC/ELSD: RT=7.07 min. MS(CI): m/z (MH⁺) 766.7 for C₄₈H₉₅NO₅. ¹H NMR (300 MHz, CDCl₃) δ: ppm 4.85 (quint., 1H, J=6.1 Hz); 4.07 (t, 2H, J=6.9 Hz); 3.50 (t, 2H, J=5.5 Hz); 2.98 (bs, 1H); 2.55 (t, 2H, J=5.2 Hz); 2.41 (t, 4H, J=7.4 Hz); 2.26 (t, 4H, J=7.4 Hz); 1.65-1.48 (m, 19H); 1.26 (br. m, 48H); 0.88-0.84 (m, 12H).

### Synthesis of heptadecan-9-yl 8-((2-hydroxyethyl)amino)octanoate

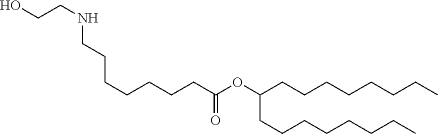 [SMILES: CCCCCCCCC(CCCCCCCC)OC(=O)CCCCCCCNCCO]

To a solution of heptadecan-9-yl 8-bromooctanoate (10 g, 21.67 mmol) and ethanolamine (39.7 g, 650 mmol) in ethanol (5 mL) was heated to 65° C. for 16 h. The reaction was cooled to room temperature, dissolved in ethyl acetate, and extracted with water (4×). The organic layer was separated, washed with brine, dried with sodium sulfate, filtered and the solvent evaporated under vacuum. The residue was purified by flash chromatography (ISCO), eluting with a solution of 20% methanol/80% dichloromethane/1% ammonium hydroxide and dichloromethane, 0-100% gradient, to obtain heptadecan-9-yl 8-((2-hydroxyethyl)amino)octanoate (7.85 g, 82%). UPLC/ELSD: RT=2.06 min. MS (ES): m z (MH⁺) 442.689 for C₂₇H₅₅NO₃. ¹H NMR (300 MHz, CDCl₃) δ: ppm 4.89 (p, 1H); 3.66 (t, 2H); 2.79 (t, 2H); 2.63 (m, 2H); 2.30 (t, 2H); 1.77-1.20 (m, 40H); 0.90 (m, 6H).

### Synthesis of 3-butylheptyl 8-((8-(heptadecan-9-yloxy)-8-oxooctyl)(2-hydroxyethyl)amino)octanoate (I-IV)

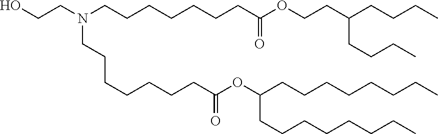 [SMILES: CCCCCCCCC(CCCCCCCC)OC(=O)CCCCCCCN(CCO)CCCCCCCC(=O)OCCC(CCCC)CCCC]

A solution of 3-butylheptyl 8-bromooctanoate (6.15 g, 16.31 mmol) and heptadecan-9-yl 8-[(2-hydroxyethyl)amino]octanoate (6.86 g, 15.53 mmol) in a mixture of cyclopentylmethyl ether (15 mL) and acetonitrile (6 mL) was treated with potassium carbonate (8.59 g, 62.12 mmol) and potassium iodide (2.84 g, 17.08 mmol), and the reaction was stirred at 77° C. for 16 h. The reaction was cooled and filtered, and the volatiles were evaporated under vacuum. The residue was purified by flash chromatography (ISCO) eluting with a solution of 20% methanol/80% dichloromethane/1% ammonium hydroxide in dichloromethane, 0-100% gradient, to obtain 3-butylheptyl 8-((8-(heptadecan-9-yloxy)-8-oxooctyl)(2-hydroxyethyl)amino)octanoate (4.53 g, 37.8%). UPLC/ELSD: RT=3.04 min. MS (ES): m z (MH⁺) 739.464 for C₄₆H₉₁NO₅. ¹H NMR (300 MHz, CDCl₃) δ: ppm 4.89 (p, 1H); 4.11 (m, 2H), 3.57 (bm, 2H); 2.73-2.39 (m, 6H); 2.30 (m, 4H); 1.72-1.17 (m, 64H); 0.92 (m, 12H).

### OTHER EMBODIMENTS

It is to be understood that while the present disclosure has been described in conjunction with the detailed description thereof, the foregoing description is intended to illustrate and not limit the scope of the present disclosure, which is defined by the scope of the appended claims. Other aspects, advantages, and alterations are within the scope of the following claims. All references described herein are incorporated by reference in their entireties.

What is claimed is:

1. A method of modifying a stem or progenitor cell, e.g., a hematopoietic stem and progenitor cell (HSPC), e.g., modifying a parameter associated with the cell or a component associated with the cell, comprising contacting the cell with a lipid nanoparticle (LNP) composition comprising a payload, thereby modifying the cell.

2. A method of treating a subject having a disease, a disorder, a mutation, or a single nucleotide polymorphism (SNP), comprising administering to the subject an effective amount of an LNP composition comprising a payload, wherein said LNP composition results in a modification of a stem cell, e.g., a hematopoietic stem and progenitor cell (HSPC), in the subject, e.g., modification of a component associated with the cell or a parameter associated with the cell, thereby treating the subject.

3. A method of ameliorating a symptom of a subject having a disease, a disorder, a mutation, or a single nucleotide polymorphism (SNP), comprising administering to the subject an effective amount of an LNP composition comprising a payload, wherein said LNP composition results in a modification of a stem cell, e.g., a hematopoietic stem and progenitor cell (HSPC), in the subject, e.g., modification of a component associated with the cell or a parameter associated with the cell, thereby ameliorating the symptom of the subject.

4. A method of delivering an LNP composition comprising a payload to a cell (e.g., stem or progenitor cell), or tissue, e.g., in a subject, comprising contacting the cell, or tissue with the LNP composition.

5. The method of any one of the preceding claims, wherein the LNP composition results in a modification of the cell, or tissue, e.g., a component associated with the cell or tissue, or a parameter associated with the cell or tissue.

6. The method of any one of the preceding claims, wherein the component comprises: (1) a nucleic acid associated with the cell or fragment thereof, e.g., DNA (e.g., exonic, intronic, intergenic, telomeric, promoter, enhancer, insulator, repressor, coding, or non-coding) or RNA (e.g., mRNA, rRNA, tRNA, regulatory RNA, non-coding RNA, long non-coding RNA (lncRNA), guide RNA (gRNA), piwi-interacting RNA (piRNA), small nucleolar RNA (snoRNA), small nuclear RNA (snRNA), extracellular RNA (exRNA), small Cajal body-specific RNA (scaRNA), micro RNA (miRNA), circular RNA, or an RNAi molecule, e.g., small interfering RNA (siRNA) or small hairpin RNA (shRNA)); (2) a peptide or protein associated with the cell or fragment thereof; (3) a lipid component associated with the cell or fragment thereof, or a combination thereof.

7. The method of any one of the preceding claims, wherein the component is endogenous to the cell.

8. The method of any one of the preceding claims, wherein the component is exogenous to the cell, e.g., has been introduced into the cell by a method known in the field, e.g., transformation, electroporation, viral-based delivery or lipid-based delivery.

9. The method of any one of the preceding claims, wherein the parameter comprises a genotypic parameter, a phenotypic parameter. a functional parameter, an expression parameter, or a signaling parameter.

10. The method of any one of the preceding claims, wherein the genotypic parameter comprises a genotype of the cell, e.g., the presence or absence a gene or allele, or a modification of a gene or allele, e.g., a germline or somatic mutation, or a polymorphism, in the gene or allele.

11. The method of any one of the preceding claims, wherein the phenotypic parameter comprises a phenotype of the cell, e.g., expression and/or activity of a molecule, e.g., cell surface protein, lipid or adhesion molecule, on the surface of the cell.

12. The method of any one of the preceding claim, wherein the functional parameter comprises a biological function of the cell, e.g., the ability of the cell to produce a gene product (e.g., a protein), the ability of the cell to proliferate, divide, and/or renew, and/or the ability of the cell to differentiate, e.g., into one or more cell types in a lineage.

13. The method of any one of the preceding claims, wherein the expression parameter comprises one, two, three, four or all of the following:
(a) expression level (e.g., of polypeptide or protein, or polynucleotide or nucleic acid, e.g., mRNA);
(b) activity (e.g., of polypeptide or protein, or polynucleotide or nucleic acid, e.g., mRNA),
(c) post-translational modification of polypeptide or protein;
(d) folding (e.g., of polypeptide or protein, or polynucleotide or nucleic acid, e.g., mRNA), and/or
(e) stability (e.g., of polypeptide or protein, or polynucleotide or nucleic acid, e.g., mRNA).

14. The method of any one of the preceding claims, wherein the signaling parameter comprises one, two, three, four or all of the following:
(1) modulation of a signaling pathway, e.g., a cellular signaling pathway;
(2) cell fate modulation;
(3) modulation of expression level (e.g., of polypeptide or protein, or polynucleotide or nucleic acid, e.g., mRNA);
(4) modulation of activity (e.g., of polypeptide or protein, or polynucleotide or nucleic acid, e.g., mRNA), and/or
(5) modulation of stability e.g., of polypeptide or protein, or polynucleotide or nucleic acid, e.g., mRNA).

15. The method of any one of the preceding claims, wherein the cell is contacted in vitro, in vivo or ex vivo with the LNP composition.

16. The method of any one of the preceding claims, wherein the cell is contacted in vivo with the LNP formulation.

17. The method of any one of the preceding claims, wherein the cell is a stem or progenitor cell, e.g., a hematopoietic stem and progenitor cell (HSPC), e.g., an HSPC derived from an embryonic stem or progenitor cell or an HSPC derived from an induced pluripotent stem or progenitor cell.

18. The method of any one of the preceding claims, wherein the cell is an HSPC, e.g., a multipotent HSC or multipotent HPC.

19. The method of any one of the preceding claims, wherein the HSPC has one, two, three, four, five or all of the following functional characteristics:
i. ability to self-renew;
ii. unlimited proliferative potential;
iii. ability to enter and/or exit a quiescent state, e.g., a cell state where no proliferation occurs, e.g., GO phase of the cell cycle;
iv. ability to differentiate into any hematopoietic lineage, e.g., myeloid and/or lymphoid lineages, e.g., common lymphoid progenitor (CLP) or a differentiated cell thereof; and/or common myeloid progenitor (CMP) or a differentiated cell thereof;
v. ability to repopulate any hematopoietic lineage, e.g., myeloid and/or lymphoid lineages, e.g., common lymphoid progenitor (CLP) or a differentiated cell thereof, and/or common myeloid progenitor (CMP) or a differentiated cell thereof; e.g., in an organism;
vi. ability to form colony forming units (CFU).

20. The method of any one of the preceding claims, wherein the HSPC has one, two, three, four, five, six, seven, eight, or all of the following expression characteristics:
i. expression of CD45, e.g., detectable expression of CD45, e.g., cell surface expression of CD45;
ii. expression of CD34, e.g., detectable expression of CD34, e.g., cell surface expression of CD34;
iii. expression of CD38, e.g., detectable expression of CD38, e.g., cell surface expression of CD38;
iv. expression of CD90 e.g., detectable expression of CD90, e.g., cell surface expression of CD90;
v. expression of CD133 e.g., detectable expression of CD133, e.g., cell surface expression of CD133;
vi. expression of CD45RA, e.g., detectable expression of CD45RA, e.g., cell surface expression of CD45RA;
vii. no detectable or low expression of markers associated with primitive progenitor cells, e.g., CMP, MEP, GMP and/or CLP;
viii. no detectable or low expression of markers associated with lineage committed cells, e.g., TCP, NKP, GP, MP, EP and/or MkP; or
ix. no detectable or low expression of markers associated with one, two or all cell lineage markers of (vii)-(viii), e.g., lineage negative (Lin-).

21. The method of any one of the preceding claims, wherein the modified cell (e.g., population of modified cells) is a modified HSPC (e.g., a population of modified HSPCs).

22. The method of claim 21, wherein the modified HSPC has one, two, three, four, five or all of the following functional characteristics:
i. ability to self-renew;
ii. unlimited proliferative potential;
iii. ability to enter and/or exit a quiescent state, e.g., a cell state where no proliferation occurs, e.g., GO phase of the cell cycle;
iv. ability to differentiate into any hematopoietic lineage, e.g., myeloid and/or lymphoid lineages, e.g., common lymphoid progenitor (CLP) or a differentiated cell thereof, and/or common myeloid progenitor (CMP) or a differentiated cell thereof;
v. ability to repopulate any hematopoietic lineage, e.g., myeloid and/or lymphoid lineages, e.g., common lymphoid progenitor (CLP) or a differentiated cell thereof, and/or common myeloid progenitor (CMP) or a differentiated cell thereof, e.g., in an organism; or
vi. ability to form colony forming units (CFU).

23. The method of claim 22, wherein the modified HSPC has the ability to form CFU, e.g., as measured in an ex-vivo colony-forming unit (CFU) assay, e.g., as described in Example 2, e.g., as compared to an otherwise similar HSPC which has not been contacted with an LNP, or has been contacted with a different LNP.

24. The method of claim 22, wherein the modified HSPC has the ability to differentiate into myeloid cells, e.g., as measured in an ex-vivo colony-forming unit (CFU) assay, e.g., as described in Example 2, or as measured in lineage tracing experiments, e.g., as described in Example 3, e.g., as compared to an otherwise similar HSPC which has not been contacted with an LNP, or has been contacted with a different LNP.

25. The method of claim 22, wherein the modified HSPC has the ability to differentiate into lymphoid cells, e.g., as measured in lineage tracing experiments, e.g., as described in Example 3, e.g., as compared to an otherwise similar HSPC which has not been contacted with an LNP, or has been contacted with a different LNP.

26. The method of claim 22, wherein the modified HSPC differentiates into a myeloid cell or a lymphoid cell in vivo.

27. The method of claim 22, wherein the modified HSPC differentiates into a myeloid cell or a lymphoid cell in vitro.

28. The method of claim 22, wherein the modified HSPC has the ability to differentiate into an erythrocyte cell or a platelet, e.g., as measured in lineage tracing experiments, e.g., as described in Example 3, e.g., as compared to an otherwise similar HSPC which has not been contacted with an LNP, or has been contacted with a different LNP.

29. The method of claim 22, wherein the modified HSPC differentiates into an erythrocyte cell or a platelet in vivo.

30. The method of claim 22, wherein the modified HSPC differentiates into an erythrocyte cell or a platelet in vitro.

31. The method of claim 22, wherein the modified HSPC persists, e.g., in vivo, for at least 1, 2, 3, 4, 5, 6, 7, 10, 15, 20, 25, 30, 60, 90, 120, 150, 180, 240, 360, 365 days, or more.

32. The method of claim 31, wherein the in vivo persistence of the modified HSPC results in differentiation into one or more cells, e.g., cells in the myeloid and/or cells in the lymphoid lineage, e.g., as shown in Example 3.

33. The method of any one of claims 21-32, wherein the modified HSPC has one, two, three, four, five, six, seven, eight, or all of the following expression characteristics:
i. expression of CD45, e.g., detectable expression of CD45, e.g., cell surface expression of CD45;
ii. expression of CD34, e.g., detectable expression of CD34, e.g., cell surface expression of CD34;
iii. expression of CD38, e.g., detectable expression of CD38, e.g., cell surface expression of CD38;
iv. expression of CD90 e.g., detectable expression of CD90, e.g., cell surface expression of CD90;
v. expression of CD133 e.g., detectable expression of CD133, e.g., cell surface expression of CD133;
vi. expression of CD45RA, e.g., detectable expression of CD45RA, e.g., cell surface expression of CD45RA;
vii. no detectable or low expression of markers associated with primitive progenitor cells, e.g., CMP, MEP, GMP and/or CLP;
viii. no detectable or low expression of markers associated with lineage committed cells, e.g., TCP, NKP, GP, MP, EP and/or MkP; or
ix. no detectable or low expression of markers associated with one, two or all cell lineage markers of (vii)-(viii), e.g., lineage negative (Lin-).

34. The method of any one of the preceding claims, wherein the LNP composition comprising the payload modifies, e.g., increases or decreases, the component or parameter associated with the cell or tissue, resulting in a modified cell, e.g., modified HSPC, or tissue.

35. The method of any one of the preceding claims, wherein the payload comprises a nucleic-acid molecule, a peptide molecule, a lipid molecule, a low molecular weight molecule, or a combination thereof.

36. The method of claim 35, wherein the payload comprises a polynucleotide or nucleic acid, e.g., a DNA or an mRNA.

37. The method of claim 36, wherein the mRNA comprises at least one chemical modification.

38. The method of claim 37, wherein the chemical modification is selected from the group consisting of pseudouridine, N1-methylpseudouridine, 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methyluridine, 5-methoxyuridine, and 2′-O-methyl uridine.

39. The method of any one of the preceding claims, wherein the payload comprises a genetic modulator (e.g., a modulator that genetically alters the cell or tissue); an epigenetic modulator (e.g., a modulator that epigenetically alters the cell or tissue); an RNA modulator (e.g., a modulator that alters an RNA molecule in the cell or tissue); a peptide modulator (e.g., a modulator that alters a peptide molecule in the cell or tissue); a lipid modulator (e.g., a modulator that alters a lipid molecule in the cell or tissue); or a combination thereof.

40. The method of any one of the preceding claims, wherein the disease or disorder is selected from the group consisting of a hemoglobinopathy, a clotting factor disorder, a blood cell disorder, and an immune cell disorder.

41. The method of any of the preceding claims, wherein the LNP does not comprise an additional targeting moiety.

42. An LNP composition for use in the method of any one of the preceding claims.

43. A pharmaceutical composition comprising the LNP composition of claim 42.

44. The LNP composition of claim 42, or pharmaceutical composition of claim 43, wherein the LNP composition comprises: (i) an ionizable lipid, e.g., an amino lipid; (ii) a sterol or other structural lipid; (iii) a non-cationic helper lipid or phospholipid; and (iv) a PEG-lipid.

45. The LNP composition of claim 42 or 43, or pharmaceutical composition of claim 43 or 44, wherein the LNP composition comprises an amino lipid comprising a compound of Formula (I-I), a phospholipid comprising DSPC, a structural lipid comprising cholesterol, and a PEG lipid comprising a compound of Formula (VI-D).

46. A modified cell, e.g., a modified stem cell, e.g., a modified HSPC, made according to a method of any one of claims 1-40.

47. A frozen preparation of a modified cell, e.g., a modified stem cell, e.g., a modified HSPC, made according to a method of any one of claims 1-40.

48. The modified cell of 46, or frozen preparation of a modified cell of 47, for use in treating a subject having a disease or disorder.

49. The modified cell of 46, or frozen preparation of a modified cell of 47, for use in ameliorating a symptom of a subject having a disease or disorder.

50. The modified cell, or frozen preparation of a modified cell, for use of claim 48 or 49, wherein the disease or disorder is selected from the group consisting of a hemoglobinopathy, a clotting factor disorder, a blood cell disorder, and an immune cell disorder.

51. The modified cell, or frozen preparation of a modified cell, for use of any of claims 48-50, wherein the modified cell is autologous to the subject.

52. The modified cell, or frozen preparation of a modified cell, for use of any of claims 48-50, wherein the modified cell is allogeneic to the subject.

53. A composition or reaction mixture comprising:
(a) a population of stem or progenitor cells, e.g., HSPCs; and
(b) an LNP composition comprising a payload which can modify the stem or progenitor cell, e.g., a component associated with the stem cell or a parameter associated with the stem or progenitor cell, optionally wherein the LNP composition does not comprise an additional targeting moiety.

54. A pharmaceutical composition comprising a modified cell, e.g., modified HSPC, and an LNP comprising a payload which can modify the stem cell, e.g., a component associated with the stem or progenitor cell or a parameter associated with the stem or progenitor cell, optionally wherein the LNP composition does not comprise an additional targeting moiety.

55. An LNP composition comprising (i) a payload, (ii) an amino lipid comprising a compound of Formula (I-I), (iii) a phospholipid comprising DSPC, (iv) a structural lipid comprising cholesterol, and (v) a PEG lipid comprising a compound of Formula (VI-D),
wherein, when administered to a subject, the LNP composition results in a modification of a HSPC, e.g., modification of a genotype, phenotype, or function of the HSPC, thereby altering (e.g., ameliorating) a disease or disorder selected from the group consisting of a hemoglobinopathy, a clotting factor disorder, a blood cell disorder, and an immune cell disorder, in the subject.

56. An LNP composition comprising (i) a payload, (ii) an amino lipid comprising a compound of Formula (I-I), (iii) a phospholipid comprising DSPC, (iv) a structural lipid comprising cholesterol, and (v) a PEG lipid comprising a compound of Formula (VI-D),
wherein, the payload modifies an HSPC, e.g., modification of a genotype, phenotype, or function of the HSPC, e.g., in a subject.

57. The LNP composition of claim 55 or 56, wherein, the LNP composition does not comprise an additional targeting moiety.


SEQUENCE LISTING (277 sequences)

SEQ ID NO: 1-24 - skipped (24 entries)

SEQ ID NO: 25 - RNA, 100 nt, Artificial Sequence
  source: /note=“Description of Artificial Sequence: Synthetic polynucleotide”
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 26 - RNA, 120 nt, Artificial Sequence
  source: /note=“Description of Artificial Sequence: Synthetic polynucleotide”
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 27-49 - skipped (23 entries)

SEQ ID NO: 50 - RNA, 75 nt, Artificial Sequence
  source: /note=“Description of Artificial Sequence: Synthetic oligonucleotide”
ggaaaucgcaaaauuugcucuucgcguuagauuucuuuuaguuuucucgcaacuagcaagcuuuuuguucucgcc

SEQ ID NO: 51 - RNA, 203 nt, Artificial Sequence
  source: /note=“Description of Artificial Sequence: Synthetic polynucleotide”
ggaaauccccacaaccgccucauauccaggcucaagaauagagcucaguguuuuguuguuuaaucauuccgacguguuuugcgauauucgcgcaaagcagccagucgcgcgcuugcuuuuaaguagaguuguuuuuccacccguuugccaggcaucuuuaauuuaacauauuuuuauuuuucaggcuaaccuacgccgccacc

SEQ ID NO: 52 - RNA, 76 nt, Artificial Sequence
  source: /note=“Description of Artificial Sequence: Synthetic oligonucleotide”
ggaaauaagagagaaaagaagaguaagaagaaauauaagaucucccugagcuucagggagccccggcgccgccacc

SEQ ID NO: 53 - RNA, 89 nt, Artificial Sequence
  source: /note=“Description of Artificial Sequence: Synthetic oligonucleotide”
ggaaaccccccacccccguaagagagaaaagaagaguaagaagaaauauaagaucucccugagcuucagggagccccggcgccgccacc

SEQ ID NO: 54 - RNA, 59 nt, Artificial Sequence
  source: /note=“Description of Artificial Sequence: Synthetic oligonucleotide”
ggagaacuuccgcuuccguuggcgcaagcgcuuucauuuuuucugcuaccgugacuaag

SEQ ID NO: 55 - RNA, 46 nt, Artificial Sequence
  source: /note=“Description of Artificial Sequence: Synthetic oligonucleotide”
ggaaauaagagagaaaagaagaguaagaagaaauauaagagccacc

SEQ ID NO: 56 - RNA, 56 nt, Artificial Sequence
  source: /note=“Description of Artificial Sequence: Synthetic oligonucleotide”
ggaaauaagagagaaaagaagaguaagaagaaauauaagaccccggcgccgccacc

SEQ ID NO: 57 - RNA, 81 nt, Artificial Sequence
  source: /note=“Description of Artificial Sequence: Synthetic oligonucleotide”
ggaaaucgcaaaauuugcucuucgcguuagauuucuuuuaguuuucucgcaacuagcaagcuuuuuguucucgccgccgcc

SEQ ID NO: 58 - RNA, 81 nt, Artificial Sequence
  source: /note=“Description of Artificial Sequence: Synthetic oligonucleotide”
ggaaaucgcaaaauuuucuuuucgcguuagauuucuuuuaguuuucuuucaacuagcaagcuuuuuguucucgccgccgcc

SEQ ID NO: 59 - RNA, 83 nt, Artificial Sequence
  source: /note=“Description of Artificial Sequence: Synthetic oligonucleotide”
  misc_feature (14)..(18): /note=“This region may encompass 0 to 5 nucleotides”
  misc_feature (19)..(19): /note=“May or may not be present”
  misc_feature (22)..(22): /note=“May or may not be present”
  misc_feature (23)..(27): /note=“This region may encompass 0 to 5 nucleotides”
  misc_feature (81)..(83): /note=“May or may not be present”
ggaaaucgcaaaauuuuugcucuuuuucgcguuagauuucuuuuaguuuucuykcaacuagcaagcuuuuuguucucgccrcc

SEQ ID NO: 60 - RNA, 79 nt, Artificial Sequence
  source: /note=“Description of Artificial Sequence: Synthetic oligonucleotide”
ggaaaauuuuagccuggaacguuagauaacuguccuguugucuuuauauacuugguccccaaguaguuugucuuccaaa

SEQ ID NO: 61 - RNA, 79 nt, Artificial Sequence
  source: /note=“Description of Artificial Sequence: Synthetic oligonucleotide”
ggaaacuuuauuuaguguuacuuuauuuucuguuuauuuguguuucuucaguggguuuguucuaauuuccuuggccgcc

SEQ ID NO: 62 - RNA, 79 nt, Artificial Sequence
  source: /note=“Description of Artificial Sequence: Synthetic oligonucleotide”
ggaaaaucuguauuagguuggcguguucuuuggucgguuguuaguauuguuguugauucguuuguggucgguugccgcc

SEQ ID NO: 63 - RNA, 79 nt, Artificial Sequence
  source: /note=“Description of Artificial Sequence: Synthetic oligonucleotide”
ggaaaauuauuaacaucuugguauucucgauaaccauucguuggauuuuauuguauucguaguuuggguuccugccgcc

SEQ ID NO: 64 - RNA, 79 nt, Artificial Sequence
  source: /note=“Description of Artificial Sequence: Synthetic oligonucleotide”
ggaaauuauuauuauuucuagcuacaauuuaucauuguauuauuuuagcuauucaucauuauuuacuuggugaucaaca

SEQ ID NO: 65 - RNA, 79 nt, Artificial Sequence
  source: /note=“Description of Artificial Sequence: Synthetic oligonucleotide”
ggaaauagguuguuaaccaaguucaagccuaauaagcuuggauucuggugacuugcuucaccguuggcgggcaccgauc

SEQ ID NO: 66 - RNA, 74 nt, Artificial Sequence
  source: /note=“Description of Artificial Sequence: Synthetic oligonucleotide”
ggaaaucguagagagucguacuuaguacauaucgacuaucgguggacaccaucaagauuauaaaccaggccaga

SEQ ID NO: 67 - RNA, 79 nt, Artificial Sequence
  source: /note=“Description of Artificial Sequence: Synthetic oligonucleotide”
ggaaacccgcccaagcgaccccaacauaucagcaguugcccaaucccaacucccaacacaauccccaagcaacgccgcc

SEQ ID NO: 68 - RNA, 79 nt, Artificial Sequence
  source: /note=“Description of Artificial Sequence: Synthetic oligonucleotide”
ggaaagcgauugaaggcgucuuuucaacuacucgauuaagguuggguaucgucgugggacuuggaaauuuguuguuucc

SEQ ID NO: 69 - RNA, 79 nt, Artificial Sequence
  source: /note=“Description of Artificial Sequence: Synthetic oligonucleotide”
ggaaacuaaucgaaauaaaagagccccguacucuuuuauuucuauuagguuaggagccuuagcauuuguaucuuaggua

SEQ ID NO: 70 - RNA, 79 nt, Artificial Sequence
  source: /note=“Description of Artificial Sequence: Synthetic oligonucleotide”
ggaaaugugauuuccagcaacuucuuuugaauauauugaauuccuaauucaaagcgaacaaaucuacaagccauauacc

SEQ ID NO: 71 - RNA, 74 nt, Artificial Sequence
  source: /note=“Description of Artificial Sequence: Synthetic oligonucleotide”
ggaaaucguagagagucguacuuacguggucgccauugcauagcgcgcgaaagcaacaggaacaagaacgcgcc

SEQ ID NO: 72 - RNA, 74 nt, Artificial Sequence
  source: /note=“Description of Artificial Sequence: Synthetic oligonucleotide”
ggaaaucguagagagucguacuuagaauaaacagagucgggucgacuugucucugauacuacgacgucacaauc

SEQ ID NO: 73 - RNA, 79 nt, Artificial Sequence
  source: /note=“Description of Artificial Sequence: Synthetic oligonucleotide”
ggaaaauuugccuucggaguugcguauccugaacugcccagccuccugauauacaacuguuccgcuuauucgggccgcc

SEQ ID NO: 74 - RNA, 79 nt, Artificial Sequence
  source: /note=“Description of Artificial Sequence: Synthetic oligonucleotide”
ggaaaucugagcaggaauccuuugugcauugaagacuuuagauuccucucugcgguagacgugcacuuauaaguauuug

SEQ ID NO: 75 - RNA, 79 nt, Artificial Sequence
  source: /note=“Description of Artificial Sequence: Synthetic oligonucleotide”
ggaaagcgauugaaggcgucuuuucaacuacucgauuaagguuggguaucgucgugggacuuggaaauuuguugccacc

SEQ ID NO: 76 - RNA, 104 nt, Artificial Sequence
  source: /note=“Description of Artificial Sequence: Synthetic polynucleotide”
ggaaauuuuuuuuugauauuauaagaguuuuuuuuugauauuaagaaaauuuuuuuuugauauuagaagaguaagaagaaauauaagaccccggcgccgccacc

SEQ ID NO: 77 - RNA, 57 nt, Artificial Sequence
  source: /note=“Description of Artificial Sequence: Synthetic oligonucleotide”
ggaaauaagagagaaaagaagaguaagaagaaauauaagagccaaaaaaaaaaaacc

SEQ ID NO: 78 - RNA, 76 nt, Artificial Sequence
  source: /note=“Description of Artificial Sequence: Synthetic oligonucleotide”
ggaaaucucccugagcuucagggaguaagagagaaaagaagaguaagaagaaauauaagaccccggcgccgccacc

SEQ ID NO: 79 - RNA, 6 nt, Artificial Sequence
  source: /note=“Description of Artificial Sequence: Synthetic oligonucleotide”
gccrcc

SEQ ID NO: 80 - RNA, 42 nt, Artificial Sequence
  source: /note=“Description of Artificial Sequence: Synthetic oligonucleotide”
gggagacaagcuuggcauuccgguacuguugguaaagccacc

SEQ ID NO: 81 - RNA, 47 nt, Artificial Sequence
  source: /note=“Description of Artificial Sequence: Synthetic oligonucleotide”
gggaauuaacagagaaaagaagaguaagaagaaauauaagagccacc

SEQ ID NO: 82 - RNA, 47 nt, Artificial Sequence
  source: /note=“Description of Artificial Sequence: Synthetic oligonucleotide”
gggaaauuagacagaaaagaagaguaagaagaaauauaagagccacc

SEQ ID NO: 83 - RNA, 47 nt, Artificial Sequence
  source: /note=“Description of Artificial Sequence: Synthetic oligonucleotide”
gggaaauaagagaguaaagaacaguaagaagaaauauaagagccacc

SEQ ID NO: 84 - RNA, 47 nt, Artificial Sequence
  source: /note=“Description of Artificial Sequence: Synthetic oligonucleotide”
gggaaaaaagagagaaaagaagacuaagaagaaauauaagagccacc

SEQ ID NO: 85 - RNA, 47 nt, Artificial Sequence
  source: /note=“Description of Artificial Sequence: Synthetic oligonucleotide”
gggaaauaagagagaaaagaagaguaagaagauauauaagagccacc

SEQ ID NO: 86 - RNA, 47 nt, Artificial Sequence
  source: /note=“Description of Artificial Sequence: Synthetic oligonucleotide”
gggaaauaagagacaaaacaagaguaagaagaaauauaagagccacc

SEQ ID NO: 87 - RNA, 47 nt, Artificial Sequence
  source: /note=“Description of Artificial Sequence: Synthetic oligonucleotide”
gggaaauuagagaguaaagaacaguaaguagaauuaaaagagccacc

SEQ ID NO: 88 - RNA, 47 nt, Artificial Sequence
  source: /note=“Description of Artificial Sequence: Synthetic oligonucleotide”
gggaaauaagagagaauagaagaguaagaagaaauauaagagccacc

SEQ ID NO: 89 - RNA, 47 nt, Artificial Sequence
  source: /note=“Description of Artificial Sequence: Synthetic oligonucleotide”
gggaaauaagagagaaaagaagaguaagaagaaaauuaagagccacc

SEQ ID NO: 90 - RNA, 47 nt, Artificial Sequence
  source: /note=“Description of Artificial Sequence: Synthetic oligonucleotide”
gggaaauaagagagaaaagaagaguaagaagaaauuuaagagccacc

SEQ ID NO: 91 - RNA, 92 nt, Unknown
  source: /note=“Description of Unknown: 5′ UTR-018 sequence
ucaagcuuuuggacccucguacagaagcuaauacgacucacuauagggaaauaagagagaaaagaagaguaagaagaaauauaagagccacc

SEQ ID NO: 92 - RNA, 5 nt, Artificial Sequence
  source: /note=”Description of Artificial Sequence: Synthetic oligonucleotide“
ucuag

SEQ ID NO: 93-99 - skipped (7 entries)

SEQ ID NO: 100 - RNA, 119 nt, Artificial Sequence
  source: /note=”Description of Artificial Sequence: Synthetic polynucleotide“
uaauaguaagcuggagccuccugagagaccugugugaacuauugagaagaucggaacagcuccuuacucugaggaaguugguacccccguggucuuugaauaaagucugagugggcggc

SEQ ID NO: 101 - RNA, 119 nt, Artificial Sequence
  source: /note=”Description of Artificial Sequence: Synthetic polynucleotide“
uaauaguaagcuggagccucacucuccucuccaucccguauccaggcugugaauuuuucaaggaauauaaagaucgggauguacccccguggucuuugaauaaagucugagugggcggc

SEQ ID NO: 102 - RNA, 119 nt, Artificial Sequence
  source: /note=”Description of Artificial Sequence: Synthetic polynucleotide“
ugauaguaagcuggagccucuagugacggcaacagggcuugguuuuuccuuguugugaaaucgacaucucugaagacaggguacccccguggucuuugaauaaagucugagugggcggc

SEQ ID NO: 103 - RNA, 119 nt, Artificial Sequence
  source: /note=”Description of Artificial Sequence: Synthetic polynucleotide“
ugauaguaagcuggagccuccuuccaucuagucacaaagacuccuucguccccaguugccgucuaggauugggccucccaguacccccguggucuuugaauaaagucugagugggcggc

SEQ ID NO: 104 - RNA, 119 nt, Artificial Sequence
  source: /note=”Description of Artificial Sequence: Synthetic polynucleotide“
ugauaguaagcuggagccucccauaacaugacauaucuggauuuugugcuuagaaccuuaaauuggaagcauucuuaauuguacccccguggucuuugaauaaagucugagugggcggc

SEQ ID NO: 105 - RNA, 119 nt, Artificial Sequence
  source: /note=”Description of Artificial Sequence: Synthetic polynucleotide“
uaauaguaagcuggagccuccggaaaacuaaaauagagauauuucaagauuuuauaauuuucaaagaccuuugaaauauuguacccccguggucuuugaauaaagucugagugggcggc

SEQ ID NO: 106 - RNA, 119 nt, Artificial Sequence
  source: /note=”Description of Artificial Sequence: Synthetic polynucleotide“
uaauaguaagcuggagccucuacacauugcuucuaguuggcagaaauaauugauuaaaagaccagaaacugugauaacugguacccccguggucuuuaaauaaagucuaagugggcggc

SEQ ID NO: 107 - RNA, 119 nt, Artificial Sequence
  source: /note=”Description of Artificial Sequence: Synthetic polynucleotide“
ugauaauaggcuggagccucgguggccaugcuucuugccccuugggccuccccccagccccuccuccccuuccugcacccguacccccguggucuuugaauaaagucugagugggcggc

SEQ ID NO: 108 - RNA, 119 nt, Artificial Sequence
  source: /note=”Description of Artificial Sequence: Synthetic polynucleotide“
ugauaauaggcuggagccucgguggccuagcuucuugccccuugggccuccccccagccccuccuccccuuccugcacccguacccccguggucuuugaauaaagucugagugggcggc

SEQ ID NO: 109 - RNA, 141 nt, Artificial Sequence
  source: /note=”Description of Artificial Sequence: Synthetic polynucleotide“
ugauaauaggcuggagccucgguggccaugcuucuugccccuugggccuccccccagccccuccuccccuuccugcacccguaccccccaaacaccauugucacacuccaguggucuuugaauaaagucugagugggcggc

SEQ ID NO: 110 - RNA, 141 nt, Artificial Sequence
  source: /note=”Description of Artificial Sequence: Synthetic polynucleotide“
ugauaauaggcuggagccucgguggccuagcuucuugccccuugggccuccccccagccccuccuccccuuccugcacccguaccccccaaacaccauugucacacuccaguggucuuugaauaaagucugagugggcggc

SEQ ID NO: 111 - RNA, 142 nt, Artificial Sequence
  source: /note=”Description of Artificial Sequence: Synthetic polynucleotide“
ugauaauaggcuggagccucgguggccuagcuucuugccccuugggccuccccccagccccuccuccccuuccugcacccguacccccuccauaaaguaggaaacacuacaguggucuuugaauaaagucugagugggcggc

SEQ ID NO: 112 - RNA, 141 nt, Artificial Sequence
  source: /note=”Description of Artificial Sequence: Synthetic polynucleotide“
ugauaauaggcuggagccucgguggccuagcuucuugccccuugggccuccccccagccccuccuccccuuccugcacccguaccccccgcauuauuacucacgguacgaguggucuuugaauaaagucugagugggcggc

SEQ ID NO: 113 - RNA, 164 nt, Artificial Sequence
  source: /note=”Description of Artificial Sequence: Synthetic polynucleotide“
ugauaauaguccauaaaguaggaaacacuacagcuggagccucgguggccaugcuucuugccccuugggccuccccccagccccuccuccccuuccugcacccguaccccccgcauuauuacucacgguacgaguggucuuugaauaaagucugagugggcggc

SEQ ID NO: 114 - RNA, 87 nt, Unknown
  source: /note=”Description of Unknown: miR-142 sequence“
gacagugcagucacccauaaaguagaaagcacuacuaacagcacuggaggguguaguguuuccuacuuuauggaugaguguacugug

SEQ ID NO: 115 - RNA, 60 nt, Artificial Sequence
  source: /note=”Description of Artificial Sequence: Synthetic oligonucleotide“
cugagagaccugugugaacuauugagaagaucggaacagcuccuuacucugaggaaguug

SEQ ID NO: 116 - RNA, 119 nt, Artificial Sequence
  source: /note=”Description of Artificial Sequence: Synthetic polynucleotide“
ugauaauaggcuggagccucucacacaccucugccccuugggccucccacucccauggcucugggcgguccagaaggagcguacccccguggucuuugaauaaagucugagugggcggc

SEQ ID NO: 117 - RNA, 109 nt, Artificial Sequence
  source: /note=”Description of Artificial Sequence: Synthetic polynucleotide“
ugauaauaggcuggagccuccaccgcguuauccguuccucguaggcugguccuggggaacgggucggcggguacccccguggucuuugaauaaagucugagugggcggc

SEQ ID NO: 118 - RNA, 257 nt, Artificial Sequence
  source: /note=”Description of Artificial Sequence: Synthetic polynucleotide“
ugauaauaggcuggagccucugcccggcaacggccaggucugugccaaguguuugcugacgcaacccccacuggcuggggcuuggucaugggccaucagcgcgugcguggaaccuuuucggcuccucugccgauccauacugcggaacuccuagccgcuuguuuugcucgcagcaggucuggagcaaacauuaucgggacugauaacucuguuguccuguacccccguggucuuugaauaaagucugagugggcggc

SEQ ID NO: 119 - RNA, 219 nt, Artificial Sequence
  source: /note=”Description of Artificial Sequence: Synthetic polynucleotide“
ugauaauaggcuggagccucgguggccuagcuucuugccccuugggccuccccccagccccuccuccccuuccugcacccguacccuuuuuuuuuuuuuuuuuuucuucuuuucuuuuuuuucuuuuuuuuuuuucuuucuuuuuuucuuuuuuuuucuuuucuuuuuucuuuuuuuuuuuuuuuuccguggucuuugaauaaagucugagugggcggc

SEQ ID NO: 120 - RNA, 219 nt, Artificial Sequence
  source: /note=”Description of Artificial Sequence: Synthetic polynucleotide“
ugauaauaggcuggagccucgguggccuagcuucuugccccuugggccuccccccagccccuccuccccuuccugcacccguacccuuuuuuuuuuuuuuuuuuuuuuuuuuuuuuuuuuuuuuuuuuuuuuuuuuuuuuuuuuuuuuuuuuuuuuuuuuuuuuuuuuuuuuuuuuuuuuuuuuuuccguggucuuugaauaaagucugagugggcggc

SEQ ID NO: 121-132 - skipped (12 entries)

SEQ ID NO: 133 - RNA, 185 nt, Artificial Sequence
  source: /note=”Description of Artificial Sequence: Synthetic polynucleotide“
uaagucuccauaaaguaggaaacacuacagcuggagccucgguggccuagcuucuugccccuugggccuccauaaaguaggaaacacuacauccccccagccccuccuccccuuccugcacccguacccccuccauaaaguaggaaacacuacaguggucuuugaauaaagucugagugggcggc

SEQ ID NO: 134 - RNA, 139 nt, Artificial Sequence
  source: /note=”Description of Artificial Sequence: Synthetic polynucleotide“
uaaagcgcuggagccucgguggccuagcuucuugccccuugggccuccccccagccccuccuccccuuccugcacccguacccccuccauaaaguaggaaacacuacaguggucuuugaauaaagucugagugggcggc

SEQ ID NO: 135 - RNA, 194 nt, Artificial Sequence
  source: /note=”Description of Artificial Sequence: Synthetic polynucleotide“
uaaagcuccccgggguccauaaaguaggaaacacuacagcuggagccuccugagagaccugugugaacuauugagaagaucggaacagcuccuuacucugaggaaguuguccauaaaguaggaaacacuacaguacccccuccauaaaguaggaaacacuacaguggucuuugaauaaagucugagugggcggc

SEQ ID NO: 136 - RNA, 241 nt, Artificial Sequence
  source: /note=”Description of Artificial Sequence: Synthetic polynucleotide“
uaauaguaaaccucacucacggccacauugagugccaggcuccgggcugguuuauaguaguguagagcauugcagcacuuagacuggggugcuguagucuuuauuguagucuuuccacauaccugauaauucuuagauaauuucuuauuuuaauuccauaaaguaggaaacacuacauaaaucuccauaaaguaggaaacacuacauauucuuccauaaaguaggaaacacuacauaggcu

SEQ ID NO: 137 - RNA, 94 nt, Artificial Sequence
  source: /note=”Description of Artificial Sequence: Synthetic oligonucleotide“
gccuccaccgcguuauccguuccucguaggcugguccuggggaacgggucggcggguacccccguggucuuugaauaaagucugagugggcggc

SEQ ID NO: 138 - RNA, 254 nt, Artificial Sequence
  source: /note=”Description of Artificial Sequence: Synthetic polynucleotide“
caccgcguuauccguuccucguaggcugguccuggggaacgggucggcgggccucgguggccuagcuucuugccccuugggcccaccgcguuauccguuccucguaggcugguccuggggaacgggucggcgguccccccagccccuccuccccuuccugcacccguaccccccaccgcguuauccguuccucguaggcugguccuggggaacgggucggcggguggucuuugaauaaagucugagugggcggc

SEQ ID NO: 139-142 - skipped (4 entries)

SEQ ID NO: 143 - RNA, 22 nt, Unknown
  source: /note=”Description of Unknown: miR 223-3p sequence“
ugucaguuugucaaauacccca

SEQ ID NO: 144-151 - skipped (8 entries)

SEQ ID NO: 152 - RNA, 22 nt, Unknown
  source: /note=”Description of Unknown: miR-126 sequence“
cgcauuauuacucacgguacga

SEQ ID NO: 153-173 - skipped (21 entries)

SEQ ID NO: 174 - RNA, 23 nt, Unknown
  source: /note=”Description of Unknown: miR-142-3p sequence“
uccauaaaguaggaaacacuaca

SEQ ID NO: 175-196 - skipped (22 entries)

SEQ ID NO: 197 - RNA, 47 nt, Unknown
  source: /note=”Description of Unknown: 5′UTR sequence“
gggaaauaagagagaaaagaagaguaagaagaaauauaagagccacc

SEQ ID NO: 198 - RNA, 142 nt, Artificial Sequence; identical to SEQ ID NO: 111
  source: /note=”Description of Artificial Sequence: Synthetic polynucleotide“

SEQ ID NO: 199 - RNA, 164 nt, Artificial Sequence; identical to SEQ ID NO: 113
  source: /note=”Description of Artificial Sequence: Synthetic polynucleotide“

SEQ ID NO: 200 - RNA, 87 nt, Unknown; identical to SEQ ID NO: 114
  source: /note=”Description of Unknown: miR-142 sequence“

SEQ ID NO: 201 - RNA, 23 nt, Unknown
  source: /note=”Description of Unknown: miR-142-3p sequence“
uguaguguuuccuacuuuaugga

SEQ ID NO: 202 - RNA, 23 nt, Unknown
  source: /note=”Description of Unknown: miR-142-3p binding site sequence“
uccauaaaguaggaaacacuaca

SEQ ID NO: 203 - RNA, 21 nt, Unknown
  source: /note=”Description of Unknown: miR-142-5p sequence“
cauaaaguagaaagcacuacu

SEQ ID NO: 204 - RNA, 21 nt, Unknown
  source: /note=”Description of Unknown: miR-142-5p binding site sequence“
aguagugcuuucuacuuuaug

SEQ ID NO: 205 - RNA, 85 nt, Unknown
  source: /note=”Description of Unknown: miR-126 sequence“
cgcuggcgacgggacauuauuacuuuugguacgcgcugugacacuucaaacucguaccgugaguaauaaugcgccguccacggca

SEQ ID NO: 206 - RNA, 22 nt, Unknown
  source: /note=”Description of Unknown: miR-126-3p sequence“
ucguaccgugaguaauaaugcg

SEQ ID NO: 207 - RNA, 22 nt, Unknown
  source: /note=”Description of Unknown: miR-126-3p binding site sequence“
cgcauuauuacucacgguacga

SEQ ID NO: 208 - RNA, 21 nt, Unknown
  source: /note=”Description of Unknown: miR-126-5p sequence“
cauuauuacuuuugguacgcg

SEQ ID NO: 209 - RNA, 21 nt, Unknown
  source: /note=”Description of Unknown: miR-126-5p binding site sequence“
cgcguaccaaaaguaauaaug

SEQ ID NO: 210 - RNA, 9 nt, Artificial Sequence
  source: /note=”Description of Artificial Sequence: Synthetic oligonucleotide“
ugauaauag

SEQ ID NO: 211 - RNA, 9 nt, Artificial Sequence
  source: /note=”Description of Artificial Sequence: Synthetic oligonucleotide“
ugauaguaa

SEQ ID NO: 212 - RNA, 22 nt, Unknown
  source: /note=”Description of Unknown: miR-122 sequence“
caaacaccauugucacacucca

SEQ ID NO: 213 - RNA, 9 nt, Artificial Sequence
  source: /note=”Description of Artificial Sequence: Synthetic oligonucleotide“
ugauaauaa

SEQ ID NO: 214 - RNA, 9 nt, Artificial Sequence
  source: /note=”Description of Artificial Sequence: Synthetic oligonucleotide“
ugauaguag

SEQ ID NO: 215 - RNA, 9 nt, Artificial Sequence
  source: /note=”Description of Artificial Sequence: Synthetic oligonucleotide“
uaaugauga

SEQ ID NO: 216 - RNA, 9 nt, Artificial Sequence
  source: /note=”Description of Artificial Sequence: Synthetic oligonucleotide“
uaauaguag

SEQ ID NO: 217 - RNA, 9 nt, Artificial Sequence
  source: /note=”Description of Artificial Sequence: Synthetic oligonucleotide“
ugaugauga

SEQ ID NO: 218 - RNA, 9 nt, Artificial Sequence
  source: /note=”Description of Artificial Sequence: Synthetic oligonucleotide“
uaauaauaa

SEQ ID NO: 219 - RNA, 9 nt, Artificial Sequence
  source: /note=”Description of Artificial Sequence: Synthetic oligonucleotide“
uaguaguag

SEQ ID NO: 220 - RNA, 133 nt, Artificial Sequence
  source: /note=”Description of Artificial Sequence: Synthetic polynucleotide“
gcuggagccucgguggccaugcuucuugccccuugggccuccccccagccccuccuccccuuccugcacccguacccccuccauaaaguaggaaacacuacaguggucuuugaauaaagucugagugggcggc

SEQ ID NO: 221 - RNA, 22 nt, Unknown
  source: /note=”Description of Unknown: miR 146-3p sequence“
ccucugaaauucaguucuucag

SEQ ID NO: 222 - RNA, 22 nt, Unknown
  source: /note=”Description of Unknown: miR 146-5p sequence
ugagaacugaauuccauggguu

SEQ ID NO: 223 - RNA, 22 nt, Unknown
  source: /note=“Description of Unknown: miR 155-3p sequence”
cuccuacauauuagcauuaaca

SEQ ID NO: 224 - RNA, 23 nt, Unknown
  source: /note=“Description of Unknown: miR 155-5p sequence”
uuaaugcuaaucgugauaggggu

SEQ ID NO: 225 - RNA, 22 nt, Unknown
  source: /note=“Description of Unknown: miR 16-3p sequence”
ccaguauuaacugugcugcuga

SEQ ID NO: 226 - RNA, 22 nt, Unknown
  source: /note=“Description of Unknown: miR 16-5p sequence”
uagcagcacguaaauauuggcg

SEQ ID NO: 227 - RNA, 21 nt, Unknown
  source: /note=“Description of Unknown: miR 21-3p sequence”
caacaccagucgaugggcugu

SEQ ID NO: 228 - RNA, 22 nt, Unknown
  source: /note=“Description of Unknown: miR 21-5p sequence”
uagcuuaucagacugauguuga

SEQ ID NO: 229 - skipped

SEQ ID NO: 230 - RNA, 22 nt, Unknown
  source: /note=“Description of Unknown: miR 223-5p sequence”
cguguauuugacaagcugaguu

SEQ ID NO: 231 - RNA, 22 nt, Unknown
  source: /note=“Description of Unknown: miR 24-3p sequence”
uggcucaguucagcaggaacag

SEQ ID NO: 232 - RNA, 22 nt, Unknown
  source: /note=“Description of Unknown: miR 24-5p sequence”
ugccuacugagcugauaucagu

SEQ ID NO: 233 - RNA, 21 nt, Unknown
  source: /note=“Description of Unknown: miR 27-3p sequence”
uucacaguggcuaaguuccgc

SEQ ID NO: 234 - RNA, 22 nt, Unknown
  source: /note=“Description of Unknown: miR 27-5p sequence”
agggcuuagcugcuugugagca

SEQ ID NO: 235 - RNA, 141 nt, Artificial Sequence
  source: /note=“Description of Artificial Sequence: Synthetic polynucleotide”
ugauaauaggcuggagccucgguggccaugcuucuugccccuugggccuccccccagccccuccuccccuuccugcacccguaccccccgcauuauuacucacgguacgaguggucuuugaauaaagucugagugggcggc

SEQ ID NO: 236 - RNA, 119 nt, Unknown; identical to SEQ ID NO: 107
  source: /note=“Description of Unknown: 3′UTR, no miR binding sites sequence”

SEQ ID NO: 237 - RNA, 142 nt, Artificial Sequence
  source: /note=“Description of Artificial Sequence: Synthetic polynucleotide”
ugauaauaggcuggagccucgguggccaugcuucuugccccuugggccuccccccagccccuccuccccuuccugcacccguacccccuccauaaaguaggaaacacuacaguggucuuugaauaaagucugagugggcggc

SEQ ID NO: 238 - skipped

SEQ ID NO: 239 - RNA, 23 nt, Unknown
  source: /note=“Description of Unknown: miR 155-5p sequence”
uuaaugcuaauugugauaggggu

SEQ ID NO: 240 - RNA, 23 nt, Unknown
  source: /note=“Description of Unknown: miR 155-5p binding site sequence”
accccuaucacaauuagcauuaa

SEQ ID NO: 241 - RNA, 188 nt, Artificial Sequence
  source: /note=“Description of Artificial Sequence: Synthetic polynucleotide”
ugauaauaguccauaaaguaggaaacacuacagcuggagccucgguggccaugcuucuugccccuugggccuccauaaaguaggaaacacuacauccccccagccccuccuccccuuccugcacccguacccccuccauaaaguaggaaacacuacaguggucuuugaauaaagucugagugggcggc

SEQ ID NO: 242 - RNA, 140 nt, Artificial Sequence
  source: /note=“Description of Artificial Sequence: Synthetic polynucleotide”
ugauaauaggcuggagccucgguggccaugcuucuugccccuugggccuccccccagccccuccuccccuuccugcacccguacccccaguagugcuuucuacuuuaugguggucuuugaauaaagucugagugggcggc

SEQ ID NO: 243 - RNA, 182 nt, Artificial Sequence
  source: /note=“Description of Artificial Sequence: Synthetic polynucleotide”
ugauaauagaguagugcuuucuacuuuauggcuggagccucgguggccaugcuucuugccccuugggccaguagugcuuucuacuuuauguccccccagccccuccuccccuuccugcacccguacccccaguagugcuuucuacuuuaugguggucuuugaauaaagucugagugggcggc

SEQ ID NO: 244 - RNA, 184 nt, Artificial Sequence
  source: /note=“Description of Artificial Sequence: Synthetic polynucleotide”
ugauaauagaguagugcuuucuacuuuauggcuggagccucgguggccaugcuucuugccccuugggccuccauaaaguaggaaacacuacauccccccagccccuccuccccuuccugcacccguacccccaguagugcuuucuacuuuaugguggucuuugaauaaagucugagugggcggc

SEQ ID NO: 245 - RNA, 142 nt, Artificial Sequence
  source: /note=“Description of Artificial Sequence: Synthetic polynucleotide”
ugauaauaggcuggagccucgguggccaugcuucuugccccuugggccuccccccagccccuccuccccuuccugcacccguacccccaccccuaucacaauuagcauuaaguggucuuugaauaaagucugagugggcggc

SEQ ID NO: 246 - RNA, 188 nt, Artificial Sequence
  source: /note=“Description of Artificial Sequence: Synthetic polynucleotide”
ugauaauagaccccuaucacaauuagcauuaagcuggagccucgguggccaugcuucuugccccuugggccaccccuaucacaauuagcauuaauccccccagccccuccuccccuuccugcacccguacccccaccccuaucacaauuagcauuaaguggucuuugaauaaagucugagugggcggc

SEQ ID NO: 247 - RNA, 188 nt, Artificial Sequence
  source: /note=“Description of Artificial Sequence: Synthetic polynucleotide”
ugauaauagaccccuaucacaauuagcauuaagcuggagccucgguggccaugcuucuugccccuugggccuccauaaaguaggaaacacuacauccccccagccccuccuccccuuccugcacccguacccccaccccuaucacaauuagcauuaaguggucuuugaauaaagucugagugggcggc

SEQ ID NO: 248 - RNA, 142 nt, Artificial Sequence
  source: /note=“Description of Artificial Sequence: Synthetic polynucleotide”
ugauaauaguccauaaaguaggaaacacuacagcuggagccucgguggccaugcuucuugccccuugggccuccccccagccccuccuccccuuccugcacccguacccccguggucuuugaauaaagucugagugggcggc

SEQ ID NO: 249 - RNA, 142 nt, Artificial Sequence
  source: /note=“Description of Artificial Sequence: Synthetic polynucleotide”
ugauaauaggcuggagccucgguggcuccauaaaguaggaaacacuacacaugcuucuugccccuugggccuccccccagccccuccuccccuuccugcacccguacccccguggucuuugaauaaagucugagugggcggc

SEQ ID NO: 250 - RNA, 142 nt, Artificial Sequence
  source: /note=“Description of Artificial Sequence: Synthetic polynucleotide”
ugauaauaggcuggagccucgguggccaugcuucuugccccuugggccuccauaaaguaggaaacacuacauccccccagccccuccuccccuuccugcacccguacccccguggucuuugaauaaagucugagugggcggc

SEQ ID NO: 251 - RNA, 70 nt, Artificial Sequence
  source: /note=“Description of Artificial Sequence: Synthetic oligonucleotide”
gggaaauaagaguccauaaaguaggaaacacuacaagaaaagaagaguaagaagaaauauaagagccacc

SEQ ID NO: 252 - RNA, 70 nt, Artificial Sequence
  source: /note=“Description of Artificial Sequence: Synthetic oligonucleotide”
gggaaauaagagagaaaagaagaguaauccauaaaguaggaaacacuacagaagaaauauaagagccacc

SEQ ID NO: 253 - RNA, 70 nt, Artificial Sequence
  source: /note=“Description of Artificial Sequence: Synthetic oligonucleotide”
gggaaauaagagagaaaagaagaguaagaagaaauauaauccauaaaguaggaaacacuacagagccacc

SEQ ID NO: 254 - RNA, 23 nt, Unknown
  source: /note=“Description of Unknown: miR 155-5p binding site sequence”
accccuaucacaauuagcauuaa

SEQ ID NO: 255 - RNA, 181 nt, Artificial Sequence
  source: /note=“Description of Artificial Sequence: Synthetic polynucleotide”
ugauaauagaguagugcuuucuacuuuauggcuggagccucgguggccaugcuucuugccccuugggccaguagugcuuucuacuuuauguccccccagccccucuccccuuccugcacccguacccccaguagugcuuucuacuuuaugguggucuuugaauaaagucugagugggcggc

SEQ ID NO: 256 - RNA, 142 nt, Artificial Sequence
  source: /note=“Description of Artificial Sequence: Synthetic polynucleotide”
ugauaauaggcuggagccucgguggccaugcuucuugccccuuccauaaaguaggaaacacuacaugggccuccccccagccccuccuccccuuccugcacccguacccccguggucuuugaauaaagucugagugggcggc

SEQ ID NO: 257 - RNA, 142 nt, Artificial Sequence
  source: /note=“Description of Artificial Sequence: Synthetic polynucleotide”
ugauaauaggcuggagccucgguggccaugcuucuugccccuugggccuccccccaguccauaaaguaggaaacacuacaccccuccuccccuuccugcacccguacccccguggucuuugaauaaagucugagugggcggc

SEQ ID NO: 258 - RNA, 142 nt, Artificial Sequence
  source: /note=“Description of Artificial Sequence: Synthetic polynucleotide”
ugauaauaggcuggagccucgguggccaugcuucuugccccuugggccuccccccagccccuccuccccuucuccauaaaguaggaaacacuacacugcacccguacccccguggucuuugaauaaagucugagugggcggc

SEQ ID NO: 259 - RNA, 142 nt, Artificial Sequence
  source: /note=“Description of Artificial Sequence: Synthetic polynucleotide”
ugauaauaggcuggagccucgguggccaugcuucuugccccuugggccuccccccagccccuccuccccuuccugcacccguacccccguggucuuugaauaaaguuccauaaaguaggaaacacuacacugagugggcggc

SEQ ID NO: 260 - RNA, 164 nt, Artificial Sequence
  source: /note=“Description of Artificial Sequence: Synthetic polynucleotide”
ugauaauaguccauaaaguaggaaacacuacagcuggagccucgguggccuagcuucuugccccuugggccuccccccagccccuccuccccuuccugcacccguaccccccgcauuauuacucacgguacgaguggucuuugaauaaagucugagugggcggc

SEQ ID NO: 261 - RNA, 119 nt, Artificial Sequence; identical to SEQ ID NO: 108
  source: /note=“Description of Artificial Sequence: Synthetic polynucleotide”

SEQ ID NO: 262 - RNA, 141 nt, Artificial Sequence; identical to SEQ ID NO: 112
  source: /note=“Description of Artificial Sequence: Synthetic polynucleotide”

SEQ ID NO: 263 - RNA, 188 nt, Artificial Sequence
  source: /note=“Description of Artificial Sequence: Synthetic polynucleotide”
ugauaauaguccauaaaguaggaaacacuacagcuggagccucgguggccuagcuucuugccccuugggccuccauaaaguaggaaacacuacauccccccagccccuccuccccuuccugcacccguacccccuccauaaaguaggaaacacuacaguggucuuugaauaaagucugagugggcggc

SEQ ID NO: 264 - RNA, 142 nt, Artificial Sequence
  source: /note=“Description of Artificial Sequence: Synthetic polynucleotide”
ugauaauaguccauaaaguaggaaacacuacagcuggagccucgguggccuagcuucuugccccuugggccuccccccagccccuccuccccuuccugcacccguacccccguggucuuugaauaaagucugagugggcggc

SEQ ID NO: 265 - RNA, 142 nt, Artificial Sequence
  source: /note=“Description of Artificial Sequence: Synthetic polynucleotide”
ugauaauaggcuggagccucgguggcuccauaaaguaggaaacacuacacuagcuucuugccccuugggccuccccccagccccuccuccccuuccugcacccguacccccguggucuuugaauaaagucugagugggcggc

SEQ ID NO: 266 - RNA, 142 nt, Artificial Sequence
  source: /note=“Description of Artificial Sequence: Synthetic polynucleotide”
ugauaauaggcuggagccucgguggccuagcuucuugccccuugggccuccauaaaguaggaaacacuacauccccccagccccuccuccccuuccugcacccguacccccguggucuuugaauaaagucugagugggcggc

SEQ ID NO: 267 - RNA, 142 nt, Artificial Sequence
  source: /note=“Description of Artificial Sequence: Synthetic polynucleotide”
ugauaauaggcuggagccucgguggccuagcuucuugccccuugggccuccccccagccccuccuccccuuccugcacccguacccccaccccuaucacaauuagcauuaaguggucuuugaauaaagucugagugggcggc

SEQ ID NO: 268 - RNA, 188 nt, Artificial Sequence
  source: /note=“Description of Artificial Sequence: Synthetic polynucleotide”
ugauaauagaccccuaucacaauuagcauuaagcuggagccucgguggccuagcuucuugccccuugggccaccccuaucacaauuagcauuaauccccccagccccuccuccccuuccugcacccguacccccaccccuaucacaauuagcauuaaguggucuuugaauaaagucugagugggcggc

SEQ ID NO: 269 - RNA, 188 nt, Artificial Sequence
  source: /note=“Description of Artificial Sequence: Synthetic polynucleotide”
ugauaauagaccccuaucacaauuagcauuaagcuggagccucgguggccuagcuucuugccccuugggccuccauaaaguaggaaacacuacauccccccagccccuccuccccuuccugcacccguacccccaccccuaucacaauuagcauuaaguggucuuugaauaaagucugagugggcggc

SEQ ID NO: 270 - protein, 9 aa, Unknown
  source: /note=“Description of Unknown: ′LAGLIDADG′ family peptide motif sequence”
LAGLIDADG

SEQ ID NO: 271 - RNA, 47 nt, Artificial Sequence
  source: /note=“Description of Artificial Sequence: Synthetic oligonucleotide”
aggaaauaagagagaaaagaagaguaagaagaaauauaagagccacc

SEQ ID NO: 272 - RNA, 57 nt, Artificial Sequence
  source: /note=“Description of Artificial Sequence: Synthetic oligonucleotide”
gggaaauaagagagaaaagaagaguaagaagaaauauaagaccccggcgccgccacc

SEQ ID NO: 273 - RNA, 57 nt, Artificial Sequence
  source: /note=“Description of Artificial Sequence: Synthetic oligonucleotide”
aggaaauaagagagaaaagaagaguaagaagaaauauaagaccccggcgccgccacc

SEQ ID NO: 274 - RNA, 47 nt, Artificial Sequence
  source: /note=“Description of Artificial Sequence: Synthetic oligonucleotide”
gggagaucagagagaaaagaagaguaagaagaaauauaagagccacc

SEQ ID NO: 275 - RNA, 9 nt, Unknown
  source: /note=“Description of Unknown: Kozak sequence”
ccrccaugg

SEQ ID NO: 276 - RNA, 188 nt, Artificial Sequence; identical to SEQ ID NO: 263
  source: /note=“Description of Artificial Sequence: Synthetic polynucleotide”

SEQ ID NO: 277 - RNA, 9 nt, Artificial Sequence
  source: /note=“Description of Artificial Sequence: Synthetic oligonucleotide”
uaaugauag